(19) United States
(12) Patent Application Publication
(10) Pub. No.: US 2018/0142038 A1
(43) Pub. Date: May 24, 2018

(54) ANTI-GLYCOPROTEIN ANTIBODIES AND USES THEREOF

(71) Applicant: ALDER BIOPHARMACEUTICALS, INC. a/b/a ALDERBIO HOLD HOLDINGS LLC, LAS VEGAS, NV (US)

(72) Inventors: Pamela BROWN, Seattle, WA (US); Geoffrey F. LEE, Mercer Island,, WA (US); Benjamin DUTZAR, Seattle, WA (US); Jenny A. MULLIGAN, Lake Forest Park, WA (US); Daniel S. ALLISON, Lake Forest Park, WA (US); Ethan W. OJALA, Snohomish, WA (US); Amarjeet SINGH, Kirkland, WA (US)

(73) Assignee: ALDER BIOPHARMACEUTICALS, INC. a/b/a ALDERBIO HOLDINGS LLC, LAS VEGAS, NV (US)

(21) Appl. No.: 15/544,163

(22) PCT Filed: Jan. 15, 2016
(86) PCT No.: PCT/US16/13701

§ 371 Date: Nov. 30, 2017

Related U.S. Application Data
(60) Provisional application No. 62/104,407, filed on Jan. 16, 2015.

(51) Int. Cl. C07K 16/42; C07K 16/14; C07K 16/18; C12N 15/80; G01N 33/50; C07K 16/44

(52) U.S. Cl. CPC C07K 16/42; C07K 16/14; C07K 16/18; C12N 15/80; G01N 33/50; C07K 2317/51; C07K 2317/24; C07K 2317/565; C07K 2317/567; C07K 2317/64; C07K 2319/035; C07K 16/44

(57) ABSTRACT

A new class of antibodies having specificity for glycoproteins are described. The antibodies are shown to bind sensitively and specifically to mannosylated proteins, such as proteins produced by fungi. Assays using these anti-glycoprotein antibodies for monitoring the presence of glycoproteins in a sample are provided. Such methods can be used to monitor methods for production and/or purification of desired polypeptides, which may be used to modify process parameters to modify (e.g., decrease or increase) the amount of glycosylated polypeptide produced and/or present in the purified product. Also provided are methods of using the subject antibodies for detecting the level of expression and secretion of a polypeptide, and methods of using the subject antibodies to purify or deplete a glycoprotein from a sample. In exemplary embodiments, the desired polypeptide may be a multi-subunit protein, such as an antibody, which may be produced in a yeast, such as Pichia pastoris.

### CROSS-REFERENCE TO RELATED APPLICATION

This application claims the benefit of U.S. Provisional Application Ser. No. 62/104,407, filed Jan. 16, 2015, entitled “ANTI-GLYCOPROTEIN ANTIBODIES AND USES THEREOF” (Atty. Docket No. 43257.4802), which is hereby incorporated by reference in its entirety.

### SEQUENCE LISTING DISCLOSURE

This application includes, as part of its disclosure, an electronic biological sequence listing text file having the name “43257o4813.txt” which has the size 136,707 bytes and which was created on Jan. 15, 2016, which is hereby incorporated by reference in its entirety.

### FIELD OF INVENTION

The present disclosure generally relates to anti-glycoprotein antibodies. Exemplified antibodies specifically bind to mannosylated proteins, which may be produced in a microbial system, e.g., Pichia pastoris. The antibodies can be used for purification or monitoring of proteins, such as to deplete or enrich for mannosylated proteins, or to detect mannosylated proteins or determine the abundance thereof.

### BACKGROUND

Large-scale, economic purification of proteins is an increasingly important concern in the biotechnology industry. Generally, proteins are produced by cell culture using, prokaryotic, e.g., bacterial, or eukaryotic, e.g., mammalian or fungal, cell lines engineered to produce the protein of interest by insertion of a recombinant plasmid comprising the gene for that protein. Since the cell lines used are living organisms, they must be fed with a complex growth medium, comprising sugars, amino acids, and growth factors, sometimes supplied from preparations of animal serum. Separation of the desired protein from the mixture of compounds fed to the cells and from the by-products generated by the cells themselves to a purity sufficient for use as a human therapeutic poses a formidable challenge.

Multimeric proteins, irrespective of whether they are present as homogeneous or heterogeneous polymers, represent some of the most complex structural organizations found in biological molecules. Not only do the constituent polypeptide chains have to fold (into secondary structures and tertiary domains) but they must also form complementary interfaces that allow stable subunit interactions. These interactions are highly specific and can be between identical subunits or between different subunits.

In particular, conventional antibodies are tetrameric proteins composed of two identical light chains and two identical heavy chains. Pure human antibodies of a specific type can be difficult to purify from natural sources in sufficient amounts for many purposes. As a consequence, biotechnology and pharmaceutical companies have turned to recombinant DNA-based methods to prepare antibodies on a large scale. Hundreds of therapeutic monoclonal antibodies (mAbs) are either currently on the market or under development. The production of functional antibodies (including functional antibody fragments) generally involves the synthesis of the two polypeptides as well as a number of post-translational events, including proteolytic processing of the N-terminal secretion signal sequence; proper folding and assembly of the polypeptides into tetramers; formation of disulfide bonds; and typically includes a specific N-linked glycosylation.

Additionally, cytokines, as pleiotropic regulators that control proliferation, differentiation, and other cellular functions of immune and hematopoietic systems, have potential therapeutic use for a wide range of infectious and autoimmune diseases. Much like antibodies, recombinant expression methods are often used to express recombinant cytokines for subsequent use in research and pharmaceutical applications.

Recombinant synthesis of such proteins has often relied on cultures of higher eukaryotic cells to produce biologically active material, with cultured mammalian cells being very commonly used. However, mammalian tissue culture-based production systems incur significant added expense and complication relative to microbial fermentation methods. Additionally, products derived from mammalian cell culture may require additional safety testing to ensure freedom from mammalian pathogens (including viruses) that might be present in the cultured cells or animal-derived products used in culture, such as serum.

Prior work has helped to establish the yeast Pichia pastoris as a cost-effective platform for producing functional antibodies that are potentially suitable for research, diagnostic, and therapeutic use. See co-owned U.S. Pat. Nos. 7,935,340; 7,927,863 and 8,268,582, each of which is incorporated by reference herein in its entirety. Methods are also known in the literature for design of P. pastoris fermentations for expression of recombinant proteins, with optimization having been described with respect to parameters including cell density, broth volume, substrate feed rate, and the length of each phase of the reaction. See Zhang et al., “Rational Design and Optimization of Fed-Batch and Continuous Fermentations” in Cregg, J. M., Ed., 2007, Pichia Protocols (2nd edition), Methods in Molecular Biology, vol. 389, Humana Press, Totowa, N.J., pgs. 43-63, each of which is hereby incorporated by reference in its entirety. See also, US 20130045888; and US 20120277408, each of which is hereby incorporated by reference in its entirety.

Though recombinant proteins can be produced from cultured cells, undesired side-products may also be produced. For example, the cultured cells may produce the desired protein along with proteins having undesired or aberrant glycosylation. Additionally, cultured cells may produce multi-subunit protein along with free monomers and complexes having incorrect stoichiometry, potentially increasing production costs, and requiring additional purification steps which may decrease total yield of the desired complex. Moreover, even after purification, undesired side-products may be present in amounts that cause concern. For example, glycosylated side-products may be present in amounts that adversely affect properties such as stability, half-life, and specific activity, whereas aberrant complexes or aggregates may decrease specific activity and may also be potentially immunogenic.

### SUMMARY

The present disclosure provides a new class of anti-glycoprotein antibodies that are demonstrated herein to bind specifically to mannosylated polypeptides, as well as antigen-binding fragments and variants thereof, and polynucleotides encoding same, and vectors comprising same. Exemplary anti-glycoprotein antibodies of the disclosure include Ab1, Ab2, Ab3, Ab4, Ab5, and fragments and variants thereof.

In another aspect the disclosure provides a process for purifying a desired polypeptide from one or more samples (e.g., from a fermentation process), the method comprising detecting the amount and/or type of glycosylated impurities in the sample(s) using an antibody that binds to said glycosylated impurities, such as a glycovariant of the desired polypeptide resulting from, e.g., O-linked glycosylation and/or N-linked glycosylation. The method may also comprise culturing a desired cell or microbe under conditions that result in the expression and optionally secretion of the recombinant polypeptide.

In another aspect, the present disclosure provides processes of producing and/or purifying a desired polypeptide, e.g., expressed in yeast or filamentous fungal cells, which processes include using an anti-glycoprotein antibody to detect glycosylated polypeptides. As a result, the production process and/or the purification method may be adjusted to increase or decrease the amount of glycosylated polypeptide, e.g., to reduce or eliminate undesired glycoproteins. In exemplary embodiments, the desired protein is a multi-subunit protein, such as an antibody, the host cell is a yeast cell, such as P. pastoris, and the glycosylated polypeptide is a glycovariant of the desired polypeptide, such as an N-linked and/or O-linked glycovariant.

In yet another aspect, the disclosure provides an anti-glycoprotein antibody or antibody fragment which specifically binds to the same or overlapping linear or conformational epitope(s) on a glycoprotein and/or competes for binding to the same or overlapping linear or conformational epitope(s) on a glycoprotein as an anti-glycoprotein antibody selected from Ab1, Ab2, Ab3, Ab4, or Ab5. The anti-glycoprotein antibody or antibody fragment may specifically bind to the same or overlapping linear or conformational epitope(s) and/or compete for binding to the same or overlapping linear or conformational epitope(s) on a glycoprotein as the anti-glycoprotein antibody Ab1. Said fragment may be selected from a Fab fragment, a Fab′ fragment, a F(ab′)2 fragment, a monovalent antibody, or a metMab, e.g., an Fab fragment. The anti-glycoprotein antibody or antibody fragment may comprise the same CDRs as an anti-glycoprotein antibody selected from Ab1, Ab2, Ab3, Ab4, or Ab5.

The Fab fragment may comprise a variable heavy chain comprising the CDR1 sequence of SEQ ID NO:4, the CDR2 sequence of SEQ ID NO:6, and the CDR3 sequence of SEQ ID NO:8, and/or a variable light chain comprising the CDR1 sequence of SEQ ID NO:24, the CDR2 sequence of SEQ ID NO:26, and the CDR3 sequence of SEQ ID NO:28.

The anti-glycoprotein antibody or antibody fragment may comprise at least 2 complementarity determining regions (CDRs) in each of the variable light and the variable heavy regions which are identical to those contained in an anti-glycoprotein antibody selected from Ab1, Ab2, Ab3, Ab4, or Ab5.

The anti-glycoprotein antibody or antibody fragment may be a humanized, single chain, or chimeric antibody. The anti-glycoprotein antibody or antibody fragment may specifically bind to one or more glycoproteins. The anti-glycoprotein antibody or antibody fragment may specifically bind to one or more mannosylated proteins. The anti-glycoprotein antibody or antibody fragment may specifically bind to a mannosylated antibody heavy-chain or light chain.

The anti-glycoprotein antibody or antibody fragment may specifically bind to a mannosylated human IgG1 antibody or antibody fragment comprising a heavy chain constant polypeptide having the sequence of SEQ ID NO: 201, 205, or 209 or a mannosylated fragment thereof and/or a mannosylated human IgG1 antibody light chain constant polypeptide comprising the sequence of SEQ ID NO: 203, 207, or 211 or a mannosylated fragment thereof.

Said mannosylated protein may be produced in a yeast species, e.g., in a yeast species selected from the selected from the group consisting of: Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia lipolytica, and Pichia pastoris (Komagataella pastoris).

Said mannosylated protein may be produced in a filamentous fungus species, e.g., in a filamentous fungus species selected from the group consisting of: Trichoderma reesei, Aspergillus spp., Aspergillus niger, Aspergillus nidulans, Aspergillus awamori, Aspergillus oryzae, Neurospora crassa, Penicillium spp., Penicillium chrysogenum, Penicillium purpurogenum, Penicillium funiculosum, Penicillium emersonii, Rhizopus spp., Rhizopus miehei, Rhizopus oryzae, Rhizopus pusillus, Rhizopus arrhizus, Phanerochaete chrysosporium, and Fusarium graminearum.

Said mannosylated protein may be produced in Pichia pastoris.

The anti-glycoprotein antibody or antibody fragment may be directly or indirectly attached to a detectable label or therapeutic agent.

In another aspect, the disclosure provides a nucleic acid sequence or nucleic acid sequences which encode an anti-glycoprotein antibody or antibody fragment as described herein, e.g., encoding an anti-glycoprotein antibody or antibody fragment which specifically binds to the same or overlapping linear or conformational epitope(s) on a glycoprotein and/or competes for binding to the same or overlapping linear or conformational epitope(s) on a glycoprotein as an anti-glycoprotein antibody selected from Ab1, Ab2, Ab3, Ab4, or Ab5. In another aspect, the disclosure provides a vector comprising said nucleic acid sequence or sequences, e.g., a plasmid or recombinant viral vector.

In another aspect, the disclosure provides a cultured or recombinant cell which expresses an antibody or antibody fragment described herein, e.g., that expresses an anti-glycoprotein antibody or antibody fragment which specifically binds to the same or overlapping linear or conformational epitope(s) on a glycoprotein and/or competes for binding to the same or overlapping linear or conformational epitope(s) on a glycoprotein as an anti-glycoprotein antibody selected from Ab1, Ab2, Ab3, Ab4, or Ab5. The cell may be a mammalian, yeast, bacterial, fungal, or insect cell. For example, the cell may be a yeast cell, such as a diploid yeast cell. The cell may be of the genus Pichia, such as Pichia pastoris.

In another aspect, the disclosure provides an isolated anti-glycoprotein antibody or antibody fragment comprising a VH polypeptide sequence selected from: SEQ ID NO: 2, 42, 82, 122, or 162, or a variant thereof that exhibits at least 90% sequence identity therewith; and/or a VL polypeptide sequence selected from: SEQ ID NO: 22, 62, 102, 142, or 182, or a variant thereof that exhibits at least 90% sequence identity therewith, wherein said anti-glycoprotein antibody specifically binds one or more glycoproteins.

In another aspect, the disclosure provides an isolated anti-glycoprotein antibody or antibody fragment comprising a VH polypeptide sequence selected from: SEQ ID NO: 2, 42, 82, 122, or 162, or a variant thereof that exhibits at least 90% sequence identity therewith; and/or a VL polypeptide sequence selected from: SEQ ID NO: 22, 62, 102, 142, or 182, or a variant thereof that exhibits at least 90% sequence identity therewith, wherein one or more of the framework (FR) or CDR residues in said VH or VL polypeptide has been substituted with another amino acid residue resulting in an anti-glycoprotein antibody that specifically binds one or more glycoproteins.

One or more framework (FR) residues of said antibody or antibody fragment may be substituted with an amino acid present at the corresponding site in a parent rabbit anti-glycoprotein antibody from which the complementarity determining regions (CDRs) contained in said VH or VL polypeptides have been derived or by a conservative amino acid substitution.

For example, at most 1 or 2 of the residues in the CDRs of said VL polypeptide sequence may be modified. As a further example, at most 1 or 2 of the residues in the CDRs of said VH polypeptide sequence may be modified.

Said antibody may be humanized. Said antibody may be chimeric. Said antibody may comprise a single chain antibody. Said antibody may comprise a human Fc, such as a constant region of human IgG1, IgG2, IgG3, or IgG4, or a variant or modified form thereof.

Said antibody may specifically bind to one or more mannosylated proteins, such as a mannosylated antibody heavy-chain or light chain.

Said antibody may specifically bind to a mannosylated human IgG1 antibody or antibody fragment comprising a heavy chain constant polypeptide having the sequence of SEQ ID NO: 201, 205, or 209 or a mannosylated fragment thereof and/or a mannosylated human IgG1 antibody light chain constant polypeptide comprising the sequence of SEQ ID NO: 203, 207, or 211 or a mannosylated fragment thereof.

Said mannosylated protein may be produced in a yeast species or a filamentous fungus species.

In another aspect, the disclosure provides a method of detecting a glycoprotein in a sample, comprising: contacting said sample with an anti-glycoprotein antibody, and detecting the binding of said glycoprotein with said anti-glycoprotein antibody. Said anti-glycoprotein antibody may be an anti-glycoprotein antibody as described herein, e.g., an anti-glycoprotein antibody or antibody fragment which specifically binds to the same or overlapping linear or conformational epitope(s) on a glycoprotein and/or competes for binding to the same or overlapping linear or conformational epitope(s) on a glycoprotein as an anti-glycoprotein antibody selected from Ab1, Ab2, Ab3, Ab4, or Ab5.

Said mannosylated protein may be produced in a yeast species, such as a yeast species selected from the selected from the group consisting of: Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia lipolytica, and Pichia pastoris (Komagataella pastoris).

Said mannosylated protein may be produced in a filamentous fungus species, such as a filamentous fungus species selected from the group consisting of: Trichoderma reesei, Aspergillus spp., Aspergillus niger, Aspergillus nidulans, Aspergillus awamori, Aspergillus oryzae, Neurospora crassa, Penicillium spp., Penicillium chrysogenum, Penicillium purpurogenum, Penicillium funiculosum, Penicillium emersonii, Rhizopus spp., Rhizopus miehei, Rhizopus oryzae, Rhizopus pusillus, Rhizopus arrhizus, Phanerochaete chrysosporium, and Fusarium graminearum.

Said mannosylated protein may be produced in Pichia pastoris.

Said step of detecting the binding of said glycoprotein with said anti-glycoprotein antibody may comprise an ELISA assay, such as an ELISA assay that utilizes horseradish peroxidase or europium detection.

Said anti-glycoprotein antibody may be bound to a support.

The method of detecting a glycoprotein in a sample may be effected on multiple fractions from a purification column, wherein based on the detected level of glycoproteins, multiple fractions are pooled to produce a purified product depleted for glycoproteins that bind to said anti-glycoprotein antibody.

The method of detecting a glycoprotein in a sample may be effected on multiple fractions from a purification column, wherein based on the detected level of glycoproteins, multiple fractions are pooled to produce a purified product enriched for glycoproteins that bind to said anti-glycoprotein antibody.

Said detection step may use a protein-protein interaction monitoring process, such as a protein-protein interaction monitoring process that uses light interferometry, dual polarization interferometry, static light scattering, dynamic light scattering, multi-angle light scattering, surface plasmon resonance, ELISA, chemiluminescent ELISA, europium ELISA, far western, or electroluminescence.

The detected glycoprotein may be the result of O-linked glycosylation.

The sample comprise may comprise a desired polypeptide.

The detected glycoprotein may be a glycovariant of the desired polypeptide.

The desired polypeptide may be a hormone, growth factor, receptor, antibody, cytokine, receptor ligand, transcription factor or enzyme.

The desired polypeptide may be a desired antibody or desired antibody fragment, such as a desired human antibody or a desired humanized antibody or fragment thereof.

Said desired humanized antibody may be of mouse, rat, rabbit, goat, sheep, or cow origin, e.g., of rabbit origin.

Said desired antibody or desired antibody fragment may comprise a desired monovalent, bivalent, or multivalent antibody.

Said desired antibody or desired antibody fragment may specifically bind to IL-2, IL-4, IL-6, IL-10, IL-12, IL-13, IL-17, IL-18, IFN-alpha, IFN-gamma, BAFF, CXCL13, IP-10, CBP, angiotensin, angiotensin I, angiotensin II, Nav1.7, Nav1.8, VEGF, PDGF, EPO, EGF, FSH, TSH, hCG, CGRP, NGF, TNF, HGF, BMP2, BMP7, PCSK9 or HRG.

Optionally, samples or eluate or fractions thereof comprising less than 10% glycoprotein may be pooled, or samples or eluate or fractions thereof comprising less than 5% glycoprotein are pooled, or samples or eluate or fractions thereof comprising less than 1% glycoprotein are pooled, or fractions thereof comprising less than 0.5% glycoprotein are pooled.

Optionally, samples or eluate or fractions thereof comprising greater than 90% glycoprotein are pooled, or samples or eluate or fractions thereof comprising greater than 95% glycoprotein are pooled, or samples or eluate or fractions thereof comprising greater than 99% glycoprotein are pooled, or samples or eluate or fractions thereof comprising greater than 99.5% glycoprotein are pooled.

The method may further comprise pooling different samples or eluate or fractions thereof based on the purity of the desired polypeptide, e.g., wherein samples or eluate or fractions thereof comprising greater than 90%, 91%, 97%, or 99% purity are pooled.

The purity may be determined by measuring the mass of glycosylated heavy chain polypeptide and/or glycosylated light chain polypeptide as a percentage of total mass of heavy chain polypeptide and/or light chain polypeptide.

The desired polypeptide may be purified using an affinity chromatographic support. The affinity chromatographic support, may comprise immunoaffinity ligand, e.g., Protein A or a lectin. The affinity chromatographic support may comprise a mixed mode chromatographic support, such as ceramic hydroxyapatite, ceramic fluoroapatite, crystalline hydroxyapatite, crystalline fluoroapatite, CaptoAdhere, Capto MMC, HEA Hypercel, PPA Hypercel or Toyopearl® MX-Trp-650M, such as ceramic hydroxyapatite.

The affinity chromatographic support may comprise a hydrophobic interaction chromatographic support, such as Butyl Sepharose® 4 FF, Butyl-S Sepharose® FF, Octyl Sepharose® 4 FF, Phenyl Sepharose® BB, Phenyl Sepharose® HP, Phenyl Sepharose® 6 FF High Sub, Phenyl Sepharose® 6 FF Low Sub, Source 15ETH, Source 15ISO, Source 15PHE, Capto Phenyl, Capto Butyl, Streamline Phenyl, TSK Ether 5PW (20 um and 30 um), TSK Phenyl 5PW (20 um and 30 um), Phenyl 650S, M, and C, Butyl 650S, M and C, Hexyl-650M and C, Ether-6505 and M, Butyl-600M, Super Butyl-550C, Phenyl-600M, PPG-600M; YMC-Pack Octyl Columns-3, 5, 10P, 15 and 25 um with pore sizes 120, 200, 300 A, YMC-Pack Phenyl Columns-3, 5, 10P, 15 and 25 um with pore sizes 120, 200 and 300 A, YMC-Pack Butyl Columns-3, 5, 10P, 15 and 25 um with pore sizes 120, 200 and 300 A, Cellufine Butyl, Cellufine Octyl, Cellufine Phenyl; WP HI-Propyl (C3); Macroprep t-Butyl or Macroprep methyl; or High Density Phenyl—HP2 20 um, such as polypropylene glycol (PPG) 600M or Phenyl Sepharose® HP.

Size exclusion chromatography may be effected to monitor impurities. The size exclusion chromatographic support may comprise GS3000SW.

### BRIEF DESCRIPTION OF THE DRAWINGS

FIGS. 1A-G, 2A-D, 3A-S, 4-I, 5, 6, 7, 8, 9, 10, 11, and 12 provide the polypeptide and polynucleotide sequences of the anti-glycoprotein antibodies Ab1, Ab2, Ab3, Ab4, and Ab5, including the full heavy and light chains, variable heavy and light chains, CDRs, framework regions, and constant regions, as well as the subsequence coordinates and SEQ ID NOs of those individual portions of the antibodies.

FIG. 13 shows results of ELISA assays using Ab1 and Ab2 to detect glycosylation of different lots of antibody Ab-A. The assay format was anti-glycovariant (AGV) antibody down, with horseradish peroxidase (HRP) detection. Biotinylated antibodies were bound to streptavidin plates with different Ab-A lots titrated. The two antibodies Ab1 and Ab2 reacted similarly to each test sample. In this assay format the sensitivity of Ab1 and Ab2 was relatively similar, possibly due to a “super-avidity” effect with the antibody down on the plate and multi-point mannosylated Ab-A in solution.

FIG. 14 shows results of ELISA assays using Ab3, Ab4, and Ab5 to detect glycosylation of different lots of antibody Ab-A and Ab-C. The assay format was biotinylated antigen down on streptavidin plates, with the anti-glycovariant (AGV) antibody titrated. The antibodies reacted similarly (though with some differences that may be due to differences in affinity) to the different antigens.

FIG. 15 shows results of ELISA assays using Ab1 to detect glycosylation of different lots of antibody Ab-A. The assay format was anti-glycovariant (AGV) antibody down, with horseradish peroxidase (HRP) or europium (Euro) detection in the left and right panels, respectively. Biotinylated antibodies were bound to streptavidin plates with different Ab-A lots titrated. In the right panel, detection was with a europium-labeled antibody that binds Ab-A (which contains a human constant domain) but not Ab1 (which contains a rabbit constant domain). The use of europium for detection resulted in greater signal than HRP.

FIG. 16 shows that binding of DC-SIGN to Ab-A lot2 coated biosensors (grey) is precluded (black) by Ab1 presence, thus demonstrating that the epitope to which Ab1 binds at least overlaps with the binding site for DC-SIGN.

FIGS. 17A-B shows use of AGV antibody Ab1 in a high throughput assay (HTRF) to quantify the level of glycoprotein in purification fractions. Ab-B (FIG. 17A) and Ab-D (FIG. 17B) were subjected to column purification and select fractions (as numbered on horizontal axis) were assayed using the AGV antibody to determine the relative amount of glycoprotein. Amount of antibody is expressed as the percentage of control (POC), specifically the amount of glycoprotein relative to a glycoprotein-enriched preparation of Ab-A (Ab-A lot 2). For reference, the amount of glycoprotein contained in Ab-A lot 1 (which contains a relatively low amount of glycoprotein) is indicated by a horizontal line, which was at a level of about 25% of control. Based on this measurement fractions can be selected or pooled to obtain a glycoprotein enriched or glycoprotein depleted preparation as desired.

FIG. 18A shows quantification of glycoprotein contained in fractions of Ab-A eluted from a polypropylene glycol (PPG) column. Ab1 and GNA were used to evaluate the relative amount of glycoprotein (expressed as percentage of control, POC) contained in each fraction. Protein mass contained in each fraction is also shown in relative units (Mass RU). A similar pattern of reactivity was seen for detection using Ab1 and GNA.

FIG. 18B is an enlarged version of FIG. 18A with the vertical axis enlarged and truncated to POC values between 0 and 23 to show greater detail in the low range.

FIGS. 19A-D show results of glycoprotein analysis of pooled fractions from the purification shown in FIG. 18A-B. FIG. 19A shows ELISA detection of glycoproteins in different preparations using an AGV antibody Ab1 in an europium-based antibody-down ELSA assay as in FIG. 15 (Ab1 down on plate, 0.3 μg/mL Ab-A samples in solution). FIG. 19B graphically illustrates the detected level of glycoprotein detected using the ELISA assay as a percentage of a control sample (POC). FIG. 19C-D shows the detected level of glycoprotein in the same samples determined using GNA or DCSIGN, respectively. The labels “fxn12-21” and “fxn4-23” respectively indicate pooling of fractions numbered 12 through 21 or 4 through 23 from the purification shown in FIG. 18A-B. Very similar profiles were seen with the AGV antibody, GNA, and DC-SIGN assays on these samples.

FIG. 20 shows results of glycoprotein analysis of antibody preparations using ELISA detection (left panel) or a GNA assay (right panel), each expressed as percentage of a control sample (POC). Results were qualitatively similar across the six tested lots, with relative peak height forming a similar pattern for each.

FIG. 21 shows results of O-glycoform composition analysis relative to signal from AGV, GNA, and DC-SIGN. The results show that the signals obtained from an AGV mAb (Ab1), GNA, and DC-SIGN binding assays correlate with each other and with the amount of mannose on Ab1. The table shows relative units of sugar alcohol compared to GNA, Ab1 and DC-SIGN signal.

FIG. 22 shows a schematic depiction of the arrangement of capture reagents used in the experiments in Example 10.

FIGS. 23A-B shows the flow cytometric profile of cells bound to GNA (FIG. 23A) or the anti-glycoprotein antibody Ab1 (FIG. 23B) used to couple the capture reagent to the cells. Use of GNA allowed captured fluorescence to migrate to unlabeled cells, whereas Ab1 binding was more stable and allowed fluorescence signal to be retained.

FIGS. 24A-B shows the flow cytometric profile of cells cultured for varying durations. Consistently increasing signal was demonstrated with increasing incubation time for samples of an antibody-expressing cell processed after 0, 0.5, or 2 hours (FIG. 24B), whereas a control non-producing “null strain” did not show any increase in signal over the same time-points (FIG. 24A).

FIGS. 25A-C shows the flow cytometric profile of co-cultured antibody-producing and non-producing “null” strains. Using conventional culture media, cross-binding of a labeled antibody-secreting “Production strain” with matrix-labeled non-producing null strain was observed (FIG. 25A). Supplementation with 10% PEG8000 was found to limit the cross-binding without negatively impacting the productivity (FIG. 25B and FIG. 25C).

FIGS. 26A-B shows the flow cytometric profile of high- and low-producing strains cultured individually (FIG. 26A) or co-cultured (FIG. 26B). Antibody production by the individual strains was characterized by processing the cells after 0 or 2 hours in culture (FIG. 26A), confirming that the assay detected a difference in fluorescence signal between the high- and low-producing strains, which increased over culture time. Mixed cultures of the high- and low-producing strain were labeled with the surface-capture matrix, allowed to secrete the antibodies in 10% PEG8000-supplemented media, washed and stained with detection antibody, and using flow cytometry, the top 0.25% of the cells with the highest fluorescence signal were isolated from the population (FIG. 26B).

FIG. 27 shows the flow cytometric profile of high- and low-producing strains cultured individually for 0- and 2-hours, demonstrating detection of the expected differences in antibody production levels between these strains and over the duration of the cell culture.

### DETAILED DESCRIPTION

The present disclosure provides glycoprotein-binding antibodies that specifically bind to glycoproteins produced from Pichia pastoris but not to the same glycoproteins produced from mammalian cells, indicating that the antibodies specifically bind to mannosylated proteins.

Additionally, the present disclosure provides processes for producing and purifying polypeptides (e.g., recombinant polypeptides) expressed by a host cell or microbe. In particular, the present disclosure provides processes of producing and purifying polypeptides, such as homopolymeric or heteropolymeric polypeptides (e.g., antibodies), expressed in yeast or filamentous fungal cells. The present methods incorporate antibody binding as a quantitative indicator of glycosylated impurities, such that the production and/or purification process can be modified to maximize the yield of the desired protein and decrease the presence of glycosylated impurities.

Additionally, the present processes encompass purification processes comprising chromatographic separation of samples from the fermentation process in order to substantially purify the desired polypeptide from undesired product-associated impurities, such as glycosylated impurities (e.g., glycovariants), nucleic acids and aggregates/disaggregates. In some embodiments, the eluate or fractions thereof from different chromatography steps are monitored for anti-glycoprotein (e.g., Ab1, Ab2, Ab3, Ab4, or Ab5) binding activity to detect the type and/or amount of glycosylated impurities. Based on the amount and/or type of glycosylated impurities detected, certain samples from the fermentation process and/or fractions from the chromatographic purification are discarded, treated and/or selectively pooled for further purification.

In exemplary embodiments, the desired protein is an antibody or an antibody binding fragment, the yeast cell is Pichia pastoris, and the glycosylated impurity is a glycovariant of the desired polypeptide, such as an N-linked and/or O-linked glycovariant, and the glycosylated impurity is detected using antibody Ab1, Ab2, Ab3, Ab4, or Ab5.

In a preferred embodiment, the desired protein is an antibody or antibody fragment, such as a humanized or human antibody, comprised of two heavy chain subunits and two light chain subunits. Preferred fungal cells include yeasts, and particularly preferred yeasts include methylotrophic yeast strains, e.g., Pichia pastoris, Hansenula polymorpha (Pichia angusta), Pichia guillermordii, Pichia methanolica, Pichia inositovera, and others (see, e.g., U.S. Pat. Nos. 4,812,405, 4,818,700, 4,929,555, 5,736,383, 5,955,349, 5,888,768, and 6,258,559 each of which is incorporated by reference in its entirety). The yeast cell may be produced by methods known in the art. For example, a panel of diploid or tetraploid yeast cells containing differing combinations of gene copy numbers may be generated by mating cells containing varying numbers of copies of the individual subunit genes (which numbers of copies preferably are known in advance of mating).

Applicants have discovered antibodies useful for the production and purification of proteins produced in yeast or filamentous fungal cells. In particular, the processes disclosed herein incorporate purity monitoring steps into the protein production and/or purification schemes to improve the removal of product-associated impurities, e.g., glycosylated impurities, from the main protein product of interest, e.g., by selectively discarding, treating and/or purifying certain fractions from the production and/or purification schemes based on the amount and/or type of detected glycosylated impurity relative to the amount of recombinant polypeptide. The working examples demonstrate that employing such production and purification monitoring methods results in high levels of product purification while maintaining a high yield of the desired protein product.

In one embodiment, the methods include a fermentation process for producing a desired polypeptide and purifying the desired polypeptide from the fermentation medium. Generally, a yeast cell or microbe is cultured under conditions resulting in expression and secretion of the desired polypeptide as well as one or more impurities into the fermentation medium, a sample is collected, e.g., during or after the fermentation run, and the amount and/or type of glycosylated impurities in the sample(s) is monitored using an anti-glycoprotein antibody such as Ab1, Ab2, Ab3, Ab4, or Ab5, such that parameters of the fermentation process, e.g., temperature, pH, gas constituents (e.g., oxygen level, pressure, flow rate), feed constituents (e.g., glucose level or rate), agitation, aeration, antifoam (e.g., type or concentration) and duration, can be modified based on the detected glycosylated impurities.

In another embodiment, the methods include a process for purifying a desired polypeptide from one or more samples, which result from a fermentation process that comprises culturing a desired cell or microbe under conditions that result in the expression and secretion of the desired polypeptide and one or more impurities into the fermentation medium, by using an anti-glycoprotein antibody such as Ab1, Ab2, Ab3, Ab4, or Ab5 to detect the amount and/or type of glycosylated impurities in the sample(s). The inventors have determined that anti-glycoprotein antibody binding assays provide a quantitative or semi-quantitative measure of glycosylated impurities, such that the purification process can be adjusted in response to the detected level and type of impurity.

In a particular embodiment, the purification process further includes contacting one or more samples from the fermentation process (such as a fermentation medium containing the desired protein, e.g., an antibody), expressed in a host yeast or filamentous fungal cell and an impurity, with at least one chromatographic support and then selectively eluting the desired polypeptide. For example, the sample may be tested for the glycosylated impurities using an assay that detects binding to an anti-glycoprotein antibody such as Ab1, Ab2, Ab3, Ab4, or Ab5, and, depending on the type and/or amount of glycosylated impurities detected, contacted with an affinity chromatographic support (e.g., Protein A or lectin), a mixed mode chromatographic support (e.g., ceramic hydroxyapatite) and a hydrophobic interaction chromatographic support (e.g., polypropylene glycol (PPG) 600M). The desired protein is separated, e.g., selectively eluted, from each chromatographic support prior to being contacted with the subsequent chromatographic support, resulting in the eluate or a fraction thereof from hydrophobic interaction chromatographic support comprising a substantially purified desired protein.

The methods optionally further include monitoring a sample of the fermentation process and/or a portion of the eluate or a fraction thereof from at least one of the affinity chromatographic support, the mixed mode chromatographic support and the hydrophobic interaction chromatographic support for the presence of at least one product-associated impurity, such as a fungal cell protein, a fungal cell nucleic acid, an adventitious virus, an endogenous virus, an endotoxin, an aggregate, a disaggregate, or an undesired protein comprising at least one modification relative to the desired protein (e.g., an amino acid substitution, N-terminal modification, C-terminal modification, mismatched S-S bonds, folding, truncation, aggregation, multimer dissociation, denaturation, acetylation, fatty acylation, deamidation, oxidation, carbamylation, carboxylation, formylation, gamma-carboxyglutamylation, glycosylation, methylation, phosphorylation, sulphation, PEGylation and ubiquitination). In particular, the production and purification processes may include detecting the amount of aggregated and/or disaggregated impurities in the samples or fractions using size exclusion chromatography.

“Substantially purified” with regard to the desired protein or multi-subunit complex means that the sample comprises at least 90%, at least 91%, at least 92%, at least 93%, at least 94%, at least 95%, at least 96%, at least 97%, at least 98%, or at least 98.5% of the desired protein with less than 3%, less than 2.5%, less than 2%, less than 1.5% or less than 1% of impurities, i.e., aggregate, variant and low molecular weight product. In one embodiment, the substantially purified protein comprises less than 10 ng/mg, preferably less than 5 ng/mg or more preferably less than 2 ng/mg of fungal cell protein; and/or less than 10 ng/mg or preferably less than 5 ng/mg of nucleic acid.

Though much of the present disclosure describes production of antibodies, the methods described herein are readily adapted to other multi-subunit complexes as well as single subunit proteins. The methods disclosed herein may readily be utilized to improve the yield and/or purity of any single or multi-subunit complex, which may or may not be recombinantly expressed. Additionally, the present methods are not limited to production of protein complexes but may also be readily adapted for use with ribonucleoprotein (RNP) complexes including telomerase, hnRNPs, ribosomes, snRNPs, signal recognition particles, prokaryotic and eukaryotic RNase P complexes, and any other complexes that contain multiple protein and/or RNA subunits. Additionally, the cell that expresses the multi-subunit complex may be produced by methods known in the art. For example, a panel of diploid or tetraploid yeast cells containing differing combinations of gene copy numbers may be generated by mating cells containing varying numbers of copies of the individual subunit genes (which numbers of copies preferably are known in advance of mating).

Antibody Polypeptide sequences

Antibody Ab1

In one embodiment, the invention includes an antibody or antibody fragment that specifically binds glycoproteins, such as mannosylated proteins, and that comprises a heavy chain sequence comprising or consisting of the sequence set forth below:

(SEQ ID NO: 1)

QEQLVESGGGLVQPGASLTLTCTASGFSFSNTNYMCWVRQAPGRGLEWVG
CMPVGFIASTFYATWAKGRSAISKSSSTAVTLQMTSLTVADTATYFCARE
SGSGWALNLWGQGTLVTVSSGQPKAPSVFPLAPCCGDTPSSTVTLGCLVK
GYLPEPVTVTWNSGTLTNGVRTFPSVRQSSGLYSLSSVVSVTSSSQPVTC
NVAHPATNTKVDKTVAPSTCSKPTCPPPELLGGPSVFIFPPKPKDTLMIS
RTPEVTCVVVDVSQDDPEVQFTWYINNEQVRTARPPLREQQFNSTIRVVS
TLPIAHQDWLRGKEFKCKVHNKALPAPIEKTISKARGQPLEPKVYTMGPP
REELSSRSVSLTCMINGFYPSDISVEWEKNGKAEDNYKTTPAVLDSDGSY
FLYSKLSVPTSEWQRGDVFTCSVMHEALHNHYTQKSISRSPGK.

In one embodiment, the invention includes an antibody or antibody fragment that specifically binds glycoproteins, such as mannosylated proteins, and that comprises a heavy chain sequence comprising or consisting of the variable heavy chain sequence set forth below:

(SEQ ID NO: 2)

QEQLVESGGGLVQPGASLTLTCTASGFSFSNTNYMCWVRQAPGRGLEWVG
CMPVGFIASTFYATWAKGRSAISKSSSTAVTLQMTSLTVADTATYFCARE
SGSGWALNLWGQGTLVTVSS.

In one embodiment, the invention includes an antibody or antibody fragment that specifically binds glycoproteins, such as mannosylated proteins, and that possesses the same epitopic specificity as Ab1 and comprises a constant heavy chain sequence comprising or consisting of the sequence set forth below:

(SEQ ID NO: 10)

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK.

In another embodiment, the invention includes an antibody or antibody fragment that specifically binds glycoproteins, such as mannosylated proteins, and that comprises a light chain sequence comprising or consisting of the sequence set forth below:

(SEQ ID NO: 21)

DPVLTQTPSPVSAAVGGTVTISCQASESVESGNWLAWYQQKPGQPPKLLI
YYTSTLASGVPSRFKGSGSGAHFTLTISGVQCDDAATYYCQGAFYGVNTF
GGGTEVVVKRTPVAPTVLLFPPSSDEVATGTVTIVCVANKYFPDVTVTWE
VDGTTQTTGIENSKTPQNSADCTYNLSSTLTLTSTQYNSHKEYTCKVTQG
TTSVVQSFSRKNC.

In another embodiment, the invention includes an antibody or antibody fragment that specifically binds glycoproteins, such as mannosylated proteins, and that comprises a light chain sequence comprising or consisting of the variable light chain sequence set forth below:

(SEQ ID NO: 22)

DPVLTQTPSPVSAAVGGTVTISCQASESVESGNWLAWYQQKPGQPPKLLI
YYTSTLASGVPSRFKGSGSGAHFTLTISGVQCDDAATYYCQGAFYGVNTF
GGGTEVVVK.

In one embodiment, the invention includes an antibody or antibody fragment that specifically binds glycoproteins, such as mannosylated proteins, and that possesses the same epitopic specificity as Ab1 and comprises a constant light chain sequence comprising or consisting of the sequence set forth below:

(SEQ ID NO: 30)

RTPVAPTVLLFPPSSDEVATGTVTIVCVANKYFPDVTVTWEVDGTTQTTG
IENSKTPQNSADCTYNLSSTLTLTSTQYNSHKEYTCKVTQGTTSVVQSFS
RKNC.

In another embodiment, the invention includes an antibody or antibody fragment that specifically binds glycoproteins (such as mannosylated proteins) and comprises one, two, or three of the polypeptide sequences of SEQ ID NO: 4; SEQ ID NO: 6; and SEQ ID NO: 8 which correspond to the complementarity-determining regions (CDRs, or hypervariable regions) of the heavy chain sequence of SEQ ID NO: 1 or which comprises the variable heavy chain sequence of SEQ ID NO: 2, and/or which further comprises one, two, or three of the polypeptide sequences of SEQ ID NO: 24; SEQ ID NO: 26; and SEQ ID NO: 28 which correspond to the complementarity-determining regions (CDRs, or hypervariable regions) of the light chain sequence of SEQ ID NO: 21 or which comprises the variable light chain sequence of SEQ ID NO: 22, or an antibody or antibody fragment containing combinations of sequences which are at least 80%, 85%, 90%, 95%, 96%, 97%, 98% or 99% identical thereto. In another embodiment of the invention, the antibody or fragments thereof comprises, or alternatively consists of, combinations of one or more of the exemplified variable heavy chain and variable light chain sequences, or the heavy chain and light chain sequences set forth above, or sequences that are at least 90% or 95% identical thereto.

The invention further contemplates anti-glycoprotein an antibody or antibody fragment comprising one, two, three, or four of the polypeptide sequences of SEQ ID NO: 3; SEQ ID NO: 5; SEQ ID NO: 7; and SEQ ID NO: 9 which correspond to the framework regions (FRs or constant regions) of the heavy chain sequence of SEQ ID NO: 1 or the variable heavy chain sequence of SEQ ID NO: 2, and/or one, two, three, or four of the polypeptide sequences of SEQ ID NO: 23; SEQ ID NO: 25; SEQ ID NO: 27; and SEQ ID NO: 29 which correspond to the framework regions (FRs or constant regions) of the light chain sequence of SEQ ID NO: 21 or the variable light chain sequence of SEQ ID NO: 22, or combinations of these polypeptide sequences or sequences which are at least 80%, 90% or 95% identical therewith.

In another embodiment of the invention, the antibody or antibody fragment of the invention comprises, or alternatively consists of, combinations of one or more of the FRs, CDRs, the variable heavy chain and variable light chain sequences, and the heavy chain and light chain sequences set forth above, including all of them or sequences which are at least 90% or 95% identical thereto.

In another embodiment of the invention, the anti-glycoprotein antibody or antibody fragment of the invention comprises, or alternatively consists of, the polypeptide sequence of SEQ ID NO: 1 or SEQ ID NO: 2 or polypeptides that are at least 90% or 95% identical thereto. In another embodiment of the invention, antibody fragment of the invention comprises, or alternatively consists of, the polypeptide sequence of SEQ ID NO: 21 or SEQ ID NO: 22 or polypeptides that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 4; SEQ ID NO: 6; and SEQ ID NO: 8 which correspond to the complementarity-determining regions (CDRs, or hypervariable regions) of the heavy chain sequence of SEQ ID NO: 1 or the variable heavy chain sequence of SEQ ID NO: 2 or sequences that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 24; SEQ ID NO: 26; and SEQ ID NO: 28 which correspond to the complementarity-determining regions (CDRs, or hypervariable regions) of the light chain sequence of SEQ ID NO: 21 or the variable light chain sequence of SEQ ID NO: 22 or sequences that are at least 90% or 95% identical thereto.

In a further embodiment of the invention, the antibody or antibody fragment that specifically binds glycoproteins (such as mannosylated proteins) comprises, or alternatively consists of, one, two, three, or four of the polypeptide sequences of SEQ ID NO: 3; SEQ ID NO: 5; SEQ ID NO: 7; and SEQ ID NO: 9 which correspond to the framework regions (FRs or constant regions) of the heavy chain sequence of SEQ ID NO: 1 or the variable heavy chain sequence of SEQ ID NO: 2 or sequences that are at least 90% or 95% identical thereto.

In a further embodiment of the invention, the subject antibody or antibody fragment that specifically binds glycoproteins (such as mannosylated proteins) comprises, or alternatively consists of, one, two, three, or four of the polypeptide sequences of SEQ ID NO: 23; SEQ ID NO: 25; SEQ ID NO: 27; and SEQ ID NO: 29 which correspond to the framework regions (FRs or constant regions) of the light chain sequence of SEQ ID NO: 21 or the variable light chain sequence of SEQ ID NO: 22 or sequences that are at least 90% or 95% identical thereto.

The invention also contemplates an antibody or fragment thereof that comprises one or more of the antibody fragments described herein. In one embodiment of the invention, the fragment of an antibody that specifically binds glycoproteins (such as mannosylated proteins) comprises, or alternatively consists of, one, two, three or more, including all of the following antibody fragments: the variable heavy chain region of SEQ ID NO: 2; the variable light chain region of SEQ ID NO: 22; the complementarity-determining regions (SEQ ID NO: 4; SEQ ID NO: 6; and SEQ ID NO: 8) of the variable heavy chain region of SEQ ID NO: 2; and the complementarity-determining regions (SEQ ID NO: 24; SEQ ID NO: 26; and SEQ ID NO: 28) of the variable light chain region of SEQ ID NO: 22 or sequences that are at least 90% or 95% identical thereto.

The invention also contemplates an antibody or fragment thereof that comprises one or more of the antibody fragments described herein. In one embodiment of the invention, the fragment of the antibody that specifically binds glycoproteins (such as mannosylated proteins) comprises, or alternatively consists of, one, two, three or more, including all of the following antibody fragments: the variable heavy chain region of SEQ ID NO: 2; the variable light chain region of SEQ ID NO: 22; the framework regions (SEQ ID NO: 3; SEQ ID NO: 5; SEQ ID NO: 7; and SEQ ID NO: 9) of the variable heavy chain region of SEQ ID NO: 2; and the framework regions (SEQ ID NO: 23; SEQ ID NO: 25; SEQ ID NO: 27; and SEQ ID NO: 29) of the variable light chain region of SEQ ID NO: 22.

In a particularly preferred embodiment of the invention, the anti-glycoprotein antibody is Ab1, comprising, or alternatively consisting of, SEQ ID NO: 1 and SEQ ID NO: 21, or an antibody or antibody fragment comprising the CDRs of Ab1 and having at least one of the biological activities set forth herein or is an anti-glycoprotein antibody that competes with Ab1 for binding glycoproteins (such as mannosylated proteins), preferably one containing sequences that are at least 90% or 95% identical to that of Ab1 or an antibody that binds to the same or overlapping epitope(s) on glycoproteins (such as mannosylated proteins) as Ab1.

In a further particularly preferred embodiment of the invention, the antibody fragment comprises, or alternatively consists of, an Fab (fragment antigen binding) fragment having binding specificity for glycoproteins (such as mannosylated proteins). With respect to antibody Ab1, the Fab fragment preferably includes the variable heavy chain sequence of SEQ ID NO: 2 and the variable light chain sequence of SEQ ID NO: 22 or sequences that are at least 90% or 95% identical thereto. This embodiment of the invention further includes an Fab containing additions, deletions, or variants of SEQ ID NO: 2 and/or SEQ ID NO: 22 which retain the binding specificity for glycoproteins (such as mannosylated proteins).

In one embodiment of the invention described herein (infra), Fab fragments may be produced by enzymatic digestion (e.g., papain) of Ab1. In another embodiment of the invention, anti-glycoprotein antibodies such as Ab1 or Fab fragments thereof may be produced via expression in mammalian cells such as CHO, NSO or human kidney cells, fungal, insect, or microbial systems such as yeast cells (for example haploid or diploid yeast such as haploid or diploid Pichia) and other yeast strains. Suitable Pichia species include, but are not limited to, Pichia pastoris.

In an additional embodiment, the invention is further directed to polynucleotides encoding antibody polypeptides having binding specificity for glycoproteins (such as mannosylated proteins), including the heavy and/or light chains of Ab1 as well as fragments, variants, combinations of one or more of the FRs, CDRs, the variable heavy chain and variable light chain sequences, and the heavy chain and light chain sequences set forth above, including all of them or sequences which are at least 90% or 95% identical thereto.

Antibody Ab2

In one embodiment, the invention includes an antibody or antibody fragment that specifically binds glycoproteins, such as mannosylated proteins, and that comprises a heavy chain sequence comprising or consisting of the sequence set forth below:

(SEQ ID NO: 41)

QSLEESGGGLVKPEGSLTLTCKASGFSFTGAHYMCWVRQAPGKGLEWIAC
IYGGSVDITFYASWAKGRFAISKSSSTAVTLQMTSLTAADTATYVCARES
GSGWALNLWGPGTLVTVSSGQPKAPSVFPLAPCCGDTPSSTVTLGCLVKG
YLPEPVTVTWNSGTLTNGVRTFPSVRQSSGLYSLSSVVSVTSSSQPVTCN
VAHPATNTKVDKTVAPSTCSKPTCPPPELLGGPSVFIFPPKPKDTLMISR
TPEVTCVVVDVSQDDPEVQFTWYINNEQVRTARPPLREQQFNSTIRVVST
LPIAHQDWLRGKEFKCKVHNKALPAPIEKTISKARGQPLEPKVYTMGPPR
EELSSRSVSLTCMINGFYPSDISVEWEKNGKAEDNYKTTPAVLDSDGSYF
LYSKLSVPTSEWQRGDVFTCSVMHEALHNHYTQKSISRSPGK.

In one embodiment, the invention includes an antibody or antibody fragment that specifically binds glycoproteins, such as mannosylated proteins, and that comprises a heavy chain sequence comprising or consisting of the variable heavy chain sequence set forth below:

(SEQ ID NO: 42)

QSLEESGGGLVKPEGSLTLTCKASGFSFTGAHYMCWVRQAPGKGLEWIAC
IYGGSVDITFYASWAKGRFAISKSSSTAVTLQMTSLTAADTATYVCARES
GSGWALNLWGPGTLVTVSS.

In one embodiment, the invention includes an antibody or antibody fragment that specifically binds glycoproteins, such as mannosylated proteins, and that possesses the same epitopic specificity as Ab2 and comprises a constant heavy chain sequence comprising or consisting of the sequence set forth below:

(SEQ ID NO: 50)

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK.

In another embodiment, the invention includes an antibody or antibody fragment that specifically binds glycoproteins, such as mannosylated proteins, and that comprises a light chain sequence comprising or consisting of the sequence set forth below:

(SEQ ID NO: 61)

QVLTQTASPVSAAVGGTVTISCQSSQSVENGNWLAWYQQKPGQPPKLLIY
LASTLESGVPSRFKGSGSGTQFTLTISGVQCDDAATYYCQGAYSGINVFG
GGTEVVVKRTPVAPTVLLFPPSSDEVATGTVTIVCVANKYFPDVTVTWEV
DGTTQTTGIENSKTPQNSADCTYNLSSTLTLTSTQYNSHKEYTCKVTQGT
TSVVQSFSRKNC.

In another embodiment, the invention includes an antibody or antibody fragment that specifically binds glycoproteins, such as mannosylated proteins, and that comprises a light chain sequence comprising or consisting of the variable light chain sequence set forth below:

(SEQ ID NO: 62)

QVLTQTASPVSAAVGGTVTISCQSSQSVENGNWLAWYQQKPGQPPKLLIY
LASTLESGVPSRFKGSGSGTQFTLTISGVQCDDAATYYCQGAYSGINVFG
GGTEVVVK.

In one embodiment, the invention includes an antibody or antibody fragment that specifically binds glycoproteins, such as mannosylated proteins, and that possesses the same epitopic specificity as Ab2 and comprises a constant light chain sequence comprising or consisting of the sequence set forth below:

(SEQ ID NO: 70)

RTPVAPTVLLFPPSSDEVATGTVTIVCVANKYFPDVTVTWEVDGTTQTTG
IENSKTPQNSADCTYNLSSTLTLTSTQYNSHKEYTCKVTQGTTSVVQSFS
RKNC.

In another embodiment, the invention includes an antibody or antibody fragment that specifically binds glycoproteins (such as mannosylated proteins) and comprises one, two, or three of the polypeptide sequences of SEQ ID NO: 44; SEQ ID NO: 46; and SEQ ID NO: 48 which correspond to the complementarity-determining regions (CDRs, or hypervariable regions) of the heavy chain sequence of SEQ ID NO: 41 or which comprises the variable heavy chain sequence of SEQ ID NO: 42, and/or which further comprises one, two, or three of the polypeptide sequences of SEQ ID NO: 64; SEQ ID NO: 66; and SEQ ID NO: 68 which correspond to the complementarity-determining regions (CDRs, or hypervariable regions) of the light chain sequence of SEQ ID NO: 61 or which comprises the variable light chain sequence of SEQ ID NO: 62, or an antibody or antibody fragment containing combinations of sequences which are at least 80%, 85%, 90%, 95%, 96%, 97%, 98% or 99% identical thereto. In another embodiment of the invention, the antibody or fragments thereof comprises, or alternatively consists of, combinations of one or more of the exemplified variable heavy chain and variable light chain sequences, or the heavy chain and light chain sequences set forth above, or sequences that are at least 90% or 95% identical thereto.

The invention further contemplates anti-glycoprotein an antibody or antibody fragment comprising one, two, three, or four of the polypeptide sequences of SEQ ID NO: 43; SEQ ID NO: 45; SEQ ID NO: 47; and SEQ ID NO: 49 which correspond to the framework regions (FRs or constant regions) of the heavy chain sequence of SEQ ID NO: 41 or the variable heavy chain sequence of SEQ ID NO: 42, and/or one, two, three, or four of the polypeptide sequences of SEQ ID NO: 63; SEQ ID NO: 65; SEQ ID NO: 67; and SEQ ID NO: 69 which correspond to the framework regions (FRs or constant regions) of the light chain sequence of SEQ ID NO: 61 or the variable light chain sequence of SEQ ID NO: 62, or combinations of these polypeptide sequences or sequences which are at least 80%, 90% or 95% identical therewith.

In another embodiment of the invention, the antibody or antibody fragment of the invention comprises, or alternatively consists of, combinations of one or more of the FRs, CDRs, the variable heavy chain and variable light chain sequences, and the heavy chain and light chain sequences set forth above, including all of them or sequences which are at least 90% or 95% identical thereto.

In another embodiment of the invention, the anti-glycoprotein antibody or antibody fragment of the invention comprises, or alternatively consists of, the polypeptide sequence of SEQ ID NO: 41 or SEQ ID NO: 42 or polypeptides that are at least 90% or 95% identical thereto. In another embodiment of the invention, antibody fragment of the invention comprises, or alternatively consists of, the polypeptide sequence of SEQ ID NO: 61 or SEQ ID NO: 62 or polypeptides that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 44; SEQ ID NO: 46; and SEQ ID NO: 48 which correspond to the complementarity-determining regions (CDRs, or hypervariable regions) of the heavy chain sequence of SEQ ID NO: 41 or the variable heavy chain sequence of SEQ ID NO: 42 or sequences that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 64; SEQ ID NO: 66; and SEQ ID NO: 68 which correspond to the complementarity-determining regions (CDRs, or hypervariable regions) of the light chain sequence of SEQ ID NO: 61 or the variable light chain sequence of SEQ ID NO: 62 or sequences that are at least 90% or 95% identical thereto.

In a further embodiment of the invention, the antibody or antibody fragment that specifically binds glycoproteins (such as mannosylated proteins) comprises, or alternatively consists of, one, two, three, or four of the polypeptide sequences of SEQ ID NO: 43; SEQ ID NO: 45; SEQ ID NO: 47; and SEQ ID NO: 49 which correspond to the framework regions (FRs or constant regions) of the heavy chain sequence of SEQ ID NO: 41 or the variable heavy chain sequence of SEQ ID NO: 42 or sequences that are at least 90% or 95% identical thereto.

In a further embodiment of the invention, the subject antibody or antibody fragment that specifically binds glycoproteins (such as mannosylated proteins) comprises, or alternatively consists of, one, two, three, or four of the polypeptide sequences of SEQ ID NO: 63; SEQ ID NO: 65; SEQ ID NO: 67; and SEQ ID NO: 69 which correspond to the framework regions (FRs or constant regions) of the light chain sequence of SEQ ID NO: 61 or the variable light chain sequence of SEQ ID NO: 62 or sequences that are at least 90% or 95% identical thereto.

The invention also contemplates an antibody or fragment thereof that comprises one or more of the antibody fragments described herein. In one embodiment of the invention, the fragment of an antibody that specifically binds glycoproteins (such as mannosylated proteins) comprises, or alternatively consists of, one, two, three or more, including all of the following antibody fragments: the variable heavy chain region of SEQ ID NO: 42; the variable light chain region of SEQ ID NO: 62; the complementarity-determining regions (SEQ ID NO: 44; SEQ ID NO: 46; and SEQ ID NO: 48) of the variable heavy chain region of SEQ ID NO: 42; and the complementarity-determining regions (SEQ ID NO: 64; SEQ ID NO: 66; and SEQ ID NO: 68) of the variable light chain region of SEQ ID NO: 62 or sequences that are at least 90% or 95% identical thereto.

The invention also contemplates an antibody or fragment thereof that comprises one or more of the antibody fragments described herein. In one embodiment of the invention, the fragment of the antibody that specifically binds glycoproteins (such as mannosylated proteins) comprises, or alternatively consists of, one, two, three or more, including all of the following antibody fragments: the variable heavy chain region of SEQ ID NO: 42; the variable light chain region of SEQ ID NO: 62; the framework regions (SEQ ID NO: 43; SEQ ID NO: 45; SEQ ID NO: 47; and SEQ ID NO: 49) of the variable heavy chain region of SEQ ID NO: 42; and the framework regions (SEQ ID NO: 63; SEQ ID NO: 65; SEQ ID NO: 67; and SEQ ID NO: 69) of the variable light chain region of SEQ ID NO: 62.

In a particularly preferred embodiment of the invention, the anti-glycoprotein antibody is Ab2, comprising, or alternatively consisting of, SEQ ID NO: 41 and SEQ ID NO: 61, or an antibody or antibody fragment comprising the CDRs of Ab2 and having at least one of the biological activities set forth herein or is an anti-glycoprotein antibody that competes with Ab2 for binding glycoproteins (such as mannosylated proteins), preferably one containing sequences that are at least 90% or 95% identical to that of Ab2 or an antibody that binds to the same or overlapping epitope(s) on glycoproteins (such as mannosylated proteins) as Ab2.

In a further particularly preferred embodiment of the invention, the antibody fragment comprises, or alternatively consists of, an Fab (fragment antigen binding) fragment having binding specificity for glycoproteins (such as mannosylated proteins). With respect to antibody Ab2, the Fab fragment preferably includes the variable heavy chain sequence of SEQ ID NO: 42 and the variable light chain sequence of SEQ ID NO: 62 or sequences that are at least 90% or 95% identical thereto. This embodiment of the invention further includes an Fab containing additions, deletions, or variants of SEQ ID NO: 42 and/or SEQ ID NO: 62 which retain the binding specificity for glycoproteins (such as mannosylated proteins).

In one embodiment of the invention described herein (infra), Fab fragments may be produced by enzymatic digestion (e.g., papain) of Ab2. In another embodiment of the invention, anti-glycoprotein antibodies such as Ab2 or Fab fragments thereof may be produced via expression in mammalian cells such as CHO, NSO or human kidney cells, fungal, insect, or microbial systems such as yeast cells (for example haploid or diploid yeast such as haploid or diploid Pichia) and other yeast strains. Suitable Pichia species include, but are not limited to, Pichia pastoris.

In an additional embodiment, the invention is further directed to polynucleotides encoding antibody polypeptides having binding specificity for glycoproteins (such as mannosylated proteins), including the heavy and/or light chains of Ab2 as well as fragments, variants, combinations of one or more of the FRs, CDRs, the variable heavy chain and variable light chain sequences, and the heavy chain and light chain sequences set forth above, including all of them or sequences which are at least 90% or 95% identical thereto.

Antibody Ab3

In one embodiment, the invention includes an antibody or antibody fragment that specifically binds glycoproteins, such as mannosylated proteins, and that comprises a heavy chain sequence comprising or consisting of the sequence set forth below:

(SEQ ID NO: 81)

QSLEESGGGLVQPEGSLTLTCTASGFFFSGAHYMCWVRQAPGQGLEWIGC
TYGGSVDITFYASWAKGRFAISKTSSTTVTLQLTSLTAADTATYVCARES
GSGWALNLWGQGTLVTVSSGQPKAPSVFPLAPCCGDTPSSTVTLGCLVKG
YLPEPVTVTWNSGTLTNGVRTFPSVRQSSGLYSLSSVVSVTSSSQPVTCN
VAHPATNTKVDKTVAPSTCSKPTCPPPELLGGPSVFIFPPKPKDTLMISR
TPEVTCVVVDVSQDDPEVQFTWYINNEQVRTARPPLREQQFNSTIRVVST
LPIAHQDWLRGKEFKCKVHNKALPAPIEKTISKARGQPLEPKVYTMGPPR
EELSSRSVSLTCMINGFYPSDISVEWEKNGKAEDNYKTTPAVLDSDGSYF
LYSKLSVPTSEWQRGDVFTCSVMHEALHNHYTQKSISRSPGK.

In one embodiment, the invention includes an antibody or antibody fragment that specifically binds glycoproteins, such as mannosylated proteins, and that comprises a heavy chain sequence comprising or consisting of the variable heavy chain sequence set forth below:

(SEQ ID NO: 82)

QSLEESGGGLVQPEGSLTLTCTASGFFFSGAHYMCWVRQAPGQGLEWIGC
TYGGSVDITFYASWAKGRFAISKTSSTTVTLQLTSLTAADTATYVCARES
GSGWALNLWGQGTLVTVSS.

In one embodiment, the invention includes an antibody or antibody fragment that specifically binds glycoproteins, such as mannosylated proteins, and that possesses the same epitopic specificity as Ab3 and comprises a constant heavy chain sequence comprising or consisting of the sequence set forth below:

(SEQ ID NO: 90)

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK.

In another embodiment, the invention includes an antibody or antibody fragment that specifically binds glycoproteins, such as mannosylated proteins, and that comprises a light chain sequence comprising or consisting of the sequence set forth below:

(SEQ ID NO: 101)

QVLTQTPSPVSAAVGGAVTINCQSSQSVENGNWLGWYQQKPGQPPKLLIY
LASTLASGVPSRFTGSGSGTQFTLTISGVQCDDAATYYCQGAYSGINAFG
GGTEVVVKRTPVAPTVLLFPPSSDEVATGTVTIVCVANKYFPDVTVTWEV
DGTTQTTGIENSKTPQNSADCTYNLSSTLTLTSTQYNSHKEYTCKVTQGT
TSVVQSFSRKNC.

In another embodiment, the invention includes an antibody or antibody fragment that specifically binds glycoproteins, such as mannosylated proteins, and that comprises a light chain sequence comprising or consisting of the variable light chain sequence set forth below:

(SEQ ID NO: 102)

QVLTQTPSPVSAAVGGAVTINCQSSQSVENGNWLGWYQQKPGQPPKLLIY
LASTLASGVPSRFTGSGSGTQFTLTISGVQCDDAATYYCQGAYSGINAFG
GGTEVVVK.

In one embodiment, the invention includes an antibody or antibody fragment that specifically binds glycoproteins, such as mannosylated proteins, and that possesses the same epitopic specificity as Ab3 and comprises a constant light chain sequence comprising or consisting of the sequence set forth below:

(SEQ ID NO: 110)

RTPVAPTVLLFPPSSDEVATGTVTIVCVANKYFPDVTVTWEVDGTTQTTG
IENSKTPQNSADCTYNLSSTLTLTSTQYNSHKEYTCKVTQGTTSVVQSFS
RKNC.

In another embodiment, the invention includes an antibody or antibody fragment that specifically binds glycoproteins (such as mannosylated proteins) and comprises one, two, or three of the polypeptide sequences of SEQ ID NO: 84; SEQ ID NO: 86; and SEQ ID NO: 88 which correspond to the complementarity-determining regions (CDRs, or hypervariable regions) of the heavy chain sequence of SEQ ID NO: 81 or which comprises the variable heavy chain sequence of SEQ ID NO: 82, and/or which further comprises one, two, or three of the polypeptide sequences of SEQ ID NO: 104; SEQ ID NO: 106; and SEQ ID NO: 108 which correspond to the complementarity-determining regions (CDRs, or hypervariable regions) of the light chain sequence of SEQ ID NO: 101 or which comprises the variable light chain sequence of SEQ ID NO: 102, or an antibody or antibody fragment containing combinations of sequences which are at least 80%, 85%, 90%, 95%, 96%, 97%, 98% or 99% identical thereto. In another embodiment of the invention, the antibody or fragments thereof comprises, or alternatively consists of, combinations of one or more of the exemplified variable heavy chain and variable light chain sequences, or the heavy chain and light chain sequences set forth above, or sequences that are at least 90% or 95% identical thereto.

The invention further contemplates anti-glycoprotein an antibody or antibody fragment comprising one, two, three, or four of the polypeptide sequences of SEQ ID NO: 83; SEQ ID NO: 85; SEQ ID NO: 87; and SEQ ID NO: 89 which correspond to the framework regions (FRs or constant regions) of the heavy chain sequence of SEQ ID NO: 81 or the variable heavy chain sequence of SEQ ID NO: 82, and/or one, two, three, or four of the polypeptide sequences of SEQ ID NO: 103; SEQ ID NO: 105; SEQ ID NO: 107; and SEQ ID NO: 109 which correspond to the framework regions (FRs or constant regions) of the light chain sequence of SEQ ID NO: 101 or the variable light chain sequence of SEQ ID NO: 102, or combinations of these polypeptide sequences or sequences which are at least 80%, 90% or 95% identical therewith.

In another embodiment of the invention, the antibody or antibody fragment of the invention comprises, or alternatively consists of, combinations of one or more of the FRs, CDRs, the variable heavy chain and variable light chain sequences, and the heavy chain and light chain sequences set forth above, including all of them or sequences which are at least 90% or 95% identical thereto.

In another embodiment of the invention, the anti-glycoprotein antibody or antibody fragment of the invention comprises, or alternatively consists of, the polypeptide sequence of SEQ ID NO: 81 or SEQ ID NO: 82 or polypeptides that are at least 90% or 95% identical thereto. In another embodiment of the invention, antibody fragment of the invention comprises, or alternatively consists of, the polypeptide sequence of SEQ ID NO: 101 or SEQ ID NO: 102 or polypeptides that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 84; SEQ ID NO: 86; and SEQ ID NO: 88 which correspond to the complementarity-determining regions (CDRs, or hypervariable regions) of the heavy chain sequence of SEQ ID NO: 81 or the variable heavy chain sequence of SEQ ID NO: 82 or sequences that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 104; SEQ ID NO: 106; and SEQ ID NO: 108 which correspond to the complementarity-determining regions (CDRs, or hypervariable regions) of the light chain sequence of SEQ ID NO: 101 or the variable light chain sequence of SEQ ID NO: 102 or sequences that are at least 90% or 95% identical thereto.

In a further embodiment of the invention, the antibody or antibody fragment that specifically binds glycoproteins (such as mannosylated proteins) comprises, or alternatively consists of, one, two, three, or four of the polypeptide sequences of SEQ ID NO: 83; SEQ ID NO: 85; SEQ ID NO: 87; and SEQ ID NO: 89 which correspond to the framework regions (FRs or constant regions) of the heavy chain sequence of SEQ ID NO: 81 or the variable heavy chain sequence of SEQ ID NO: 82 or sequences that are at least 90% or 95% identical thereto.

In a further embodiment of the invention, the subject antibody or antibody fragment that specifically binds glycoproteins (such as mannosylated proteins) comprises, or alternatively consists of, one, two, three, or four of the polypeptide sequences of SEQ ID NO: 103; SEQ ID NO: 105; SEQ ID NO: 107; and SEQ ID NO: 109 which correspond to the framework regions (FRs or constant regions) of the light chain sequence of SEQ ID NO: 101 or the variable light chain sequence of SEQ ID NO: 102 or sequences that are at least 90% or 95% identical thereto.

The invention also contemplates an antibody or fragment thereof that comprises one or more of the antibody fragments described herein. In one embodiment of the invention, the fragment of an antibody that specifically binds glycoproteins (such as mannosylated proteins) comprises, or alternatively consists of, one, two, three or more, including all of the following antibody fragments: the variable heavy chain region of SEQ ID NO: 82; the variable light chain region of SEQ ID NO: 102; the complementarity-determining regions (SEQ ID NO: 84; SEQ ID NO: 86; and SEQ ID NO: 88) of the variable heavy chain region of SEQ ID NO: 82; and the complementarity-determining regions (SEQ ID NO: 104; SEQ ID NO: 106; and SEQ ID NO: 108) of the variable light chain region of SEQ ID NO: 102 or sequences that are at least 90% or 95% identical thereto.

The invention also contemplates an antibody or fragment thereof that comprises one or more of the antibody fragments described herein. In one embodiment of the invention, the fragment of the antibody that specifically binds glycoproteins (such as mannosylated proteins) comprises, or alternatively consists of, one, two, three or more, including all of the following antibody fragments: the variable heavy chain region of SEQ ID NO: 82; the variable light chain region of SEQ ID NO: 102; the framework regions (SEQ ID NO: 83; SEQ ID NO: 85; SEQ ID NO: 87; and SEQ ID NO: 89) of the variable heavy chain region of SEQ ID NO: 82; and the framework regions (SEQ ID NO: 103; SEQ ID NO: 105; SEQ ID NO: 107; and SEQ ID NO: 109) of the variable light chain region of SEQ ID NO: 102.

In a particularly preferred embodiment of the invention, the anti-glycoprotein antibody is Ab3, comprising, or alternatively consisting of, SEQ ID NO: 81 and SEQ ID NO: 101, or an antibody or antibody fragment comprising the CDRs of Ab3 and having at least one of the biological activities set forth herein or is an anti-glycoprotein antibody that competes with Ab3 for binding glycoproteins (such as mannosylated proteins), preferably one containing sequences that are at least 90% or 95% identical to that of Ab3 or an antibody that binds to the same or overlapping epitope(s) on glycoproteins (such as mannosylated proteins) as Ab3.

In a further particularly preferred embodiment of the invention, the antibody fragment comprises, or alternatively consists of, an Fab (fragment antigen binding) fragment having binding specificity for glycoproteins (such as mannosylated proteins). With respect to antibody Ab3, the Fab fragment preferably includes the variable heavy chain sequence of SEQ ID NO: 82 and the variable light chain sequence of SEQ ID NO: 102 or sequences that are at least 90% or 95% identical thereto. This embodiment of the invention further includes an Fab containing additions, deletions, or variants of SEQ ID NO: 82 and/or SEQ ID NO: 102 which retain the binding specificity for glycoproteins (such as mannosylated proteins).

In one embodiment of the invention described herein (infra), Fab fragments may be produced by enzymatic digestion (e.g., papain) of Ab3. In another embodiment of the invention, anti-glycoprotein antibodies such as Ab3 or Fab fragments thereof may be produced via expression in mammalian cells such as CHO, NSO or human kidney cells, fungal, insect, or microbial systems such as yeast cells (for example haploid or diploid yeast such as haploid or diploid Pichia) and other yeast strains. Suitable Pichia species include, but are not limited to, Pichia pastoris.

In an additional embodiment, the invention is further directed to polynucleotides encoding antibody polypeptides having binding specificity for glycoproteins (such as mannosylated proteins), including the heavy and/or light chains of Ab3 as well as fragments, variants, combinations of one or more of the FRs, CDRs, the variable heavy chain and variable light chain sequences, and the heavy chain and light chain sequences set forth above, including all of them or sequences which are at least 90% or 95% identical thereto.

Antibody Ab4

In one embodiment, the invention includes an antibody or antibody fragment that specifically binds glycoproteins, such as mannosylated proteins, and that comprises a heavy chain sequence comprising or consisting of the sequence set forth below:

(SEQ ID NO: 121)

QSLEESGGDLVKPGASLTLTCTASGFSFSSGYDMCWVRQAPGKGLEWIAC
IYPNNPVTYYASWAKGRFTISKTSSTTVTLQMTSLTAADTATYFCGRSDS
NGHTFNLWGQGTLVTVSSGQPKAPSVFPLAPCCGDTPSSTVTLGCLVKGY
LPEPVTVTWNSGTLTNGVRTFPSVRQSSGLYSLSSVVSVTSSSQPVTCNV
AHPATNTKVDKTVAPSTCSKPTCPPPELLGGPSVFIFPPKPKDTLMISRT
PEVTCVVVDVSQDDPEVQFTWYFNNEQVRTARPPLREQQFNSTIRVVSTL
PIAHQDWLRGKEFKCKVHNKALPAPIEKTISKARGQPLEPKVYTMGPPRE
ELSSRSVSLTCMINGFYPSDISVEWEKNGKAEDNYKTTPAVLDSDGSYFL
YSKLSVPTSEWQRGDVFTCSVMHEALHNHYTQKSISRSPGK.

In one embodiment, the invention includes an antibody or antibody fragment that specifically binds glycoproteins, such as mannosylated proteins, and that comprises a heavy chain sequence comprising or consisting of the variable heavy chain sequence set forth below:

(SEQ ID NO: 122)

QSLEESGGDLVKPGASLTLTCTASGFSFSSGYDMCWVRQAPGKGLEWIAC
IYPNNPVTYYASWAKGRFTISKTSSTTVTLQMTSLTAADTATYFCGRSDS
NGHTFNLWGQGTLVTVSS.

In one embodiment, the invention includes an antibody or antibody fragment that specifically binds glycoproteins, such as mannosylated proteins, and that possesses the same epitopic specificity as Ab4 and comprises a constant heavy chain sequence comprising or consisting of the sequence set forth below:

(SEQ ID NO: 130)

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK.

In another embodiment, the invention includes an antibody or antibody fragment that specifically binds glycoproteins, such as mannosylated proteins, and that comprises a light chain sequence comprising or consisting of the sequence set forth below:

(SEQ ID NO: 141)

DPVMTQTPSSVSAAVGGTVTINCQSSQSVNQNDLSWYQQKPGQPPKRLIY
YASTLASGVSSRFKGSGSGTQFTLTISDMQCDDAATYYCQGSFRVSGWYW
AFGGGTEVVVKRTPVAPTVLLFPPSSDEVATGTVTIVCVANKYFPDVTVT
WEVDGTTQTTGIENSKTPQNSADCTYNLSSTLTLTSTQYNSHKEYTCKVT
QGTTSVVQSFSRKNC.

In another embodiment, the invention includes an antibody or antibody fragment that specifically binds glycoproteins, such as mannosylated proteins, and that comprises a light chain sequence comprising or consisting of the variable light chain sequence set forth below:

(SEQ ID NO: 142)

DPVMTQTPSSVSAAVGGTVTINCQSSQSVNQNDLSWYQQKPGQPPKRLIY
YASTLASGVSSRFKGSGSGTQFTLTISDMQCDDAATYYCQGSFRVSGWYW
AFGGGTEVVVK.

In one embodiment, the invention includes an antibody or antibody fragment that specifically binds glycoproteins, such as mannosylated proteins, and that possesses the same epitopic specificity as Ab4 and comprises a constant light chain sequence comprising or consisting of the sequence set forth below:

(SEQ ID NO: 150)

RTPVAPTVLLFPPSSDEVATGTVTIVCVANKYFPDVTVTWEVDGTTQTTG
IENSKTPQNSADCTYNLSSTLTLTSTQYNSHKEYTCKVTQGTTSVVQSFS
RKNC.

In another embodiment, the invention includes an antibody or antibody fragment that specifically binds glycoproteins (such as mannosylated proteins) and comprises one, two, or three of the polypeptide sequences of SEQ ID NO: 124; SEQ ID NO: 126; and SEQ ID NO: 128 which correspond to the complementarity-determining regions (CDRs, or hypervariable regions) of the heavy chain sequence of SEQ ID NO: 121 or which comprises the variable heavy chain sequence of SEQ ID NO: 122, and/or which further comprises one, two, or three of the polypeptide sequences of SEQ ID NO: 144; SEQ ID NO: 146; and SEQ ID NO: 148 which correspond to the complementarity-determining regions (CDRs, or hypervariable regions) of the light chain sequence of SEQ ID NO: 141 or which comprises the variable light chain sequence of SEQ ID NO: 142, or an antibody or antibody fragment containing combinations of sequences which are at least 80%, 85%, 90%, 95%, 96%, 97%, 98% or 99% identical thereto. In another embodiment of the invention, the antibody or fragments thereof comprises, or alternatively consists of, combinations of one or more of the exemplified variable heavy chain and variable light chain sequences, or the heavy chain and light chain sequences set forth above, or sequences that are at least 90% or 95% identical thereto.

The invention further contemplates anti-glycoprotein an antibody or antibody fragment comprising one, two, three, or four of the polypeptide sequences of SEQ ID NO: 123; SEQ ID NO: 125; SEQ ID NO: 127; and SEQ ID NO: 129 which correspond to the framework regions (FRs or constant regions) of the heavy chain sequence of SEQ ID NO: 121 or the variable heavy chain sequence of SEQ ID NO: 122, and/or one, two, three, or four of the polypeptide sequences of SEQ ID NO: 143; SEQ ID NO: 145; SEQ ID NO: 147; and SEQ ID NO: 149 which correspond to the framework regions (FRs or constant regions) of the light chain sequence of SEQ ID NO: 141 or the variable light chain sequence of SEQ ID NO: 142, or combinations of these polypeptide sequences or sequences which are at least 80%, 90% or 95% identical therewith.

In another embodiment of the invention, the antibody or antibody fragment of the invention comprises, or alternatively consists of, combinations of one or more of the FRs, CDRs, the variable heavy chain and variable light chain sequences, and the heavy chain and light chain sequences set forth above, including all of them or sequences which are at least 90% or 95% identical thereto.

In another embodiment of the invention, the anti-glycoprotein antibody or antibody fragment of the invention comprises, or alternatively consists of, the polypeptide sequence of SEQ ID NO: 121 or SEQ ID NO: 122 or polypeptides that are at least 90% or 95% identical thereto. In another embodiment of the invention, antibody fragment of the invention comprises, or alternatively consists of, the polypeptide sequence of SEQ ID NO: 141 or SEQ ID NO: 142 or polypeptides that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 124; SEQ ID NO: 126; and SEQ ID NO: 128 which correspond to the complementarity-determining regions (CDRs, or hypervariable regions) of the heavy chain sequence of SEQ ID NO: 121 or the variable heavy chain sequence of SEQ ID NO: 122 or sequences that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 144; SEQ ID NO: 146; and SEQ ID NO: 148 which correspond to the complementarity-determining regions (CDRs, or hypervariable regions) of the light chain sequence of SEQ ID NO: 141 or the variable light chain sequence of SEQ ID NO: 142 or sequences that are at least 90% or 95% identical thereto.

In a further embodiment of the invention, the antibody or antibody fragment that specifically binds glycoproteins (such as mannosylated proteins) comprises, or alternatively consists of, one, two, three, or four of the polypeptide sequences of SEQ ID NO: 123; SEQ ID NO: 125; SEQ ID NO: 127; and SEQ ID NO: 129 which correspond to the framework regions (FRs or constant regions) of the heavy chain sequence of SEQ ID NO: 121 or the variable heavy chain sequence of SEQ ID NO: 122 or sequences that are at least 90% or 95% identical thereto.

In a further embodiment of the invention, the subject antibody or antibody fragment that specifically binds glycoproteins (such as mannosylated proteins) comprises, or alternatively consists of, one, two, three, or four of the polypeptide sequences of SEQ ID NO: 143; SEQ ID NO: 145; SEQ ID NO: 147; and SEQ ID NO: 149 which correspond to the framework regions (FRs or constant regions) of the light chain sequence of SEQ ID NO: 141 or the variable light chain sequence of SEQ ID NO: 142 or sequences that are at least 90% or 95% identical thereto.

The invention also contemplates an antibody or fragment thereof that comprises one or more of the antibody fragments described herein. In one embodiment of the invention, the fragment of an antibody that specifically binds glycoproteins (such as mannosylated proteins) comprises, or alternatively consists of, one, two, three or more, including all of the following antibody fragments: the variable heavy chain region of SEQ ID NO: 122; the variable light chain region of SEQ ID NO: 142; the complementarity-determining regions (SEQ ID NO: 124; SEQ ID NO: 126; and SEQ ID NO: 128) of the variable heavy chain region of SEQ ID NO: 122; and the complementarity-determining regions (SEQ ID NO: 144; SEQ ID NO: 146; and SEQ ID NO: 148) of the variable light chain region of SEQ ID NO: 142 or sequences that are at least 90% or 95% identical thereto.

The invention also contemplates an antibody or fragment thereof that comprises one or more of the antibody fragments described herein. In one embodiment of the invention, the fragment of the antibody that specifically binds glycoproteins (such as mannosylated proteins) comprises, or alternatively consists of, one, two, three or more, including all of the following antibody fragments: the variable heavy chain region of SEQ ID NO: 122; the variable light chain region of SEQ ID NO: 142; the framework regions (SEQ ID NO: 123; SEQ ID NO: 125; SEQ ID NO: 127; and SEQ ID NO: 129) of the variable heavy chain region of SEQ ID NO: 122; and the framework regions (SEQ ID NO: 143; SEQ ID NO: 145; SEQ ID NO: 147; and SEQ ID NO: 149) of the variable light chain region of SEQ ID NO: 142.

In a particularly preferred embodiment of the invention, the anti-glycoprotein antibody is Ab4, comprising, or alternatively consisting of, SEQ ID NO: 121 and SEQ ID NO: 141, or an antibody or antibody fragment comprising the CDRs of Ab4 and having at least one of the biological activities set forth herein or is an anti-glycoprotein antibody that competes with Ab4 for binding glycoproteins (such as mannosylated proteins), preferably one containing sequences that are at least 90% or 95% identical to that of Ab4 or an antibody that binds to the same or overlapping epitope(s) on glycoproteins (such as mannosylated proteins) as Ab4.

In a further particularly preferred embodiment of the invention, the antibody fragment comprises, or alternatively consists of, an Fab (fragment antigen binding) fragment having binding specificity for glycoproteins (such as mannosylated proteins). With respect to antibody Ab4, the Fab fragment preferably includes the variable heavy chain sequence of SEQ ID NO: 122 and the variable light chain sequence of SEQ ID NO: 142 or sequences that are at least 90% or 95% identical thereto. This embodiment of the invention further includes an Fab containing additions, deletions, or variants of SEQ ID NO: 122 and/or SEQ ID NO: 142 which retain the binding specificity for glycoproteins (such as mannosylated proteins).

In one embodiment of the invention described herein (infra), Fab fragments may be produced by enzymatic digestion (e.g., papain) of Ab4. In another embodiment of the invention, anti-glycoprotein antibodies such as Ab4 or Fab fragments thereof may be produced via expression in mammalian cells such as CHO, NSO or human kidney cells, fungal, insect, or microbial systems such as yeast cells (for example haploid or diploid yeast such as haploid or diploid Pichia) and other yeast strains. Suitable Pichia species include, but are not limited to, Pichia pastoris.

In an additional embodiment, the invention is further directed to polynucleotides encoding antibody polypeptides having binding specificity for glycoproteins (such as mannosylated proteins), including the heavy and/or light chains of Ab4 as well as fragments, variants, combinations of one or more of the FRs, CDRs, the variable heavy chain and variable light chain sequences, and the heavy chain and light chain sequences set forth above, including all of them or sequences which are at least 90% or 95% identical thereto.

Antibody Ab5

In one embodiment, the invention includes an antibody or antibody fragment that specifically binds glycoproteins, such as mannosylated proteins, and that comprises a heavy chain sequence comprising or consisting of the sequence set forth below:

(SEQ ID NO: 161)

QQQLLESGGGLVQPEGSLALTCTASGFSFSSGYDMCWVRQPPGKGLEWVG
CIYSGDDNDITYYASWARGRFTISNPSSTTVTLQMTSLTVADTATYFCAR
GHAIYDNYDSVHLWGQGTLVTVSSGQPKAPSVFPLAPCCGDTPSSTVTLG
CLVKGYLPEPVTVTWNSGTLTNGVRTFPSVRQSSGLYSLSSVVSVTSSSQ
PVTCNVAHPATNTKVDKTVAPSTCSKPTCPPPELLGGPSVFIFPPKPKDT
LMISRTPEVTCVVVDVSQDDPEVQFTWYINNEQVRTARPPLREQQFNSTI
RVVSTLPIAHQDWLRGKEFKCKVHNKALPAPIEKTISKARGQPLEPKVYT
MGPPREELSSRSVSLTCMINGFYPSDISVEWEKNGKAEDNYKTTPAVLDS
DGSYFLYSKLSVPTSEWQRGDVFTCSVMHEALHNHYTQKSISRSPGK.

In one embodiment, the invention includes an antibody or antibody fragment that specifically binds glycoproteins, such as mannosylated proteins, and that comprises a heavy chain sequence comprising or consisting of the variable heavy chain sequence set forth below:

(SEQ ID NO: 162)

QQQLLESGGGLVQPEGSLALTCTASGFSFSSGYDMCWVRQPPGKGLEWVG
CIYSGDDNDITYYASWARGRFTISNPSSTTVTLQMTSLTVADTATYFCAR
GHAIYDNYDSVHLWGQGTLVTVSS.

In one embodiment, the invention includes an antibody or antibody fragment that specifically binds glycoproteins, such as mannosylated proteins, and that possesses the same epitopic specificity as Ab5 and comprises a constant heavy chain sequence comprising or consisting of the sequence set forth below:

(SEQ ID NO: 170)

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK.

In another embodiment, the invention includes an antibody or antibody fragment that specifically binds glycoproteins, such as mannosylated proteins, and that comprises a light chain sequence comprising or consisting of the sequence set forth below:

(SEQ ID NO: 181)

IVMTQTPSSRSVPVGGTVTINCQASEIVNRNNRLAWFQQKPGQPPKLLMY
LASTPASGVPSRFRGSGSGTQFTLTISDVVCDDAATYYCTAYKSSNTDGI
AFGGGTEVVVKRTPVAPTVLLFPPSSDEVATGTVTIVCVANKYFPDVTVT
WEVDGTTQTTGIENSKTPQNSADCTYNLSSTLTLTSTQYNSHKEYTCKVT
QGTTSVVQSFSRKNC.

In another embodiment, the invention includes an antibody or antibody fragment that specifically binds glycoproteins, such as mannosylated proteins, and that comprises a light chain sequence comprising or consisting of the variable light chain sequence set forth below:

(SEQ ID NO: 182)

IVMTQTPSSRSVPVGGTVTINCQASEIVNRNNRLAWFQQKPGQPPKLLMY
LASTPASGVPSRFRGSGSGTQFTLTISDVVCDDAATYYCTAYKSSNTDGI
AFGGGTEVVVK.

In one embodiment, the invention includes an antibody or antibody fragment that specifically binds glycoproteins, such as mannosylated proteins, and that possesses the same epitopic specificity as Ab5 and comprises a constant light chain sequence comprising or consisting of the sequence set forth below:

(SEQ ID NO: 190)

RTPVAPTVLLFPPSSDEVATGTVTIVCVANKYFPDVTVTWEVDGTTQTTG
IENSKTPQNSADCTYNLSSTLTLTSTQYNSHKEYTCKVTQGTTSVVQSFS
RKNC.

In another embodiment, the invention includes an antibody or antibody fragment that specifically binds glycoproteins (such as mannosylated proteins) and comprises one, two, or three of the polypeptide sequences of SEQ ID NO: 164; SEQ ID NO: 166; and SEQ ID NO: 168 which correspond to the complementarity-determining regions (CDRs, or hypervariable regions) of the heavy chain sequence of SEQ ID NO: 161 or which comprises the variable heavy chain sequence of SEQ ID NO: 162, and/or which further comprises one, two, or three of the polypeptide sequences of SEQ ID NO: 184; SEQ ID NO: 186; and SEQ ID NO: 188 which correspond to the complementarity-determining regions (CDRs, or hypervariable regions) of the light chain sequence of SEQ ID NO: 181 or which comprises the variable light chain sequence of SEQ ID NO: 182, or an antibody or antibody fragment containing combinations of sequences which are at least 80%, 85%, 90%, 95%, 96%, 97%, 98% or 99% identical thereto. In another embodiment of the invention, the antibody or fragments thereof comprises, or alternatively consists of, combinations of one or more of the exemplified variable heavy chain and variable light chain sequences, or the heavy chain and light chain sequences set forth above, or sequences that are at least 90% or 95% identical thereto.

The invention further contemplates anti-glycoprotein an antibody or antibody fragment comprising one, two, three, or four of the polypeptide sequences of SEQ ID NO: 163; SEQ ID NO: 165; SEQ ID NO: 167; and SEQ ID NO: 169 which correspond to the framework regions (FRs or constant regions) of the heavy chain sequence of SEQ ID NO: 161 or the variable heavy chain sequence of SEQ ID NO: 162, and/or one, two, three, or four of the polypeptide sequences of SEQ ID NO: 183; SEQ ID NO: 185; SEQ ID NO: 187; and SEQ ID NO: 189 which correspond to the framework regions (FRs or constant regions) of the light chain sequence of SEQ ID NO: 181 or the variable light chain sequence of SEQ ID NO: 182, or combinations of these polypeptide sequences or sequences which are at least 80%, 90% or 95% identical therewith.

In another embodiment of the invention, the antibody or antibody fragment of the invention comprises, or alternatively consists of, combinations of one or more of the FRs, CDRs, the variable heavy chain and variable light chain sequences, and the heavy chain and light chain sequences set forth above, including all of them or sequences which are at least 90% or 95% identical thereto.

In another embodiment of the invention, the anti-glycoprotein antibody or antibody fragment of the invention comprises, or alternatively consists of, the polypeptide sequence of SEQ ID NO: 161 or SEQ ID NO: 162 or polypeptides that are at least 90% or 95% identical thereto. In another embodiment of the invention, antibody fragment of the invention comprises, or alternatively consists of, the polypeptide sequence of SEQ ID NO: 181 or SEQ ID NO: 182 or polypeptides that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 164; SEQ ID NO: 166; and SEQ ID NO: 168 which correspond to the complementarity-determining regions (CDRs, or hypervariable regions) of the heavy chain sequence of SEQ ID NO: 161 or the variable heavy chain sequence of SEQ ID NO: 162 or sequences that are at least 90% or 95% identical thereto.

In a further embodiment of the invention, the antibody or antibody fragment that specifically binds glycoproteins (such as mannosylated proteins) comprises, or alternatively consists of, one, two, or three of the polypeptide sequences of SEQ ID NO: 184; SEQ ID NO: 186; and SEQ ID NO: 188 which correspond to the complementarity-determining regions (CDRs, or hypervariable regions) of the light chain sequence of SEQ ID NO: 181 or the variable light chain sequence of SEQ ID NO: 182 or sequences that are at least 90% or 95% identical thereto.

In a further embodiment of the invention, the antibody or antibody fragment that specifically binds glycoproteins (such as mannosylated proteins) comprises, or alternatively consists of, one, two, three, or four of the polypeptide sequences of SEQ ID NO: 163; SEQ ID NO: 165; SEQ ID NO: 167; and SEQ ID NO: 169 which correspond to the framework regions (FRs or constant regions) of the heavy chain sequence of SEQ ID NO: 161 or the variable heavy chain sequence of SEQ ID NO: 162 or sequences that are at least 90% or 95% identical thereto.

In a further embodiment of the invention, the subject antibody or antibody fragment that specifically binds glycoproteins (such as mannosylated proteins) comprises, or alternatively consists of, one, two, three, or four of the polypeptide sequences of SEQ ID NO: 183; SEQ ID NO: 185; SEQ ID NO: 187; and SEQ ID NO: 189 which correspond to the framework regions (FRs or constant regions) of the light chain sequence of SEQ ID NO: 181 or the variable light chain sequence of SEQ ID NO: 182 or sequences that are at least 90% or 95% identical thereto.

The invention also contemplates an antibody or fragment thereof that comprises one or more of the antibody fragments described herein. In one embodiment of the invention, the fragment of an antibody that specifically binds glycoproteins (such as mannosylated proteins) comprises, or alternatively consists of, one, two, three or more, including all of the following antibody fragments: the variable heavy chain region of SEQ ID NO: 162; the variable light chain region of SEQ ID NO: 182; the complementarity-determining regions (SEQ ID NO: 164; SEQ ID NO: 166; and SEQ ID NO: 168) of the variable heavy chain region of SEQ ID NO: 162; and the complementarity-determining regions (SEQ ID NO: 184; SEQ ID NO: 186; and SEQ ID NO: 188) of the variable light chain region of SEQ ID NO: 182 or sequences that are at least 90% or 95% identical thereto.

The invention also contemplates an antibody or fragment thereof that comprises one or more of the antibody fragments described herein. In one embodiment of the invention, the fragment of the antibody that specifically binds glycoproteins (such as mannosylated proteins) comprises, or alternatively consists of, one, two, three or more, including all of the following antibody fragments: the variable heavy chain region of SEQ ID NO: 162; the variable light chain region of SEQ ID NO: 182; the framework regions (SEQ ID NO: 163; SEQ ID NO: 165; SEQ ID NO: 167; and SEQ ID NO: 169) of the variable heavy chain region of SEQ ID NO: 162; and the framework regions (SEQ ID NO: 183; SEQ ID NO: 185; SEQ ID NO: 187; and SEQ ID NO: 189) of the variable light chain region of SEQ ID NO: 182.

In a particularly preferred embodiment of the invention, the anti-glycoprotein antibody is Ab5, comprising, or alternatively consisting of, SEQ ID NO: 161 and SEQ ID NO: 181, or an antibody or antibody fragment comprising the CDRs of Ab5 and having at least one of the biological activities set forth herein or is an anti-glycoprotein antibody that competes with Ab5 for binding glycoproteins (such as mannosylated proteins), preferably one containing sequences that are at least 90% or 95% identical to that of Ab5 or an antibody that binds to the same or overlapping epitope(s) on glycoproteins (such as mannosylated proteins) as Ab5.

In a further particularly preferred embodiment of the invention, the antibody fragment comprises, or alternatively consists of, an Fab (fragment antigen binding) fragment having binding specificity for glycoproteins (such as mannosylated proteins). With respect to antibody Ab5, the Fab fragment preferably includes the variable heavy chain sequence of SEQ ID NO: 162 and the variable light chain sequence of SEQ ID NO: 182 or sequences that are at least 90% or 95% identical thereto. This embodiment of the invention further includes an Fab containing additions, deletions, or variants of SEQ ID NO: 162 and/or SEQ ID NO: 182 which retain the binding specificity for glycoproteins (such as mannosylated proteins).

In one embodiment of the invention described herein (infra), Fab fragments may be produced by enzymatic digestion (e.g., papain) of Ab5. In another embodiment of the invention, anti-glycoprotein antibodies such as Ab5 or Fab fragments thereof may be produced via expression in mammalian cells such as CHO, NSO or human kidney cells, fungal, insect, or microbial systems such as yeast cells (for example haploid or diploid yeast such as haploid or diploid Pichia) and other yeast strains. Suitable Pichia species include, but are not limited to, Pichia pastoris.

In an additional embodiment, the invention is further directed to polynucleotides encoding antibody polypeptides having binding specificity for glycoproteins (such as mannosylated proteins), including the heavy and/or light chains of Ab5 as well as fragments, variants, combinations of one or more of the FRs, CDRs, the variable heavy chain and variable light chain sequences, and the heavy chain and light chain sequences set forth above, including all of them or sequences which are at least 90% or 95% identical thereto.

Antibody Polynucleotide Sequences

Antibody Ab1

In one embodiment, the invention is further directed to polynucleotides encoding antibody polypeptides having binding specificity to glycoproteins. In one embodiment of the invention, polynucleotides of the invention comprise, or alternatively consist of, the following polynucleotide sequence encoding the heavy chain sequence of SEQ ID NO: 1:

(SEQ ID NO: 11)

caggagcagttggtggagtccgggggaggcctggtccagcctggggcatc
cctgacactcacctgcacagcttctggattctccttcagtaacaccaatt
acatgtgctgggtccgccaggctccagggaggggcctggagtgggtcgga
tgcatgcccgttggttttattgccagcactttctacgcgacctgggcgaa
aggccgatccgccatctccaagtcctcgtcgaccgcggtgactctgcaaa
tgaccagtctgacagtcgcggacacggccacctatttctgtgcgagagaa
agcggtagtggctgggcgcttaacttgtggggccaagggaccctggtcac
cgtctcgagcgggcaacctaaggctccatcagtcttcccactggccccct
gctgcggggacacaccctctagcacggtgaccttgggctgcctggtcaaa
ggctacctcccggagccagtgaccgtgacctggaactcgggcaccctcac
caatggggtacgcaccttcccgtccgtccggcagtcctcaggcctctact
cgctgagcagcgtggtgagcgtgacctcaagcagccagcccgtcacctgc
aacgtggcccacccagccaccaacaccaaagtggacaagaccgttgcgcc
ctcgacatgcagcaagcccacgtgcccaccccctgaactcctggggggac
cgtctgtcttcatcttccccccaaaacccaaggacaccctcatgatctca
cgcacccccgaggtcacatgcgtggtggtggacgtgagccaggatgaccc
cgaggtgcagttcacatggtacataaacaacgagcaggtgcgcaccgccc
ggccgccgctacgggagcagcagttcaacagcacgatccgcgtggtcagc
accctccccatcgcgcaccaggactggctgaggggcaaggagttcaagtg
caaagtccacaacaaggcactcccggcccccatcgagaaaaccatctcca
aagccagagggcagcccctggagccgaaggtctacaccatgggccctccc
cgggaggagctgagcagcaggtcggtcagcctgacctgcatgatcaacgg
cttctacccttccgacatctcggtggagtgggagaagaacgggaaggcag
aggacaactacaagaccacgccggccgtgctggacagcgacggctcctac
ttcctctacagcaagctctcagtgcccacgagtgagtggcagcggggcga
cgtcttcacctgctccgtgatgcacgaggccttgcacaaccactacacgc
agaagtccatctcccgctctccgggtaaa.

In another embodiment of the invention, the polynucleotides of the invention comprise, or alternatively consist of, the following polynucleotide sequence encoding the variable heavy chain polypeptide sequence of SEQ ID NO: 2:

(SEQ ID NO: 12)

caggagcagttggtggagtccgggggaggcctggtccagcctggggcatc
cctgacactcacctgcacagcttctggattctccttcagtaacaccaatt
acatgtgctgggtccgccaggctccagggaggggcctggagtgggtcgga
tgcatgcccgttggttttattgccagcactttctacgcgacctgggcgaa
aggccgatccgccatctccaagtcctcgtcgaccgcggtgactctgcaaa
tgaccagtctgacagtcgcggacacggccacctatttctgtgcgagagaa
agcggtagtggctgggcgcttaacttgtggggccaagggaccctggtcac
cgtctcgagc.

In another embodiment of the invention, polynucleotides of the invention comprise, or alternatively consist of, the following polynucleotide sequence encoding the constant heavy chain polypeptide sequence of SEQ ID NO: 10:

(SEQ ID NO: 20)

gggcaacctaaggctccatcagtcttcccactggccccctgctgcgggga
cacaccctctagcacggtgaccttgggctgcctggtcaaaggctacctcc
cggagccagtgaccgtgacctggaactcgggcaccctcaccaatggggta
cgcaccttcccgtccgtccggcagtcctcaggcctctactcgctgagcag
cgtggtgagcgtgacctcaagcagccagcccgtcacctgcaacgtggccc
acccagccaccaacaccaaagtggacaagaccgttgcgccctcgacatgc
agcaagcccacgtgcccaccccctgaactcctggggggaccgtctgtctt
catcttccccccaaaacccaaggacaccctcatgatctcacgcacccccg
aggtcacatgcgtggtggtggacgtgagccaggatgaccccgaggtgcag
ttcacatggtacataaacaacgagcaggtgcgcaccgcccggccgccgct
acgggagcagcagttcaacagcacgatccgcgtggtcagcaccctcccca
tcgcgcaccaggactggctgaggggcaaggagttcaagtgcaaagtccac
aacaaggcactcccggcccccatcgagaaaaccatctccaaagccagagg
gcagcccctggagccgaaggtctacaccatgggccctccccgggaggagc
tgagcagcaggtcggtcagcctgacctgcatgatcaacggcttctaccct
tccgacatctcggtggagtgggagaagaacgggaaggcagaggacaacta
caagaccacgccggccgtgctggacagcgacggctcctacttcctctaca
gcaagctctcagtgcccacgagtgagtggcagcggggcgacgtcttcacc
tgctccgtgatgcacgaggccttgcacaaccactacacgcagaagtccat
ctcccgctctccgggtaaa.

In another embodiment of the invention, polynucleotides of the invention comprise, or alternatively consist of, the following polynucleotide sequence encoding the light chain polypeptide sequence of SEQ ID NO: 21:

(SEQ ID NO: 31)

gaccctgtgctgacccagactccatcccccgtgtctgcagctgtgggagg
cacagtcaccatcagttgccaggccagtgagagtgttgagagtggcaact
ggttagcctggtatcagcagaaaccagggcagcctcccaagctcctgatc
tattatacatccactctggcatctggggtcccatcgcggttcaaaggcag
tggatctggggcacacttcactctcaccatcagcggcgtgcagtgtgacg
atgctgccacttactactgtcaaggcgctttttatggtgtgaatactttc
ggcggagggaccgaggtggtggtcaaacgtacgccagttgcacctactgt
cctcctcttcccaccatctagcgatgaggtggcaactggaacagtcacca
tcgtgtgtgtggcgaataaatactttcccgatgtcaccgtcacctgggag
gtggatggcaccacccaaacaactggcatcgagaacagtaaaacaccgca
gaattctgcagattgtacctacaacctcagcagcactctgacactgacca
gcacacagtacaacagccacaaagagtacacctgcaaggtgacccagggc
acgacctcagtcgtccagagcttcagtaggaagaactgt.

In another embodiment of the invention, polynucleotides of the invention comprise, or alternatively consist of, the following polynucleotide sequence encoding the variable light chain polypeptide sequence of SEQ ID NO: 22:

(SEQ ID NO: 32)

gaccctgtgctgacccagactccatcccccgtgtctgcagctgtgggagg
cacagtcaccatcagttgccaggccagtgagagtgttgagagtggcaact
ggttagcctggtatcagcagaaaccagggcagcctcccaagctcctgatc
tattatacatccactctggcatctggggtcccatcgcggttcaaaggcag
tggatctggggcacacttcactctcaccatcagcggcgtgcagtgtgacg
atgctgccacttactactgtcaaggcgctttttatggtgtgaatactttc
ggcggagggaccgaggtggtggtcaaa.

In another embodiment of the invention, polynucleotides of the invention comprise, or alternatively consist of, the following polynucleotide sequence encoding the constant light chain polypeptide sequence of SEQ ID NO: 30:

(SEQ ID NO: 40)

cgtacgccagttgcacctactgtcctcctcttcccaccatctagcgatga
ggtggcaactggaacagtcaccatcgtgtgtgtggcgaataaatactttc
ccgatgtcaccgtcacctgggaggtggatggcaccacccaaacaactggc
atcgagaacagtaaaacaccgcagaattctgcagattgtacctacaacct
cagcagcactctgacactgaccagcacacagtacaacagccacaaagagt
acacctgcaaggtgacccagggcacgacctcagtcgtccagagcttcagt
aggaagaactgt.

In a further embodiment of the invention, polynucleotides encoding antibody fragments having binding specificity for glycoproteins comprise, or alternatively consist of, one or more of the polynucleotide sequences of SEQ ID NO: 14; SEQ ID NO: 16; and SEQ ID NO: 18, which correspond to polynucleotides encoding the complementarity-determining regions (CDRs, or hypervariable regions) of the heavy chain sequence of SEQ ID NO: 1 or the variable heavy chain sequence of SEQ ID NO: 2, and/or one or more of the polynucleotide sequences of SEQ ID NO: 34; SEQ ID NO: 36; and SEQ ID NO: 38, which correspond to the complementarity-determining regions (CDRs, or hypervariable regions) of the light chain sequence of SEQ ID NO: 21 or the variable light chain sequence of SEQ ID NO: 22, or combinations of these polynucleotide sequences. In another embodiment of the invention, the polynucleotides encoding the antibodies of the invention or fragments thereof comprise, or alternatively consist of, combinations of polynucleotides encoding one or more of the CDRs, the variable heavy chain and variable light chain sequences, and the heavy chain and light chain sequences set forth above, including all of them.

In a further embodiment of the invention, polynucleotides encoding antibody fragments having binding specificity for glycoproteins comprise, or alternatively consist of, one or more of the polynucleotide sequences of SEQ ID NO: 13; SEQ ID NO: 15; SEQ ID NO: 17; and SEQ ID NO: 19, which correspond to polynucleotides encoding the framework regions (FRs or constant regions) of the heavy chain sequence of SEQ ID NO: 1 or the variable heavy chain sequence of SEQ ID NO: 2, and/or one or more of the polynucleotide sequences of SEQ ID NO: 33; SEQ ID NO: 35; SEQ ID NO: 37; and SEQ ID NO: 39, which correspond to the framework regions (FRs or constant regions) of the light chain sequence of SEQ ID NO: 21 or the variable light chain sequence of SEQ ID NO: 22, or combinations of these polynucleotide sequences. In another embodiment of the invention, the polynucleotides encoding the antibodies of the invention or fragments thereof comprise, or alternatively consist of, combinations of one or more of the FRs, the variable heavy chain and variable light chain sequences, and the heavy chain and light chain sequences set forth above, including all of them.

The invention also contemplates polynucleotide sequences including one or more of the polynucleotide sequences encoding antibody fragments described herein. In one embodiment of the invention, polynucleotides encoding antibody fragments having binding specificity for glycoproteins comprise, or alternatively consist of, one, two, three or more, including all of the following polynucleotides encoding antibody fragments: the polynucleotide SEQ ID NO: 11 encoding the heavy chain sequence of SEQ ID NO: 1; the polynucleotide SEQ ID NO: 12 encoding the variable heavy chain sequence of SEQ ID NO: 2; the polynucleotide SEQ ID NO: 31 encoding the light chain sequence of SEQ ID NO: 21; the polynucleotide SEQ ID NO: 32 encoding the variable light chain sequence of SEQ ID NO: 22; polynucleotides encoding the complementarity-determining regions (SEQ ID NO: 14; SEQ ID NO: 16; and SEQ ID NO: 18) of the heavy chain sequence of SEQ ID NO: 1 or the variable heavy chain sequence of SEQ ID NO: 2; polynucleotides encoding the complementarity-determining regions (SEQ ID NO: 34; SEQ ID NO: 36; and SEQ ID NO: 38) of the light chain sequence of SEQ ID NO: 21 or the variable light chain sequence of SEQ ID NO: 22; polynucleotides encoding the framework regions (SEQ ID NO: 13; SEQ ID NO: 15; SEQ ID NO: 17; and SEQ ID NO: 19) of the heavy chain sequence of SEQ ID NO: 1 or the variable heavy chain sequence of SEQ ID NO: 2; and polynucleotides encoding the framework regions (SEQ ID NO: 33; SEQ ID NO: 35; SEQ ID NO: 37; and SEQ ID NO: 39) of the light chain sequence of SEQ ID NO: 21 or the variable light chain sequence of SEQ ID NO: 22.

In a preferred embodiment of the invention, polynucleotides of the invention comprise, or alternatively consist of, polynucleotides encoding Fab (fragment antigen binding) fragments having binding specificity for glycoproteins. With respect to antibody Ab1, the polynucleotides encoding the full length Ab1 antibody comprise, or alternatively consist of, the polynucleotide SEQ ID NO: 11 encoding the heavy chain sequence of SEQ ID NO: 1 and the polynucleotide SEQ ID NO: 31 encoding the light chain sequence of SEQ ID NO: 21.

Another embodiment of the invention contemplates these polynucleotides incorporated into an expression vector for expression in mammalian cells such as CHO, NSO, human kidney cells, or in fungal, insect, or microbial systems such as yeast cells such as the yeast Pichia. Suitable Pichia species include, but are not limited to, Pichia pastoris. In one embodiment of the invention described herein (infra), Fab fragments may be produced by enzymatic digestion (e.g., papain) of Ab1 following expression of the full-length polynucleotides in a suitable host. In another embodiment of the invention, anti-glycoprotein antibodies such as Ab1 or Fab fragments thereof may be produced via expression of Ab1 polynucleotides in mammalian cells such as CHO, NSO or human kidney cells, fungal, insect, or microbial systems such as yeast cells (for example diploid yeast such as diploid Pichia) and other yeast strains. Suitable Pichia species include, but are not limited to, Pichia pastoris.

Antibody Ab2 12071 In one embodiment, the invention is further directed to polynucleotides encoding antibody polypeptides having binding specificity to glycoproteins. In one embodiment of the invention, polynucleotides of the invention comprise, or alternatively consist of, the following polynucleotide sequence encoding the heavy chain sequence of SEQ ID NO: 41:

(SEQ ID NO: 51)

cagtcgttggaggagtccgggggaggcctggtcaagcctgagggatccct
gacactcacctgcaaagcctctggattctccttcactggcgcccactaca
tgtgctgggtccgccaggctccagggaaggggctggagtggatcgcatgt
atttatggtggtagtgttgatataactttctacgcgagctgggcgaaagg
ccgattcgccatctccaagtcctcgtcgaccgcggtgactctgcaaatga
ccagtctgacagccgcggacacggccacctatgtctgtgcgagagaaagc
ggtagtggctgggcgcttaacttgtggggcccggggaccctagtcaccgt
ctcgagcgggcaacctaaggctccatcagtcttcccactggccccctgct
gcggggacacaccctctagcacggtgaccttgggctgcctggtcaaaggc
tacctcccggagccagtgaccgtgacctggaactcgggcaccctcaccaa
tggggtacgcaccttcccgtccgtccggcagtcctcaggcctctactcgc
tgagcagcgtggtgagcgtgacctcaagcagccagcccgtcacctgcaac
gtggcccacccagccaccaacaccaaagtggacaagaccgttgcgccctc
gacatgcagcaagcccacgtgcccaccccctgaactcctggggggaccgt
ctgtcttcatcttccccccaaaacccaaggacaccctcatgatctcacgc
acccccgaggtcacatgcgtggtggtggacgtgagccaggatgaccccga
ggtgcagttcacatggtacataaacaacgagcaggtgcgcaccgcccggc
cgccgctacgggagcagcagttcaacagcacgatccgcgtggtcagcacc
ctccccatcgcgcaccaggactggctgaggggcaaggagttcaagtgcaa
agtccacaacaaggcactcccggcccccatcgagaaaaccatctccaaag
ccagagggcagcccctggagccgaaggtctacaccatgggccctccccgg
gaggagctgagcagcaggtcggtcagcctgacctgcatgatcaacggctt
ctacccttccgacatctcggtggagtgggagaagaacgggaaggcagagg
acaactacaagaccacgccggccgtgctggacagcgacggctcctacttc
ctctacagcaagctctcagtgcccacgagtgagtggcagcggggcgacgt
cttcacctgctccgtgatgcacgaggccttgcacaaccactacacgcaga
agtccatctcccgctctccgggtaaa.

In another embodiment of the invention, the polynucleotides of the invention comprise, or alternatively consist of, the following polynucleotide sequence encoding the variable heavy chain polypeptide sequence of SEQ ID NO: 42:

(SEQ ID NO: 52)

cagtcgttggaggagtccgggggaggcctggtcaagcctgagggatccct
gacactcacctgcaaagcctctggattctccttcactggcgcccactaca
tgtgctgggtccgccaggctccagggaaggggctggagtggatcgcatgt
atttatggtggtagtgttgatataactttctacgcgagctgggcgaaagg
ccgattcgccatctccaagtcctcgtcgaccgcggtgactctgcaaatga
ccagtctgacagccgcggacacggccacctatgtctgtgcgagagaaagc
ggtagtggctgggcgcttaacttgtggggcccggggaccctagtcaccgt
ctcgagc.

In another embodiment of the invention, polynucleotides of the invention comprise, or alternatively consist of, the following polynucleotide sequence encoding the constant heavy chain polypeptide sequence of SEQ ID NO: 50:

(SEQ ID NO: 60)

gggcaacctaaggctccatcagtcttcccactggccccctgctgcgggga
cacaccctctagcacggtgaccttgggctgcctggtcaaaggctacctcc
cggagccagtgaccgtgacctggaactcgggcaccctcaccaatggggta
cgcaccttcccgtccgtccggcagtcctcaggcctctactcgctgagcag
cgtggtgagcgtgacctcaagcagccagcccgtcacctgcaacgtggccc
acccagccaccaacaccaaagtggacaagaccgttgcgccctcgacatgc
agcaagcccacgtgcccaccccctgaactcctggggggaccgtctgtctt
catcttccccccaaaacccaaggacaccctcatgatctcacgcacccccg
aggtcacatgcgtggtggtggacgtgagccaggatgaccccgaggtgcag
ttcacatggtacataaacaacgagcaggtgcgcaccgcccggccgccgct
acgggagcagcagttcaacagcacgatccgcgtggtcagcaccctcccca
tcgcgcaccaggactggctgaggggcaaggagttcaagtgcaaagtccac
aacaaggcactcccggcccccatcgagaaaaccatctccaaagccagagg
gcagcccctggagccgaaggtctacaccatgggccctccccgggaggagc
tgagcagcaggtcggtcagcctgacctgcatgatcaacggcttctaccct
tccgacatctcggtggagtgggagaagaacgggaaggcagaggacaacta
caagaccacgccggccgtgctggacagcgacggctcctacttcctctaca
gcaagctctcagtgcccacgagtgagtggcagcggggcgacgtcttcacc
tgctccgtgatgcacgaggccttgcacaaccactacacgcagaagtccat
ctcccgctctccgggtaaa.

In another embodiment of the invention, polynucleotides of the invention comprise, or alternatively consist of, the following polynucleotide sequence encoding the light chain polypeptide sequence of SEQ ID NO: 61:

(SEQ ID NO: 71)

caagtgctgacccagactgcatcgcccgtgtctgccgctgtgggaggcac
agtcaccatcagttgccagtccagtcagagtgttgagaatggcaactggt
tagcctggtatcagcagaaaccagggcagcctcccaagctcctgatctat
ctggcatccactctggaatctggggtcccatcgcggttcaaaggcagtgg
atctgggacacagttcactctcaccatcagcggcgtacagtgtgacgatg
ctgccacttactactgtcagggcgcttatagtggtattaatgttttcggc
ggagggaccgaggtggtggtcaaacgtacgccagttgcacctactgtcct
cctcttcccaccatctagcgatgaggtggcaactggaacagtcaccatcg
tgtgtgtggcgaataaatactttcccgatgtcaccgtcacctgggaggtg
gatggcaccacccaaacaactggcatcgagaacagtaaaacaccgcagaa
ttctgcagattgtacctacaacctcagcagcactctgacactgaccagca
cacagtacaacagccacaaagagtacacctgcaaggtgacccagggcacg
acctcagtcgtccagagcttcagtaggaagaactgt.

In another embodiment of the invention, polynucleotides of the invention comprise, or alternatively consist of, the following polynucleotide sequence encoding the variable light chain polypeptide sequence of SEQ ID NO: 62:

(SEQ ID NO: 72)

caagtgctgacccagactgcatcgcccgtgtctgccgctgtgggaggcac
agtcaccatcagttgccagtccagtcagagtgttgagaatggcaactggt
tagcctggtatcagcagaaaccagggcagcctcccaagctcctgatctat
ctggcatccactctggaatctggggtcccatcgcggttcaaaggcagtgg
atctgggacacagttcactctcaccatcagcggcgtacagtgtgacgatg
ctgccacttactactgtcagggcgcttatagtggtattaatgttttcggc
ggagggaccgaggtggtggtcaaa.

In another embodiment of the invention, polynucleotides of the invention comprise, or alternatively consist of, the following polynucleotide sequence encoding the constant light chain polypeptide sequence of SEQ ID NO: 70:

(SEQ ID NO: 80)

cgtacgccagttgcacctactgtcctcctcttcccaccatctagcgatga
ggtggcaactggaacagtcaccatcgtgtgtgtggcgaataaatactttc
ccgatgtcaccgtcacctgggaggtggatggcaccacccaaacaactggc
atcgagaacagtaaaacaccgcagaattctgcagattgtacctacaacct
cagcagcactctgacactgaccagcacacagtacaacagccacaaagagt
acacctgcaaggtgacccagggcacgacctcagtcgtccagagcttcagt
aggaagaactgt.

In a further embodiment of the invention, polynucleotides encoding antibody fragments having binding specificity for glycoproteins comprise, or alternatively consist of, one or more of the polynucleotide sequences of SEQ ID NO: 54; SEQ ID NO: 56; and SEQ ID NO: 58, which correspond to polynucleotides encoding the complementarity-determining regions (CDRs, or hypervariable regions) of the heavy chain sequence of SEQ ID NO: 41 or the variable heavy chain sequence of SEQ ID NO: 42, and/or one or more of the polynucleotide sequences of SEQ ID NO: 74; SEQ ID NO: 76; and SEQ ID NO: 78, which correspond to the complementarity-determining regions (CDRs, or hypervariable regions) of the light chain sequence of SEQ ID NO: 61 or the variable light chain sequence of SEQ ID NO: 62, or combinations of these polynucleotide sequences. In another embodiment of the invention, the polynucleotides encoding the antibodies of the invention or fragments thereof comprise, or alternatively consist of, combinations of polynucleotides encoding one or more of the CDRs, the variable heavy chain and variable light chain sequences, and the heavy chain and light chain sequences set forth above, including all of them.

In a further embodiment of the invention, polynucleotides encoding antibody fragments having binding specificity for glycoproteins comprise, or alternatively consist of, one or more of the polynucleotide sequences of SEQ ID NO: 53; SEQ ID NO: 55; SEQ ID NO: 57; and SEQ ID NO: 59, which correspond to polynucleotides encoding the framework regions (FRs or constant regions) of the heavy chain sequence of SEQ ID NO: 41 or the variable heavy chain sequence of SEQ ID NO: 42, and/or one or more of the polynucleotide sequences of SEQ ID NO: 73; SEQ ID NO: 75; SEQ ID NO: 77; and SEQ ID NO: 79, which correspond to the framework regions (FRs or constant regions) of the light chain sequence of SEQ ID NO: 61 or the variable light chain sequence of SEQ ID NO: 62, or combinations of these polynucleotide sequences. In another embodiment of the invention, the polynucleotides encoding the antibodies of the invention or fragments thereof comprise, or alternatively consist of, combinations of one or more of the FRs, the variable heavy chain and variable light chain sequences, and the heavy chain and light chain sequences set forth above, including all of them.

The invention also contemplates polynucleotide sequences including one or more of the polynucleotide sequences encoding antibody fragments described herein. In one embodiment of the invention, polynucleotides encoding antibody fragments having binding specificity for glycoproteins comprise, or alternatively consist of, one, two, three or more, including all of the following polynucleotides encoding antibody fragments: the polynucleotide SEQ ID NO: 51 encoding the heavy chain sequence of SEQ ID NO: 41; the polynucleotide SEQ ID NO: 52 encoding the variable heavy chain sequence of SEQ ID NO: 42; the polynucleotide SEQ ID NO: 71 encoding the light chain sequence of SEQ ID NO: 61; the polynucleotide SEQ ID NO: 72 encoding the variable light chain sequence of SEQ ID NO: 62; polynucleotides encoding the complementarity-determining regions (SEQ ID NO: 54; SEQ ID NO: 56; and SEQ ID NO: 58) of the heavy chain sequence of SEQ ID NO: 41 or the variable heavy chain sequence of SEQ ID NO: 42; polynucleotides encoding the complementarity-determining regions (SEQ ID NO: 74; SEQ ID NO: 76; and SEQ ID NO: 78) of the light chain sequence of SEQ ID NO: 61 or the variable light chain sequence of SEQ ID NO: 62; polynucleotides encoding the framework regions (SEQ ID NO: 53; SEQ ID NO: 55; SEQ ID NO: 57; and SEQ ID NO: 59) of the heavy chain sequence of SEQ ID NO: 41 or the variable heavy chain sequence of SEQ ID NO: 42; and polynucleotides encoding the framework regions (SEQ ID NO: 73; SEQ ID NO: 75; SEQ ID NO: 77; and SEQ ID NO: 79) of the light chain sequence of SEQ ID NO: 61 or the variable light chain sequence of SEQ ID NO: 62.

In a preferred embodiment of the invention, polynucleotides of the invention comprise, or alternatively consist of, polynucleotides encoding Fab (fragment antigen binding) fragments having binding specificity for glycoproteins. With respect to antibody Ab2, the polynucleotides encoding the full length Ab2 antibody comprise, or alternatively consist of, the polynucleotide SEQ ID NO: 51 encoding the heavy chain sequence of SEQ ID NO: 41 and the polynucleotide SEQ ID NO: 71 encoding the light chain sequence of SEQ ID NO: 61.

Another embodiment of the invention contemplates these polynucleotides incorporated into an expression vector for expression in mammalian cells such as CHO, NSO, human kidney cells, or in fungal, insect, or microbial systems such as yeast cells such as the yeast Pichia. Suitable Pichia species include, but are not limited to, Pichia pastoris. In one embodiment of the invention described herein (infra), Fab fragments may be produced by enzymatic digestion (e.g., papain) of Ab2 following expression of the full-length polynucleotides in a suitable host. In another embodiment of the invention, anti-glycoprotein antibodies such as Ab2 or Fab fragments thereof may be produced via expression of Ab2 polynucleotides in mammalian cells such as CHO, NSO or human kidney cells, fungal, insect, or microbial systems such as yeast cells (for example diploid yeast such as diploid Pichia) and other yeast strains. Suitable Pichia species include, but are not limited to, Pichia pastoris.

Antibody Ab3

In one embodiment, the invention is further directed to polynucleotides encoding antibody polypeptides having binding specificity to glycoproteins. In one embodiment of the invention, polynucleotides of the invention comprise, or alternatively consist of, the following polynucleotide sequence encoding the heavy chain sequence of SEQ ID NO: 81:

(SEQ ID NO: 91)

cagtcgttggaggagtccgggggaggcctggtccagcctgagggatccct
gacactcacctgtacagcctctggattcttcttcagtggcgcccactaca
tgtgctgggtccgccaggctccagggcaggggctggagtggatcggatgc
acttatggtggtagtgttgatatcactttctacgcgagctgggcgaaagg
ccgattcgccatctccaaaacctcgtcgaccacggtgactctgcaactga
ccagtctgacagccgcggacacggccacctatgtctgtgcgagagaaagc
ggtagtggctgggcacttaacttgtggggccaggggaccctcgtcaccgt
ctcgagcgggcaacctaaggctccatcagtcttcccactggccccctgct
gcggggacacaccctctagcacggtgaccttgggctgcctggtcaaaggc
tacctcccggagccagtgaccgtgacctggaactcgggcaccctcaccaa
tggggtacgcaccttcccgtccgtccggcagtcctcaggcctctactcgc
tgagcagcgtggtgagcgtgacctcaagcagccagcccgtcacctgcaac
gtggcccacccagccaccaacaccaaagtggacaagaccgttgcgccctc
gacatgcagcaagcccacgtgcccaccccctgaactcctggggggaccgt
ctgtcttcatcttccccccaaaacccaaggacaccctcatgatctcacgc
acccccgaggtcacatgcgtggtggtggacgtgagccaggatgaccccga
ggtgcagttcacatggtacataaacaacgagcaggtgcgcaccgcccggc
cgccgctacgggagcagcagttcaacagcacgatccgcgtggtcagcacc
ctccccatcgcgcaccaggactggctgaggggcaaggagttcaagtgcaa
agtccacaacaaggcactcccggcccccatcgagaaaaccatctccaaag
ccagagggcagcccctggagccgaaggtctacaccatgggccctccccgg
gaggagctgagcagcaggtcggtcagcctgacctgcatgatcaacggctt
ctacccttccgacatctcggtggagtgggagaagaacgggaaggcagagg
acaactacaagaccacgccggccgtgctggacagcgacggctcctacttc
ctctacagcaagctctcagtgcccacgagtgagtggcagcggggcgacgt
cttcacctgctccgtgatgcacgaggccttgcacaaccactacacgcaga
agtccatctcccgctctccgggtaaa.

In another embodiment of the invention, the polynucleotides of the invention comprise, or alternatively consist of, the following polynucleotide sequence encoding the variable heavy chain polypeptide sequence of SEQ ID NO: 82:

(SEQ ID NO: 92)

cagtcgttggaggagtccgggggaggcctggtccagcctgagggatccct
gacactcacctgtacagcctctggattcttcttcagtggcgcccactaca
tgtgctgggtccgccaggctccagggcaggggctggagtggatcggatgc
acttatggtggtagtgttgatatcactttctacgcgagctgggcgaaagg
ccgattcgccatctccaaaacctcgtcgaccacggtgactctgcaactga
ccagtctgacagccgcggacacggccacctatgtctgtgcgagagaaagc
ggtagtggctgggcacttaacttgtggggccaggggaccctcgtcaccgt
ctcgagc.

In another embodiment of the invention, polynucleotides of the invention comprise, or alternatively consist of, the following polynucleotide sequence encoding the constant heavy chain polypeptide sequence of SEQ ID NO: 90:

(SEQ ID NO: 100)

gggcaacctaaggctccatcagtcttcccactggccccctgctgcgggga
cacaccctctagcacggtgaccttgggctgcctggtcaaaggctacctcc
cggagccagtgaccgtgacctggaactcgggcaccctcaccaatggggta
cgcaccttcccgtccgtccggcagtcctcaggcctctactcgctgagcag
cgtggtgagcgtgacctcaagcagccagcccgtcacctgcaacgtggccc
acccagccaccaacaccaaagtggacaagaccgttgcgccctcgacatgc
agcaagcccacgtgcccaccccctgaactcctggggggaccgtctgtctt
catcttccccccaaaacccaaggacaccctcatgatctcacgcacccccg
aggtcacatgcgtggtggtggacgtgagccaggatgaccccgaggtgcag
ttcacatggtacataaacaacgagcaggtgcgcaccgcccggccgccgct
acgggagcagcagttcaacagcacgatccgcgtggtcagcaccctcccca
tcgcgcaccaggactggctgaggggcaaggagttcaagtgcaaagtccac
aacaaggcactcccggcccccatcgagaaaaccatctccaaagccagagg
gcagcccctggagccgaaggtctacaccatgggccctccccgggaggagc
tgagcagcaggtcggtcagcctgacctgcatgatcaacggcttctaccct
tccgacatctcggtggagtgggagaagaacgggaaggcagaggacaacta
caagaccacgccggccgtgctggacagcgacggctcctacttcctctaca
gcaagctctcagtgcccacgagtgagtggcagcggggcgacgtcttcacc
tgctccgtgatgcacgaggccttgcacaaccactacacgcagaagtccat
ctcccgctctccgggtaaa.

In another embodiment of the invention, polynucleotides of the invention comprise, or alternatively consist of, the following polynucleotide sequence encoding the light chain polypeptide sequence of SEQ ID NO: 101:

(SEQ ID NO: 111)

caggtgctgacccagactccatcccccgtgtctgcagctgtgggaggcgc
agtcaccatcaattgccagtccagtcagagtgttgagaatggcaactggt
taggctggtatcagcagaaaccagggcagcctcccaagctcctgatctat
ctggcatccactctggcatctggggtcccttcgcggttcaccggcagcgg
atctgggacacagttcactctcaccatcagcggcgtgcagtgtgacgatg
ctgccacttactattgtcaaggcgcttatagtggtattaatgctttcggc
ggagggaccgaggtggtggtcaaacgtacgccagttgcacctactgtcct
cctcttcccaccatctagcgatgaggtggcaactggaacagtcaccatcg
tgtgtgtggcgaataaatactttcccgatgtcaccgtcacctgggaggtg
gatggcaccacccaaacaactggcatcgagaacagtaaaacaccgcagaa
ttctgcagattgtacctacaacctcagcagcactctgacactgaccagca
cacagtacaacagccacaaagagtacacctgcaaggtgacccagggcacg
acctcagtcgtccagagcttcagtaggaagaactgt.

In another embodiment of the invention, polynucleotides of the invention comprise, or alternatively consist of, the following polynucleotide sequence encoding the variable light chain polypeptide sequence of SEQ ID NO: 102:

(SEQ ID NO: 112)

caggtgctgacccagactccatcccccgtgtctgcagctgtgggaggcgc
agtcaccatcaattgccagtccagtcagagtgttgagaatggcaactggt
taggctggtatcagcagaaaccagggcagcctcccaagctcctgatctat
ctggcatccactctggcatctggggtcccttcgcggttcaccggcagcgg
atctgggacacagttcactctcaccatcagcggcgtgcagtgtgacgatg
ctgccacttactattgtcaaggcgcttatagtggtattaatgctttcggc
ggagggaccgaggtggtggtcaaa.

In another embodiment of the invention, polynucleotides of the invention comprise, or alternatively consist of, the following polynucleotide sequence encoding the constant light chain polypeptide sequence of SEQ ID NO: 110:

(SEQ ID NO: 120)

cgtacgccagttgcacctactgtcctcctcttcccaccatctagcgatga
ggtggcaactggaacagtcaccatcgtgtgtgtggcgaataaatactttc
ccgatgtcaccgtcacctgggaggtggatggcaccacccaaacaactggc
atcgagaacagtaaaacaccgcagaattctgcagattgtacctacaacct
cagcagcactctgacactgaccagcacacagtacaacagccacaaagagt
acacctgcaaggtgacccagggcacgacctcagtcgtccagagcttcagt
aggaagaactgt.

In a further embodiment of the invention, polynucleotides encoding antibody fragments having binding specificity for glycoproteins comprise, or alternatively consist of, one or more of the polynucleotide sequences of SEQ ID NO: 94; SEQ ID NO: 96; and SEQ ID NO: 98, which correspond to polynucleotides encoding the complementarity-determining regions (CDRs, or hypervariable regions) of the heavy chain sequence of SEQ ID NO: 81 or the variable heavy chain sequence of SEQ ID NO: 82, and/or one or more of the polynucleotide sequences of SEQ ID NO: 114; SEQ ID NO: 116; and SEQ ID NO: 118, which correspond to the complementarity-determining regions (CDRs, or hypervariable regions) of the light chain sequence of SEQ ID NO: 101 or the variable light chain sequence of SEQ ID NO: 102, or combinations of these polynucleotide sequences. In another embodiment of the invention, the polynucleotides encoding the antibodies of the invention or fragments thereof comprise, or alternatively consist of, combinations of polynucleotides encoding one or more of the CDRs, the variable heavy chain and variable light chain sequences, and the heavy chain and light chain sequences set forth above, including all of them.

In a further embodiment of the invention, polynucleotides encoding antibody fragments having binding specificity for glycoproteins comprise, or alternatively consist of, one or more of the polynucleotide sequences of SEQ ID NO: 93; SEQ ID NO: 95; SEQ ID NO: 97; and SEQ ID NO: 99, which correspond to polynucleotides encoding the framework regions (FRs or constant regions) of the heavy chain sequence of SEQ ID NO: 81 or the variable heavy chain sequence of SEQ ID NO: 82, and/or one or more of the polynucleotide sequences of SEQ ID NO: 113; SEQ ID NO: 115; SEQ ID NO: 117; and SEQ ID NO: 119, which correspond to the framework regions (FRs or constant regions) of the light chain sequence of SEQ ID NO: 101 or the variable light chain sequence of SEQ ID NO: 102, or combinations of these polynucleotide sequences. In another embodiment of the invention, the polynucleotides encoding the antibodies of the invention or fragments thereof comprise, or alternatively consist of, combinations of one or more of the FRs, the variable heavy chain and variable light chain sequences, and the heavy chain and light chain sequences set forth above, including all of them.

The invention also contemplates polynucleotide sequences including one or more of the polynucleotide sequences encoding antibody fragments described herein. In one embodiment of the invention, polynucleotides encoding antibody fragments having binding specificity for glycoproteins comprise, or alternatively consist of, one, two, three or more, including all of the following polynucleotides encoding antibody fragments: the polynucleotide SEQ ID NO: 91 encoding the heavy chain sequence of SEQ ID NO: 81; the polynucleotide SEQ ID NO: 92 encoding the variable heavy chain sequence of SEQ ID NO: 82; the polynucleotide SEQ ID NO: 111 encoding the light chain sequence of SEQ ID NO: 101; the polynucleotide SEQ ID NO: 112 encoding the variable light chain sequence of SEQ ID NO: 102; polynucleotides encoding the complementarity-determining regions (SEQ ID NO: 94; SEQ ID NO: 96; and SEQ ID NO: 98) of the heavy chain sequence of SEQ ID NO: 81 or the variable heavy chain sequence of SEQ ID NO: 82; polynucleotides encoding the complementarity-determining regions (SEQ ID NO: 114; SEQ ID NO: 116; and SEQ ID NO: 118) of the light chain sequence of SEQ ID NO: 101 or the variable light chain sequence of SEQ ID NO: 102; polynucleotides encoding the framework regions (SEQ ID NO: 93; SEQ ID NO: 95; SEQ ID NO: 97; and SEQ ID NO: 99) of the heavy chain sequence of SEQ ID NO: 81 or the variable heavy chain sequence of SEQ ID NO: 82; and polynucleotides encoding the framework regions (SEQ ID NO: 113; SEQ ID NO: 115; SEQ ID NO: 117; and SEQ ID NO: 119) of the light chain sequence of SEQ ID NO: 101 or the variable light chain sequence of SEQ ID NO: 102.

In a preferred embodiment of the invention, polynucleotides of the invention comprise, or alternatively consist of, polynucleotides encoding Fab (fragment antigen binding) fragments having binding specificity for glycoproteins. With respect to antibody Ab3, the polynucleotides encoding the full length Ab3 antibody comprise, or alternatively consist of, the polynucleotide SEQ ID NO: 91 encoding the heavy chain sequence of SEQ ID NO: 81 and the polynucleotide SEQ ID NO: 111 encoding the light chain sequence of SEQ ID NO: 101.

Another embodiment of the invention contemplates these polynucleotides incorporated into an expression vector for expression in mammalian cells such as CHO, NSO, human kidney cells, or in fungal, insect, or microbial systems such as yeast cells such as the yeast Pichia. Suitable Pichia species include, but are not limited to, Pichia pastoris. In one embodiment of the invention described herein (infra), Fab fragments may be produced by enzymatic digestion (e.g., papain) of Ab3 following expression of the full-length polynucleotides in a suitable host. In another embodiment of the invention, anti-glycoprotein antibodies such as Ab3 or Fab fragments thereof may be produced via expression of Ab3 polynucleotides in mammalian cells such as CHO, NSO or human kidney cells, fungal, insect, or microbial systems such as yeast cells (for example diploid yeast such as diploid Pichia) and other yeast strains. Suitable Pichia species include, but are not limited to, Pichia pastoris.

Antibody Ab4

In one embodiment, the invention is further directed to polynucleotides encoding antibody polypeptides having binding specificity to glycoproteins. In one embodiment of the invention, polynucleotides of the invention comprise, or alternatively consist of, the following polynucleotide sequence encoding the heavy chain sequence of SEQ ID NO: 121:

(SEQ ID NO: 131)

cagtcgttggaggagtccgggggagacctggtcaagcctggggcatccct
gacactcacctgcacagcctctggattctccttcagtagcggctacgaca
tgtgttgggtccgccaggctccagggaaggggctggagtggatcgcctgt
atttaccctaataatcctgtcacttactacgcgagctgggcgaaaggccg
attcaccatctccaaaacctcgtcgaccacggtgactctgcaaatgacca
gtctgacagccgcggacacggccacctatttctgtgggagatctgatagt
aatggtcatacctttaacttgtggggccaaggcaccctcgtcaccgtctc
gagcgggcaacctaaggctccatcagtcttcccactggccccctgctgcg
gggacacaccctctagcacggtgaccttgggctgcctggtcaaaggctac
ctcccggagccagtgaccgtgacctggaactcgggcaccctcaccaatgg
ggtacgcaccttcccgtccgtccggcagtcctcaggcctctactcgctga
gcagcgtggtgagcgtgacctcaagcagccagcccgtcacctgcaacgtg
gcccacccagccaccaacaccaaagtggacaagaccgttgcgccctcgac
atgcagcaagcccacgtgcccaccccctgaactcctggggggaccgtctg
tcttcatcttccccccaaaacccaaggacaccctcatgatctcacgcacc
cccgaggtcacatgcgtggtggtggacgtgagccaggatgaccccgaggt
gcagttcacatggtacataaacaacgagcaggtgcgcaccgcccggccgc
cgctacgggagcagcagttcaacagcacgatccgcgtggtcagcaccctc
cccatcgcgcaccaggactggctgaggggcaaggagttcaagtgcaaagt
ccacaacaaggcactcccggcccccatcgagaaaaccatctccaaagcca
gagggcagcccctggagccgaaggtctacaccatgggccctccccgggag
gagctgagcagcaggtcggtcagcctgacctgcatgatcaacggcttcta
cccttccgacatctcggtggagtgggagaagaacgggaaggcagaggaca
actacaagaccacgccggccgtgctggacagcgacggctcctacttcctc
tacagcaagctctcagtgcccacgagtgagtggcagcggggcgacgtctt
cacctgctccgtgatgcacgaggccttgcacaaccactacacgcagaagt
ccatctcccgctctccgggtaaa.

In another embodiment of the invention, the polynucleotides of the invention comprise, or alternatively consist of, the following polynucleotide sequence encoding the variable heavy chain polypeptide sequence of SEQ ID NO: 122:

(SEQ ID NO: 132)

cagtcgttggaggagtccgggggagacctggtcaagcctggggcatccct
gacactcacctgcacagcctctggattctccttcagtagcggctacgaca
tgtgttgggtccgccaggctccagggaaggggctggagtggatcgcctgt
atttaccctaataatcctgtcacttactacgcgagctgggcgaaaggccg
attcaccatctccaaaacctcgtcgaccacggtgactctgcaaatgacca
gtctgacagccgcggacacggccacctatttctgtgggagatctgatagt
aatggtcatacctttaacttgtggggccaaggcaccctcgtcaccgtctc
gagc.

In another embodiment of the invention, polynucleotides of the invention comprise, or alternatively consist of, the following polynucleotide sequence encoding the constant heavy chain polypeptide sequence of SEQ ID NO: 130:

(SEQ ID NO: 140)

gggcaacctaaggctccatcagtcttcccactggccccctgctgcgggga
cacaccctctagcacggtgaccttgggctgcctggtcaaaggctacctcc
cggagccagtgaccgtgacctggaactcgggcaccctcaccaatggggta
cgcaccttcccgtccgtccggcagtcctcaggcctctactcgctgagcag
cgtggtgagcgtgacctcaagcagccagcccgtcacctgcaacgtggccc
acccagccaccaacaccaaagtggacaagaccgttgcgccctcgacatgc
agcaagcccacgtgcccaccccctgaactcctggggggaccgtctgtctt
catcttccccccaaaacccaaggacaccctcatgatctcacgcacccccg
aggtcacatgcgtggtggtggacgtgagccaggatgaccccgaggtgcag
ttcacatggtacataaacaacgagcaggtgcgcaccgcccggccgccgct
acgggagcagcagttcaacagcacgatccgcgtggtcagcaccctcccca
tcgcgcaccaggactggctgaggggcaaggagttcaagtgcaaagtccac
aacaaggcactcccggcccccatcgagaaaaccatctccaaagccagagg
gcagcccctggagccgaaggtctacaccatgggccctccccgggaggagc
tgagcagcaggtcggtcagcctgacctgcatgatcaacggcttctaccct
tccgacatctcggtggagtgggagaagaacgggaaggcagaggacaacta
caagaccacgccggccgtgctggacagcgacggctcctacttcctctaca
gcaagctctcagtgcccacgagtgagtggcagcggggcgacgtcttcacc
tgctccgtgatgcacgaggccttgcacaaccactacacgcagaagtccat
ctcccgctctccgggtaaa.

In another embodiment of the invention, polynucleotides of the invention comprise, or alternatively consist of, the following polynucleotide sequence encoding the light chain polypeptide sequence of SEQ ID NO: 141:

(SEQ ID NO: 151)

gaccctgtgatgacccagactccatcctccgtgtctgcagctgtgggagg
cacagtcaccatcaattgccagtccagtcagagtgttaatcagaacgact
tatcctggtatcagcagaaaccagggcagcctcccaagcgcctgatctat
tatgcatccactctggcatctggggtctcatcgcggttcaaaggcagtgg
atctgggacacagttcactctcaccatcagcgacatgcagtgtgacgatg
ctgccacttactactgtcaaggcagttttcgtgttagtggttggtactgg
gctttcggcggagggaccgaggtggtggtcaaacgtacgccagttgcacc
tactgtcctcctcttcccaccatctagcgatgaggtggcaactggaacag
tcaccatcgtgtgtgtggcgaataaatactttcccgatgtcaccgtcacc
tgggaggtggatggcaccacccaaacaactggcatcgagaacagtaaaac
accgcagaattctgcagattgtacctacaacctcagcagcactctgacac
tgaccagcacacagtacaacagccacaaagagtacacctgcaaggtgacc
cagggcacgacctcagtcgtccagagcttcagtaggaagaactgt.

In another embodiment of the invention, polynucleotides of the invention comprise, or alternatively consist of, the following polynucleotide sequence encoding the variable light chain polypeptide sequence of SEQ ID NO: 142:

(SEQ ID NO: 152)

gaccctgtgatgacccagactccatcctccgtgtctgcagctgtgggagg
cacagtcaccatcaattgccagtccagtcagagtgttaatcagaacgact
tatcctggtatcagcagaaaccagggcagcctcccaagcgcctgatctat
tatgcatccactctggcatctggggtctcatcgcggttcaaaggcagtgg
atctgggacacagttcactctcaccatcagcgacatgcagtgtgacgatg
ctgccacttactactgtcaaggcagttttcgtgttagtggttggtactgg
gctttcggcggagggaccgaggtggtggtcaaa.

In another embodiment of the invention, polynucleotides of the invention comprise, or alternatively consist of, the following polynucleotide sequence encoding the constant light chain polypeptide sequence of SEQ ID NO: 150:

(SEQ ID NO: 160)

cgtacgccagttgcacctactgtcctcctcttcccaccatctagcgatga
ggtggcaactggaacagtcaccatcgtgtgtgtggcgaataaatactttc
ccgatgtcaccgtcacctgggaggtggatggcaccacccaaacaactggc
atcgagaacagtaaaacaccgcagaattctgcagattgtacctacaacct
cagcagcactctgacactgaccagcacacagtacaacagccacaaagagt
acacctgcaaggtgacccagggcacgacctcagtcgtccagagcttcagt
aggaagaactgt.

In a further embodiment of the invention, polynucleotides encoding antibody fragments having binding specificity for glycoproteins comprise, or alternatively consist of, one or more of the polynucleotide sequences of SEQ ID NO: 134; SEQ ID NO: 136; and SEQ ID NO: 138, which correspond to polynucleotides encoding the complementarity-determining regions (CDRs, or hypervariable regions) of the heavy chain sequence of SEQ ID NO: 121 or the variable heavy chain sequence of SEQ ID NO: 122, and/or one or more of the polynucleotide sequences of SEQ ID NO: 154; SEQ ID NO: 156; and SEQ ID NO: 158, which correspond to the complementarity-determining regions (CDRs, or hypervariable regions) of the light chain sequence of SEQ ID NO: 141 or the variable light chain sequence of SEQ ID NO: 142, or combinations of these polynucleotide sequences. In another embodiment of the invention, the polynucleotides encoding the antibodies of the invention or fragments thereof comprise, or alternatively consist of, combinations of polynucleotides encoding one or more of the CDRs, the variable heavy chain and variable light chain sequences, and the heavy chain and light chain sequences set forth above, including all of them.

In a further embodiment of the invention, polynucleotides encoding antibody fragments having binding specificity for glycoproteins comprise, or alternatively consist of, one or more of the polynucleotide sequences of SEQ ID NO: 133; SEQ ID NO: 135; SEQ ID NO: 137; and SEQ ID NO: 139, which correspond to polynucleotides encoding the framework regions (FRs or constant regions) of the heavy chain sequence of SEQ ID NO: 121 or the variable heavy chain sequence of SEQ ID NO: 122, and/or one or more of the polynucleotide sequences of SEQ ID NO: 153; SEQ ID NO: 155; SEQ ID NO: 157; and SEQ ID NO: 159, which correspond to the framework regions (FRs or constant regions) of the light chain sequence of SEQ ID NO: 141 or the variable light chain sequence of SEQ ID NO: 142, or combinations of these polynucleotide sequences. In another embodiment of the invention, the polynucleotides encoding the antibodies of the invention or fragments thereof comprise, or alternatively consist of, combinations of one or more of the FRs, the variable heavy chain and variable light chain sequences, and the heavy chain and light chain sequences set forth above, including all of them.

The invention also contemplates polynucleotide sequences including one or more of the polynucleotide sequences encoding antibody fragments described herein. In one embodiment of the invention, polynucleotides encoding antibody fragments having binding specificity for glycoproteins comprise, or alternatively consist of, one, two, three or more, including all of the following polynucleotides encoding antibody fragments: the polynucleotide SEQ ID NO: 131 encoding the heavy chain sequence of SEQ ID NO: 121; the polynucleotide SEQ ID NO: 132 encoding the variable heavy chain sequence of SEQ ID NO: 122; the polynucleotide SEQ ID NO: 151 encoding the light chain sequence of SEQ ID NO: 141; the polynucleotide SEQ ID NO: 152 encoding the variable light chain sequence of SEQ ID NO: 142; polynucleotides encoding the complementarity-determining regions (SEQ ID NO: 134; SEQ ID NO: 136; and SEQ ID NO: 138) of the heavy chain sequence of SEQ ID NO: 121 or the variable heavy chain sequence of SEQ ID NO: 122; polynucleotides encoding the complementarity-determining regions (SEQ ID NO: 154; SEQ ID NO: 156; and SEQ ID NO: 158) of the light chain sequence of SEQ ID NO: 141 or the variable light chain sequence of SEQ ID NO: 142; polynucleotides encoding the framework regions (SEQ ID NO: 133; SEQ ID NO: 135; SEQ ID NO: 137; and SEQ ID NO: 139) of the heavy chain sequence of SEQ ID NO: 121 or the variable heavy chain sequence of SEQ ID NO: 122; and polynucleotides encoding the framework regions (SEQ ID NO: 153; SEQ ID NO: 155; SEQ ID NO: 157; and SEQ ID NO: 159) of the light chain sequence of SEQ ID NO: 141 or the variable light chain sequence of SEQ ID NO: 142.

In a preferred embodiment of the invention, polynucleotides of the invention comprise, or alternatively consist of, polynucleotides encoding Fab (fragment antigen binding) fragments having binding specificity for glycoproteins. With respect to antibody Ab4, the polynucleotides encoding the full length Ab4 antibody comprise, or alternatively consist of, the polynucleotide SEQ ID NO: 131 encoding the heavy chain sequence of SEQ ID NO: 121 and the polynucleotide SEQ ID NO: 151 encoding the light chain sequence of SEQ ID NO: 141.

Another embodiment of the invention contemplates these polynucleotides incorporated into an expression vector for expression in mammalian cells such as CHO, NSO, human kidney cells, or in fungal, insect, or microbial systems such as yeast cells such as the yeast Pichia. Suitable Pichia species include, but are not limited to, Pichia pastoris. In one embodiment of the invention described herein (infra), Fab fragments may be produced by enzymatic digestion (e.g., papain) of Ab4 following expression of the full-length polynucleotides in a suitable host. In another embodiment of the invention, anti-glycoprotein antibodies such as Ab4 or Fab fragments thereof may be produced via expression of Ab4 polynucleotides in mammalian cells such as CHO, NSO or human kidney cells, fungal, insect, or microbial systems such as yeast cells (for example diploid yeast such as diploid Pichia) and other yeast strains. Suitable Pichia species include, but are not limited to, Pichia pastoris.

Antibody Ab5

In one embodiment, the invention is further directed to polynucleotides encoding antibody polypeptides having binding specificity to glycoproteins. In one embodiment of the invention, polynucleotides of the invention comprise, or alternatively consist of, the following polynucleotide sequence encoding the heavy chain sequence of SEQ ID NO: 161:

(SEQ ID NO: 171)

cagcagcagttgctggagtccgggggaggcctggtccagcctgagggatc
cctggcactcacctgcacagcttctggattctccttcagtagcggctacg
acatgtgctgggtccgccagcctccagggaaggggctggagtgggtcggc
tgcatttatagtggtgatgataatgatattacttattacgcgagctgggc
gagaggccgattcaccatctccaacccctcgtcgaccactgtgactctgc
aaatgaccagtctgacagtcgcggacacggccacctatttctgtgcgcga
ggtcatgctatttatgataattatgatagtgtccacttgtggggccaggg
gaccctcgtcaccgtctcgagcgggcaacctaaggctccatcagtcttcc
cactggccccctgctgcggggacacaccctctagcacggtgaccttgggc
tgcctggtcaaaggctacctcccggagccagtgaccgtgacctggaactc
gggcaccctcaccaatggggtacgcaccttcccgtccgtccggcagtcct
caggcctctactcgctgagcagcgtggtgagcgtgacctcaagcagccag
cccgtcacctgcaacgtggcccacccagccaccaacaccaaagtggacaa
gaccgttgcgccctcgacatgcagcaagcccacgtgcccaccccctgaac
tcctggggggaccgtctgtcttcatcttccccccaaaacccaaggacacc
ctcatgatctcacgcacccccgaggtcacatgcgtggtggtggacgtgag
ccaggatgaccccgaggtgcagttcacatggtacataaacaacgagcagg
tgcgcaccgcccggccgccgctacgggagcagcagttcaacagcacgatc
cgcgtggtcagcaccctccccatcgcgcaccaggactggctgaggggcaa
ggagttcaagtgcaaagtccacaacaaggcactcccggcccccatcgaga
aaaccatctccaaagccagagggcagcccctggagccgaaggtctacacc
atgggccctccccgggaggagctgagcagcaggtcggtcagcctgacctg
catgatcaacggcttctacccttccgacatctcggtggagtgggagaaga
acgggaaggcagaggacaactacaagaccacgccggccgtgctggacagc
gacggctcctacttcctctacagcaagctctcagtgcccacgagtgagtg
gcagcggggcgacgtcttcacctgctccgtgatgcacgaggccttgcaca
accactacacgcagaagtccatctcccgctctccgggtaaa.

In another embodiment of the invention, the polynucleotides of the invention comprise, or alternatively consist of, the following polynucleotide sequence encoding the variable heavy chain polypeptide sequence of SEQ ID NO: 162:

(SEQ ID NO: 172)

cagcagcagttgctggagtccgggggaggcctggtccagcctgagggatc
cctggcactcacctgcacagcttctggattctccttcagtagcggctacg
acatgtgctgggtccgccagcctccagggaaggggctggagtgggtcggc
tgcatttatagtggtgatgataatgatattacttattacgcgagctgggc
gagaggccgattcaccatctccaacccctcgtcgaccactgtgactctgc
aaatgaccagtctgacagtcgcggacacggccacctatttctgtgcgcga
ggtcatgctatttatgataattatgatagtgtccacttgtggggccaggg
gaccctcgtcaccgtctcgagc.

In another embodiment of the invention, polynucleotides of the invention comprise, or alternatively consist of, the following polynucleotide sequence encoding the constant heavy chain polypeptide sequence of SEQ ID NO: 170:

(SEQ ID NO: 180)

gggcaacctaaggctccatcagtcttcccactggccccctgctgcgggga
cacaccctctagcacggtgaccttgggctgcctggtcaaaggctacctcc
cggagccagtgaccgtgacctggaactcgggcaccctcaccaatggggta
cgcaccttcccgtccgtccggcagtcctcaggcctctactcgctgagcag
cgtggtgagcgtgacctcaagcagccagcccgtcacctgcaacgtggccc
acccagccaccaacaccaaagtggacaagaccgttgcgccctcgacatgc
agcaagcccacgtgcccaccccctgaactcctggggggaccgtctgtctt
catcttccccccaaaacccaaggacaccctcatgatctcacgcacccccg
aggtcacatgcgtggtggtggacgtgagccaggatgaccccgaggtgcag
ttcacatggtacataaacaacgagcaggtgcgcaccgcccggccgccgct
acgggagcagcagttcaacagcacgatccgcgtggtcagcaccctcccca
tcgcgcaccaggactggctgaggggcaaggagttcaagtgcaaagtccac
aacaaggcactcccggcccccatcgagaaaaccatctccaaagccagagg
gcagcccctggagccgaaggtctacaccatgggccctccccgggaggagc
tgagcagcaggtcggtcagcctgacctgcatgatcaacggcttctaccct
tccgacatctcggtggagtgggagaagaacgggaaggcagaggacaacta
caagaccacgccggccgtgctggacagcgacggctcctacttcctctaca
gcaagctctcagtgcccacgagtgagtggcagcggggcgacgtcttcacc
tgctccgtgatgcacgaggccttgcacaaccactacacgcagaagtccat
ctcccgctctccgggtaaa.

In another embodiment of the invention, polynucleotides of the invention comprise, or alternatively consist of, the following polynucleotide sequence encoding the light chain polypeptide sequence of SEQ ID NO: 181:

(SEQ ID NO: 191)

atcgtgatgacccagactccatcttccaggtctgtccctgtgggaggcac
agtcaccatcaattgccaggccagtgaaattgttaatagaaacaaccgct
tagcctggtttcaacagaaaccagggcagcctcccaagctcctgatgtat
ctggcttccactccggcatctggggtcccatcgcggtttagaggcagtgg
atctgggacacagttcactctcaccatcagcgatgtggtgtgtgacgatg
ctgccacttattattgtacagcatataagagtagtaatactgatggtatt
gctttcggcggagggaccgaggtggtggtcaaacgtacgccagttgcacc
tactgtcctcctcttcccaccatctagcgatgaggtggcaactggaacag
tcaccatcgtgtgtgtggcgaataaatactttcccgatgtcaccgtcacc
tgggaggtggatggcaccacccaaacaactggcatcgagaacagtaaaac
accgcagaattctgcagattgtacctacaacctcagcagcactctgacac
tgaccagcacacagtacaacagccacaaagagtacacctgcaaggtgacc
cagggcacgacctcagtcgtccagagcttcagtaggaagaactgt.

In another embodiment of the invention, polynucleotides of the invention comprise, or alternatively consist of, the following polynucleotide sequence encoding the variable light chain polypeptide sequence of SEQ ID NO: 182:

(SEQ ID NO: 192)

atcgtgatgacccagactccatcttccaggtctgtccctgtgggaggcac
agtcaccatcaattgccaggccagtgaaattgttaatagaaacaaccgct
tagcctggtttcaacagaaaccagggcagcctcccaagctcctgatgtat
ctggcttccactccggcatctggggtcccatcgcggtttagaggcagtgg
atctgggacacagttcactctcaccatcagcgatgtggtgtgtgacgatg
ctgccacttattattgtacagcatataagagtagtaatactgatggtatt
gctttcggcggagggaccgaggtggtggtcaaa.

In another embodiment of the invention, polynucleotides of the invention comprise, or alternatively consist of, the following polynucleotide sequence encoding the constant light chain polypeptide sequence of SEQ ID NO: 190:

(SEQ ID NO: 200)

cgtacgccagttgcacctactgtcctcctcttcccaccatctagcgatga
ggtggcaactggaacagtcaccatcgtgtgtgtggcgaataaatactttc
ccgatgtcaccgtcacctgggaggtggatggcaccacccaaacaactggc
atcgagaacagtaaaacaccgcagaattctgcagattgtacctacaacct
cagcagcactctgacactgaccagcacacagtacaacagccacaaagagt
acacctgcaaggtgacccagggcacgacctcagtcgtccagagcttcagt
aggaagaactgt.

In a further embodiment of the invention, polynucleotides encoding antibody fragments having binding specificity for glycoproteins comprise, or alternatively consist of, one or more of the polynucleotide sequences of SEQ ID NO: 174; SEQ ID NO: 176; and SEQ ID NO: 178, which correspond to polynucleotides encoding the complementarity-determining regions (CDRs, or hypervariable regions) of the heavy chain sequence of SEQ ID NO: 161 or the variable heavy chain sequence of SEQ ID NO: 162, and/or one or more of the polynucleotide sequences of SEQ ID NO: 194; SEQ ID NO: 196; and SEQ ID NO: 198, which correspond to the complementarity-determining regions (CDRs, or hypervariable regions) of the light chain sequence of SEQ ID NO: 181 or the variable light chain sequence of SEQ ID NO: 182, or combinations of these polynucleotide sequences. In another embodiment of the invention, the polynucleotides encoding the antibodies of the invention or fragments thereof comprise, or alternatively consist of, combinations of polynucleotides encoding one or more of the CDRs, the variable heavy chain and variable light chain sequences, and the heavy chain and light chain sequences set forth above, including all of them.

In a further embodiment of the invention, polynucleotides encoding antibody fragments having binding specificity for glycoproteins comprise, or alternatively consist of, one or more of the polynucleotide sequences of SEQ ID NO: 173; SEQ ID NO: 175; SEQ ID NO: 177; and SEQ ID NO: 179, which correspond to polynucleotides encoding the framework regions (FRs or constant regions) of the heavy chain sequence of SEQ ID NO: 161 or the variable heavy chain sequence of SEQ ID NO: 162, and/or one or more of the polynucleotide sequences of SEQ ID NO: 193; SEQ ID NO: 195; SEQ ID NO: 197; and SEQ ID NO: 199, which correspond to the framework regions (FRs or constant regions) of the light chain sequence of SEQ ID NO: 181 or the variable light chain sequence of SEQ ID NO: 182, or combinations of these polynucleotide sequences. In another embodiment of the invention, the polynucleotides encoding the antibodies of the invention or fragments thereof comprise, or alternatively consist of, combinations of one or more of the FRs, the variable heavy chain and variable light chain sequences, and the heavy chain and light chain sequences set forth above, including all of them.

The invention also contemplates polynucleotide sequences including one or more of the polynucleotide sequences encoding antibody fragments described herein. In one embodiment of the invention, polynucleotides encoding antibody fragments having binding specificity for glycoproteins comprise, or alternatively consist of, one, two, three or more, including all of the following polynucleotides encoding antibody fragments: the polynucleotide SEQ ID NO: 171 encoding the heavy chain sequence of SEQ ID NO: 161; the polynucleotide SEQ ID NO: 172 encoding the variable heavy chain sequence of SEQ ID NO: 162; the polynucleotide SEQ ID NO: 191 encoding the light chain sequence of SEQ ID NO: 181; the polynucleotide SEQ ID NO: 192 encoding the variable light chain sequence of SEQ ID NO: 182; polynucleotides encoding the complementarity-determining regions (SEQ ID NO: 174; SEQ ID NO: 176; and SEQ ID NO: 178) of the heavy chain sequence of SEQ ID NO: 161 or the variable heavy chain sequence of SEQ ID NO: 162; polynucleotides encoding the complementarity-determining regions (SEQ ID NO: 194; SEQ ID NO: 196; and SEQ ID NO: 198) of the light chain sequence of SEQ ID NO: 181 or the variable light chain sequence of SEQ ID NO: 182; polynucleotides encoding the framework regions (SEQ ID NO: 173; SEQ ID NO: 175; SEQ ID NO: 177; and SEQ ID NO: 179) of the heavy chain sequence of SEQ ID NO: 161 or the variable heavy chain sequence of SEQ ID NO: 162; and polynucleotides encoding the framework regions (SEQ ID NO: 193; SEQ ID NO: 195; SEQ ID NO: 197; and SEQ ID NO: 199) of the light chain sequence of SEQ ID NO: 181 or the variable light chain sequence of SEQ ID NO: 182.

In a preferred embodiment of the invention, polynucleotides of the invention comprise, or alternatively consist of, polynucleotides encoding Fab (fragment antigen binding) fragments having binding specificity for glycoproteins. With respect to antibody Ab5, the polynucleotides encoding the full length Ab5 antibody comprise, or alternatively consist of, the polynucleotide SEQ ID NO: 171 encoding the heavy chain sequence of SEQ ID NO: 161 and the polynucleotide SEQ ID NO: 191 encoding the light chain sequence of SEQ ID NO: 181.

Another embodiment of the invention contemplates these polynucleotides incorporated into an expression vector for expression in mammalian cells such as CHO, NSO, human kidney cells, or in fungal, insect, or microbial systems such as yeast cells such as the yeast Pichia. Suitable Pichia species include, but are not limited to, Pichia pastoris. In one embodiment of the invention described herein (infra), Fab fragments may be produced by enzymatic digestion (e.g., papain) of Ab5 following expression of the full-length polynucleotides in a suitable host. In another embodiment of the invention, anti-glycoprotein antibodies such as Ab5 or Fab fragments thereof may be produced via expression of Ab5 polynucleotides in mammalian cells such as CHO, NSO or human kidney cells, fungal, insect, or microbial systems such as yeast cells (for example diploid yeast such as diploid Pichia) and other yeast strains. Suitable Pichia species include, but are not limited to, Pichia pastoris.

### Expression of Desired Proteins

Desired proteins (e.g., recombinant proteins), including homopolymeric or heteropolymeric polypeptides, e.g., an antibody or an antibody fragment, can be expressed in yeast and filamentous fungal cells. In one embodiment, the desired protein is recombinantly expressed in yeast, and particularly preferred yeasts include methylotrophic yeast strains, e.g., Pichia pastoris, Hansenula polymorpha (Pichia angusta), Pichia guillermordii, Pichia methanolica, Pichia inositovera, and others (see, e.g., U.S. Pat. Nos. 4,812,405, 4,818,700, 4,929,555, 5,736,383, 5,955,349, 5,888,768, and 6,258,559 each of which is incorporated by reference in its entirety). Other exemplary yeast include Arxiozyma; Ascobotryozyma; Citeromyces; Debaryomyces; Dekkera; Eremothecium; Issatchenkia; Kazachstania; Kluyveromyces; Kodamaea; Lodderomyces; Pachysolen; Pichia; Saccharomyces; Saturnispora; Tetrapisispora; Torulaspora; Williopsis; Zygosaccharomyces; Yarrowia; Rhodosporidium; Candida; Hansenula; Filobasium; Sporidiobolus; Bullera; Leucosporidium and Filobasidella.

The yeast cell may be produced by methods known in the art. For example, a panel of diploid or tetraploid yeast cells containing differing combinations of gene copy numbers may be generated by mating cells containing varying numbers of copies of the individual subunit genes (which numbers of copies preferably are known in advance of mating).

In one embodiment, the yeast cell may comprise more than one copy of one or more of the genes encoding the desired protein or subunits of the desired multi-subunit protein. For example, multiple copies of a subunit gene may be integrated in tandem into one or more chromosomal loci. Tandemly integrated gene copies are preferably retained in a stable number of copies during culture for the production of the desired protein or multi-subunit complex. For example, in prior work described by the present applicants, gene copy numbers were generally stable for P. pastoris strains containing three to four tandemly integrated copies of light and heavy chain antibody genes (see, U.S. 20130045888).

One or more of the genes encoding the desired protein or subunits thereof are preferably integrated into one or more chromosomal loci of a fungal cell. Any suitable chromosomal locus may be utilized for integration, including intergenic sequences, promoters sequences, coding sequences, termination sequences, regulatory sequences, etc. Exemplary chromosomal loci that may be used in P. pastoris include PpURA5; OCH1; AOX1; HIS4; and GAP. The encoding genes may also be integrated into one or more random chromosomal loci rather than being targeted. In preferred embodiments, the chromosomal loci are selected from the group consisting of the pGAP locus, the 3′AOX TT locus and the HIS4 TT locus. In additional exemplary embodiments, the genes encoding the heterologous protein subunits may be contained in one or more extrachromosomal elements, for example one or more plasmids or artificial chromosomes.

In exemplary embodiments, the desired protein may be a multi-subunit protein that, e.g., comprises two, three, four, five, six, or more identical and/or non-identical subunits. Additionally, each subunit may be present one or more times in each multi-subunit protein. For example, the multi-subunit protein may be a multi-specific antibody such as a bi-specific antibody comprising two non-identical light chains and two non-identical heavy chains. A panel of diploid or tetraploid yeast cells containing differing combinations of gene copy numbers may be quickly generated by mating cells containing varying copy numbers of the individual subunit genes. Antibody production from each strain in the panel may then be assessed to identify a strain for further use based on a characteristic such as yield of the desired multi-subunit protein or purity of the desired multi-subunit protein relative to undesired side-products.

The subunits of a multi-subunit protein may be expressed from monocistronic genes, polycistronic genes, or any combination thereof. Each polycistronic gene may comprise multiple copies of the same subunit, or may comprise one or more copies of each different subunit.

Exemplary methods that may be used for manipulation of Pichia pastoris (including methods of culturing, transforming, and mating) are disclosed in Published Applications including U.S. 20080003643, U.S. 20070298500, and U.S. 20060270045, and in Higgins, D. R., and Cregg, J. M., Eds. 1998. Pichia Protocols. Methods in Molecular Biology. Humana Press, Totowa, N.J., and Cregg, J. M., Ed., 2007, Pichia Protocols (2nd edition), Methods in Molecular Biology. Humana Press, Totowa, N.J., each of which is incorporated by reference in its entirety.

An exemplary expression cassette that may be utilized is composed of the glyceraldehyde dehydrogenase gene (GAP gene) promoter, fused to sequences encoding a secretion signal, followed by the sequence of the gene to be expressed, followed by sequences encoding a P. pastoris transcriptional termination signal from the P. pastoris alcohol oxidase I gene (AOX1). The Zeocin resistance marker gene may provide a means of enrichment for strains that contain multiple integrated copies of an expression vector in a strain by selecting for transformants that are resistant to higher levels of Zeocin. Similarly, G418 or Kanamycin resistance marker genes may be used to provide a means of enrichment for strains that contain multiple integrated copies of an expression vector in a strain by selecting for transformants that are resistant to higher levels of Geneticin or Kanamycin.

Yeast strains that may be utilized include auxotrophic P. pastoris or other Pichia strains, for example, strains having mutations in met1, lys3, ura3 and ade1 or other auxotrophy-associated genes. Preferred mutations are incapable of giving rise to revertants at any appreciable frequency and are preferably partial or even more preferably full deletion mutants. Preferably, prototrophic diploid or tetraploid strains are produced by mating complementing sets of auxotrophic strains.

Prior to transformation, each expression vector may be linearized by restriction enzyme cleavage within a region homologous to the target genomic locus (e.g., the GAP promoter sequence) to direct the integration of the vectors into the target locus in the fungal cell. Samples of each vector may then be individually transformed into cultures of the desired strains by electroporation or other methods, and successful transformants may be selected by means of a selectable marker, e.g., antibiotic resistance or complementation of an auxotrophy. Isolates may be picked, streaked for single colonies under selective conditions and then examined to confirm the number of copies of the gene encoding the desired protein or subunit of the multi-subunit complex (e.g., a desired antibody) by Southern Blot or PCR assay on genomic DNA extracted from each strain. Optionally, expression of the expected subunit gene product may be confirmed, e.g., by FACS, Western Blot, colony lift and immunoblot, and other means known in the art. Optionally, haploid isolates are transformed additional times to introduce additional heterologous genes, e.g., additional copies of the same subunit integrated at a different locus, and/or copies of a different subunit. The haploid strains are then mated to generate diploid strains (or strains of higher ploidy) able to synthesize the multi-protein complex. Presence of each expected subunit gene may be confirmed by Southern blotting, PCR, and other detection means known in the art. Where the desired multi-protein complex is an antibody, its expression may also be confirmed by a colony lift/immunoblot method (Wung et al. Biotechniques 21 808-812 (1996)) and/or by FACS.

This transformation protocol is optionally repeated to target a heterologous gene into a second locus, which may be the same gene or a different gene than was targeted into the first locus. When the construct to be integrated into the second locus encodes a protein that is the same as or highly similar to the sequence encoded by the first locus, its sequence may be varied to decrease the likelihood of undesired integration into the first locus. For example, the sequence to be integrated into the second locus may have differences in the promoter sequence, termination sequence, codon usage, and/or other tolerable sequence differences relative to the sequence integrated into the first locus.

Transformation of haploid P. pastoris strains and genetic manipulation of the P. pastoris sexual cycle may be performed as described in Pichia Protocols (1998, 2007), supra.

Expression vectors for use in the methods of the invention may further include yeast specific sequences, including a selectable auxotrophic or drug marker for identifying transformed yeast strains. A drug marker may further be used to amplify copy number of the vector in a yeast cell, e.g., by culturing a population of cells in an elevated concentration of the drug, thereby selecting transformants that express elevated levels of the resistance gene.

The polypeptide coding sequence of interest is typically operably linked to transcriptional and translational regulatory sequences that provide for expression of the polypeptide in yeast cells. These vector components may include, but are not limited to, one or more of the following: an enhancer element, a promoter, and a transcription termination sequence. Sequences for the secretion of the polypeptide may also be included, e.g. a signal sequence, and the like. A yeast origin of replication is optional, as expression vectors are often integrated into the yeast genome.

In an exemplary embodiment, one or more of the genes encoding the desired protein or subunits thereof are coupled to an inducible promoter. Suitable exemplary promoters include the alcohol oxidase 1 gene promoter, formaldehyde dehydrogenase genes (FLD; see U.S. Pub. No. 2007/0298500), and other inducible promoters known in the art. The alcohol oxidase 1 gene promoter, is tightly repressed during growth of the yeast on most common carbon sources, such as glucose, glycerol, or ethanol, but is highly induced during growth on methanol (Tschopp et al., 1987; U.S. Pat. No. 4,855,231 to Stroman, D. W., et al). For production of foreign proteins, strains may be initially grown on a repressing carbon source to generate biomass and then shifted to methanol as the sole (or main) carbon and energy source to induce expression of the foreign gene. One advantage of this regulatory system is that P. pastoris strains transformed with foreign genes whose expression products are toxic to the cells can be maintained by growing under repressing conditions.

In another exemplary embodiment, one or more of the desired genes may be coupled to a regulated promoter, whose expression level can be upregulated under appropriate conditions. Examples of suitable promoters from Pichia include the CUP1 (induced by the level of copper in the medium), tetracycline inducible promoters, thiamine inducible promoters, AOX1 promoter (Cregg et al. (1989) Mol. Cell. Biol. 9:1316-1323); ICL1 promoter (Menendez et al. (2003) Yeast 20(13):1097-108); glyceraldehyde-3-phosphate dehydrogenase promoter (GAP) (Waterham et al. (1997) Gene 186(1):37-44); and FLD1 promoter (Shen et al. (1998) Gene 216(1):93-102). The GAP promoter is a strong constitutive promoter and the CUP1, AOX and FLD1 promoters are inducible. Each foregoing reference is incorporated by reference herein in its entirety.

Other yeast promoters include ADH1, alcohol dehydrogenase II, GAL4, PHO3, PHO5, Pyk, and chimeric promoters derived therefrom. Additionally, non-yeast promoters may be used in the invention such as mammalian, insect, plant, reptile, amphibian, viral, and avian promoters. Most typically the promoter will comprise a mammalian promoter (potentially endogenous to the expressed genes) or will comprise a yeast or viral promoter that provides for efficient transcription in yeast systems.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the fungal cell. The S. cerevisiae alpha factor pre-pro signal has proven effective in the secretion of a variety of recombinant proteins from P. pastoris. Other yeast signal sequences include the alpha mating factor signal sequence, the invertase signal sequence, and signal sequences derived from other secreted yeast polypeptides. Additionally, these signal peptide sequences may be engineered to provide for enhanced secretion in diploid yeast expression systems. Other secretion signals of interest also include mammalian signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et. al. Protein Eng 11(2) 75 (1998); and Kobayashi et. al. Therapeutic Apheresis 2(4) 257 (1998), each of which is incorporated by reference herein in its entirety.

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Though optional, in one embodiment, one or more subunit of the desired protein or multi-subunit complex is operably linked, or fused, to a secretion sequence that provides for secretion of the expressed polypeptide into the culture media, which can facilitate harvesting and purification of the desired protein or multi-subunit complex. Even more preferably, the secretion sequences provide for optimized secretion of the polypeptide from the fungal cells (e.g., yeast diploid cells), such as through selecting preferred codons and/or altering the percentage of AT base pairs through codon selection. It is known in the art that secretion efficiency and/or stability can be affected by the choice of secretion sequence and the optimal secretion sequence can vary between different proteins (see, e.g., Koganesawa et al., Protein Eng. 2001 September; 14(9):705-10, which is incorporated by reference herein in its entirety). Many potentially suitable secretion signals are known in the art and can readily be tested for their effect upon yield and/or purity of a particular desired protein or multi-subunit complex. Any secretion sequences may potentially be used, including those present in secreted proteins of yeasts and other species, as well as engineered secretion sequences. See Hashimoto et al., Protein Engineering vol. 11 no. 2 pp.75-77, 1998; Oka et al., Biosci Biotechnol Biochem. 1999 November; 63(11):1977-83; Gellissen et al., FEMS Yeast Research 5 (2005) 1079-1096; Ma et al., Hepatology. 2005 December; 42(6):1355-63; Raemaekers et al., Eur J Biochem. 1999 Oct. 1; 265(1):394-403; Koganesawa et al., Protein Eng. (2001) 14 (9): 705-710; Daly et al., Protein Expr Purif. 2006 April; 46(2):456-67; Damasceno et al., Appl Microbiol Biotechnol (2007) 74:381-389; and Felgenhauer et al., Nucleic Acids Res. 1990 Aug. 25; 18(16):4927, each of which is incorporated by reference herein in its entirety).

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking may be accomplished by ligation at convenient restriction sites or alternatively via a PCR/recombination method familiar to those skilled in the art (Gateway® Technology; Invitrogen, Carlsbad Calif.). If such sites do not exist, the synthetic oligonucleotide adapters or linkers may be used in accordance with conventional practice. Desired nucleic acids (including nucleic acids comprising operably linked sequences) may also be produced by chemical synthesis.

The protein may also be secreted into the culture media without being operably linked or fused to a secretion signal. For example, it has been demonstrated that some desired polypeptides are secreted into the culture media when expressed in P. pastoris even without being linked or fused to a secretion signal. Additionally, the protein may be purified from fungal cells (which, for example, may be preferable if the protein is poorly secreted) using methods known in the art.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

As used herein the terms “filamentous fungal cell”, “filamentous fungal host cell”, and “filamentous fungus” are used interchangeably and are intended to mean any cell from any species from the genera Aspergillus, Trichoderma, Penicillium, Rhizopus, Paecilomyces, Fusarium, Neurospora and Claviceps. The filamentous fungi include but are not limited to Trichoderma reesei, Aspergillus spp., Aspergillus niger, Aspergillus nidulans, Aspergillus awamori, Aspergillus oryzae, Neurospora crassa, Penicillium spp., Penicillium chrysogenum, Penicillium purpurogenum, Penicillium funiculosum, Penicillium emersonii, Rhizopus spp., Rhizopus miehei, Rhizopus oryzae, Rhizopus pusillus, Rhizopus arrhizus, Phanerochaete chrysosporium, and Fusarium graminearum. In the present invention this is intended to broadly encompass any filamentous fungal cell that can be grown in culture.

As used herein the term “yeast cell” refers to any cell from any species from the genera Arxiozyma; Ascobonyozyma; Citeromyces; Debaryomyces; Dekkera; Eremothecium; Issatchenkia; Kazachstania; Kluyveromyces; Kodamaea; Lodderomyces; Pachysolen; Pichia; Saccharomyces; Saturnispora; Tetrapisispora; Torulaspora; Williopsis; Zygosaccharomyces; Yarrowia; Rhodosporidium; Candida; Hansenula; Filobasium; Sporidiobolus; Bullera; Leucosporidium and Filobasidella. The yeasts include but are not limited to Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia lipolytica, and Pichia pastoris (Komagataella pastoris). In the present invention, this is intended to broadly encompass any yeast cell that can be grown in culture.

In a preferred embodiment of the invention, the yeast cell is a member of the genus Pichia or is another methylotroph. In a further preferred embodiment of the invention, the fungal cell is of the genus Pichia is one of the following species: Pichia pastoris, Pichia methanolica, and Hansenula polymorpha (Pichia angusta). In a particularly preferred embodiment of the invention, the fungal cell of the genus Pichia is the species Pichia pastoris.

Such species may exist in a haploid, diploid, or other polyploid form. The cells of a given ploidy may, under appropriate conditions, proliferate for an indefinite number of generations in that form. Diploid cells can also sporulate to form haploid cells. Sequential mating can result in tetraploid strains through further mating or fusion of diploid strains. The present invention contemplates the use of haploid yeast, as well as diploid or other polyploid yeast cells produced, for example, by mating or fusion (e.g., spheroplast fusion).

As used herein “haploid yeast cell” refers to a cell having a single copy of each gene of its normal genomic (chromosomal) complement.

As used herein, “polyploid yeast cell” refers to a cell having more than one copy of its normal genomic (chromosomal) complement.

As used herein, “diploid yeast cell” refers to a cell having two copies (alleles) of essentially every gene of its normal genomic complement, typically formed by the process of fusion (mating) of two haploid cells.

As used herein, “tetraploid yeast cell” refers to a cell having four copies (alleles) of essentially every gene of its normal genomic complement, typically formed by the process of fusion (mating) of two diploid cells. Tetraploids may carry two, three, four, or more different expression cassettes. Such tetraploids might be obtained in S. cerevisiae by selective mating homozygotic heterothallic a/a and alpha/alpha diploids and in Pichia by sequential mating of haploids to obtain auxotrophic diploids. For example, a [met his] haploid can be mated with [ade his] haploid to obtain diploid [his]; and a [met arg] haploid can be mated with [ade arg] haploid to obtain diploid [arg]; then the diploid [his] can be mated with the diploid [arg] to obtain a tetraploid prototroph. It will be understood by those of skill in the art that reference to the benefits and uses of diploid cells may also apply to tetraploid cells.

As used herein, “yeast mating” refers to the process by which two yeast cells fuse to form a single yeast cell. The fused cells may be haploid cells or cells of higher ploidy (e.g., mating two diploid cells to produce a tetraploid cell).

As used herein, “meiosis” refers to the process by which a diploid yeast cell undergoes reductive division to form four haploid spore products. Each spore may then germinate and form a haploid vegetatively growing cell line.

As used herein, “folding” refers to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

The expression host may be further modified by the introduction of sequences encoding one or more enzymes that enhance folding and disulfide bond formation, i.e. foldases, chaperonins, etc. Such sequences may be constitutively or inducibly expressed in the yeast host cell, using vectors, markers, etc. as known in the art. Preferably the sequences, including transcriptional regulatory elements sufficient for the desired pattern of expression, are stably integrated in the yeast genome through a targeted methodology.

For example, the eukaryotic Protein Disulfide Isomerase (PDI) is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of BIP (immunoglobulin heavy chain binding protein); cyclophilin; and the like. In one embodiment of the invention, the desired protein or multi-subunit complex may be expressed from a yeast strain produced by mating, wherein each of the haploid parental strains expresses a distinct folding enzyme, e.g. one strain may express BIP, and the other strain may express PDI or combinations thereof.

The terms “desired protein” and “desired polypeptide” are used interchangeably and refer generally to a protein (typically a heterologous or recombinantly expressed protein) expressed in a host yeast or filamentous fungal cell comprising a particular primary structure (i.e., sequence). The desired protein may be a homopolymeric or heteropolymeric multi-subunit protein complex. Exemplary multimeric recombinant proteins include, but are not limited to, a multimeric hormone (e.g., insulin family, relaxin family and other peptide hormones), growth factor, receptor, antibody, cytokine, receptor ligand, transcription factor or enzyme.

Preferably, the desired protein is an antibody or an antibody fragment, such as a humanized or human antibody or a binding portion thereof. In one aspect, the humanized antibody is of mouse, rat, rabbit, goat, sheep, or cow origin. Preferably, the humanized antibody is of rabbit origin. In another aspect, the antibody or antibody fragment comprises a monovalent, bivalent, or multivalent antibody. In yet another aspect, the antibody or antibody fragment specifically binds to IL-2, IL-4, IL-6, IL-10, IL-12, IL-13, IL-17, IL-18, IFN-alpha, IFN-gamma, BAFF, CXCL13, IP-10, CBP, angiotensin (angiotensin I and angiotensin II), Nav1.7, Nav1.8, VEGF, PDGF, EPO, EGF, FSH, TSH, hCG, CGRP, NGF, TNF, HGF, BMP2, BMP7, PCSK9 or HRG.

As used herein, the term “molecular crowding agent” refers to agents that can decrease the volume of accessible solvent, including macromolecular molecular crowding agents and kosmotropic molecular crowding agents. Molecular crowding agents include volume occupying agents that can greatly increase the effective concentration of solutes due to steric repulsion resulting in volume exclusion, such that the solute is restricted to a lesser volume. Additional exemplary molecular crowding agents include lower molecular weight agents thought to operate by structuring water (i.e., kosmotropes) resulting in a volume exclusion effect. The impact of the volume exclusion effect typically increases with the size of the solute, as larger molecules are less able to fit into spaces between molecular crowding agent molecules or structured water. Molecular crowding agents are described in the literature to mimic intracellular conditions, in which reaction kinetics can be greatly altered as a result of the increased effective concentration of agents (see, e.g., Cheung et al., PNAS, 2005 Mar. 29; 102(13):4753-8; Ellis, Trends Biochem Sci. 2001 October; 26(10):597-604; and Ellis, Curr Opin Struct Biol. 2001 February; 11(1):114-9, each of which is hereby incorporated by reference in its entirety). Without intent to be limited by theory, it is believed that the presence of molecular crowding agents can increase the rate at which polypeptides (such as antibodies) exported from a cell can interact with a molecule at the cell surface (such as a capture reagent), thereby increasing the rate of capture of exported polypeptides by the particular cell that exported them. Also without intent to be limited by theory, it is believed that molecular crowding agents may decrease the rate at which a polypeptide exported from a cell can diffuse to the proximity of a different cell, which would decrease “cross-binding” effects wherein a polypeptide exported from one cell could bind to a capture reagent at the surface of another cell. Molecular crowding agents include natural and synthetic molecules. Exemplary macromolecular molecular crowding agents include macromolecules such as polymers (including without limitation polyethylene glycols, polypropylene glycols, and polyvinyl alcohols), hemoglobins, serum albumins (including bovine serum albumin (BSA) and human serum albumin (HSA), among others), ovalbumins, dextrans (such as dextran 70), and Ficoll™. Ficoll™ refers to a group of neutral, highly branched, high-mass, hydrophilic polysaccharides, which typically are inert and polar, and generally do not interact with proteins. An exemplary Ficoll™ is Ficoll™ 70, a sucrose epichlorohydrin copolymer having an average molecular mass of 74 kDa. Additionally, as noted, molecular crowding agents include kosmotropic molecules that can increase the stability and structure of water-water interactions, such as ionic kosmotropes including CO₂⁻³, SO₂⁻⁴, HPO₂⁻⁴, magnesium(2+), lithium (1+), zinc (2+) and aluminium (+3), as well as salts thereof, as well as non-ionic kosmotropes, including sugars (such as trehalose and glucose) as well as proline and tert-butanol. Macromolecular molecular crowding agents can be included in the compositions in amounts from about 5% to about 50% w/v (e.g., about 5%, about 10%, about 15%, about 20%, about 25%, about 30%, about 35%, about 40%, about 45%, or about 50% w/v, or any range between.) The concentration of a given molecular crowding agent that can decrease “cross-binding” effects may be determined through routine experimentation, for example using the experimental methodologies described in Example 10 herein.

The term “antibody” includes any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” A preferred source for producing antibodies useful as starting material according to the invention is rabbits. Numerous antibody coding sequences have been described; and others may be raised by methods well-known in the art. Examples thereof include chimeric antibodies, human antibodies and other non-human mammalian antibodies, humanized antibodies, human antibodies, single chain antibodies such as scFvs, camelbodies, nanobodies, IgNAR (single-chain antibodies derived from sharks), small-modular immunophannaceuticals (SMIPs), and antibody fragments such as Fabs, Fab′, F(ab′)₂ and the like. See Streltsov V A, et al., Structure of a shark IgNAR antibody variable domain and modeling of an early-developmental isotype, Protein Sci. 2005 November; 14(11):2901-9. Epub 2005 Sep. 30; Greenberg A S, et al., A new antigen receptor gene family that undergoes rearrangement and extensive somatic diversification in sharks, Nature. 1995 Mar. 9; 374(6518):168-73; Nuttall S D, et al., Isolation of the new antigen receptor from wobbegong sharks, and use as a scaffold for the display of protein loop libraries, Mol Immunol. 2001 August; 38(4):313-26; Hamers-Casterman C, et al., Naturally occurring antibodies devoid of light chains, Nature. 1993 Jun. 3; 363(6428):446-8; Gill D S, et al., Biopharmaceutical drug discovery using novel protein scaffolds, Curr Opin Biotechnol. 2006 December; 17(6):653-8. Epub 2006 Oct. 19. Each foregoing reference is incorporated by reference herein in its entirety.

For example, antibodies or antigen binding fragments may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions (V_{L} and V_{H}), obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the invention may be selected from IgG 1, IgG2, IgG3 or IgG4 constant regions.

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference. Methods of humanizing antibodies have been described previously in issued U.S. Pat. No. 7935340, the disclosure of which is incorporated herein by reference in its entirety. In some instances, a determination of whether additional rabbit framework residues are required to maintain activity is necessary. In some instances the humanized antibodies still requires some critical rabbit framework residues to be retained to minimize loss of affinity or activity. In these cases, it is necessary to change single or multiple framework amino acids from human germline sequences back to the original rabbit amino acids in order to have desired activity. These changes are determined experimentally to identify which rabbit residues are necessary to preserve affinity and activity.

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in the present invention may be produced by synthesizing a fused variable light chain region and a variable heavy chain region. Combinations of antibodies are also of interest, e.g. diabodies, which comprise two distinct Fv specificities. In another embodiment of the invention, SMIPs (small molecule immunopharmaceuticals), camelbodies, nanobodies, and IgNAR are encompassed by immunoglobulin fragments.

Immunoglobulins and fragments thereof may be modified post-translationally, e.g. to add effector moieties such as chemical linkers, detectable moieties, such as fluorescent dyes, enzymes, toxins, substrates, bioluminescent materials, radioactive materials, chemiluminescent moieties and the like, or specific binding moieties, such as streptavidin, avidin, or biotin, and the like may be utilized in the methods and compositions of the present invention. Examples of additional effector molecules are provided infra.

As used herein, “half antibody”, “half-antibody species” or “H1L1” refer to a protein complex that includes a single heavy and single light antibody chain, but lacks a covalent linkage to a second heavy and light antibody chain. Two half antibodies may remain non-covalently associated under some conditions (which may give behavior similar to a full antibody, e.g., apparent molecular weight determined by size exclusion chromatography). Similarly, H2L1 refers to a protein complex that includes two heavy antibody chains and single light antibody chain, but lacks a covalent linkage to a second light antibody chain; these complexes may also non-covalently associate with another light antibody chain (and likewise give similar behavior to a full antibody). Like full antibodies, half antibody species and H2L1 species can dissociate under reducing conditions into individual heavy and light chains. Half antibody species and H2L1 species can be detected on a non-reduced SDS-PAGE gel as a species migrating at a lower apparent molecular weight than the full antibody, e.g., H1L1 migrates at approximately half the apparent molecular weight of the full antibody (e.g., about 75 kDa).

As used herein, “polyploid yeast that stably expresses or expresses a desired polypeptide for prolonged time” refers to a yeast culture that secretes said polypeptide for at least several days to a week, more preferably at least a month, still more preferably at least 1-6 months, and even more preferably for more than a year at threshold expression levels, typically at least 50-500 mg/liter (after about 90 hours in culture) and preferably substantially greater.

As used herein, “polyploidal yeast culture that secretes desired amounts of desired polypeptide” refers to cultures that stably or for prolonged periods secrete at least at least 50-500 mg/liter, and most preferably 500-1000 mg/liter or more.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “coding sequence” is an in-frame sequence of codons that (in view of the genetic code) correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of binding RNA polymerase in a cell and initiating transcription of a downstream (3′ direction) coding sequence. Promoter sequences typically contain additional sites for binding of regulatory molecules (e.g., transcription factors) which affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

Vectors are used to introduce a foreign substance, such as DNA, RNA or protein, into an organism or host cell. Typical vectors include recombinant viruses (for polynucleotides) and liposomes (for polypeptides). A “DNA vector” is a replicon, such as plasmid, phage or cosmid, to which another polynucleotide segment may be attached so as to bring about the replication of the attached segment. An “expression vector” is a DNA vector which contains regulatory sequences which will direct polypeptide synthesis by an appropriate host cell. This usually means a promoter to bind RNA polymerase and initiate transcription of mRNA, as well as ribosome binding sites and initiation signals to direct translation of the mRNA into a polypeptide(s). Incorporation of a polynucleotide sequence into an expression vector at the proper site and in correct reading frame, followed by transformation of an appropriate host cell by the vector, enables the production of a polypeptide encoded by said polynucleotide sequence.

“Amplification” of polynucleotide sequences is the in vitro production of multiple copies of a particular nucleic acid sequence. The amplified sequence is usually in the form of DNA. A variety of techniques for carrying out such amplification are described in the following review articles, each of which is incorporated by reference herein in its entirety: Van Brunt 1990, Bio/Technol., 8(4):291-294; and Gill and Ghaemi, Nucleosides Nucleotides Nucleic Acids. 2008 March; 27(3):224-43. Polymerase chain reaction or PCR is a prototype of nucleic acid amplification, and use of PCR herein should be considered exemplary of other suitable amplification techniques.

The general structure of antibodies in most vertebrates (including mammals) is now well understood (Edelman, G. M., Ann. N.Y. Acad. Sci., 190: 5 (1971)). Conventional antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the F_{ab} region; the stem portion of the “Y” configuration is designated the F_{C} region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to gamma, mu, alpha, delta, and epsilon heavy chain constant regions). The constant region or class determines subsequent effector function of the antibody, including activation of complement (Kabat, E. A., Structural Concepts in Immunology and Immunochemistry, 2nd Ed., p. 413-436, Holt, Rinehart, Winston (1976)), and other cellular responses (Andrews, D. W., et al., Clinical Immunobiology, pp 1-18, W. B. Sanders (1980); Kohl, S., et al., Immunology, 48: 187 (1983)); while the variable region determines the antigen with which it will react. Light chains are classified as either kappa or lambda. Each heavy chain class can be paired with either kappa or lambda light chain. The light and heavy chains are covalently bonded to each other, and the “tail” portions of the two heavy chains are bonded to each other by covalent disulfide linkages when the immunoglobulins are generated either by hybridomas or by B cells.

The expression “variable region” or “VR” refers to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

The expressions “complementarity determining region,” “hypervariable region,” or “CDR” refer to one or more of the hyper-variable or complementarity determining regions (CDRs) found in the variable regions of light or heavy chains of an antibody (See Kabat, E. A. et al., Sequences of Proteins of Immunological Interest, National Institutes of Health, Bethesda, Md., (1987)). These expressions include the hypervariable regions as defined by Kabat et al. (“Sequences of Proteins of Immunological Interest,” Kabat E., et al., US Dept. of Health and Human Services, 1983) or the hypervariable loops in 3-dimensional structures of antibodies (Chothia and Lesk, J Mol. Biol. 196 901-917 (1987)). The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction (Kashmiri, S., Methods, 36:25-34 (2005)).

The expressions “framework region” or “FR” refer to one or more of the framework regions within the variable regions of the light and heavy chains of an antibody (See Kabat, E. A. et al., Sequences of Proteins of Immunological Interest, National Institutes of Health, Bethesda, Md., (1987)). These expressions include those amino acid sequence regions interposed between the CDRs within the variable regions of the light and heavy chains of an antibody.

The expression “stable copy number” refers to a host cell that substantially maintains the number of copies of a gene (such as an antibody chain gene) over a prolonged period of time (such as at least a day, at least a week, or at least a month, or more) or over a prolonged number of generations of propagation (e.g., at least 30, 40, 50, 75, 100, 200, 500, or 1000 generations, or more). For example, at a given time point or number of generations, at least 50%, and preferably at least 70%, 75%, 85%, 90%, 95%, or more of cells in the culture may maintain the same number of copies of the gene as in the starting cell. In a preferred embodiment, the host cell contains a stable copy number of the gene encoding the desired protein or encoding each subunit of the desired multi-subunit complex (e.g., antibody).

The expression “stably expresses” refers to a host cell that maintains similar levels of expression of a gene or protein (such as an antibody) over a prolonged period of time (such as at least a day, at least a week, or at least a month, or more) or over a prolonged number of generations of propagation (e.g., at least 30, 40, 50, 75, 100, 200, 500, or 1000 generations, or more). For example, at a given time point or number of generations, the rate of production or yield of the gene or protein may be at least 50%, and preferably at least 70%, 75%, 85%, 90%, 95%, or more of the initial rate of production. In a preferred embodiment, the host cell stably expresses the desired protein or multi-subunit complex (e.g., antibody).

### Recovery and Purification of Desired Proteins

Monoclonal antibodies have become prominent therapeutic agents, but their purification process needs to reliably and predictably produce a product suitable for use in humans. Impurities such as host cell protein, DNA, adventitious and endogenous viruses, endotoxin, aggregates and other species, e.g., glycovariants, typically are controlled while maintaining an acceptable yield of the desired antibody product. In addition, impurities introduced during the purification process (e.g., leached Protein A, extractables from resins and filters, process buffers and agents such as detergents) typically are removed as well before the antibody can be used as a therapeutic agent.

### Primary Recovery Processes

The first step in the recovery of an antibody from cell culture is harvest. Cells and cell debris are removed to yield a clarified, filtered fluid suitable for chromatography, i.e., harvested cell culture fluid (HCCF). Exemplary methods for primary recovery include centrifugation, depth filtration and sterile filtration, flocculation, precipitation and/or other applicable approaches depending on scale and facility capability.

### Centrifugation

In one embodiment, cells and flocculated debris are removed from broth by centrifugation. Centrifugation can be used for pilot and commercial scale manufacturing. Preferably, centrifugation is used in large-scale manufacturing to provide harvested cell culture fluid from cell cultures with percent solids of >3% (i.e., increased levels of sub-micron debris).

Standard non-hermetic disc-stack centrifuges as well fully hermetic centrifuges as are capable of removing cells and large cell debris, although fully hermetic centrifuges can significantly reduce the amount of cell lysis that is incurred during this unit operation, e.g., by at least 50%, by preventing overflow and minimizing shear.

The clarification efficiency of the centrifugation process is affected by harvest parameters such as centrifuge feed rate, G-force, bowl geometry, operating pressures, discharge frequency and ancillary equipment used in the transfer of cell culture fluid to the centrifuge. The cell culture process characteristics such as peak cell density, total cell density and culture viability during the culture process and at harvest can also affect separation performance. The centrifugation process can be optimized to select the feed rate and bowl rotational speed using the scaling factors of feed rate (Q) and equivalent settling area (Σ) in the centrifuge. The optimized process can minimize cell lysis and debris generation while maximizing the sedimentation of submicron particles and product yield.

Filtration

Tangential flow microfiltration can also be used in cell harvest. In particular, the cell culture fluid flows tangential to the microporous membrane, and pressure driven filtrate flow separates the soluble product from the larger, insoluble cells. Membrane fouling is limited by the inertial lift and shear-induced diffusion generated by the turbulent flow across the membrane surface.

A high yielding harvest can be achieved by a series of concentration and diafiltration steps. In the former, the volume of the cell culture fluid is reduced, which results in concentrating the solid mass. The diafiltration step then washes the product from the concentrated cell culture fluid mixture.

By way of example, a 0.22 μm pore size may be employed for the TFF membrane as it produces the target quality harvested cell culture fluid (suitable for chromatography) without the need for further clarification. Alternatively, more open pore sizes at the TFF barrier may be used to better manage fouling; however, more open pore sizes may require an additional clarification step (e.g., normal flow depth filtration) downstream of the TFF system. Preferably, TFF is used for cell cultures with percent solids of <3%.

Depth filters can also be used in the clarification of cell culture broths, to maintain capacity on membrane filters or to protect chromatography columns or virus filters. Depth filters may be composed of, e.g., cellulose, a porous filter-aid such as diatomaceous earth, an ionic charged resin binder and a binding resin (present at a small weight percent to covalently bind dissimilar construction materials together, giving the resultant media wet strength and conferring positive charge to the media surfaces). Depth filters rely on both size exclusion and adsorptive binding to effect separation. Exemplary depth filters are approximately 2-4 mm thick.

For harvesting applications, depth filters can be applied directly with the whole cell broth or in conjunction with a primary separator, e.g., TPF or centrifugation. For example, when used for whole-cell broth depth filter harvest, the filtration train contains three stages of filters: (1) the primary stage with a coarse or open depth filter with a pore size of up to 10 μm to remove whole cells and large particles; (2) the secondary stage with a tighter depth filter to clear colloidal and submicron particles; and (3) the third stage with a 0.2 μm pore size membrane filter. Although the filtration process generally scales linearly, a safety factor of 1.5× to >3× can be employed for each stage to ensure adequate filter capacity.

In one embodiment, a depth filter is employed after centrifugation to further clarify the harvested broth, e.g., because there is a practical lower limit to the particle size that can be removed by centrifugation. For example, the depth filter may comprise two distinct layers (with the upstream zone being a coarser grade compared with the downstream) and have a pore size range of 0.1-4 μm. The larger particles are trapped in the coarse grade filter media and smaller particles are trapped in the tighter media, reducing premature plugging and increasing filtration capacity.

Optimization of filter type, pore size, surface area and flux can be done at lab bench scale and then scaled up to pilot scale based on, e.g., the centrate turbidity and particle size distribution. Depth filter sizing experiments are generally performed at constant flux using pressure endpoints in any one or combination of filtration stages. Preferably, a 0.22 μm grade filter is used to filter the supernatant at the end of harvest process to control bioburden. The 0.22 μm-filtered supernatant can be stored at 2-8° C. for several days or longer without changing the antibody product-related variant profile.

Without being bound by theory, it is believed that the adsorptive mechanism of depth filters allows for their extensive use as a purification tool to remove a wide range of process contaminants and impurities. In particular, the electrostatic interactions between the positive charges of depth filters and DNA molecules as well as hydrophobic interactions between depth filter media and DNA molecules may play important roles in the adsorptive reduction of DNA. For example, charged depth filters have been used to remove DNA, and the level of charges on Zeta Plus® (Cuno) 90SP has been correlated with its ability to remove DNA. Additionally, by way of example, positively charged depth filters have been used to remove Escherichia coli-derived and other endogenous endotoxins and viruses many times smaller than the average pore size of the filter, and Zeta Plus® (Cuno) VR series depth filters were found to bind enveloped retrovirus and non-enveloped parvovirus by adsorption. Depth filtration was also employed to remove spiked prions from an immunoglobin solution. Moreover, the removal of host cell proteins through depth filtration prior to a Protein A affinity chromatography column has been shown to significantly reduce precipitation during the pH adjustment of the Protein A pool.

Flocculation and Precipitation

In one embodiment, precipitation/flocculation-based pretreatment steps are used to reduce the quantity of cell debris and colloids in the cell culture fluid, which can exceed the existing filtration train equipment capability. Flocculation involves polymer adsorption, e.g., electrostatic attraction, to the cell and cell debris by, e.g., cationic, neutral and anionic polymers, to clear cellular contaminants resulting in improved clarification efficiency and high recovery yield. Flocculation reagents, e.g., calcium chloride and potassium phosphate, at very low levels, e.g., 20-60 mM calcium chloride with an equimolar amount of phosphate added to form calcium phosphate, are believed to contribute to co-precipitation of calcium phosphate with cells, cell debris and impurities.

In one embodiment, the disclosed purification processes include treatment of the whole cell broth with ethylene diamine tetraacetic acid (EDTA) to 3 mM final concentration and with a flocculating agent, subsequent removal of cells and flocculated debris by centrifugation, followed by clarification through depth and 0.2 μm filters.

### Chromatography

In the biopharmaceutical industry, chromatography is a critical and widely used separation and purification technology due to its high resolution. Chromatography exploits the physical and chemical differences between biomolecules for separation. For example, protein A chromatography may follow harvest to yield a relatively pure product that requires removal of only a small proportion of process and product related impurities. One or two additional chromatography steps can then be employed as polishing steps, e.g., incorporating ion exchange chromatography, hydrophobic interaction chromatography, mixed mode chromatography and/or hydroxyapatite chromatography. These steps can provide additional viral, host cell protein and DNA clearance, as well as removing aggregates, unwanted product variant species and other minor contaminants. Lastly, the purified product may be concentrated and diafiltered into the final formulation buffer.

Antibody purification involves selective enrichment or specific isolation of antibodies from serum (polyclonal antibodies), ascites fluid or cell culture supernatant of a cell line (monoclonal antibodies). Purification methods range from very crude to highly specific and can be classified as follows:

Physicochemical fractionation—differential precipitation, size-exclusion or solid-phase binding of immunoglobulins based on size, charge or other shared chemical characteristics of antibodies in typical samples. This isolates a subset of sample proteins that includes the immunoglobulins.

Affinity fractionation—binding of particular antibody classes (e.g., IgG) by immobilized biological ligands (e.g., proteins) that have specific affinity to immunoglobulins (which purifies all antibodies of the target class without regard to antigen specificity) or affinity purification of only those antibodies in a sample that bind to a particular antigen molecule through their specific antigen-binding domains (which purifies all antibodies that bind the antigen without regard to antibody class or isotype).

The main classes of serum immunoglobulins (e.g., IgG and IgM) share the same general structure, including overall amino acid composition and solubility characteristics. These general properties are sufficiently different from most other abundant proteins in serum, e.g., albumin and transferrin, that the immunoglobulins can be selected and enriched for on the basis of these differentiating physicochemical properties.

Physiochemical Fractionation Antibody Purification

Ammonium Sulfate Precipitation

Ammonium sulfate precipitation is frequently used to enrich and concentrate antibodies from serum, ascites fluid or cell culture supernatant. As the concentration of the lyotropic salt is increased in a sample, proteins and other macromolecules become progressively less soluble until they precipitate, i.e., the lyotropic effect is referred to as “salting out.” Antibodies precipitate at lower concentrations of ammonium sulfate than most other proteins and components of serum.

At about 40 to about 50% ammonium sulfate saturation (100% saturation being equal to 4.32M), immunoglobulins precipitate while other proteins remain in solution. See, e.g., Harlow, E. and Lane, D. (1988). Antibodies: A Laboratory Manual. Cold Spring Harbor Laboratory Press, Cold Spring Harbor, N.Y., Gagnon, P. (1996). By way of example, an equal volume of saturated ammonium sulfate solution is slowly added to a neutralized antibody sample, followed by incubation for several hours at room temperature or 4° C. After centrifugation and removal of the supernatant, the antibody-pellet is dissolved in buffer, such as phosphate-buffered saline (PBS).

The selectivity, yield, purity and reproducibility of precipitation each depend upon several factors including, but not limited to, time, temperature, pH and rate of salt addition. See, e.g., Gagnon, P. S. (1996), Purification Tools for Monoclonal Antibodies, Validated Biosystems, Tucson, Ariz. Ammonium sulfate precipitation may provide sufficient purification for some antibody applications, but often it is performed as a preliminary step before column chromatography or other purification methods. Using partially purified antibody samples can improve the performance and extend the life of affinity columns.

Suitable antibody precipitation reagents other than ammonium sulfate for antibody purification situations include, by way of example, octonoic acid, polyethylene glycol and ethacridine.

Numerous chemically based, solid-phase chromatography methods have been adapted and optimized to achieve antibody purification in particular situations.

Ion Exchange Chromatography (IEC)

Ion exchange chromatography (IEC) uses positively or negatively charged resins to bind proteins based on their net charges in a given buffer system (pH). Conditions for IEC can be determined that bind and release the target antibody with a high degree of specificity, which may be especially important in commercial operations involving production of monoclonal antibodies. Conversely, conditions can be found that bind nearly all other sample components except antibodies. Once optimized, IEC is a cost-effective, gentle and reliable method for antibody purification.

Anion exchange chromatography uses a positively charged group immobilized to the resin. For example, weakly basic groups such as diethylamino ethyl (DEAE) or dimethylamino ethyl (DMAE), or strongly basic groups such as quaternary amino ethyl (Q) or trimethylammonium ethyl (TMAE) or quaternary aminoethyl (QAE)) can be used in anion exchange. Exemplary anion exchange media include, but are not limited to, GE Healthcare Q-Sepharose® FF, Q-Sepharose® BB, Q-Sepharose® XL, Q-Sepharose® HP, Mini Q, Mono Q, Mono P, DEAE Sepharose® FF, Source 15Q, Source 30Q, Capto Q, Streamline DEAE, Streamline QXL; Applied Biosystems Poros HQ 10 and 20 um self pack, Poros HQ 20 and 50 um, Poros PI 20 and 50 um, Poros D 50 um; Tosohaas Toyopearl® DEAE 650S M and C, Super Q 650, QAE 550C; Pall Corporation DEAE Hyper D, Q Ceramic Hyper D, Mustang Q membrane absorber; Merck KG2A Fractogel® DMAE, FractoPrep DEAE, Fractoprep TMAE, Fractogel® EMD DEAE, Fractogel® EMD TMAE; Sartorious Sartobind® Q membrane absorber.

Anion exchange is particularly useful for removing process-related impurities (e.g., host cell proteins, endogenous retrovirus and adventitious viruses such as parvovirus or pseudorabies virus, DNA, endotoxin and leached Protein A) as well as product-related impurities (e.g., dimer/aggregate). It can be used either in flow-through mode or in bind and elute mode, depending on the pI of the antibody and impurities to be removed. For example, flow-through mode is preferably used to remove impurities from antibodies having a pI above 7.5, e.g., most humanized or human IgG1 and IgG2 antibodies, because the impurities bind to the resin and the product of interest flows through. The column loading capacity, i.e., mass of antibody to mass of resin, can be quite high since the binding sites on the resin are occupied only by the impurities. Anion exchange chromatography in flow-through mode may be used as a polishing step in monoclonal antibody purification processes designed with two or three unit operations to remove residual impurities such as host cell protein, DNA, leached Protein A and a variety of viruses. By way of example, the operating pH is about 8 to about 8.2, with a conductivity of up to 10 mS/cm in the product load and equilibration and wash buffers.

Alternatively, bind and elute mode is preferably used to remove process-related and product-related impurities from antibodies having a pI in the acidic to neutral range, e.g., most humanized or human IgG4s. For bind-and-elute mode, the antibody product pool is first loaded onto an anion exchange column and the product of interest is then eluted with a higher salt concentration in a step or linear gradient, leaving the majority of impurities bound to the column. The impurities are eluted from the column during the cleaning or regeneration step. Generally, the operating pH should be above or close to the pI of the product in order to obtain a net negative charge or higher negative charge number on the surface of the antibody molecules, and, thus, to achieve a higher binding capacity during the chromatography step. Similarly, the ionic strength for the load is preferably in the low range and the pH is preferably less than pH 9.

Additionally, weak partitioning chromatography (WPC) may be used to enable a two chromatography recovery process comprising Protein A and anion exchange. Generally, the process is run isocratically (as with flow-through chromatography) but the conductivity and pH are chosen such that the binding of both the product and impurities are enhanced (in contrast to flow-through mode), attaining an antibody partition coefficient (Kp) between 0.1-20, and preferably between 1 and 3. Both antibody and impurities bind to the anion exchange resin, but the impurities are much more tightly bound than in flow-through mode, which can lead to an increase in impurity removal. Product yield in weak partitioning mode can be maximized by including a short wash at the end of the load, e.g., averaged 90% for clinical production.

Cation exchange chromatography uses a resin modified with negatively charged functional groups. For example, strong acidic ligands (e.g., sulfopropyl, sulfoethyl and sulfoisobutyl groups) or weak acidic ligands (e.g., carboxyl group) can be used in cation exchange. Exemplary cation exchange resins include, but are not limited to, GE Healthcare SP-Sepharose® FF, SP-Sepharose® BB, SP-Sepharose® XL, SP-Sepharose®HP, Mini S, Mono S, CM Sepharose® FF, Source 15S, Source 30S, Capto S, MacroCap SP, Streamline SP-XL, Streamline CST-1; Tosohaas Resins Toyopearl® Mega Cap TI SP-550 EC, Toyopearl® Giga Cap S-650M, Toyopearl® 650S, M and C, Toyopeal SP650S, M, and C, Toyopeal SP550C; JT Baker Resins Carboxy-Sulphon-5, 15 and 40 um, Sulfonic-5, 15, and 40 um; YMC BioPro S; Applied Biosystems Poros HS 20 and 50 um, Poros S 10 and 20 um; Pall Corp S Ceramic Hyper D, CM Ceramic Hyper D; Merck KGgA Resins Fractogel® EMD SO₃, Fractogel® EMD COO—, Fractogel® EMD SE Hicap, Fracto Prep SO3; Eshmuno S; Biorad Resin Unosphere S; Sartorius Membrane Sartobind® S membrane absorber.

Cation exchange chromatography is particularly suited for purification processes for many monoclonal antibodies with pI values ranging from neutral to basic, e.g., human or humanized IgG1 and IgG2 subclasses. In general, the antibody is bound onto the resin during the loading step and eluted through either increasing conductivity or increasing pH in the elution buffer. The most negatively charged process-related impurities such as DNA, some host cell protein, leached Protein A and endotoxin are removed in the load and wash fraction. Cation exchange chromatography can also reduce antibody variants from the target antibody product such as deamidated products, oxidized species and N-terminal truncated forms, as well as high molecular weight species.

The maximum binding capacity attained can be as high as >100 g/L of resin volume depending on the loading conditions, resin ligand and density, but impurity removal depends highly on the loading density. The same principles described for anion exchange chromatography regarding development of the elution program apply to cation exchange chromatography as well.

The development of elution conditions is linked to impurity removal and characteristics of the product pool that can be processed easily in the subsequent unit operation. Generally, a linear salt or pH gradient elution program can be conducted to determine the best elution condition. For example, linear gradient elution conditions may range from 5 mM to 250 mM NaCl at pH 6 and linear pH gradient elution runs may range from pH 6 to pH 8.

Immobilized Metal Chelate Chromatography (IMAC)

Immobilized metal chelate chromatography (IMAC) uses chelate-immobilized divalent metal ions (e.g., nickel Ni2+) to bind proteins or peptides that contain clusters of three or more consecutive histidine residues. This strategy can be particularly useful for purification of recombinant proteins that have been engineered to contain a terminal 6× His fusion tag. Mammalian IgGs are one of the few abundant proteins in serum (or monoclonal cell culture supernatant) that possess histidine clusters capable of being bound by immobilized nickel. Like IEC, IMAC conditions for binding and elution can be optimized for particular samples to provide gentle and reliable antibody purification. For example, IMAC may be used to separate AP- or HRP-labeled (enzyme-conjugated) antibody from excess, non-conjugated enzyme following a labeling procedure.

Hydrophobic Interaction Chromatography (HIC)

Hydrophobic interaction chromatography (HIC) separates proteins based on their hydrophobicity, and is complementary to other techniques that separate proteins based on charge, size or affinity. For example, a sample loaded on the HIC column in a high salt buffer which reduces solvation of the protein molecules in solution, thereby exposing hydrophobic regions in the sample protein molecules that consequently bind to the HIC resin. Generally, the more hydrophobic the molecule, the less salt is needed to promote binding. A gradient of decreasing salt concentration can then be used to elute samples from the HIC column. In particular, as the ionic strength decreases, the exposure of the hydrophilic regions of the molecules increases and molecules elute from the column in order of increasing hydrophobicity.

HIC in flow-through mode can be efficient in removing a large percentage of aggregates with a relatively high yield. HIC in bind-and-elute mode may provide effective separation of process-related and product-related impurities from antibody product. In particular, the majority of host cell protein, DNA and aggregates can be removed from the antibody product through selection of a suitable salt concentration in the elution buffer or use of a gradient elution method.

Exemplary HIC resins include, but are not limited to, GE Healthcare HIC Resins (Butyl Sepharose® 4 FF, Butyl-S Sepharose® FF, Octyl Sepharose® 4 FF, Phenyl Sepharose® BB, Phenyl Sepharose® HP, Phenyl Sepharose® 6 FF High Sub, Phenyl Sepharose® 6 FF Low Sub, Source 15ETH, Source 15ISO, Source 15PHE, Capto Phenyl, Capto Butyl, Sreamline Phenyl); Tosohaas HIC Resins (TSK Ether 5PW (20 um and 30 um), TSK Phenyl 5PW (20 um and 30 um), Phenyl 650S, M, and C, Butyl 650S, M and C, Hexyl-650M and C, Ether-650S and M, Butyl-600M, Super Butyl-550C, Phenyl-600M; PPG-600M); Waters HIC Resins (YMC-Pack Octyl Columns-3, 5, 10P, 15 and 25 um with pore sizes 120, 200, 300 A, YMC-Pack Phenyl Columns-3, 5, 10P, 15 and 25 um with pore sizes 120, 200 and 300 A, YMC-Pack Butyl Columns-3, 5, 10P, 15 and 25 um with pore sizes 120, 200 and 300 A); CHISSO Corporation HIC Resins (Cellufine Butyl, Cellufine Octyl, Cellufine Phenyl); JT Baker HIC Resin (WP HI-Propyl (C3)); Biorad HIC Resins (Macroprep t-Butyl, Macroprep methyl); and Applied Biosystems HIC Resin (High Density Phenyl—HP2 20 um). For example, PPG 600-M is characterized by an exclusion limit molecular weight of approximately 8×10⁵ Dalton, a polypropylene glycol PPG ligand, a 45-90 μm particle size, hydrophobicity given by the relationship Ether >PPG >Phenyl, and Dynamic Binding capacity (MAb: Anti LH) of 38 mg/mL-gel.

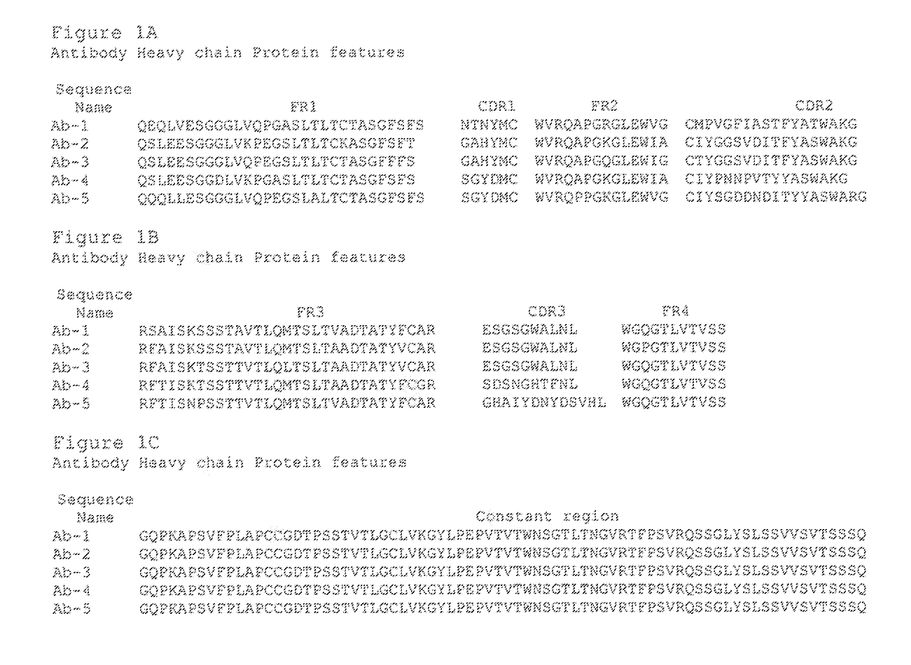

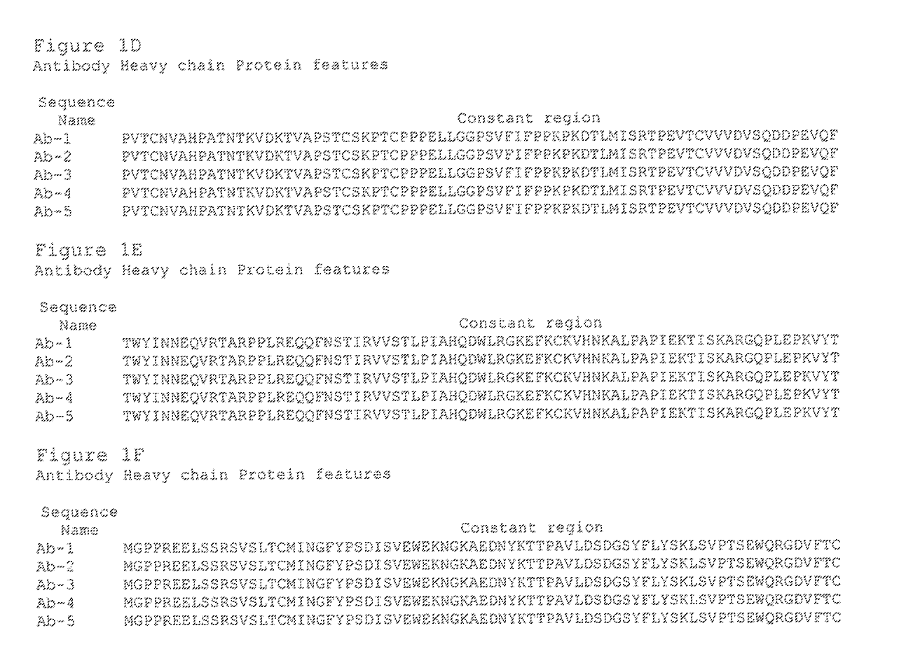

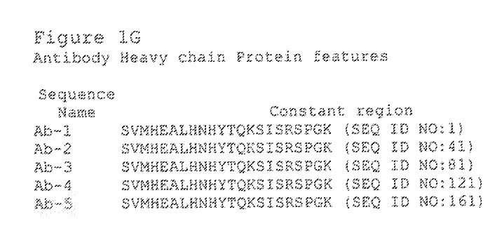

In one embodiment, the disclosed purification processes employ hydrophobic interaction chromatography (HIC) as a polish purification step after affinity chromatography (e.g., Protein A) and mixed mode chromatography (e.g., hydroxyapatite). See, FIG. 1. Preferably, polypropylene glycol (PPG-600M) or Phenyl-600M is the HIC resin. In one embodiment, the elution is performed as a linear gradient (0-100%) from about 0.7 M to 0 M sodium sulfate in a 20 mM sodium phosphate, pH 7, buffer. Optionally the OD₂₈₀ of the effluent is monitored and a series of fractions, e.g., about one-third of the collection volume, is collected for further purity analysis. Preferably, the fractions collected include from 0.1 OD on the front flank to 0.1 OD on the rear flank.

Hydrophobic Charge Induction Chromatography (HCIC)

Hydrophobic charge induction chromatography (HCIC) is based on the pH-dependent behavior of ligands that ionize at low pH. This technique employs heterocyclic ligands at high densities so that adsorption can occur via hydrophobic interactions without the need for high concentrations of lyotropic salts. Desorption in HCIC is facilitated by lowering the pH to produce charge repulsion between the ionizable ligand and the bound protein. An exemplary commercial HCIC resin is MEP-Hypercel (Pall Corporation), which is a cellulose-based media with 4-mercaptoethyl pyridine as the functional group. The ligand is a hydrophobic moiety with an N-heterocyclic ring that acquires a positive charge at low pH.

Thiophilic Adsorption

Thiophilic adsorption is a highly selective type of protein-ligand interaction, combining the properties of hydrophobic interaction chromatography (HIC) and ammonium sulfate precipitation (i.e., the lyotropic effect), that involves the binding of proteins to a sulfone group in close proximity to a thioether. In contrast to strict HIC, thiophilic adsorption depends upon a high concentration of lyotropic salt (e.g., potassium sulfate as opposed to sodium chloride). For example, binding is quite specific for a typical antibody sample that has been equilibrated with potassium sulfate. After non-bound components are washed away, the antibodies are easily recovered with gentle elution conditions (e.g., 50 mM sodium phosphate buffer, pH 7 to 8). Thiophilic Adsorbent (also called T-Gel) is 6% beaded agarose modified to contain the sulfone-thioether ligand, which has a high binding capacity and broad specificity toward immunoglobulin from various animal species.

### Affinity Purification of Antibodies

Affinity chromatography (also called affinity purification) makes use of specific binding interactions between molecules. Generally, a particular ligand is chemically immobilized or “coupled” to a solid support so that when a complex mixture is passed over the column, those molecules having specific binding affinity to the ligand become bound. After other sample components are washed away, the bound molecule is stripped from the support, resulting in its purification from the original sample.

Supports

Affinity purification involves the separation of molecules in solution (mobile phase) based on differences in binding interaction with a ligand that is immobilized to a stationary material (solid phase). A support or matrix in affinity purification is any material to which a biospecific ligand is covalently attached. Typically, the material to be used as an affinity matrix is insoluble in the system in which the target molecule is found. Usually, but not always, the insoluble matrix is a solid.

Useful affinity supports are those with a high surface-area to volume ratio, chemical groups that are easily modified for covalent attachment of ligands, minimal nonspecific binding properties, good flow characteristics and mechanical and chemical stability.

Immobilized ligands or activated affinity support chemistries are available for use in several different formats, including, e.g., cross-linked beaded agarose or polyacrylamide resins and polystyrene microplates.

Porous gel supports provide a loose matrix in which sample molecules can freely flow past a high surface area of immobilized ligand, which is also useful for affinity purification of proteins. These types of supports are usually sugar- or acrylamide-based polymer resins that are produced in solution (i.e., hydrated) as 50-150 μm diameter beads. The beaded format allows these resins to be supplied as wet slurries that can be easily dispensed to fill and “pack” columns with resin beds of any size. The beads are extremely porous and large enough that biomolecules (proteins, etc.) can flow as freely into and through the beads as they can between and around the surface of the beads. Ligands are covalently attached to the bead polymer (external and internal surfaces) by various means.

For example, cross-linked beaded agarose is typically available in 4% and 6% densities (i.e., a 1 ml resin-bed is more than 90% water by volume.) Beaded agarose may be suitable for gravity-flow, low-speed-centrifugation, and low-pressure procedures. Alternatively, polyacrylamide-based, beaded resins generally do not compress and may be used in medium pressure applications with a peristaltic pump or other liquid chromatography systems. Both types of porous support have generally low non-specific binding characteristics. A summary of the physical properties of these affinity chromatography resins is provided in Table 1 below.

TABLE 1
Physical properties of affinity chromatography resins
Physical properties of affinity chromatography resins

| | 4% crosslinked | 6% crosslinked | Acrylamide- |
|---|---|---|---|
| Support | beaded agarose | beaded agarose | azlactone polymer |
| Bead size | 45-165 μm | 45-165 μm | 50-80 μm |
| Exclusion | 20,000 kDa | 4,000 kDa | 2,000 kDa |
| limit | | | |
| Durability | crushes under | crushes under | sturdy (>100 psi, |
| | high pressure | high pressure | 6.9 bar) |
| Methods | gravity-flow or | gravity-flow or | FPLC Systems, |
| | low-speed | low-speed | HPLC, gravity |
| | centrifugation | centrifugation | flow |
| Coupling | medium | Medium | high |
| Capacity | | | |
| pH range | 3-11 | 3-11 | 1-13 |
| Form | pre-swollen | pre-swollen | dry or pre-swollen |

Magnetic particles are yet another type of solid affinity support. They are much smaller (typically 1-4 μm diameter), which provides the sufficient surface area-to-volume ratio needed for effective ligand immobilization and affinity purification. Affinity purification with magnetic particles is performed in-batch, e.g., a few microliters of beads is mixed with several hundred microliters of sample as a loose slurry. During mixing, the beads remain suspended in the sample solution, allowing affinity interactions to occur with the immobilized ligand. After sufficient time for binding has been given, the beads are collected and separated from the sample using a powerful magnet. Typically, simple bench-top procedures are done in microcentrifuge tubes, and pipetting or decanting is used to remove the sample (or wash solutions, etc.) while the magnetic beads are held in place at the bottom or side of the tube with a suitable magnet.

Magnetic particles are particularly well suited for high-throughput automation and, unlike porous resins, can be used in lieu of cell separation procedures.

Each specific affinity system requires its own set of conditions and presents its own peculiar challenges for a given research purpose. However, affinity purification generally involves the following steps:

1. Incubate crude sample with the affinity support to allow the target molecule in the sample to bind to the immobilized ligand;

2. Wash away non-bound sample components from the support; and

3. Elute (dissociate and recover) the target molecule from the immobilized ligand by altering the buffer conditions so that the binding interaction no longer occurs.

Ligands that bind to general classes of proteins (e.g., antibodies) or commonly used fusion protein tags (e.g., 6× His) are commercially available in pre-immobilized forms ready to use for affinity purification. Alternatively, more specialized ligands such as specific antibodies or antigens of interest can be immobilized using one of several commercially available activated affinity supports; for example, a peptide antigen can be immobilized to a support and used to purify antibodies that recognize the peptide.

Most commonly, ligands are immobilized or “coupled” directly to solid support material by formation of covalent chemical bonds between particular functional groups on the ligand (e.g., primary amines, sulfhydryls, carboxylic acids, aldehydes) and reactive groups on the support. However, indirect coupling approaches are also possible. For example, a GST-tagged fusion protein can be first captured to a glutathione support via the glutathione-GST affinity interaction and then secondarily chemically crosslinked to immobilize it. The immobilized GST-tagged fusion protein can then be used to affinity purify binding partner(s) of the fusion protein.

Binding and Elution Buffers for Affinity Purification

Most affinity purification procedures involving protein:ligand interactions use binding buffers at physiologic pH and ionic strength, such as phosphate buffered saline (PBS), particularly when the antibody:antigen or native protein:protein interactions are the basis for the affinity purification. Once the binding interaction occurs, the support is washed with additional buffer to remove non-bound components of the sample. Non-specific (e.g., simple ionic) binding interactions can be minimized by adding low levels of detergent or by moderate adjustments to salt concentration in the binding and/or wash buffer. Finally, elution buffer (e.g., 0.1M glycine.HCl, pH 2.5-3.0) is added to break the binding interaction (without permanently affecting the protein structure) and release the target molecule, which is then collected in its purified form. Elution buffer can dissociate binding partners by extremes of pH (low or high), high salt (ionic strength), the use of detergents or chaotropic agents that denature one or both of the molecules, removal of a binding factor or competition with a counter ligand. In some cases, subsequent dialysis or desalting may be required to exchange the purified protein from elution buffer into a more suitable buffer for storage or downstream processing.

Additionally, some antibodies and proteins are damaged by low pH, so eluted protein fractions should be neutralized immediately by addition of 1/10th volume of alkaline buffer, e.g., 1M Tris.HCl, pH 8.5. Other exemplary elution buffers for affinity purification of proteins are provided in Table 2 below.

TABLE 2
Exemplary elution buffer systems for protein affinity purification
Exemplary elution buffer systems for protein affinity purification

| Condition | Buffer |
|---|---|
| pH | 100 mM glycine•HCl, pH 2.5-3.0 |
| | 100 mM citric acid, pH 3.0 |
| | 50-100 mM triethylamine or triethanolamine, pH 11.5 |
| | 150 mM ammonium hydroxide, pH 10.5 |
| | 1M arginine, pH 4.0 |
| Ionic strength | 3.5-4.0M magnesium chloride, pH 7.0 in 10 mM Tris |
| and/or | 5M lithium chloride in 10 mM phosphate buffer, pH 7.2 |
| chaotrophic | 2.5M sodium iodide, pH 7.5 |
| effects | 0.2-3.0 sodium thiocyanate |
| Denaturing | 2-6M guanidine•HCl |
| | 2-8M urea |
| | 1% deoxycholate |
| | 1% SDS |
| Organic | 10% dioxane |
| | 50% ethylene glycol, pH 8-11.5 (also chaotropic) |
| Competitor | >0.1M counter ligand or analog |

Several methods of antibody purification involve affinity purification techniques. Exemplary approaches to affinity purification include precipitation with ammonium sulfate (crude purification of total immunoglobulin from other serum proteins); affinity purification with immobilized Protein A, G, A/G or L (bind to most species and subclasses of IgG) or recombinant Protein A, G, A/G, or L derivatives in bind & elute mode; and affinity purification with immobilized antigen (covalently immobilized purified antigen to an affinity support to isolate specific antibody from crude samples) in bind & elute mode.

Protein A, Protein G and Protein L are three bacterial proteins whose antibody-binding properties have been well characterized. These proteins have been produced recombinantly and used routinely for affinity purification of key antibody types from a variety of species. Most commercially-available, recombinant forms of these proteins have unnecessary sequences removed (e.g., the HSA-binding domain from Protein G) and are therefore smaller than their native counterparts. A genetically-engineered recombinant form of Protein A and Protein G, called Protein A/G, is also available. All four recombinant Ig-binding proteins are used routinely by researchers in numerous immunodetection and immunoaffinity applications.

To accomplish antibody purification, with Protein A, Protein G, Protein A/G are covalently immobilized onto a support, e.g., porous resins (such as beaded agarose) or magnetic beads. Because these proteins contain several antibody-binding domains, nearly every individual immobilized molecule, no matter its orientation maintains at least one functional and unhindered binding domain. Furthermore, because the proteins bind to antibodies at sites other than the antigen-binding domain, the immobilized forms of these proteins can be used in purification schemes, such as immunoprecipitation, in which antibody binding protein is used to purify an antigen from a sample by binding an antibody while it is bound to its antigen.

The high affinity of Protein A for the Fc region of IgG-type antibodies is the basis for the purification of IgG, IgG fragments and subclasses. Generally, Protein A chromatography involves passage of clarified cell culture supernatant over the column at pH about 6.0 to about 8.0, such that the antibodies bind and unwanted components, e.g., host cell proteins, cell culture media components and putative viruses, flow through the column. An optional intermediate wash step may be carried out to remove non-specifically bound impurities from the column, followed by elution of the product at pH about 2.5 to about pH 4.0. The elution step may be performed as a linear gradient or a step method or a combination of gradient and step. In one embodiment, the eluate is immediately neutralized with a neutralization buffer (e.g. 1 M Tris, pH 8), and then adjusted to a final pH 6.5 using, e.g., 5% hydrochloric acid or 1 M sodium hydroxide. Preferably, the neutralized eluate is filtered prior to subsequent chromatography. In one embodiment, the neutralized eluate is passed through a 0.2 μm filter prior to the subsequent hydroxyapatite chromatography step.

Because of its high selectivity, high flow rate and cost effective binding capacity and its capacity for extensive removal of process-related impurities such as host cell proteins, DNA, cell culture media components and endogenous and adventitious virus particles, Protein A chromatography is typically used as the first step in an antibody purification process. After this step, the antibody product is highly pure and more stable due to the elimination of proteases and other media components that may cause degradation.

There are currently three major types of Protein A resins, classified based on their resin backbone composition: glass or silica-based, e.g., AbSolute HiCap (NovaSep), Prosep vA, Prosep vA Ultra (Millipore); agarose-based, e.g., Protein A Sepharose® Fast Flow, MabSelect and MabSelect SuRe (GE Healthcare); and organic polymer based, e.g., polystyrene-divinylbenzene Poros A and MabCapture (Applied Biosystems). Preferably, the Protein A resin is an agarose-based resin, i.e., MabSelect SuRe resin. All three resin types are resistant to high concentrations of guanidinium hydrochloride, urea, reducing agents and low pH.

The column bed height employed at large scale is between 10 and 30 cm, depending on the resin particle properties such as pore size, particle size and compressibility. Preferably, the column bed height is about 25 cm. Flow rate and column dimensions determine antibody residence time on the column. In one embodiment, the linear velocity employed for Protein A is about 150 to about 500 cm/hr, preferably about 200 cm/h to about 400 cm/h, more preferably about 200 cm/h to about 300 cm/h, and most preferably about 250 cm/h. Dynamic binding capacity ranges from 15-50 g of antibody per liter of resin, and depends on the flow rate, the particular antibody to be purified, as well as the Protein A matrix used. Preferably, the column is loaded with no more than 45 g of antibody per liter of resin. A method for determining dynamic binding capacities of Protein A resins has been described by Fahr\dot{n}er et al. Biotechnol Appl BioChem. 30:121-128 (1999). A lower loading flow rate may increase antibody residence time and promote higher binding capacity. It also results in a longer processing time per cycle, requires fewer cycles and consumes less buffer per batch of harvested cell culture fluid.

Other exemplary approaches to affinity purification include lectin affinity chromatography, which can be performed in flow-through mode (product with undesired glycosylation binds to support while product without undesired glycosylation passes through the support) or bind & elute mode (product with desired glycosylation binds to support while product without desired glycosylation passes through the support).

Proteins expressed in lower eukaryotes, e.g., P. pastoris, can be modified with O-oligosaccharides solely or mainly composed of mannose (Man) residues. Additionally, proteins expressed in lower eukaryotes, e.g., P. pastoris, can be modified with N-oligosaccharides. N-glycosylation in P. pastoris and other fungi is different than in higher eukaryotes. Even within fungi, N-glycosylation differs. In particular, the N-linked glycosylation pathways in P. pastoris are substantially different from those found in S. cerevisiae, with shorter Man(alpha 1,6) extensions to the core Man8GN2 and the apparent lack of significant Man(alpha 1,3) additions representing the major processing modality of N-linked glycans in P. pastoris. In some respects, P. pastoris may be closer to the typical mammalian high-mannose glycosylation pattern. Moreover, Pichia and other fungi may be engineered to produce “humanized glycoproteins” (i.e., genetically modify yeast strains to be capable of replicating the essential glycosylation pathways found in mammals, such as galactosylation.

Based on the desired or undesired O-linked and/or N-linked glycosylation modification of a protein product, one or more lectins can be selected for affinity chromatography in flow-through mode or bind & elute mode. For example, if a desired protein lacks particular O-linked and/or N-linked mannose modifications (i.e., desired protein is unmodified), a lectin that binds to mannose moieties, e.g., Con A, LCH, GNA, DC-SIGN and L-SIGN, can be selected for affinity purification in flow-through mode, such that the desired unmodified product passes through the support and is available for further purification or processing. Conversely, if a desired protein contains particular O-linked and/or N-linked mannose modifications (i.e., desired protein is unmodified), a lectin that binds to mannose moieties, e.g., Con A, LCH, GNA, DC-SIGN and L-SIGN, can be selected for affinity purification in bind & elute mode, such that the desired modified product binds to the support and the undesired unmodified product passes through. In the later example, the flow through can be discarded while the desired modified product is eluted from the support for further purification or processing. The same principle applies to recombinant protein products containing other glycosylation modifications introduced by the fungal expression system.

Another pseudo-affinity purification tool is ‘mixed-mode’ chromatography. As used herein, the term “mixed mode chromatography” refers to chromatographic methods that utilize more than one form of interactions between the stationary phase and analytes in order to achieve their separation, e.g., secondary interactions in mixed mode chromatography contribute to the retention of the solutes. Advantages of mixed mode chromatography include high selectivity, e.g., positive, negative and neutral substances could be separated in a single run, and higher loading capacity.

Mixed mode chromatography can be performed on ceramic or crystalline apatite media, such as hydroxyapatite (HA) chromatography and fluoroapatite (FA) chromatography. Other mixed mode resins include, but are not limited to, CaptoAdhere, Capto MMC (GE Healthcare); HEA Hypercel, and PPA Hypercel (Pall); and Toyopearl® MX-Trp-650M (Tosoh BioScience). These chromatography resins provide biomolecule selectivity complementary to more traditional ion exchange or hydrophobic interaction techniques.

Ceramic hydroxyapatite (Ca₅(PO4)₃OH)₂ is a form of calcium phosphate that can be used for the separation and purification of proteins, enzymes, nucleic acids, viruses and other macromolecules. Hydroxyapatite has unique separation properties and excellent selectivity and resolution. For example, it often separates proteins that appear to be homogeneous by other chromatographic and electrophoretic techniques. Ceramic hydroxyapatite (CHT) chromatography with a sodium chloride or sodium phosphate gradient elution may be used as polishing step in monoclonal antibody purification processes to remove dimers, aggregates and leached Protein A.

Exemplary hydroxyapatite (HA) sorbents of type I and type II are selected from ceramic and crystalline materials. HA sorbents are available in different particle sizes (e.g. type 1, Bio-Rad Laboratories). In an exemplary embodiment, the particle size of the HA sorbent is between about 10 μm and about 200 μm, between about 20 μm and about 100 μm or between about 30 μm and about 50 μm. In a particular example, the particle size of the HA sorbent is about 40 μm (e.g., CHT, Type I).

Exemplary type I and type II fluoroapatite (FA) sorbents are selected from ceramic (e.g., bead-like particles) and crystalline materials. Ceramic FA sorbents are available in different particle sizes (e.g. type 1 and type 2, Bio-Rad Laboratories). In an exemplary embodiment the particle size of the ceramic FA sorbent is from about 20 μm to about 180 μm, preferably about 20 to about 100 μm, more preferably about 20 μm to about 80 μm. In one example, the particle size of the ceramic FA medium is about 40 μm (e.g., type 1 ceramic FA). In another example, the FA medium includes HA in addition to FA.

The selection of the flow velocity used for loading the sample onto the hydroxyapatite or fluoroapatite column, as well as the elution flow velocity depends on the type of hydroxyapatite or fluoroapatite sorbent and on the column geometry. In one exemplary embodiment, at process scale, the loading flow velocity is selected from about 50 to about 900 cm/h, from about 100 to about 500 cm/h, preferably from about 150 to about 300 cm/h and, more preferably, about 200 cm/h.

In an exemplary embodiment, the pH of the elution buffer is selected from about pH 5 to about pH 9, preferably from about pH 6 to about pH 8, and more preferably about pH 6.5.

In one embodiment, the disclosed purification processes employ hydroxyapatite (HA) chromatography on CHT resin after protein A chromatography. Preferably, the elution is performed as a linear gradient (0-100%) from about 0 M to 1.5 M sodium chloride in a 5 mM sodium phosphate buffer at pH 6.5. The OD₂₈₀ of the effluent can be monitored. In one embodiment, during elution, a single fraction from 0.1 OD on the front flank to the peak maximum is collected and then a series of fractions, e.g., about one-third of the column volume, are collected from the peak maximum to 0.1 OD on the rear flank are collected for further purity analysis. In another preferred embodiment, the elution is performed as a linear gradient (0-100%) from about 5 mM to 0.25 M sodium phosphate buffer at pH 6.5. The OD₂₈₀ of the effluent can be monitored. During elution, fractions of ˜½ CV can be collected from 0.1 OD on the front flank to 0.1 OD on the rear flank for further purity analysis.

Polyclonal antibodies (e.g., serum samples) require antigen-specific affinity purification to prevent co-purification of non-specific immunoglobulins. For example, generally only 2-5% of total IgG in mouse serum is specific for the antigen used to immunize the animal. The type(s) and degree of purification that are necessary to obtain usable antibody depend upon the intended application(s) for the antibody. However, monoclonal antibodies that were developed using cell lines, e.g., hybridomas or recombinant expression systems, and produced as ascites fluid or cell culture supernatant can be fully purified without using an antigen-specific affinity method because the target antibody is (for most practical purposes) the only immunoglobulin in the production sample.

### Monitoring Impurities

Profiling of impurities in biopharmaceutical products and their associated intermediates and excipients is a regulatory expectation. See, e.g., US Food and Drug Administration, Genotoxic and Carcinogenic Impurities in Drug Substances and Products: Recommended Approaches. This guidance provides recommendations on how to evaluate the safety of these impurities and exposure thresholds. The European Medicines Agency's (EMEA committee for Medicinal Products for Human Use (CHMP) also published the Guideline on the Limits of Genotoxic Impurities, which is being applied by European authorities for new drug products and in some cases also to drug substances in drug development. These guidelines augment the International Conference on Harmonization (ICH) guidances for industry: Q3A(R2) Impurities in New Drug Substances, Q3B(R2) Impurities in New Drug Products, and Q3C(R3) Impurities: Residual Solvents that address impurities in a more general approach.

Although some impurities are related to the drug product (i.e., product-associated variant), others are added during synthesis, processing, and manufacturing. These impurities fall into several broad classes: product-associated variants; process-related substances introduced upstream; residual impurities throughout the process; process-related residual impurities introduced downstream; and residual impurities introduced from disposables.

As used herein, “product-associated variant” refers to a product other than the desired product (e.g., the desired multi-subunit complex) which is present in a preparation of the desired product and related to the desired product. Exemplary product-associated variants include truncated or elongated peptides, products having different glycosylation than the desired glycosylation (e.g., if an aglycosylated product is desired then any glycosylated product would be considered to be a product-associated variant), complexes having abnormal stoichiometry, improper assembly, abnormal disulfide linkages, abnormal or incomplete folding, aggregation, protease cleavage, or other abnormalities. Exemplary product-associated variants may exhibit alterations in one or more of molecular mass (e.g., detected by size exclusion chromatography), isoelectric point (e.g., detected by isoelectric focusing), electrophoretic mobility (e.g., detected by gel electrophoresis), phosphorylation state (e.g., detected by mass spectrometry), charge to mass ratio (e.g., detected by mass spectrometry), mass or identity of proteolytic fragments (e.g., detected by mass spectrometry or gel electrophoresis), hydrophobicity (e.g., detected by HPLC), charge (e.g., detected by ion exchange chromatography), affinity (e.g., in the case of an antibody, detected by binding to protein A, protein G, and/or an epitope to which the desired antibody binds), and glycosylation state (e.g., detected by binding to an anti-glycoprotein antibody such as Ab1, Ab2, Ab3, Ab4, or Ab5). Where the desired protein is an antibody, the term product-associate variant may include a glyco-heavy variant and/or half antibody species (described below).

Exemplary product-associated variants include variant forms that contain aberrant disulfide bonds. For example, most IgG1 antibody molecules are stabilized by a total of 16 intra-chain and inter-chain disulfide bridges, which stabilize the folding of the IgG domains in both heavy and light chains, while the inter-chain disulfide bridges stabilize the association between heavy and light chains. Other antibody types likewise contain characteristic stabilizing intra-chain and inter-chain disulfide bonds. Further, some antibodies (including Ab-A disclosed herein) contain additional disulfide bonds referred to as non-canonical disulfide bonds. Thus, aberrant inter-chain disulfide bonds may result in abnormal complex stoichiometry, due to the absence of a stabilizing covalent linkage, and/or disulfide linkages to additional subunits. Additionally, aberrant disulfide bonds (whether inter-chain or intra-chain) may decrease structural stability of the antibody, which may result in decreased activity, decreased stability, increased propensity to form aggregates, and/or increased immunogenicity. Product-associated variants containing aberrant disulfide bonds may be detected in a variety of ways, including non-reduced denaturing SDS-PAGE, capillary electrophoresis, cIEX, mass spectrometry (optionally with chemical modification to produce a mass shift in free cysteines), size exclusion chromatography, HPLC, changes in light scattering, and any other suitable methods known in the art. See, e.g., The Protein Protocols Handbook 2002, Part V, 581-583, DOI: 10.1385/1-59259-169-8:581.

Generally, dialysis, desalting and diafiltration can be used to exchange antibodies into particular buffers and remove undesired low-molecular weight (MW) components. In particular, dialysis membranes, size-exclusion resins, and diafiltration devices that feature high-molecular weight cut-offs (MWCO) can be used to separate immunoglobulins (>140kDa) from small proteins and peptides. See, e.g., Grodzki, A. C. and Berenstein, E. (2010). Antibody purification: ammonium sulfate fractionation or gel filtration. In: C. Oliver and M. C. Jamur (eds.), Immunocytochemical Methods and Protocols, Methods in Molecular Biology, Vol. 588:15-26. Humana Press.

Size-exclusion chromatography can be used to detect antibody aggregates, monomer, and fragments. In addition, size-exclusion chromatography coupled to mass spectrometry may be used to measure the molecular weights of antibody; antibody conjugates, and antibody light chain and heavy chain.

Exemplary size exclusion resins for use in the purification and purity monitoring methods include TSKgel G3000SW and TSKgel G3000SWx1 from Tosoh Biosciences (Montgomeryville, Pa., USA); Shodex KW-804, Protein-Pak 300SW, and BioSuite 250 from Waters (Milford, Mass., USA); MAbPac™ SEC-1 and MAbPac™ SCX-10 from Thermo Scientific (Sunnyvale, Calif., USA).

In one embodiment, size exclusion chromatography is used to monitor impurity separation during the purification process. By way of example, an equilibrated TSKgel GS3000SW 17.8×300 mm column connected with a TSKgel Guard SWx16×40 mm from Tosoh Bioscience (King of Prussia, PA) may be loaded with sample, using a SE-HPLC buffer comprising 100 mM sodium phosphate, 200 mM sodium chloride pH 6.5 as a mobile phase with a flow rate of 0.5 mL/min in isocratic mode. Using an Agilent (Santa Clara, Calif.) 1200 Series HPLC with UV detection instrument, absorbance at UV 215 nm can be monitored. Samples can then be collected and diluted to a desired concentration, e.g., 1 mg/mL. The diluted sample of a fraction thereof, e.g., 30 can then be loaded onto the SE-HPLC column. Preferably, column performance is monitored using gel filtration standards (e.g., BioRad).

Product-associated variants include glycovariants. As used herein, “glycovariant” refers to a glycosylated product-associated variant sometimes present in antibody preparations and which contains at least a partial Fe sequence. The glycovariant contains glycans covalently attached to polypeptide side chains of the desired protein. The glycovariant may be “glyco-heavy” or “glyco-light” in comparison to the desired protein product, i.e., contains additional glycosylation modifications compared to the desired protein or contains less glycosylation modifications than the desired protein, respectively. Exemplary glycosylation modifications include, but are not limited to, N-linked glycosylation, O-linked glycosylation, C-glycosylation and phosphoglycosylation.

The glycovariant is characterized by increased or decreased electrophoretic mobility observable by SDS-PAGE (relative to a normal polypeptide chain), lectin binding affinity, binding to an anti-glycoprotein antibody (such as Ab1, Ab2, Ab3, Ab4, or Ab5) binding to an anti-Fc antibody, and apparent higher or lower molecular weight of antibody complexes containing the glycovariant as determined by size exclusion chromatography. See, e.g., U.S. Provisional Application Ser. No. 61/525,307, filed Aug. 31, 2011, which is incorporated by reference herein in its entirety.

As used herein “glycosylation impurity” refers to a material that has a different glycosylation pattern than the desired protein. The glycosylation impurity may contain the same or different primary, secondary, tertiary and/or quaternary structure as the desired protein. Therefore, a glycovariant is a type of glycosylation impurity.

Analytical methods for monitoring glycosylation of mAbs are important because bioprocess conditions can cause, e.g., variation in high mannose type, truncated forms, reduction of tetra-antennary and increase in tri- and biantennary structures, less sialyated glycans and less glycosylation. The presence of glycovariants in a sample may be monitored using analytical means known in the art, such as glycan staining or labeling, glycoproteome and glycome analysis by mass spectrometry and/or glycoprotein purification or enrichment. In one embodiment, glycovariants are analyzed using anti-glycoprotein antibody (such as Ab1, Ab2, Ab3, Ab4, or Ab5) binding assays, e.g., ELISA, light interferometry (which may be performed using a ForteBio Octet®), dual polarization interferometry (which may be performed using a Farfield AnaLight®), static light scattering (which may be performed using a Wyatt DynaPro NanoStar™), dynamic light scattering (which may be performed using a Wyatt DynaPro NanoStar™), composition-gradient multi-angle light scattering (which may be performed using a Wyatt Calypso II), surface plasmon resonance (which may be performed using ProteOn XPR36 or Biacore T100), europium ELISA, chemoelectroluminescent ELISA, far western analysis, electrochemiluminescence (which may be performed using a MesoScale Discovery) or other binding assay.

In one embodiment, glycan staining or labeling is used to detect glycovariants. For example, glycan sugar groups can be chemically restructured with periodic acid to oxidize vicinal hydroxyls on sugars to aldehydes or ketones so that they are reactive to dyes, e.g., periodic acid-Schiff (PAS) stain, to detect and quantify glycoproteins in a given sample. Periodic acid can also be used to make sugars reactive toward crosslinkers, which can be covalently bound to labeling molecules (e.g., biotin) or immobilized support (e.g., streptavidin) for detection or purification.

In another embodiment, mass spectrometry is used to identify and quantitate glycovariants in a sample. For example, enzymatic digestion may be used to release oligosaccharides from the immunoglycoprotein, where the oligosaccharide is subsequently derivatized with a fluorescent modifier, resolved by normal phase chromatography coupled with fluorescence detection, and analyzed by mass spectrometry (e.g., MALDI-TOF). The basic pipeline for glycoproteomic analysis includes glycoprotein or glycopeptides enrichment, multidimensional separation by liquid chromatography (LC), tandem mass spectrometry and data analysis via bioinformatics.

Spectrometric analysis can be performed before or after enzymatic cleavage of glycans by, e.g., endoglycanase H (endo H) or peptide-N4-(N-acetyl-beta-glucosaminyl)asparagine amidase (PNGase), depending on the experiment. Additionally, quantitative comparative glycoproteome analysis may be performed by differential labeling with stable isotope labeling by amino acids in cell culture (SILAC) reagents. Moreover, absolute quantitation by selected reaction monitoring (SRM) can be performed on targeted glycoproteins using isotopically labeled, “heavy” reference peptides.

In one embodiment, lectins for affinity purification to deplete or selectively enrich glycovariants of the desired protein during the purification process. Lectins are glycan-binding proteins have high specificity for distinct sugar moieties. A non-limiting list of commercially available lectins is provided in Table 3 below.

TABLE 3
Exemplary commercially available lectins.

| | | | |
|---|---|---|---|
| Lectin | | | |
| Symbol | Lectin Name | Source | Ligand motif |

Mannose binding lectins

| | | | |
|---|---|---|---|
| ConA | Concanavalin | Canavalia | α-D-mannosyl and α-D-glucosyl residues |
| | A | ensiformis | branched α-mannosidic structures (high α- |
| | | | mannose type, or hybrid type and biantennary |
| | | | complex type N-Glycans) |
| LCH | Lentil lectin | Lens culinaris | Fucosylated core region of bi- and |
| | | | triantennary complex type N-Glycans |
| GNA | Snowdrop | Galanthus | α 1-2, α 1-3 and α 1-6 linked high mannose |
| | lectin | nivalis | structures |
| DC-SIGN | Dendritic Cell- | Human | Calcium-dependent mannose-type |
| | Specific | Murine | carbohydrates |
| | Intercellular | | |
| | adhesion | | |
| | molecule-3- | | |
| | Grabbing Non- | | |
| | integrin | | |
| L-SIGN | Liver/lymph | Human | Calcium-dependent mannose-type |
| | node-specific | Murine | carbohydrates |
| | intercellular | | |
| | adhesion | | |
| | molecule-3- | | |
| | grabbing | | |
| | integrin | | |

Galactose/N-acetylgalactosamine binding lectins

| | | | |
|---|---|---|---|
| RCA | Ricin, Ricinus | Ricinus | Galβ1-4GlcNAcβ1-R |
| | communis | communis | |
| | Agglutinin, | | |
| | RCA120 | | |
| PNA | Peanut | Arachis | Galβ1-3GalNAcα1-Ser/Thr (T-Antigen) |
| | agglutinin | hypogaea | |
| AIL | Jacalin | Artocarpus | (Sia)Galβ1-3GalNAcα1-Ser/Thr (T-Antigen) |
| | | integrifolia | |
| VVL | Hairy vetch | Vicia villosa | GalNAcα-Ser/Thr (Tn-Antigen) |
| | lectin | | |

N-acetylglucosamine binding lectins

| | | | |
|---|---|---|---|
| WGA | Wheat Germ | Triticum | GlcNAcβ1-4GlcNAcβ1-4GlcNAc, Neu5Ac |
| | Agglutinin, | vulgaris | (sialic acid) |
| | WGA | | |

N-acetylneursminic acid binding lectins

| | | | |
|---|---|---|---|
| SNA | Elderberry | Sambucus | Neu5Acα2-6Gal(NAc)-R |
| | lectin | nigra | |
| MAL | Maackia | Maackia | Neu5Ac/Gcα2,3Galβ1,4Glc(NAc) |
| | amurensis | amurensis | |
| | leukoagglutinin | | |
| MAH | Maackia | Maackia | Neu5Ac/Gcα2,3Galβ1,3(Neu5Acα2,6)GalNac |
| | amurensis | amurensis | |
| | hemoagglutinin | | |

Fucose binding lectins

| | | | |
|---|---|---|---|
| UEA | Ulex europaeus | Ulex | Fucα1-2Gal-R |
| | agglutinin | europaeus | |
| AAL | Aleuria | Aleuria | Fucα1-2Galβ1-4(Fucα1-3/4)Galβ1-4GlcNAc, |
| | aurantia lectin | aurantia | R2-GlcNAcβ1-4(Fucα1-6)GlcNAc-R1 |

In one embodiment, a sample obtained from the fermentation process, e.g., during the run or after the run is completed, is subject to anti-glycoprotein antibody (such as Ab1, Ab2, Ab3, Ab4, or Ab5) binding assay to detect the amount and/or type of glycosylated impurities in the sample(s). Similarly, in other embodiments, the purification process includes detecting the amount and/or type of glycosylated impurities in a sample from which the desired protein is purified. For example, in a particular embodiment, a portion of the eluate or a fraction thereof from at least one chromatographic step in the purification process may be contacted with an anti-glycoprotein antibody (such as Ab1, Ab2, Ab3, Ab4, or Ab5).

The level of anti-glycoprotein antibody (such as Ab1, Ab2, Ab3, Ab4, or Ab5) binding typically correlates with the level of the product-associated glycovariant impurity present in the eluate or a fraction thereof (based on conventional size exclusion chromatography methods), such that one or more fractions of the eluate can be selected for further purification and processing based on the content of glycovariant impurities, e.g., select fractions of the eluate with less than 10% glycovariant for further chromatographic purification. In some embodiments, multiple anti-glycoprotein antibody (such as Ab1, Ab2, Ab3, Ab4, or Ab5) (i.e., two or more thereof) may be used to monitor purity of the product associated glycovariant impurities.

In an alternate embodiment, certain samples or eluate or fractions thereof are discarded based on the amount and/or type of detected glycosylated impurities. In yet another embodiment, certain samples or fractions are treated to reduce and/or remove the glycosylated impurities based on the amount and/or type of detected glycosylated impurities. Exemplary treatment includes one or more of the following: (i) addition of an enzyme or other chemical moiety that removes glycosylation, (ii) removal of the glycosylated impurities by effecting one or more lectin binding steps, (iii) effecting size exclusion chromatography to remove the glycosylated impurities.

In a particular embodiment, the anti-glycoprotein antibody (such as Ab1, Ab2, Ab3, Ab4, or Ab5) is conjugated to a probe and then immobilized to a support. The support may be in batch or packed into a column, e.g., for HPLC. Exemplary probes include biotin, alkaline phosphatase (AP), horseradish peroxidase (HRP), luciferase, fluorescein (fluorescein isothiocyanate, FITC) and rhodamine (tetramethyl rhodamine isothiocyanate, TRITC), green fluorescent protein (GFP) and phycobiliproteins (e.g., allophycocyanin, phycocyanin, phycoerythrin and phycoerythrocyanin). Exemplary supports include avidin, streptavidin, NeutrAvidin (deglycosylated avidin) and magnetic beads. It should be noted that the invention is not limited by coupling chemistry. Preferably, the anti-glycoprotein antibody (such as Ab1, Ab2, Ab3, Ab4, or Ab5) is biotinylated and immobilized onto a streptavidin sensor.

Standard protein-protein interaction monitoring processes may be used to analyze the interaction between the anti-glycoprotein antibody (such as Ab1, Ab2, Ab3, Ab4, or Ab5) and glycosylation impurities in samples from various steps of the purification process. Exemplary protein-protein interaction monitoring process include, but are not limited to, light interferometry (which may be performed using a ForteBio Octet®), dual polarization interferometry (which may be performed using a Farfield AnaLight®), static light scattering (which may be performed using a Wyatt DynaPro NanoStar™), dynamic light scattering (which may be performed using a Wyatt DynaPro NanoStar™), composition-gradient multi-angle light scattering (which may be performed using a Wyatt Calypso II), surface plasmon resonance (which may be performed using ProteOn XPR36 or Biacore T100), ELISA, chemoelectroluminescent ELISA, europium ELISA, far western analysis, chemoluminescence (which may be performed using a MesoScale Discovery) or other binding assay.

Light interferometry is an optical analytical technique that analyzes the interference pattern of white light reflected from two surfaces (a layer of immobilized protein on the biosensor tip, and an internal reference layer) to measure biomolecular interactions in real-time based on a shift in the interference pattern (i.e., caused by a change in the number of molecules bound to the biosensor tip), thereby providing information about binding specificity, rates of association and dissociation, or concentration.

Dual polarization interferometry is based on a dual slab wave guide sensor chip that has an upper sensing wave guide as well as a lower optical reference wave guide lit up with an alternating orthogonal polarized laser beam. Two differing wave guide modes are created—specifically, the transverse magnetic (TM) mode and the transverse electric (TE) mode. Both modes generate an evanescent field at the top sensing wave guide surface and probe the materials that contact with this surface. As material interacts with the sensor surface, it leads to phase changes in interference fringes. Then, the interference fringe pattern for each mode is mathematically resolved into RI and thickness values. Thus, the sensor is able to measure extremely subtle molecular changes on the sensor surface.

Static light scattering (SLS) is a non-invasive technique whereby an absolute molecular mass of a protein sample in solution may be experimentally determined to an accuracy of better than 5% through exposure to low intensity laser light (690 nm). The intensity of the scattered light is measured as a function of angle and may be analyzed to yield the molar mass, root mean square radius, and second virial coefficient (A₂). The results of an SLS experiments can be used as a quality control in protein preparation (e.g. for structural studies) in addition to the determination of solution oligomeric state (monomer/dimer etc.). SLS experiments may be performed in either batch or chromatography modes.

Dynamic light scattering (also known as quasi-elastic light scattering, QELS, or photon correlation spectroscopy, PCS) is a technique for measuring the hydrodynamic size of molecules and submicron particles based on real-time intensities (compared to time-average intensities, as measured by static light scattering). Fluctuations (temporal variation, typically in a us to ms time scale) of the scattered light from a particle in a medium are recorded and analyzed in correlation delay time domain. The particles can be solid particles (e.g., metal oxides, mineral debris, and latex particles) or soft particles (e.g., vesicles and micelles) in suspension, or macromolecular chains (e.g., synthetic polymers and biomaterials) in solution. Since the diffusion rate of particles is determined by their sizes in a given environment, information about their size is contained in the rate of fluctuation of the scattered light.

The scattering intensity of a small molecule is proportional to the square of the molecular weight. As such, dynamic and static light scattering techniques are very sensitive to the onset of protein aggregation and other changes in protein structure arising from subtle changes in conditions.

Composition-gradient multi-angle light scattering (CG-MALS) employs a series of unfractionated samples of different composition or concentration in order to characterize macromolecular interactions such as reversible self- and hetero-association of proteins, reaction rates and affinities of irreversible aggregation, or virial coefficients. Such measurements provide information about specific reversible complex binding (e.g., K_{d}, stoichiometry, self and/or heteroassociations), non-specific interactions (e.g., self- and cross-virial coefficients), aggregation and other time-dependent reactions (e.g., stop-flow kinetics and t) and Zimm plots (e.g., concentration gradients for determining A_{W}, A₂, A₃ (second and third virial coefficients), or r_{g}).

The surface plasmon resonance (SPR) phenomenon occurs when polarized light, under conditions of total internal reflection, strikes an electrically conducting (e.g., gold) layer at the interface between media of different refractive index (i.e., glass of a sensor surface (high refractive index) and a buffer (low refractive index)). A wedge of polarized light, covering a range of incident angles, is directed toward the glass face of the sensor surface. An electric field intensity (i.e., evanescent wave), which is generated when the light strikes the glass, interacts with, and is absorbed by, free electron clouds in the gold layer, generating electron charge density waves called plasmons and causing a reduction in the intensity of the reflected light. The resonance angle at which this intensity minimum occurs is a function of the refractive index of the solution close to the gold layer on the opposing face of the sensor surface. Reflected light is detected within a monitoring device, e.g., ProteOn XPR36 or Biacore system. The kinetics (i.e. rates of complex formation (kₐ) and dissociation (k_{d}), affinity (e.g., K_{D}), and concentration information can be determined based on the plasmon readout.

Information obtained from these and other protein-protein interaction monitoring processes can be used to, e.g., quantify binding affinity and stoichiometry of enzyme/inhibitor or antibody/antigen interactions or glycoprotein/lectin interactions; study the impact of small molecules on protein-protein interactions; adjust buffer parameters to improve formulation stability and viscosity; optimize antibody purification and understand the effects of large excipients on formulations; quantify impact of solvent ionic strength, pH, or excipients on polymerization or protein associations; measure kinetics of self-assembly and aggregation; and characterize macromolecular binding affinity and associated complex stoichiometry over a wide range of buffer compositions, time, and temperature scales.

In a preferred embodiment, the level of anti-glycoprotein antibody (such as Ab1, Ab2, Ab3, Ab4, or Ab5) binding (which correlates with the amount of glycovariant impurity) is determined using ELISA, optionally with horseradish peroxidase or europium detection.

Exemplary process-related impurities introduced upstream include nucleic acids (e.g., DNA and RNA) and host cell proteins (HCP) that are unwanted cell components found with the protein of interest after cell lysis. These process-related impurities also include antibiotics that are added upstream to the cell-culture media to control bacterial contamination and maintain selective pressure on the host organisms. Exemplary antibiotics include kanamycin, ampicillin, penicillin, amphotericin B, tetracyline, gentamicin sulfate, hygromycin B, and plasmocin.

Exemplary residual impurities incurred throughout the process include process enhancing agents or catalysts, which are added throughout the process to make some of the steps more efficient and increase yield of the product. For example, guanidine and urea are added for solubilization of the fermentation output, and glutathione and dithiothreitol (DTT) are used during reduction and refolding of proteins.

Exemplary process-related impurities introduced downstream include chemicals and reagents (e.g., alcohols and glycols) required for chromatographic purification of target proteins that must be cleared from the process, as well as surfactants (e.g., Triton-X, Pluronic, Antifoam—A, B, C, Tween, or Polysorbate) that are added during downstream processing to aid in separating the protein, peptide, and nucleic acids from the process stream by lowering the interfacial tension by adsorbing at the liquid-liquid interface.

Exemplary residual impurities introduced from disposables include “extractables,” which are compounds that can be extracted from a component under exaggerated conditions (e.g., harsh solvents or at elevated temperatures) and have the potential to contaminate the drug product, and “leachables,” which are compounds that leach into the drug product formulation from the component as a result of direct contact with the formulation under normal conditions or sometimes at accelerated conditions. Leachables may be a subset of extractables. Extractables must be controlled to the extent that components used are appropriate. Leachables must be controlled so that the drug products are not adulterated.

To further articulate the invention described above, the following non-limiting examples are provided.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Immunization of Rabbits to Produce Anti-Glycoprotein Antibodies

Ab-A is a humanized IgG1 antibody that was expressed in P. pastoris (further described in the examples below). Some preparation of Ab-A, depending on culture conditions and purification steps utilized, were observed to contain varying, detectable levels of mannosylated Ab-A. As further described below, these mannosylated antibodies could be detected using lectin-based binding assays or using the anti-glycoprotein antibodies disclosed herein.

Ab-A lot 2 was prepared in order to produce an antibody preparation highly enriched for the Ab-A glycovariant. Clarified fermentation broth was subject to Protein A affinity purification, followed by ceramic hydroxyapatite (CHT) chromatography. Fractions were assessed to determine relative glycovariant content by analytical SE-HPLC (by quantifying fractions from the SE-HPLC step know to be highly enriched in mannosylated antibody). CHT fractions that were enriched for the glycovariant were further enriched by preparative gel-filtration chromatography on a Superdex 200 (GE healthcare) column using DPBS (Hyclone) as the isocratic elution buffer.

Ab-A lot 2 is then used to immunize rabbits. Immunization consists of a first subcutaneous (sc) injection of 100 μg of antigen mixed with 100 μg of keyhole limpet hemocyanin (KLH) in complete Freund's adjuvant (CFA) (Sigma) followed by two boosts, two weeks apart each containing 50 μg antigen mixed with 50 μg in incomplete Freund's adjuvant (IFA) (Sigma). Animals are bled on day 55, and serum titers are determined by ELISA (antigen recognition).

Antibody Selection Titer Assessment

To identify and characterize antibodies that bind to mannosylated proteins, antibody-containing solutions are tested by ELISA. Briefly, neutravidin coated plates (Thermo Scientific), are coated with biotinylated mannosylated antibody (50 μL per well, 1 μg/mL) diluted in ELISA buffer (0.5% fish skin gelatin in PBS pH 7.4) either for approximately 1 hr at room temperature or alternatively overnight at 4 degrees C. The plates are then further blocked with ELISA buffer for one hour at room temperature and washed using wash buffer (PBS, 0.05% tween 20). Serum samples tested are serially diluted using ELISA buffer. Fifty microliters of diluted serum samples are transferred onto the wells and incubated for one hour at room temperature. After this incubation, the plate is washed with wash buffer. For development, a goat anti-rabbit Fc-specific HRP conjugated polyclonal antibody (1:5000 dilution in ELISA buffer) is added onto the wells and incubated for 45 min at RT. After a 3× wash step with wash solution, the plate is developed using TMB substrate for two minutes at room temperature and the reaction is quenched using 0.5M HCl. The well absorbance is read at 450 nm.

Tissue Harvesting

Once acceptable titers are established, the rabbit(s) are sacrificed. Spleen, lymph nodes, and whole blood are harvested and processed as follows:

Spleen and lymph nodes are processed into a single cell suspension by disassociating the tissue and pushing through sterile wire mesh at 70 um (Fisher) with a plunger of a 20 cc syringe. Cells are collected in PBS. Cells are washed twice by centrifugation. After the last wash, cell density is determined by trypan blue. Cells are centrifuged at 1500 rpm for 10 minutes; the supernatant is discarded. Cells are resuspended in the appropriate volume of 10% dimethyl sulfoxide (DMSO, Sigma) in FBS (Hyclone) and dispensed at 1 ml/vial. Vials are stored at −70 degrees C. in a slow freezing chamber for 24 hours and stored in liquid nitrogen.

Peripheral blood mononuclear cells (PBMCs) are isolated by mixing whole blood with equal parts of the low glucose medium described above without FBS. 35 ml of the whole blood mixture is carefully layered onto 8 ml of Lympholyte Rabbit (Cedarlane) into a 45 ml conical tube (Corning) and are centrifuged 30 minutes at 2500 rpm at room temperature without brakes. After centrifugation, the PBMC layers are carefully removed using a glass Pasteur pipette (VWR), combined, and placed into a clean 50 ml vial. Cells are washed twice with the modified medium described above by centrifugation at 1500 rpm for 10 minutes at room temperature, and cell density is determined by trypan blue staining. After the last wash, cells are resuspended in an appropriate volume of 10% DMSO/FBS medium and frozen as described above.

B Cell Selection, Enrichment and Culture Conditions

On the day of setting up B cell culture, PBMC, splenocyte, or lymph node vials are thawed for use. Vials are removed from LN2 tank and placed in a 37 degrees C. water bath until thawed. Contents of vials are transferred into 15 ml conical centrifuge tube (Corning) and 10 ml of modified RPMI described above is slowly added to the tube. Cells are centrifuged for 5 minutes at 2K RPM, and the supernatant is discarded. Cells are resuspended in 10 ml of fresh media. Cell density and viability is determined by trypan blue.

Cells are pre-mixed with the biotinylated mannosylated protein as follows. Cells are washed again and resuspended at 1E07 cells/80 μL medium. Biotinylated mannosylated protein is added to the cell suspension at the final concentration of 5 μg/mL and incubated for 30 minutes at 4 degrees C. Unbound biotinylated mannosylated protein is removed performing two 10 ml washes using PBF (Ca/Mg free PBS (Hyclone), 2 mM ethylenediamine tetraacetic acid (EDTA), 0.5% bovine serum albumin (BSA) (Sigma-biotin free)). After the second wash, cells are resuspended at 1E07 cells/80 μL PBF and 20 μL of MACS® streptavidin beads (Miltenyi Biotec, Auburn Calif.) per 10E7 cells are added to the cell suspension. Cells and beads are incubated at 4 degrees C. for 15 minutes and washed once with 2 ml of PBF per 10E7 cells.

Alternatively, mannosylated protein is pre-loaded onto the streptavidin beads as follows. Seventy-five microliters of streptavidin beads (Miltenyi Biotec, Auburn Calif.) are mixed with N-terminally biotinylated mannosylated protein (10 μg/ml final concentration) and 300 μL PBF. This mixture is incubated at 4 degrees C. for 30 min and unbound mannosylated protein is removed using a MACS separation column (Miltenyi Biotec), with a 1 ml rinse to remove unbound material. Then material is plunged out, then used to resuspend cells from above in 100 ul per 1E7 cells, the mixture is then incubated at 4 degrees C. for 30 min and washed once with 10 ml of PBF.

For both protocols the following applied: After washing, the cells are resuspended in 500 μL of PBF and set aside. A MACS® MS column (Miltenyi Biotec, Auburn Calif.) is pre-rinsed with 500 ml of PBF on a magnetic stand (Miltenyi). Cell suspension is applied to the column through a pre-filter, and unbound fraction is collected. The column is washed with 2.5 ml of PBF buffer. The column is removed from the magnet stand and placed onto a clean, sterile 1.5 ml Eppendorf tube. 1 ml of PBF buffer is added to the top of the column, and positive selected cells are collected. The yield and viability of positive cell fraction is determined by trypan blue staining. Positive selection yielded an average of 1% of the starting cell concentration.

A pilot cell screen is established to provide information on seeding levels for the culture. Plates are seeded at 10, 25, 50, 100, or 200 enriched B cells/well. In addition, each well contained 50K cells/well of irradiated EL-4.B5 cells (5,000 Rads) and an appropriate level of activated rabbit T cell supernatant (See U.S. Patent Application Publication No. 20070269868) (ranging from 1-5% depending on preparation) in high glucose modified RPMI medium at a final volume of 250 μL/well. Cultures are incubated for 5 to 7 days at 37 degrees C. in 4% CO₂.

B-Cell Culture Screening by Antigen-Recognition (ELISA)

To identify wells producing antibodies specific for mannosylated protein, a two-step procedure was used. In a first step, the same protocol as described for titer determination of serum samples by antigen-recognition (ELISA) is used with the following changes. Briefly, neutravidin coated plates are coated with biotinylated mannosylated protein (50 μL per well, 1μg/mL each). B-cell supernatant samples (50 μL) are tested without prior dilution. In a second step, biotinylated protein of identical sequence to that used in the first step, but without mannose, is used to coat neutravidin plates. Protein without mannosylation can be produced using mammalian cells (e.g., CHO cells, human kidney cells, or others) or using a bacterial expression system. Reactivity in the second assay would indicate the antibody specificity is for the protein rather than the mannose structure and such antibodies would then be discarded.

Isolation of Antigen-Specific B-Cells

Plates containing wells of interest are removed from −70 degrees C., and the cells from each well are recovered using five washes of 200 microliters of medium (10% RPMI complete, 55 μM BME) per well. The recovered cells are pelleted by centrifugation and the supernatant is carefully removed. Pelleted cells are resuspended in 100 μL of medium. To identify antibody expressing cells, streptavidin coated magnetic beads (M280 Dynabeads, Invitrogen) are coated with a combination of both N- and C-terminal biotinylated mannosylated protein. Individual biotinylated mannosylated protein lots are optimized by serial dilution. One hundred microliters containing approximately 4×10E7 coated beads are then mixed with the resuspended cells. To this mixture 15 microliters of goat anti-rabbit H&L IgG-FITC (Jackson Immunoresearch) diluted 1:100 in medium are added.

Twenty microliters of cell/beads/anti-rabbit H&L suspension are removed and microliter droplets are dispensed on a one-well glass slide previously treated with Sigmacote (Sigma) totaling 35 to 40 droplets per slide. An impermeable barrier of paraffin oil (JT Baker) is used to submerge the droplets, and the slide is incubated for 90 minutes at 37 degrees C. in a 4% CO2 incubator in the dark.

Specific B cells that produce antibody can be identified by the fluorescent ring around the cells produced by the antibody secretion, recognition of the bead-associated biotinylated antigen, and subsequent detection by the fluorescent-IgG detection reagent. Once a cell of interest is identified it is recovered via a micromanipulator (Eppendorf). The single cell synthesizing and secreting the antibody is transferred into a microcentrifuge tube, frozen using dry ice and stored at −70 degrees C.

Amplification and Sequence Determination of Antibody Sequences from Antigen-Specific B Cells

Antibody sequences are recovered using a combined RT-PCR based method from a single isolated B-cell. Primers containing restriction enzymes are designed to anneal in conserved and constant regions of the target immunoglobulin genes (heavy and light), such as rabbit immunoglobulin sequences, and a two-step nested PCR recovery is used to amplify the antibody sequence. Amplicons from each well are analyzed for recovery and size integrity. The resulting fragments are then digested with Alu1 to fingerprint the sequence clonality. Identical sequences displayed a common fragmentation pattern in their electrophoretic analysis. The original heavy and light chain amplicon fragments are then digested using the restriction enzyme sites contained within the PCR primers and cloned into an expression vector. Vector containing subcloned DNA fragments are amplified and purified. Sequence of the subcloned heavy and light chains are verified prior to expression.

Recombinant Production of Monoclonal Antibody of Desired Antigen Specificity and/or Functional Properties

To determine antigen specificity and functional properties of recovered antibodies from specific B-cells, vectors driving the expression of the desired paired heavy and light chain sequences are transfected into CHO cells, human kidney cells or other mammalian cells.

Antigen-Recognition of Recombinant Antibodies by ELISA

To characterize recombinant expressed antibodies for their ability to bind to mannosylated polypeptides, antibody-containing solutions are tested by ELISA. All incubations are done at room temperature. Briefly, Neutravidin plates (Thermo Scientific) are coated with mannosylated polypeptide-containing solution (1 μg/mL in PBS) for 2 hours. Mannosylated biotinylated, polypeptide-coated plates are then washed three times in wash buffer (PBS, 0.05% Tween-20). The plates are then blocked using a blocking solution (PBS, 0.5% fish skin gelatin, 0.05% Tween-20) for approximately one hour. The blocking solution is then removed and the plates are then incubated with a dilution series of the antibody being tested for approximately one hour. At the end of this incubation, the plate is washed three times with wash buffer and further incubated with a secondary antibody containing solution (Peroxidase conjugated affinipure Fc fragment-specific goat anti-rabbit IgG (Jackson Immunoresearch) for approximately 45 minutes and washed three times. At that point a substrate solution (TMB peroxidase substrate, BioFx) and incubated for 3 to 5 minutes in the dark. The reaction is stopped by addition of a HCl containing solution (0.5M) and the plate is read at 450 nm in a plate-reader.

### Example 2

### Cloning and Sequencing of Five Anti-Glycoprotein Antibodies

The variable heavy and light chains of five rabbit anti-glycoprotein antibodies were amplified from isolated rabbit B cells and each was cloned in frame with a rabbit IgG constant domain. The five anti-glycoprotein antibodies are referred to herein as Ab1, Ab2, Ab3, Ab4, and Ab5; their heavy and light chain polypeptide and polynucleotide sequences are provided in FIGS. 1-4, and the subsequences thereof and SEQ ID NOs of the variable regions, framework regions (FR), complementarity-determining region (CDR), and constant domains are provided in FIGS. 5-12. The full-length Ab1 polypeptide is made up of the heavy chain polypeptide of SEQ ID NO:1 and the light chain polypeptide of SEQ ID NO:21. The full-length Ab2 polypeptide is made up of the heavy chain polypeptide of SEQ ID NO:41 and the light chain polypeptide of SEQ ID NO:61. The full-length Ab3 polypeptide is made up of the heavy chain polypeptide of SEQ ID NO:81 and the light chain polypeptide of SEQ ID NO:101. The full-length Ab4 polypeptide is made up of the heavy chain polypeptide of SEQ ID NO:121 and the light chain polypeptide of SEQ ID NO:141. The full-length Ab5 polypeptide is made up of the heavy chain polypeptide of SEQ ID NO:161 and the light chain polypeptide of SEQ ID NO:181.

### Example 3

### Expression of Anti-Glycoprotein Antibodies

The antibodies Ab1, Ab2, Ab3, Ab4, and Ab5 are expressed in cultured mammalian cells (e.g., CHO cells, human kidney cell lines or the like). Additionally, the antibodies are expressed in Pichia pastoris essentially as follows. A P. pastoris strain is prepared containing integrated genes encoding the heavy and light chains of each respective antibody under control of a suitable promoter, optionally containing more than one copy of each gene (see U.S. Pub. No. 2013/0045888, which is hereby incorporated by reference in its entirety). Correct integration is verified by Southern blotting, and antibody expression and secretion is verified by Western blotting. For antibody production, an inoculum is expanded using medium containing the following nutrients (%w/v): yeast extract 3%, anhydrous dextrose 4%, YNB 1.34%, Biotin 0.004% and 100 mM potassium phosphate. To generate the inoculum for the fermenters, the cells are expanded for approximately 24 hours in a shaking incubator at 30° C. and 300 rpm. A 10% inoculum is then added to Labfors 2.5L working volume vessels containing 1 L sterile growth medium. The growth medium contains the following nutrients: potassium sulfate 18.2 g/L, ammonium phosphate monobasic 36.4 g/L, potassium phosphate dibasic 12.8 g/L, magnesium sulfate heptahydrate 3.72 g L, sodium citrate dihydrate 10 g/L, glycerol 40 g/L, yeast extract 30 g/L, PTM1 trace metals 4.35 mL/L, and antifoam 204 1.67 mL/L. The PTM1 trace metal solution contains the following components: cupric sulfate pentahydrate 6 g/L, sodium iodide 0.08 g/L, manganese sulfate hydrate 3 g/L, sodium molybdate dihyrate 0.2 g/L, boric acid 0.02 g/L, cobalt chloride 0.5 g/L, zinc chloride 20 g/L, ferrous sulfate heptahydrate 65 g/L, biotin 0.2 g/L, and sulfuric acid 5 mL/L.

The bioreactor process control parameters are set as follows: Agitation 1000 rpm, airflow 1.35 standard liters per minute, temperature 28° C. and pH is controlled (at 6) using ammonium hydroxide. No oxygen supplementation is provided.

Fermentation cultures are grown for approximately 12 to 16 hours until the initial glycerol is consumed as denoted by a dissolved oxygen spike. The cultures are optionally starved for approximately three hours after the dissolved oxygen spike. After this optional starvation period, a bolus addition of ethanol is added to the reactor to reach 1% ethanol (w/v). The fermentation cultures are optionally allowed to equilibrate for 15 to 30 minutes, after which feed addition is initiated and set at a constant rate of 1 mL/min for 40 minutes, then the feed pump is controlled by an ethanol sensor keeping the concentration of ethanol at 1% for the remainder of the run using an ethanol sensing probe (Raven Biotech). The feed is comprised of the following components: yeast extract 50 g/L, dextrose monohydrate 500 g/L, magnesium sulfate heptahydrate 3 g/L, and PTM1 trace metals 12 mL/L. Optionally, sodium citrate dihydrate (0.5 g/L) is also added to the feed. The total fermentation time is approximately 80-90 hours.

Antibodies are then purified by Protein A affinity. Clarified supernatants from harvested fermentation or other cell culture broth are diluted with the same volume of equilibration buffer (20 mM Histidine, pH 6). From this diluted broth, 20 mL is then loaded onto a pre-equilibrated 1 mL HiTrap MabSelect Sure column (GE, Piscataway, N.J.). The column is subsequently washed using 20 column volumes (CV) of DPBS. The antibody bound onto the column is eluted using a 2 CV gradient into and 8 CV hold in 100% elution buffer (100 mM Citric Acid pH 3.0). One CV fractions are collected and immediately neutralized using 2M Tris buffer pH 8.0. Protein-containing fractions are determined by measuring absorbance at 280 nM and protein-containing fractions are pooled and dialyzed to DPBS.

Antibody purity is optionally determined by size exclusion high-pressure liquid chromatography (SE-HPLC). Briefly, an Agilent (Santa Clara, Calif.) 1200 Series HPLC with UV detection instrument is used. For sample separation, a TSKgel GS3000SW 1 7.8×300 mM column connected with a TSKgel Guard SWx1 6×40 mM from Tosoh Bioscience (King of Prussia, PA) is used. A 100 mM sodium phosphate, 200 mM sodium chloride pH 6.5 is used as mobile phase with a flow rate of 0.5 mL/min in isocratic mode and absorbance at UV 215 nm is monitored. Before injection of samples the column is equilibrated until a stable baseline is achieved. Samples are diluted to a concentration of 1 mg/mL using mobile phase and a 30 μL volume is injected. To monitor column performance, BioRad (Hercules, Calif.) gel filtration standards are used.

### Example 4

### ELISA Assay Using Anti-Glycoprotein Antibodies

This example describes the use of the antibodies Ab1, Ab2, Ab3, Ab4, and Ab5 for the detection of glycoproteins (specifically, mannose-containing antibodies) in ELISA assays. The results demonstrate sensitive detection of mannosylated antibodies, with Ab1 exhibiting the greatest sensitivity, and europium-based detection exhibiting greater signaling than HRP-based detection.

Methods

Antigen Down HRP ELISA

Briefly, Streptavidin plates (Thermo Scientific) were coated with biotinylated antigen solution (control antibodies of varied mannosylation, lug/mL in PBS) for 1 hour. Antigen-coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antibody being tested for approximately one hour. At the end of this incubation, the plate was washed three times with wash buffer and further incubated with a secondary antibody containing solution (Peroxidase conjugated affinipure anti-rabbit IgG, Fc fragment specific (Jackson Immunoresearch) for approximately 45 minutes and washed three times. At that point a substrate solution (TMB peroxidase substrate, BioFx) was added and incubated for 3 to 5 minutes in the dark. The reaction was stopped by addition of 0.5M HCl and the plate was read at 450 nm in a plate-reader.

AGV Antibody Down Horseradish Peroxidase (HRP) ELISA

Briefly, Streptavidin plates (Thermo Scientific) were coated with biotinylated antibody solution (Ab1-5, lug/mL in PBS) for 1 hour. Antibody coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antigen being tested for approximately one hour. At the end of this incubation, the plate was washed three times with wash buffer and further incubated with a secondary antibody containing solution (Peroxidase conjugated affinipure F(ab′)2 fragment goat anti-human IgG, Fc fragment specific (Jackson Immunoresearch) for approximately 45 minutes and washed three times. At that point a substrate solution (TMB peroxidase substrate, BioFx) was added and incubated for 3 to 5 minutes in the dark. The reaction was stopped by addition of 0.5M HCl and the plate was read at 450 nm in a plate-reader.

Antibody Down Europium ELISA

Briefly, White streptavidin plates (Thermo Scientific) were coated with biotinylated antibody solution (Ab1-5, lug/mL in PBS) for 1 hour. Antibody coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antigen being tested for approximately one hour. At the end of this incubation, the plate was washed three times with wash buffer and further incubated with a secondary antibody containing solution (Europium conjugated anti-human IgG (Cisbio) for approximately 45 minutes and washed three times. At that point 200 μl of HTRF buffer (Cisbio) was added and plates read at with excitation at 330/emission at 620 nm.

The antibodies tested in this example were Ab-A, Ab-B, and Ab-C, which are three different humanized IgG1 antibodies that were expressed in P. pastoris. Each humanized antibody tested in these examples was raised against a different immunogen and specifically binds to a different target molecule than the others.

Results

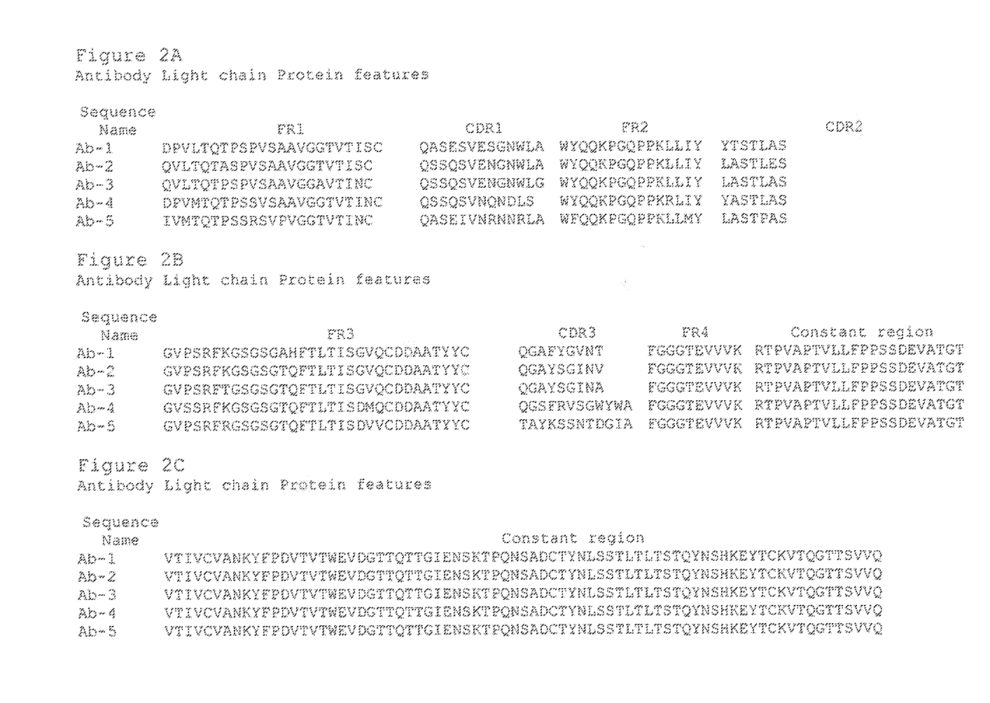

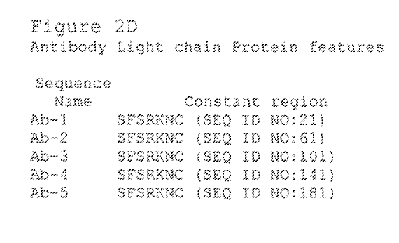

FIG. 13 shows results of ELISA assays using Ab1 and Ab2 to detect glycosylation of different lots of antibody Ab-A. The assay format was anti-glycovariant (AGV) antibody down, with horseradish peroxidase (HRP) detection. Biotinylated antibodies were bound to streptavidin plates with different Ab-A lots titrated. The two antibodies Ab1 and Ab2 reacted similarly to each test sample. In this assay format the sensitivity of Ab1 and Ab2 was relatively similar, possibly due to a “super-avidity” effect with the antibody down on the plate and multi-point mannosylated Ab-A in solution.

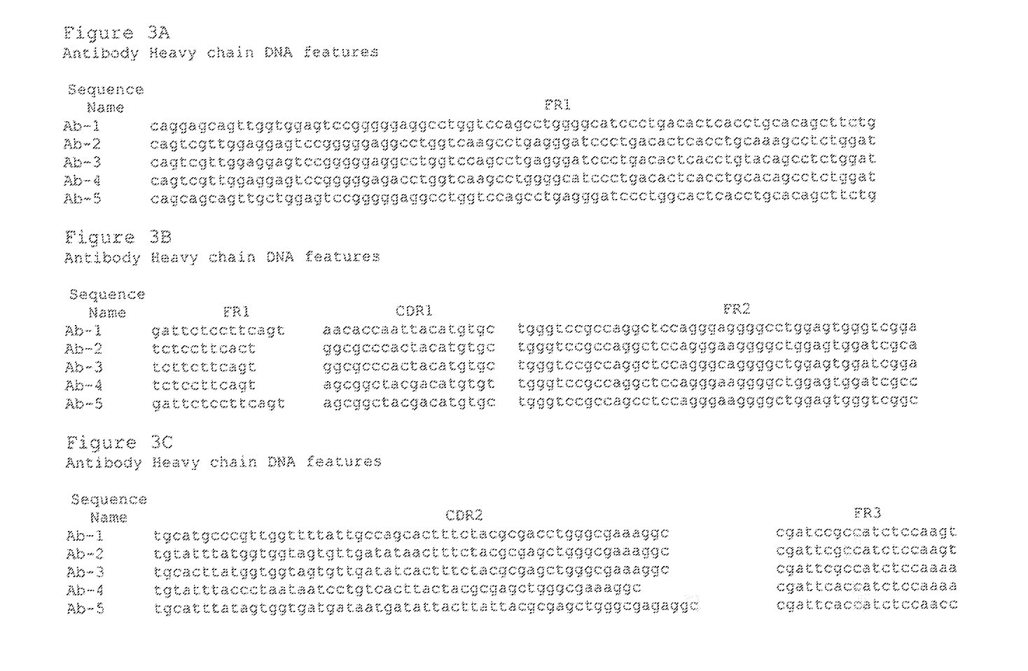

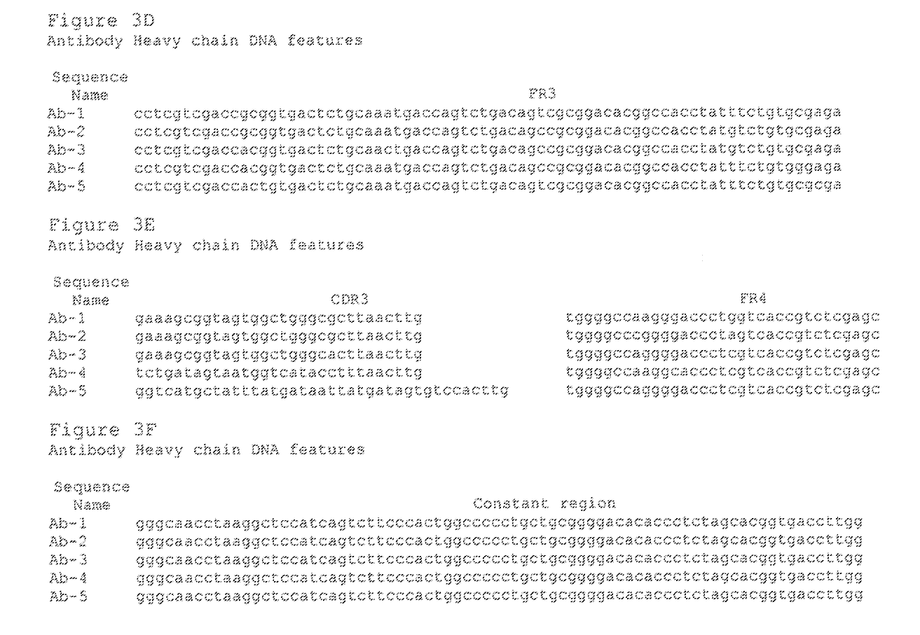

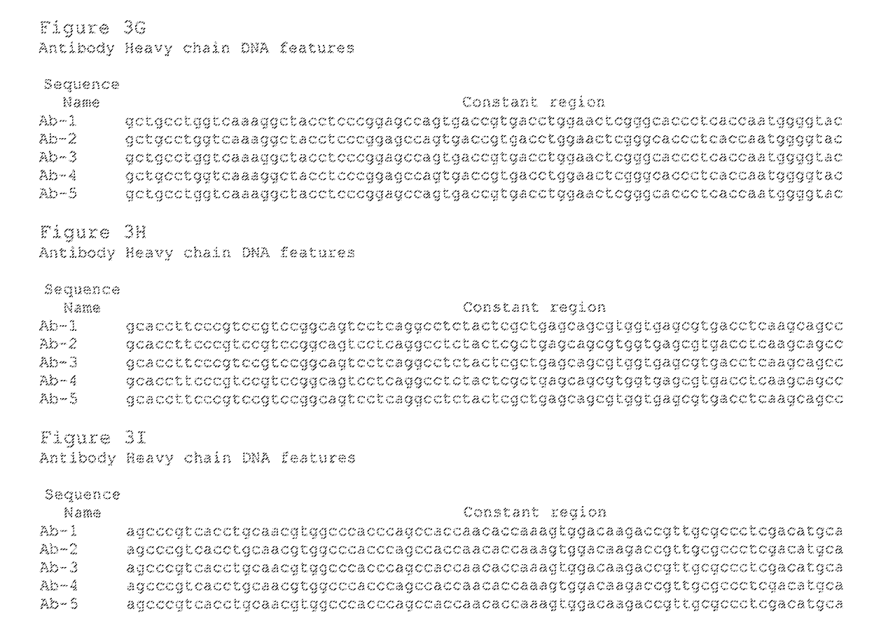

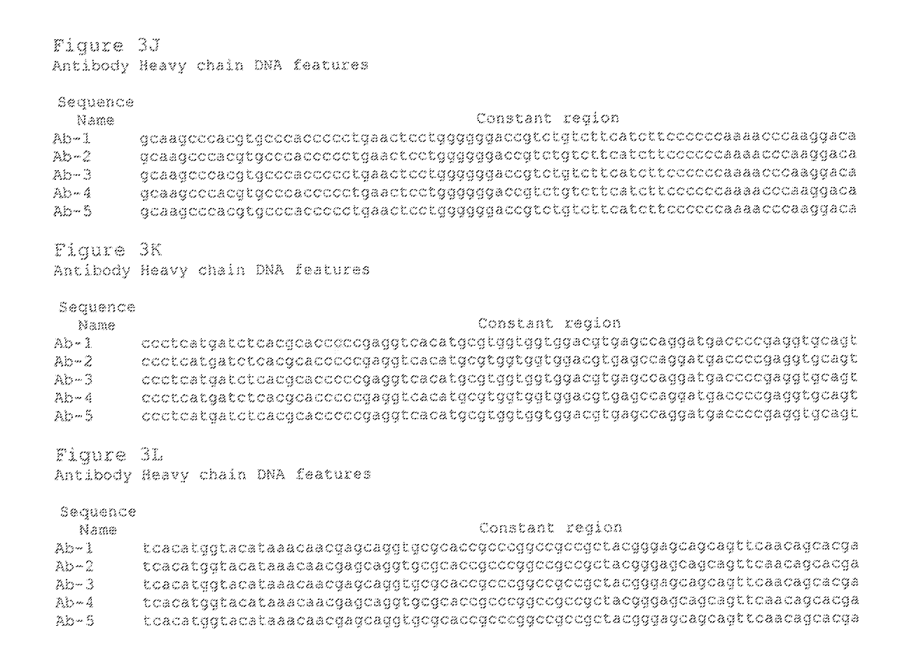

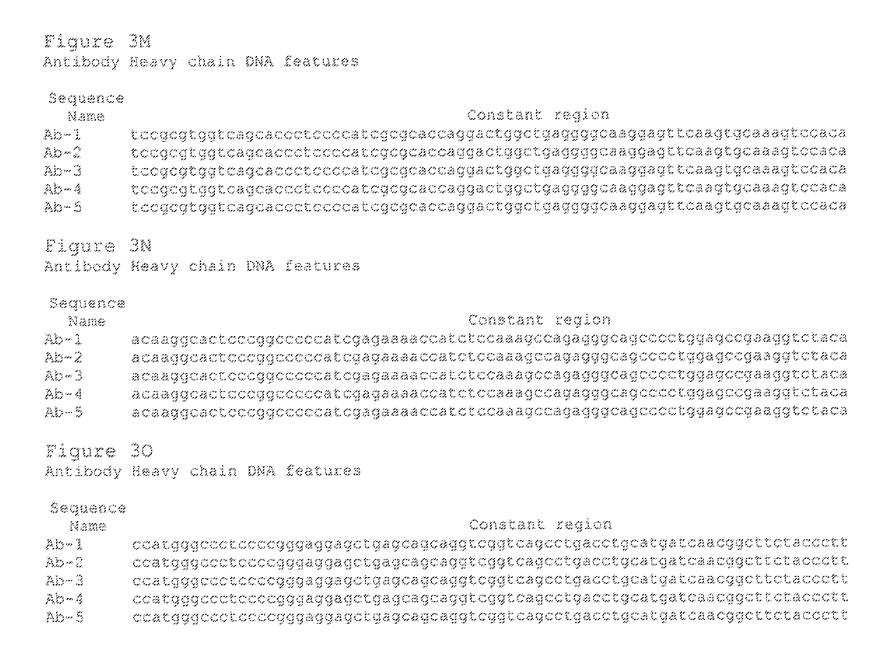

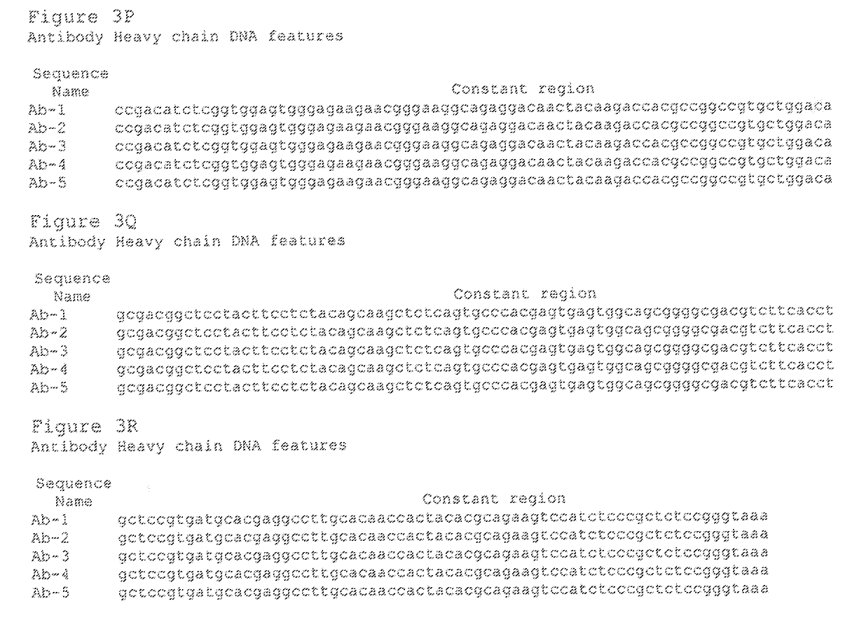

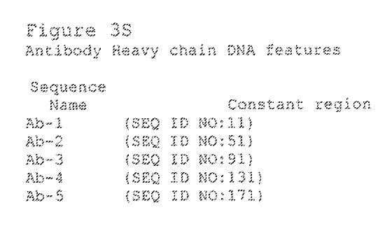

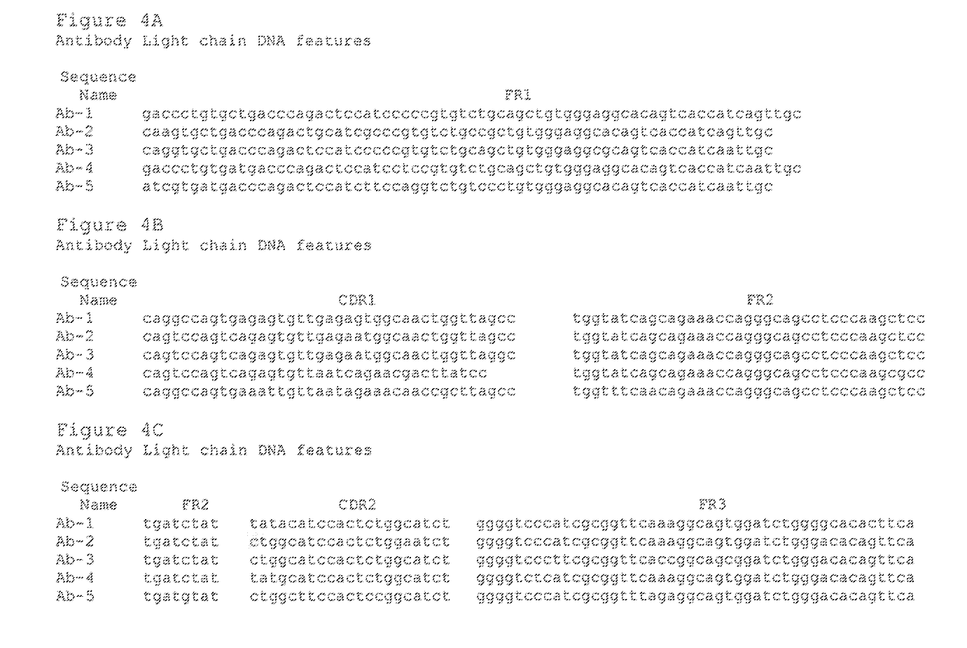

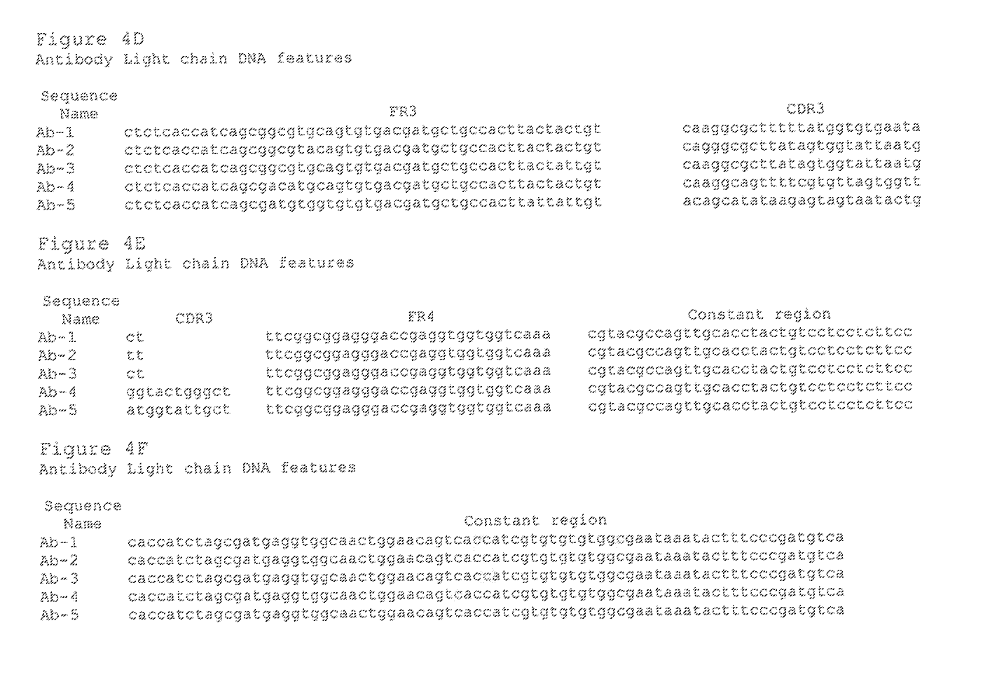

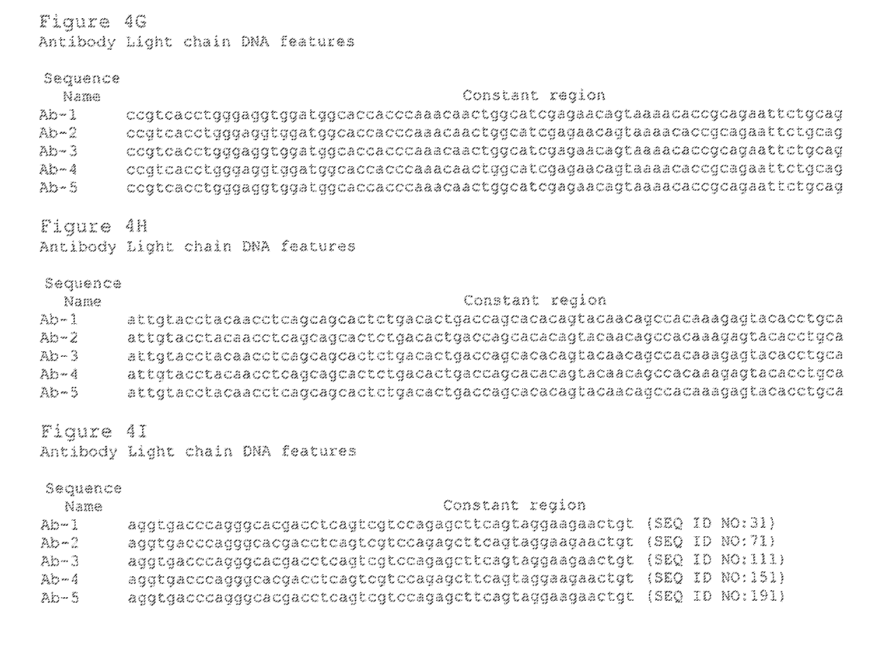

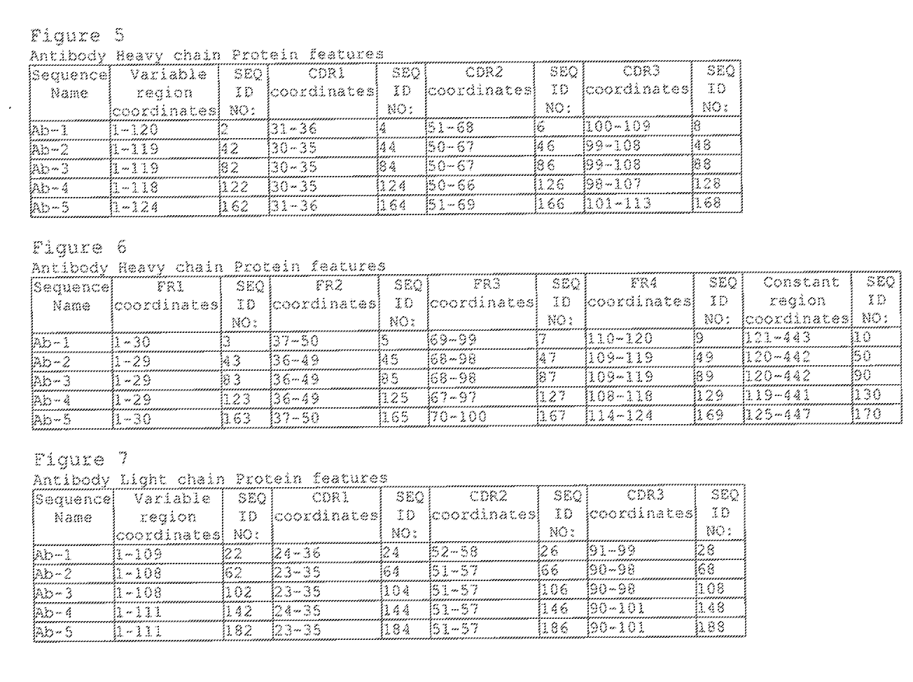

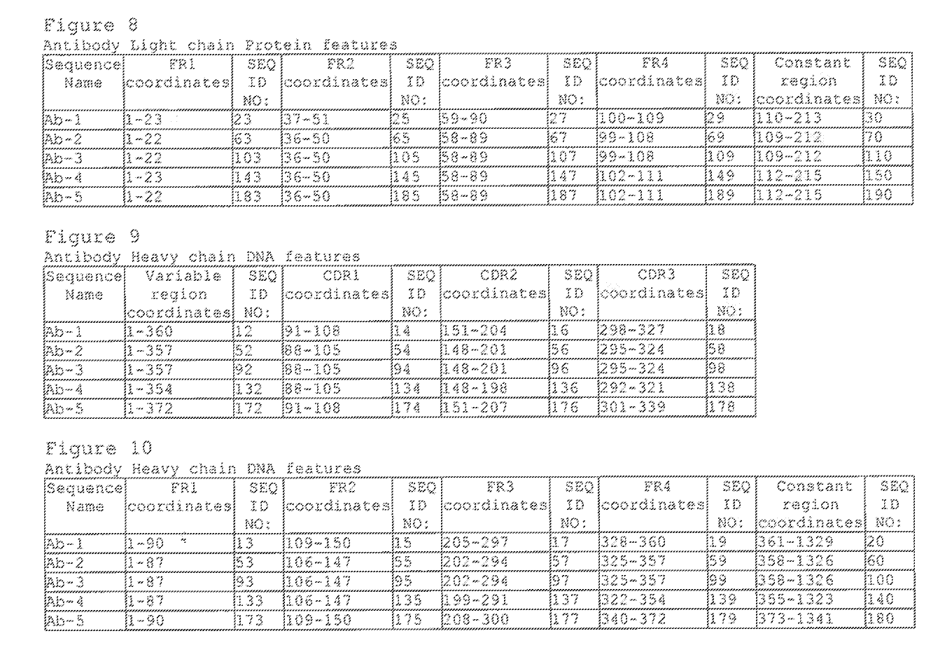

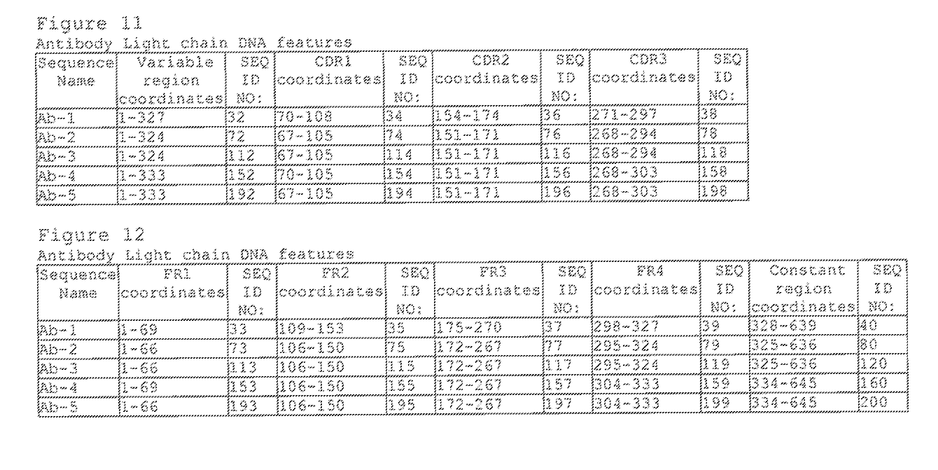

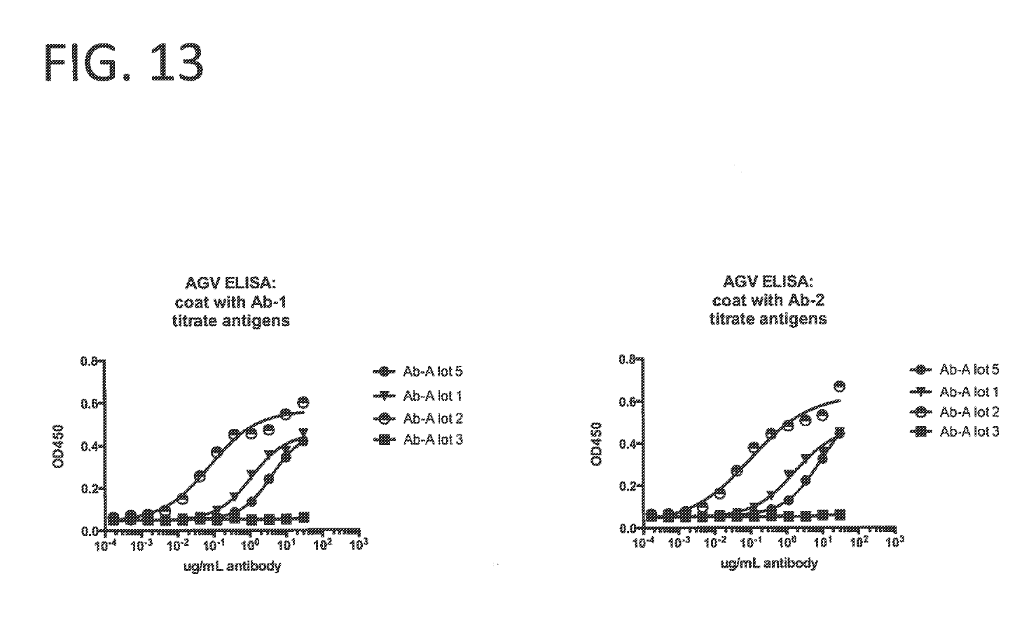

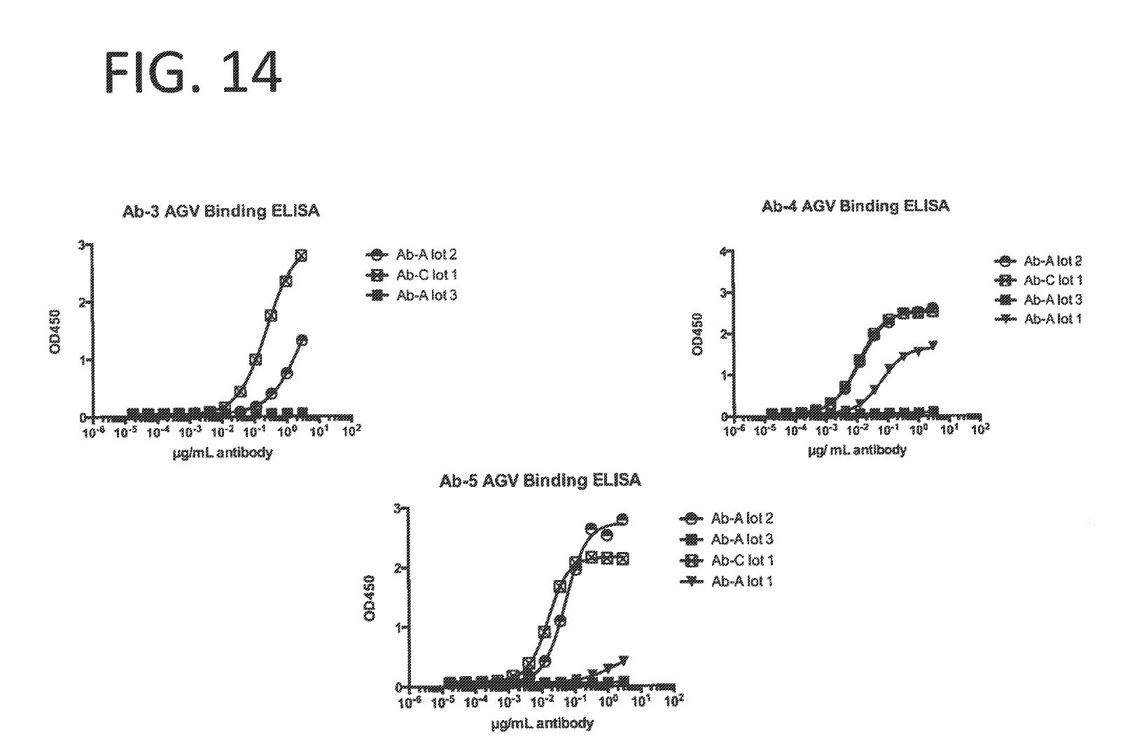

FIG. 14 shows results of ELISA assays using Ab3, Ab4, and Ab5 to detect glycosylation of different lots of antibody Ab-A and Ab-C. The assay format was biotinylated antigen down on streptavidin plates, with the anti-glycovariant (AGV) antibody titrated. The antibodies reacted similarly (though with some differences that may be due to differences in affinity) to the different antigens.

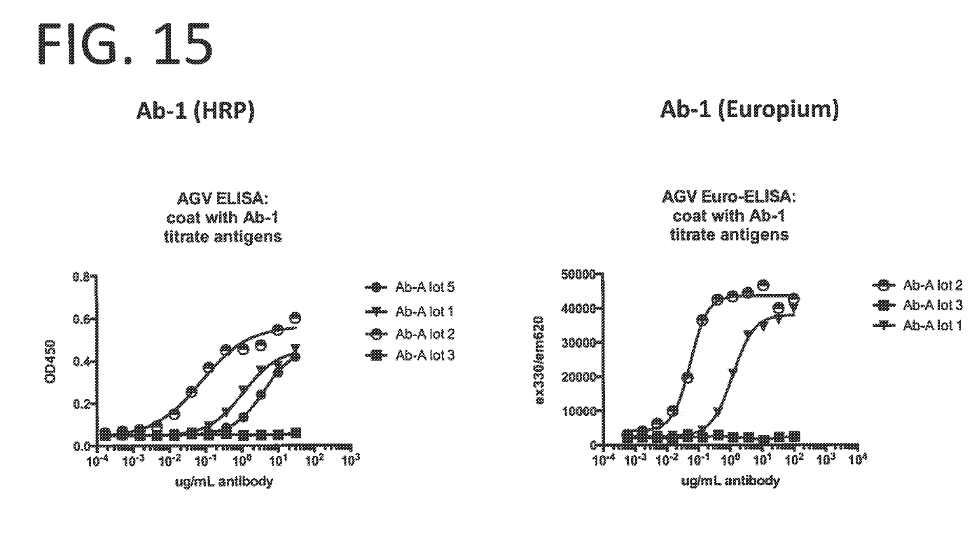

FIG. 15 shows results of ELISA assays using Ab1 to detect glycosylation of different lots of antibody Ab-A. The assay format was anti-glycovariant (AGV) antibody down, with horseradish peroxidase (HRP) or europium (Euro) detection in the left and right panels, respectively. Biotinylated antibodies were bound to streptavidin plates with different Ab-A lots titrated. In the right panel, detection was with a europium-labeled antibody that binds Ab-A (which contains a human constant domain) but not Ab1 (which contains a rabbit constant domain). The use of europium for detection resulted in greater sensitivity than HRP.

### Example 5

### Ab1 Competes for Binding with the Lectin DC-SIGN

This example demonstrates that Ab1 competed with the lectin DC-SIGN for binding to a glycoprotein (specifically, a mannosylated antibody). The results demonstrate that the epitope bound by Ab1 at least overlaps with the binding site for DC-SIGN.

DC-SIGN Blocked by Ab1

A sample of Ab-A lot 2 (a glycoprotein-enriched antibody sample whose preparation is described above in Example 1) was biotinylated with LC-LC-biotin (Pierce cat #21338), bound to streptavidin sensors (Forte Bio Cat. No. 18-5019) for 150 sec at 10 ug/ml and then subjected to pretreatment with Ab1 at 20 ug/ml or 0 ug/ml in 1× Kinetics buffer for 1500 seconds to achieve saturation. Pretreatment signal (not shown) was then normalized to zero on both X- and Y-Axes. The next step of the experiment maintained the same Ab1 concentrations of 20 ug/ml and 0 ug/ml but with the inclusion of DC-SIGN (R&D Systems Cat. No. 161-DC-050) at 15 ug/ml. These conditions were held for 500 seconds and no apparent DC-SIGN binding signal was observed in the condition with pretreatment of Ab1 at 20 ug/ml. Strong signal was observed in the DC-SIGN condition without Ab1 treatment. Sensors were then moved to dissociation conditions in 1× kinetics buffer. DC-SIGN appeared to remain bound, while in the condition with Ab1 bound in pretreatment, signal was observed to decay from its previous level.

Results

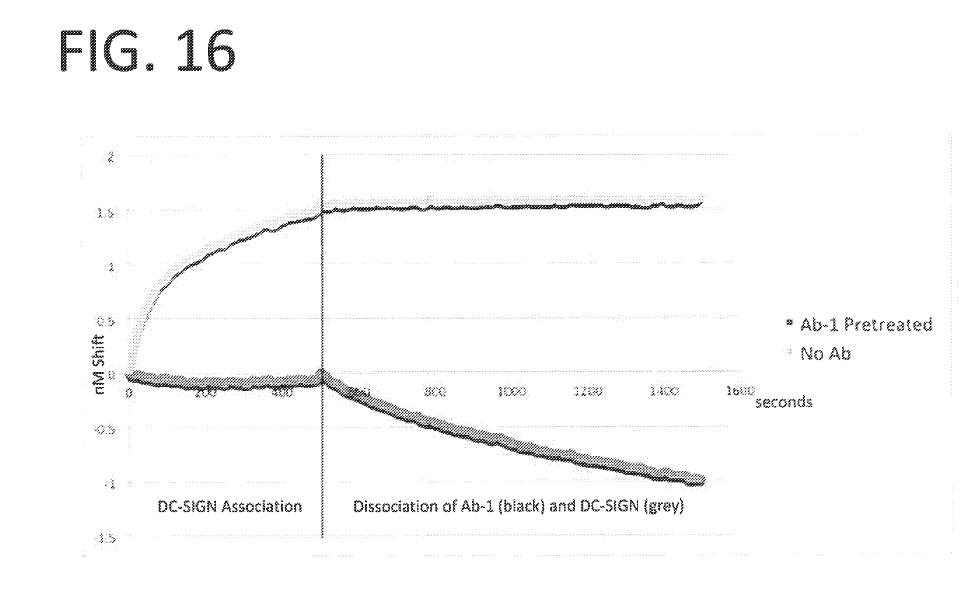

As shown in FIG. 16, binding of DC-SIGN to Ab-A lot2 coated biosensors (upper grey line) is precluded (lower black line) by Ab1 pre-treatment. These results demonstrate that the epitope to which Ab1 binds on the mannosylated protein at least overlaps with the binding site for DC-SIGN.

### Example 6

### A High-Throughput Assay for Detection of Glycoproteins

This assay describes a high-throughput HTRF-based assay for detection of glycoproteins.

Methods

AGV HTRF Assay

Briefly, half area white 96 well plates (Perkin Elmer) were used to read antibody/antigen interactions. Antibody (3 nM), antigen (1 nM), Europium labeled anti rabbit Fc (1 nM donor-Cisbio), and anti human XL665 (30 nM acceptor-Cisbio) are combined in assay buffer (Cisbio) in 60 μl per well. Samples are incubated for 1 hr at room temperature. Upon incubation plates are read at excitation 330 nm, emission 620/665 nm with a delay of 300 microseconds. Data are reported as a ratio of 665/620. The antibodies tested in this example were Ab-B and Ab-D, which are two different humanized IgG1 antibodies that were expressed in P. pastoris. Each humanized antibody tested in these examples was raised against a different immunogen and specifically binds to a different target molecule than the others.

Results

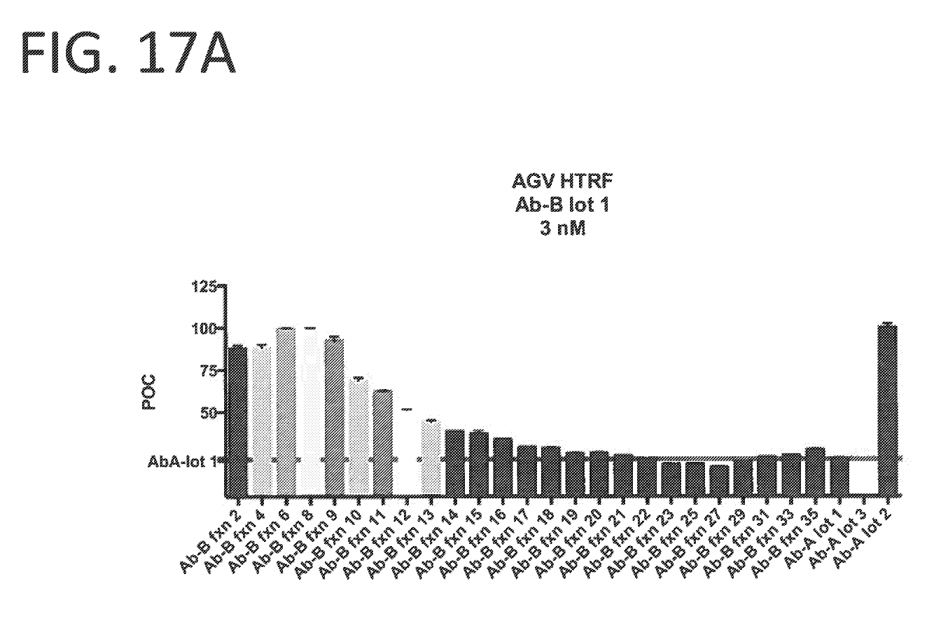

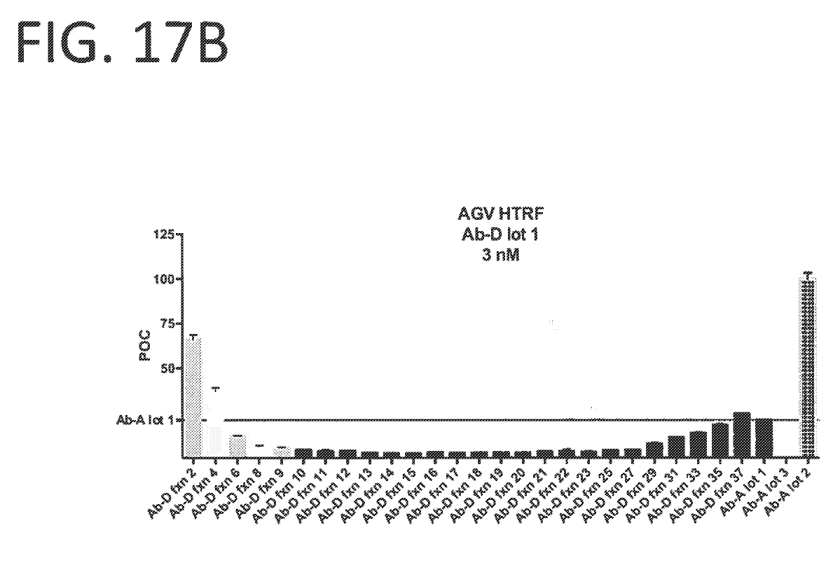

FIG. 17A-B shows use of AGV antibody Ab1 in the high throughput assay (HTRF) to quantify the level of glycoprotein in purification fractions. Ab-B (FIG. 17A) and Ab-D (FIG. 17B) were subjected to column purification and every other collected fraction (as numbered on horizontal axis) was assayed using the AGV antibody to determine the relative amount of glycoprotein. Amount of antibody is expressed as the percentage of control (POC), specifically the amount of glycoprotein relative to a glycoprotein-enriched preparation of Ab-A (Ab-A lot 2). For reference, the amount of glycoprotein contained in Ab-A lot 1 (which contains a relatively low amount of glycoprotein) is indicated by a horizontal line, which was at a level of about 25% of control.

Using this assay, fractions can be selected or pooled to obtain a glycoprotein enriched or glycoprotein depleted preparation as desired.

### Example 7

### Relative Quantification of Glycoproteins in Purification Fractions

This example demonstrates glycoprotein analysis of chromatographic purification fractions of a glycoprotein-containing antibody. Glycoproteins were detected using the anti-glycoprotein antibody Ab1 or GNA.

Methods

Chromatographic fractions of Ab-A eluted from a polypropylene glycol (PPG) column were subject to glycoprotein analysis using Ab1 or GNA. Detection based on Ab1 was performed by the HTRF method described in Example 6.

For GNA analysis, streptavidin Biosensors with Biotinylated Galanthus nivalis agglutinin were used to determine the concentration of glycovariants in solution relative to a standard. In particular, an Octet interferometer (ForteBio, Menlo Park, Calif.) with Streptavidin Biosensors (ForteBio) functionalized with biotinylated Galanthus nivalis Lectin (GNL, also referred to as GNA, Cat B-1245, Vector Labs, Burlingame, Calif.) was used to determine the level of activity of a biomolecule in solution relative to a standard. Briefly, sensors were functionalized by pre-wetting in 1× kinetics buffer (a 1:10 dilution in Dulbecco's Phosphate Buffered Saline of 10× kinetics buffer from ForteBio, Part No: 18-5032) then immersed in a dilution of biotinylated GNL lectin and placed on a shaking platform for a prescribed length of time.

Sample storage and handling: Samples and standards were stored at 4° C. or −20° C. depending on existing stability data. While preparing the assay, samples were kept on ice. Kinetics buffers (Forte Bio Catalog No. 18-5032, 10× and 1×, containing PBS+0.1% BSA, 0.02% Tween20 and 0.05% sodium azide) were stored at 4° C. GNL is stored at 4° C.

Functionalizing the sensors: Streptavidin sensors (Forte Bio Catalog No. 18-5019, tray of 96 biosensors coated with streptavidin) were soaked in 1× Kinetics buffer for at least 5 minutes. Biotinylated GNL was diluted 1/1000 into 1× kinetics buffer to obtain the volume calculated in step below. 1× kinetics buffer was prepared from 10× kinetics buffer and Hyclone DPBS+Ca+Mg. 120 ul of kinetics buffer was aliquoted per well for each sensor needed into a half area black plate, e.g., 96-Well Black Half Area Plates Medium & High Binding (Greiner Bio-One Cat 675076 or VWR Cat 82050-044). The sensors were transferred to plates with Biotinylated GNL, and the plates were incubated with shaking for at least 30 minutes.

Preparation of the sensors and samples: Sensors were handled with a multichannel pipettor with particular care for the tips of the sensors since damage (e.g., scraping) to these tips can affect the assay results. A medium binding black plate was used for sensors with sensor tray. A separate black plate was used for samples and standards. 150 μl was added per well for unknowns, controls and standards. A media blank or a solution containing a known glycovariant concentration can be optionally included as a control sample. A new sensor was used for each standard well of the assay. Each sensor was rinsed in 1× kinetics buffer before use. A duplicate 3-fold dilution series of 8 points was sufficient for a standard curve. The dilutions were made using 1× kinetics buffer. 1× kinetics buffer was also used as a blank sample.

The Octet conditions were set as follows: Quantitation Time (s) 250; Shake speed 1000 rpm. The plate was defined by assigning the sample wells and the sensors. In particular, the sample wells were assigned by selecting the wells corresponding to the samples and entering their identity, e.g., “unknown” to input a dilution factor or “standard” to input a known concentration. The sensors were not reused for this assay. The program optionally included a delay and/or shaking before processing the sample (e.g., plate was equilibrated to 30° C. while shaking at 200RPM for 300 seconds).

Standards, unknowns and controls for measurement were diluted in IX kinetics buffer and arrayed in a black microtiter plate, with replicates as appropriate. The plate with sample dilutions was read on the Octet using the GNL-functionalized sensors and standard quantitation assay methods (such as for Protein A sensors) as described by the manufacturer (ForteBio).

Data Analysis was performed with a ForteBio Analysis software module. Standard curve linearity and reproducibility of known samples were evaluated. Well activity levels were appropriately adjusted for sample concentration/dilution factor to determine mass—normalized specific activity levels, termed Relative Units (RU) or Percent of Control (POC)

Results

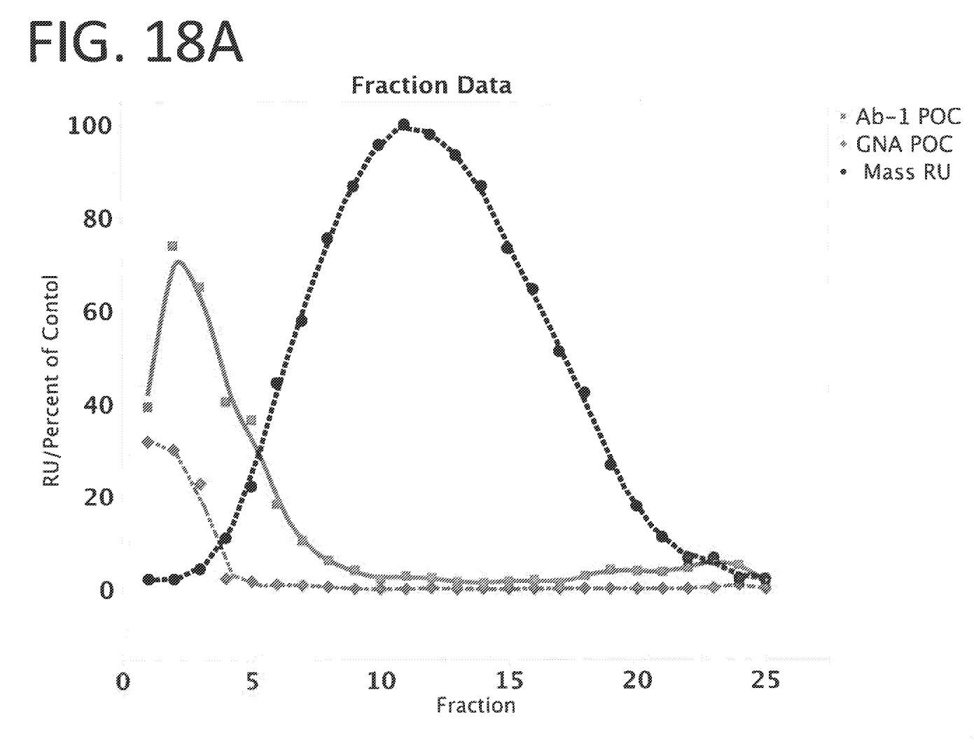

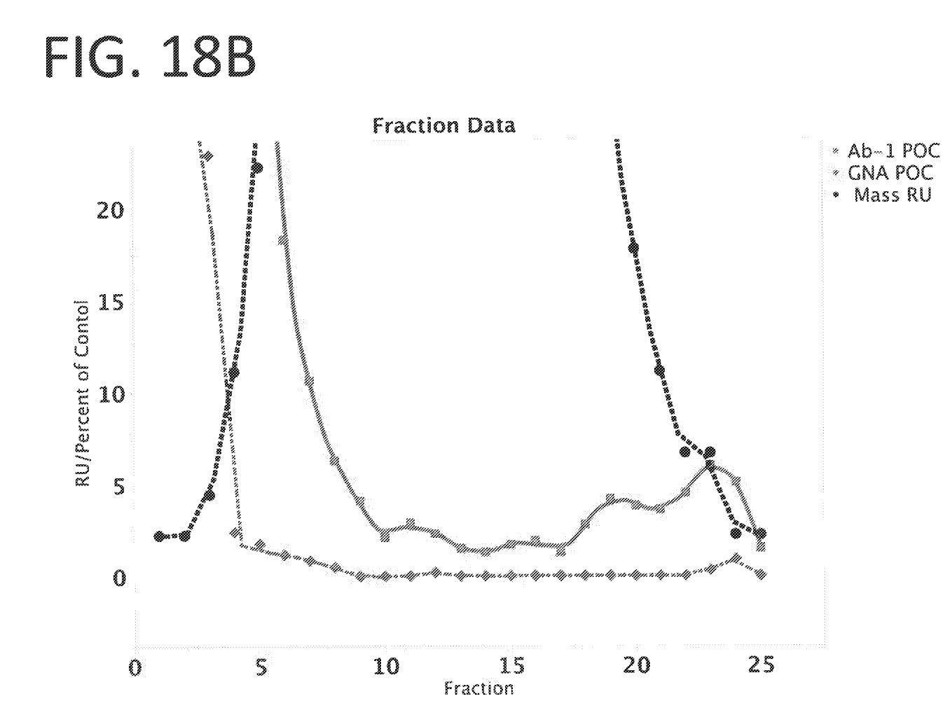

FIG. 18A-B shows quantification of glycoprotein contained in fractions of Ab-A eluted from a polypropylene glycol (PPG) column. Ab1 and GNA were used to evaluate the relative amount of glycoprotein (expressed as percentage of control, POC) contained in each fraction. Protein mass contained in each fraction is also shown in relative units (Mass RU). A similar pattern of reactivity was seen for detection using Ab1 and GNA.

Results were similar Ab1 and GNA, indicating that Ab1 provides a viable detection method for detecting presence of glycoproteins in purification fractions.

### Example 8

### Head-to-Head Comparison of Ab1, GNA, and DC-SIGN for Glycoprotein Detection

This example shows detection of glycoproteins in multiple lots of an antibody by Ab1, GNA, and DC-SIGN detection methods. The relative levels of glycoprotein detected by each method were similar, further confirming suitability of methods using of Ab1 for detecting presence of glycoproteins.

Methods

Sample storage and handling: Samples and standards were stored at 4° C. or −20° C. depending on existing stability data. While preparing the assay, samples were kept on ice. Kinetics buffers (Forte Bio Catalog No. 18-5032, 10× and 1×, containing PBS+0.1% BSA, 0.02% Tween20 and 0.05% sodium azide) were stored at 4° C. GNL is stored at 4° C.

Functionalizing the sensors: Streptavidin sensors (Forte Bio Catalog No. 18-5019, tray of 96 biosensors coated with streptavidin) were soaked in 1× Kinetics buffer for at least 5 minutes. Biotinylated GNL was diluted 1/1000 into 1× kinetics buffer to obtain the volume calculated in step below. 1× kinetics buffer was prepared from 10× kinetics buffer and Hyclone DPBS+Ca+Mg. 120 ul of kinetics buffer was aliquoted per well for each sensor needed into a half area black plate, e.g., 96-Well Black Half Area Plates Medium & High Binding (Greiner Bio-One Cat 675076 or VWR Cat 82050-044). The sensors were transferred to plates with Biotinylated GNL, and the plates were incubated with shaking for at least 30 minutes.

Preparation of the sensors and samples: Sensors were handled with a multichannel pipettor with particular care for the tips of the sensors since damage (e.g., scraping) to these tips can affect the assay results. A medium binding black plate was used for sensors with sensor tray. A separate black plate was used for samples and standards. 150 μl was added per well for unknowns, controls and standards. A media blank or a solution containing a known glycovariant concentration can be optionally included as a control sample. A new sensor was used for each standard well of the assay. Each sensor was rinsed in 1× kinetics buffer before use. A duplicate 3-fold dilution series of 8 points was sufficient for a standard curve. The dilutions were made using 1× kinetics buffer. 1× kinetics buffer was also used as a blank sample.

The Octet conditions were set as follows: Quantitation Time (s) 250; Shake speed 1000 rpm. The plate was defined by assigning the sample wells and the sensors. In particular, the sample wells were assigned by selecting the wells corresponding to the samples and entering their identity, e.g., “unknown” to input a dilution factor or “standard” to input a known concentration. The sensors were not reused for this assay. The program optionally included a delay and/or shaking before processing the sample (e.g., plate was equilibrated to 30° C. while shaking at 200RPM for 300 seconds).

A different lectin, DC-SIGN (R&D Systems cat #161-DC-050) was biotinylated with LC-LC-biotin (Pierce cat #21338) and used to functionalize streptavidin sensors that were employed in a similar assay as described above.

Results

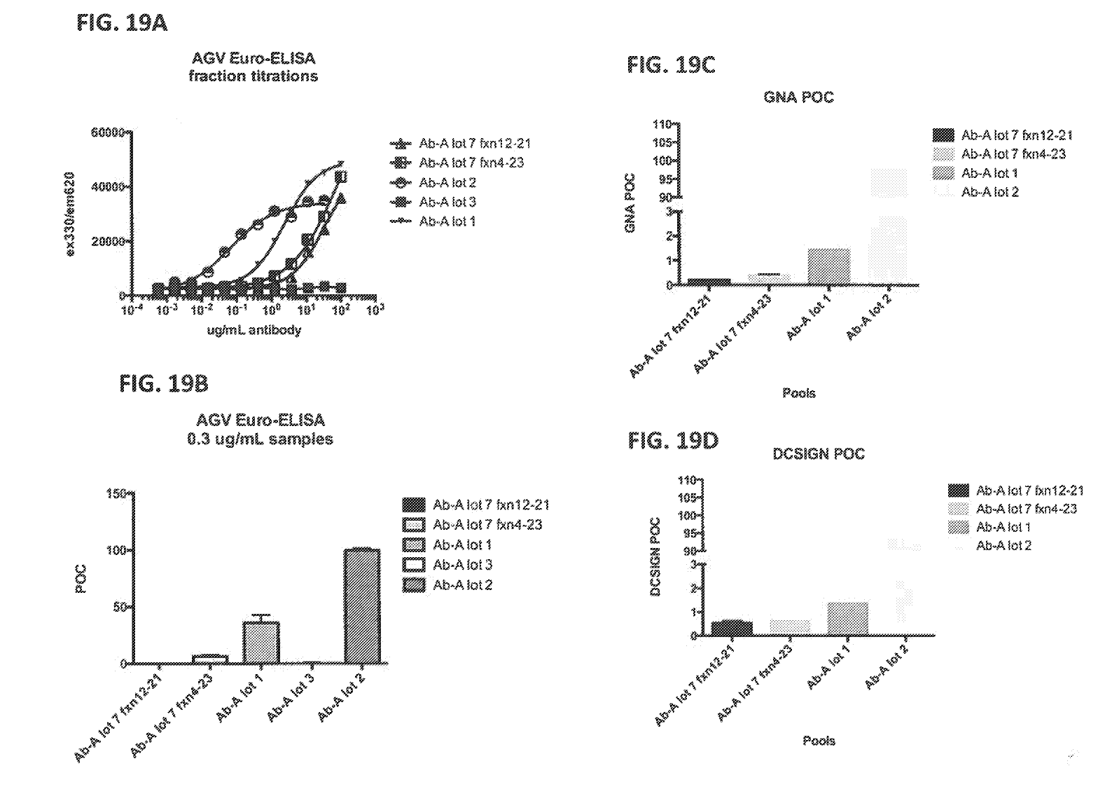

FIG. 19A-D shows results of glycoprotein analysis of pooled fractions from the purification shown in FIG. 18A-B. FIG. 19A shows ELISA detection of glycoproteins in different preparations using an AGV antibody Ab1 in an europium-based antibody-down ELISA assay as in FIG. 15 (Ab1 down on plate, 0.3 μg/mL Ab-A samples in solution). FIG. 19B graphically illustrates the detected level of glycoprotein detected using the ELISA assay as a percentage of a control sample (POC). FIG. 19C-D shows the detected level of glycoprotein in the same samples determined using GNA or DC-SIGN, respectively. The labels “fxn12-21” and “fxn4-23” respectively indicate pooling of fractions numbered 12 through 21 or 4 through 23 from the purification shown in FIG. 18A-B.

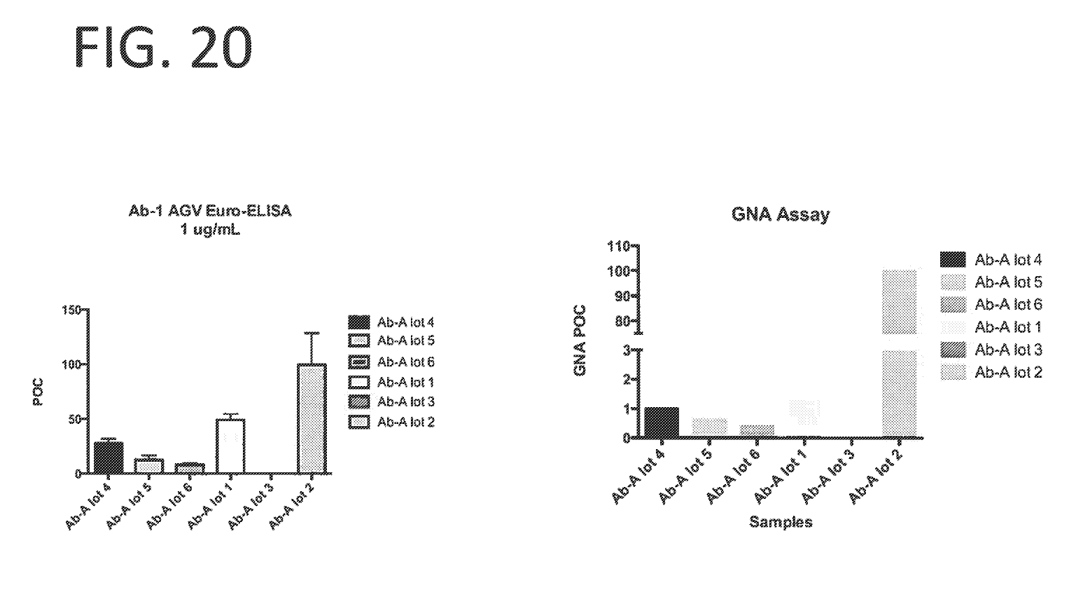

FIG. 20 shows results of glycoprotein analysis of antibody preparations using ELISA detection (left panel) or a GNA assay (right panel), each expressed as percentage of a control sample (POC). Results were qualitatively similar across the six tested lots, with relative peak height forming a similar pattern for each.

Very similar profiles were seen with the AGV antibody, GNA, and DC-SIGN assays on these samples. Notwithstanding some differences in absolute peak height (as percentage of control values), these results further validate the use of Ab1 for detection of glycoproteins.

### Example 9

### O-Glycoform Composition Analysis

This example shows the correlation between signals obtained using antibody Ab1, GNA, and DC-SIGN and the amounts of mannose.

Methods

Three lots of Ab-A were subjected to O-glycoform analysis. Relative quantities of mono-, di-, and tri-mannose contained in each preparation were determined generally as described in Stadheim et al., “Use of high-performance anion exchange chromatography with pulsed amperometric detection for O-glycan determination in yeast,” Nature Protocols, 2008 3:1026. Each lot was subject to glycoprotein analysis using GNA as described in Example 7 and DC-SIGN, as described in Example 8. Additionally, for each lot, glycoprotein analysis using Ab1 was performed by the HTRF method described in Example 6. Signals for each detection method were quantified as a percentage of control (POC).

Results

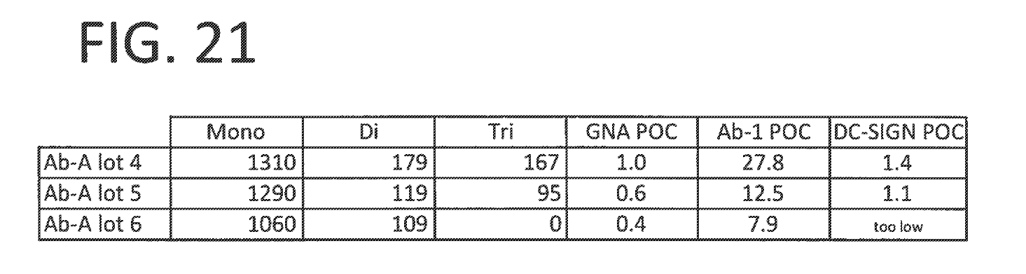

FIG. 21 shows results of O-glycoform composition analysis relative to signal from AGV, GNA, and DC-SIGN. The table shows relative units of sugar alcohol, specifically levels of mono-, di-, and tri-mannose, as well as GNA, Ab1 and DC-SIGN signal for each sample.

The results show that the signals obtained from an AGV mAb (Ab1), GNA, and DC-SIGN binding assays correlate with each other and with the amount of mannose on Ab-A.

### Example 10

### Enrichment and Screening of Yeast Strains Using Ab1

Introduction

Low productivity of the cells can be a limiting factor in recombinant protein production. Isolating high performing strains represents a powerful approach for increasing productivity. Several molecular biology techniques can be used to create genetic diversity, including mutagenesis (random or semi-rational) and recombinant DNA methods, or spontaneously arising strains can also be used. The library size created via such techniques is typically very large (>10⁵), rendering the isolation of the desired mutant a typical “needle in a haystack” problem. High-throughput screening can be used to enrich the variants with desired properties, such as increased productivity.

This example describes the use of a cell-surface affinity (or “capture”) matrix to enrich for high-producing cells. The general principle of operation is that the secreted antibody can be retained on the surface of the secreting cell (its “capture”), allowing its subsequent detection. Use of a fluorescent detection reagent allows enrichment of high-producing cells by cell sorting. The exemplified capture matrix makes use of the strong Biotin-Avidin interaction. The cell surface is labeled with a biotin-conjugated cell-binding agent, specifically, an anti-glycoprotein antibody. The cells labeled with biotinylated anti-glycoprotein antibody are then mixed with Avidin (or Streptavidin), which provides a bridge to attach a biotinylated “capture antibody” capable of binding the secreted product. Subsequently, the cells are allowed to secrete their products under defined conditions, resulting in retention (capture) of the secreted product by the cell-surface capture matrix. The cells can then be washed, stained and assayed for the secreted product using flow cytometry.

Methods

The reagents used were: FACS buffer (PBS with 2% FBS); Biotinylated Ab1 (described in the examples above) as a stock solution with a concentration of 1 mg/ml; Streptavidin (Jackson Immunoresearch Catalog #016-000-084) as a stock solution with a concentration of 5 mg/ml; Biotinylated Donkey Anti-Human IgG (H+L) ML* “Capture Antibody” (Jackson Immunoresearch Catalog #709-065-149 as a stock solution with a concentration of 1 mg/ml; Fluorescent-labeled Donkey Anti-Human IgG (H+L) ML* “Detection Antibody”: (Jackson Immunoresearch Catalog #709-545-149) as a stock solution with a concentration of 0.5 mg/ml; Propidium Iodide 50 ug/ml (BD Pharmingen 51-66211E); and acid free media (AFM) supplemented with 10% PEG8000.

Cells were grown in BYEG media overnight at 30° C. Cell density was determined by measuring optical density at 600 nm using a spectrophotometer, with dilution if needed to obtain a concentration in the linear range (0.1 to 0.9 OD). Cell density was calculated by multiplying the OD600 by the dilution factor times 5×10⁹ to give the approximate cells/ml. Cells were spun down by centrifugation at 3000 rpm for 5 minutes. The cell pellet was resuspended in 200 μl FACS buffer and centrifuged, and this was repeated twice. To the cells was added 1 μl of Biotinylated Ab1 (1 mg/ml) and incubated on ice for 15 minutes. Cells were spun down and washed with FACS buffer at 3000 rpm for 5 minutes, which was repeated twice. Cells were resuspended in 200 μl FACS buffer. Then 1 μl of Streptavidin (5 mg/ml) was added and incubated on ice for 15 minutes. Cells were again spun down and washed with FACS buffer at 3000 rpm for 5 minutes, which was repeated twice. The cells were resuspended in 200 μl FACS buffer. Then 10 μl of “Capture Antibody” (1 mg/ml) was added and incubated on ice for 30 minutes. The cells were spun down and washed with FACS buffer at 3000 rpm for 5 minutes, which was repeated twice. The cells were resuspended in 200 μl AFM media supplemented with 10% PEG8000 and divided into two tubes (Tube A and B). Tube A was spun down immediately and used as the starting time point (“0 hr”) sample and immediately processed. For Tube B, the media was transferred to a 24-well low well plate (LWP) and incubated at 30° C., without shaking, for 2 hours or up to 4 hours to allow for antibody secretion. The higher durations were used in some instances to allow for higher signal accumulation, in which case the media was supplemented with hydroxyurea, to a final concentration to 0.2M, to inhibit cell growth.

The cells were then processed as follows. Cells were spun down and washed with FACS buffer at 3000 rpm for 5 minutes, which was repeated twice. The cells were resuspended in 200 μl FACS buffer. Then 30 μl of Detection Antibody (0.5 mg/ml) was added and incubated on ice for 20 minutes. The cells were then spun down and washed with FACS buffer at 3000 rpm for 5 minutes, which was repeated twice. The cells were resuspended in 200 μl FACS buffer. After the final wash, 0.5 μl Propidium Iodide was added. The tubes were vortexed and kept on ice and covered until FACS analysis/sorting.

Cell sorting was performed on a BD Influx flow cytometer (BD Biosciences, San Jose, Calif., USA), equipped with a 200 mW Argon laser (Coherent, Santa Clara, Calif., USA) for 488 nm excitation and an automatic cell deposition unit for sorting into 96-well plates or FACS tubes. FITC Fluorescence was measured in Fl1 using the standard 528BP filter, Propidium Iodide fluorescence in F13 with a 610BP filter. Data acquisition and analysis were performed with BD Sortware and FlowJo software.

Results

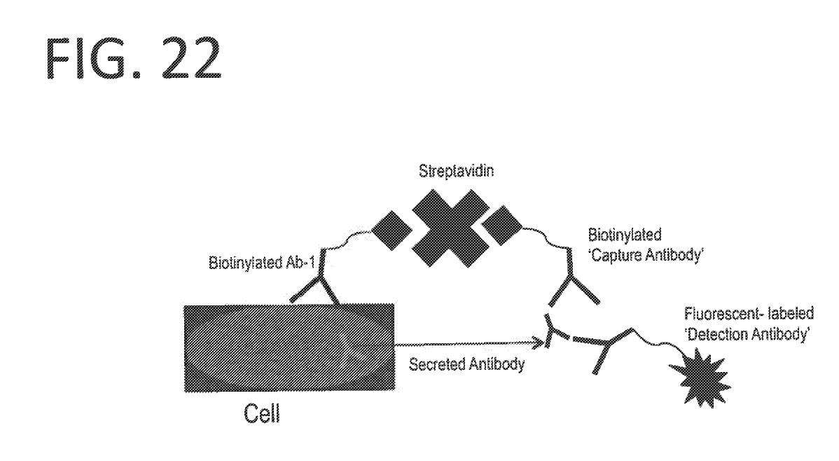

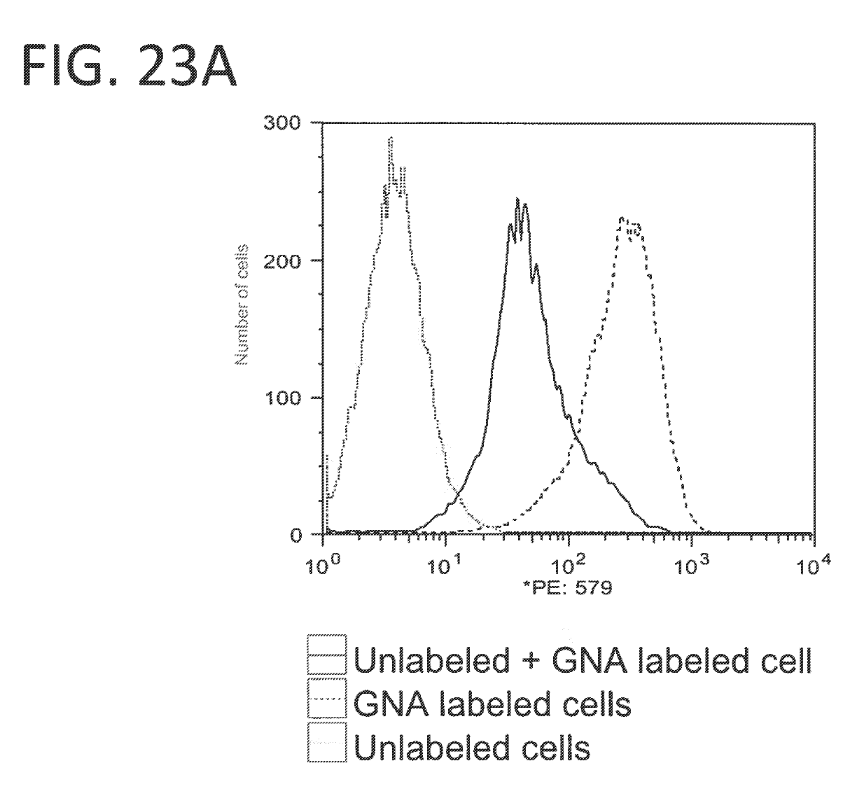

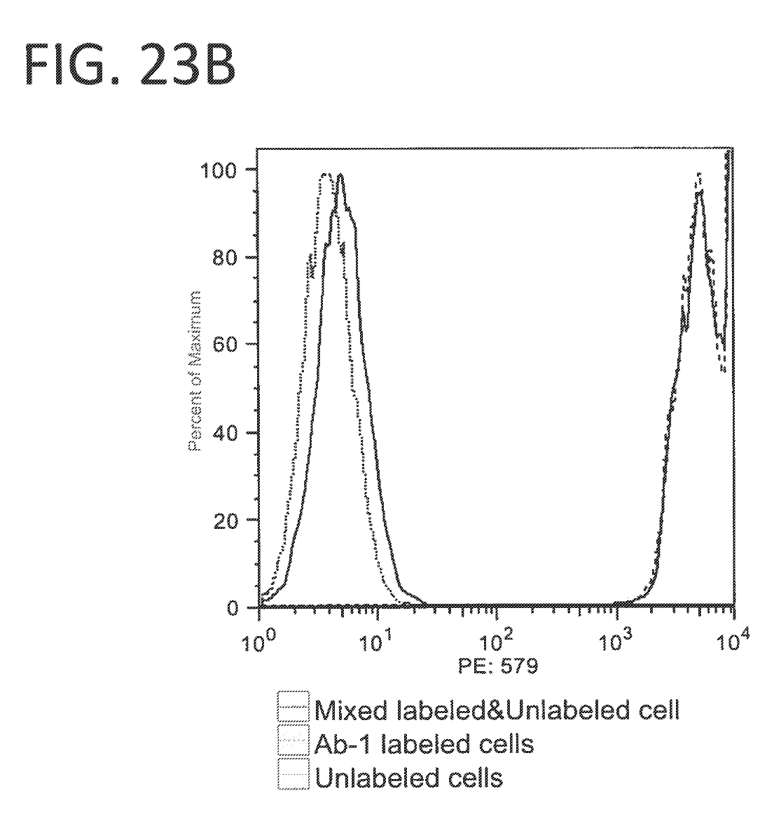

The arrangement of the capture reagents used in this example is illustrated in FIG. 22. Two different cell-binding agents were tested to biotinylate the cell surface: Biotinylated Galanthus nivalis agglutinin (GNA, Vector Laboratories, Burlingame, Calif.) and a biotinylated antibody (Ab1) that binds to mannosylated proteins. Labeling of cell surface with GNA was found to have the disadvantage that the interaction was relatively weak, and upon mixing with unlabeled cells GNA from labeled cells was found to migrate to unlabeled cells, resulting in a single peak for fluorescent signal on flow cytometric profile (FIG. 23A). In contrast, Ab1 was found to bind to the cells strongly and essentially irreversibly, resulting in two fluorescent signal peaks corresponding to the two starting cell populations (FIG. 23B). Thus, the use of an anti-glycoprotein antibody such as Ab1 allows the construction of a stable capture matrix.

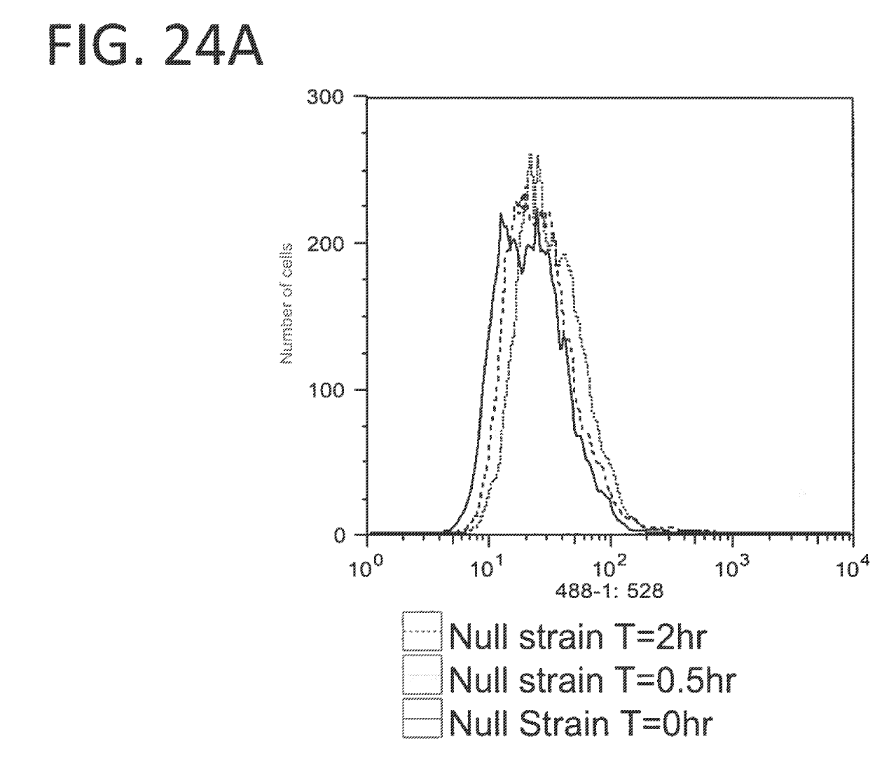

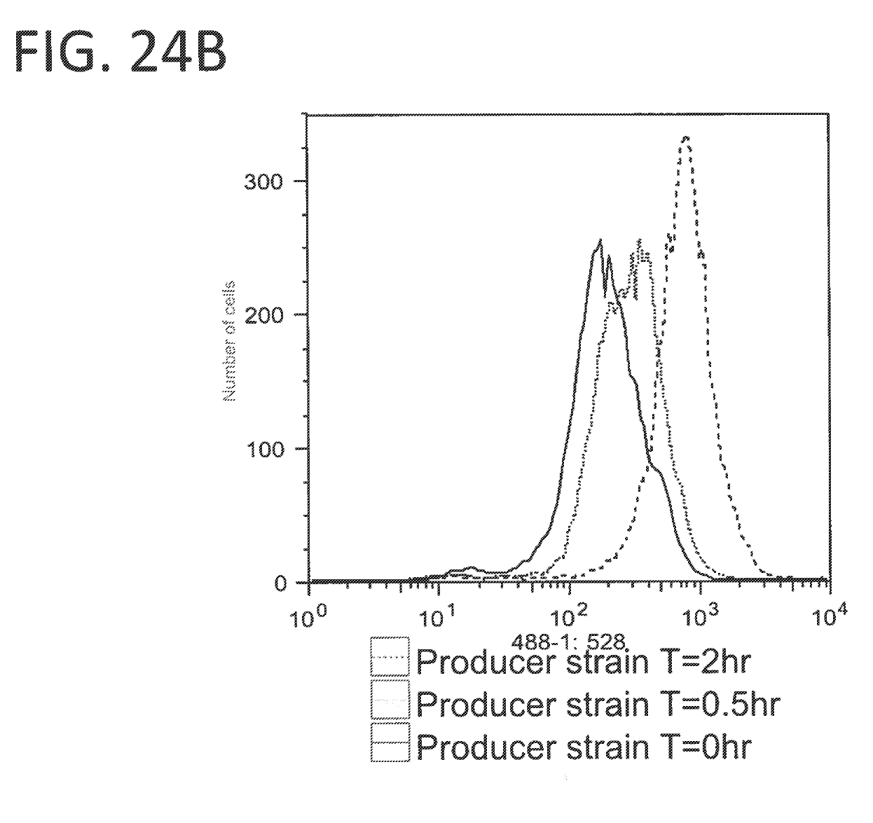

A commercially purchased biotinylated polyclonal anti-human antiserum (Donkey Anti-Human IgG (H+L), Jackson Immunoresearch Catalog #709-065-149) was used as a “capture antibody” with streptavidin as a bridge to link it with the biotinylated Ab l on the cell surface. The labeled cells were then transferred to the production media and allowed to secrete Ab-A for varying amounts of time. Upon subsequent detection of secreted and captured Ab-A with fluorescent-labeled “detection antibody” (Donkey Anti-Human IgG (H+L) ML*, Jackson Immunoresearch Catalog #709-545-149), a consistently increasing signal with incubation time was observed for samples processed after 0, 0.5, or 2 hours (FIG. 24B). A control non-producing “null strain” did not show any increase in signal over the same time-points (FIG. 24A). These results demonstrate the dependence of the fluorescent signal on successful capture of the secreted product.

Mitigating Cross-Binding of Secreted Antibody

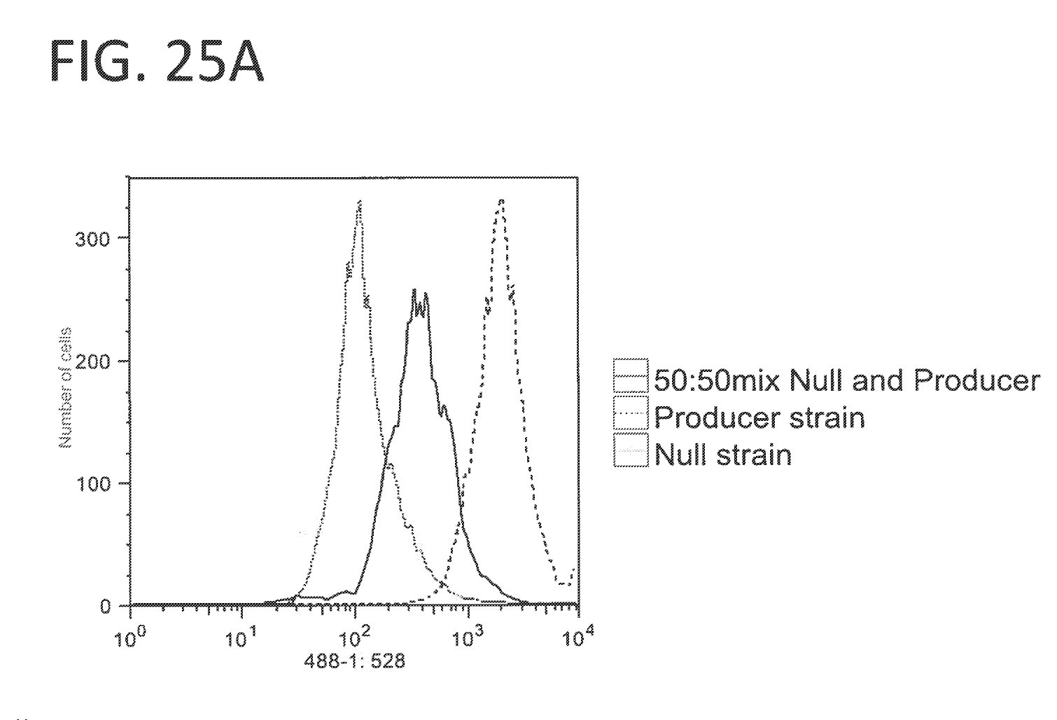

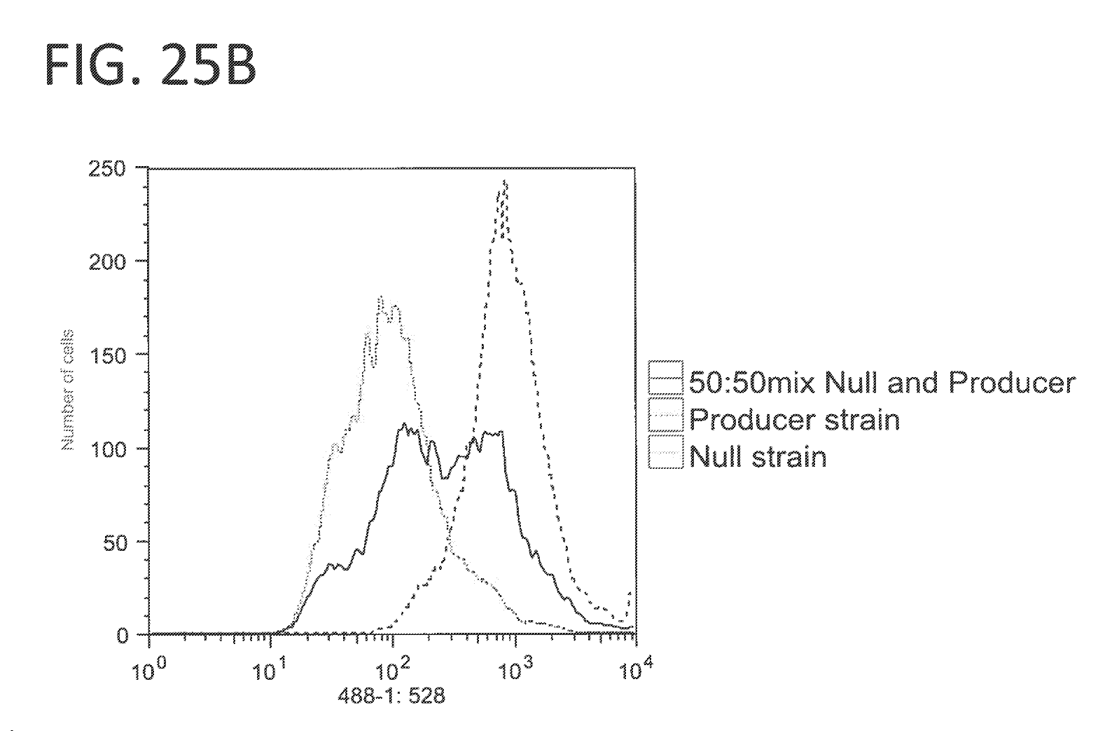

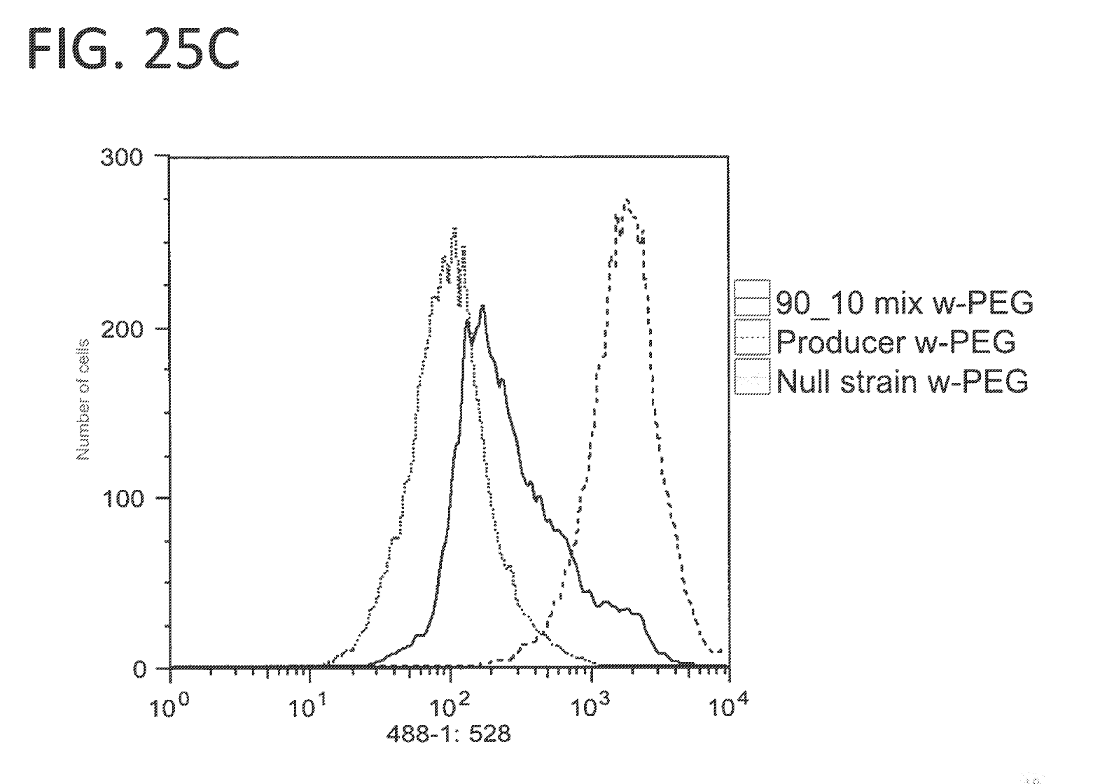

Upon mixing the matrix-labeled Ab-A-secreting “Production strain” with matrix-labeled non-producing null strain, cross-binding of the secreted product was observed (FIG. 25A). From these results, it was inferred that antibody secreted from a high-producing cell (and not captured by the matrix) can diffuse and bind to the capture matrix on low- or non-secreting cells, resulting in a single peak for fluorescent signal on flow cytometric profile. Such cross-binding was addressed by decreasing the permeability of the media. One tested agent was gelatin, however, it was observed that gelatin supplementation, even at concentrations as low as 10%, was found to have a severely negative impact on cell viability and productivity (data not shown). It was hypothesized that the gelatin adversely impacted oxygen and nutrient uptake. Supplementation of media with a molecular crowding agent was tested, specifically 10-15% Polyethylene glycol (PEG8000). It is contemplated that prevention of cross-binding could be attained with other molecular crowding agents such as Dextrans, Ficoll, BSA, and others. PEG molecules of different molecular weights or at differing concentrations could also be used. Supplementation with 10% PEG8000 was found to limit the cross-binding without negatively impacting the productivity (FIG. 25B and FIG. 25C). The results from culturing a mixture of non-producing (“Null strain”) and antibody-producing strain (“Producer strain”) in media containing 10% PEG8000 indicating limited migration of antibody from the antibody-producing cells to the null cells. A mixture of equal numbers of null and producer cells (“50:50 mix Null and Producer”) resulted in two fluorescent signal peaks on the flow cytometric profile (FIG. 25B), while a mixture of 90% null cells with 10% producer cells (“90_10 mix w-PEG”) yielded fluorescence signal distribution including a low peak or shoulder of cells showing similar fluorescence intensity to the peak fluorescence intensity obtained from the producer strain (FIG. 25C). These results show that inclusion of 10% PEG 8000 decreased the amount of migration of antibody between cells, such that the level of signal on a given cell more closely reflects the level of antibody production by that individual cell.

Enrichment of High-Producing Strains

The flow cytometry-enabled cell-surface capture matrix approach described above was used to enrich highly productive cells in mixed culture in two proof-of-concept experiments. In these experiments, cells producing different humanized IgG1 antibodies (two antibodies from among Ab-A, Ab-D, Ab-E, and Ab-F) were mixed in defined ratios, and the described methods were used to capture, stain, and enrich for the higher-producing strain. The higher-producing strains were enriched by between about 20-fold and about 150-fold in the experiments. The results indicate that successful enrichment of higher-producing strains could be carried out in the context of a screening assay to isolate higher-producing cells.

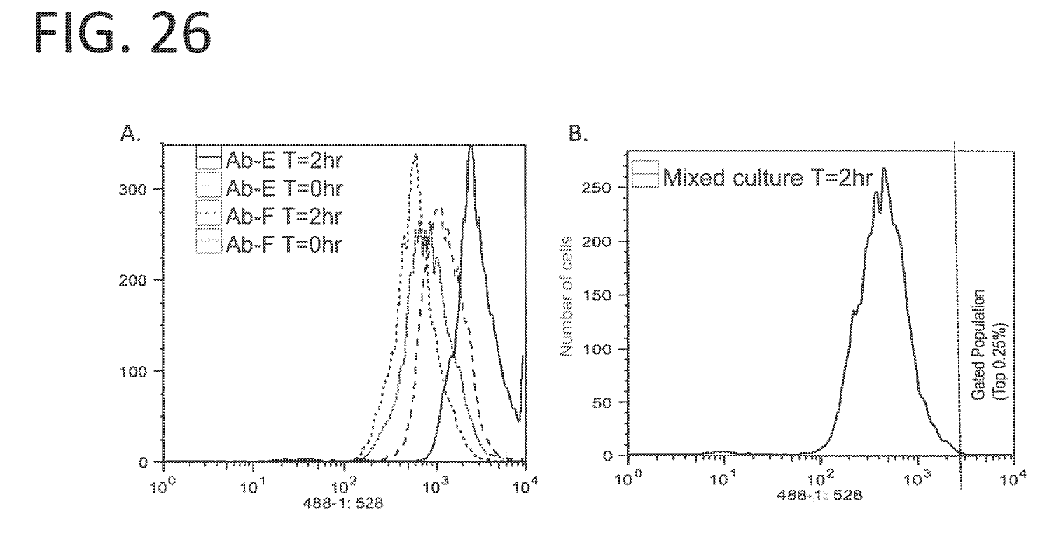

In a first experiment, a 99:1 mixed strain culture was prepared by adding “high-producing” Ab-E-secreting yeast cells to about 99 times the number of “low-producing” Ab-F secreting cells. Ab-E secreting cells had previously been observed to secrete a much higher-level of antibody than the Ab-F secreting cells. To confirm that this difference in production was detectable in the capture and sorting assay used in this example, antibody production by the individual strains was characterized by processing the cells after 0 or 2 hours in culture (FIG. 26A). The results demonstrated that Ab-E producing cells showed higher fluorescence intensity at each time-point, confirming that the higher production by Ab-E was detectable in this assay. The mixed culture was labeled with the surface-capture matrix, allowed to secrete the antibodies in 10% PEG8000-supplemented media, washed and stained with detection antibody. Using flow cytometry, the top 0.25% of the cells with the highest fluorescence signal were isolated from the population (FIG. 26B). The selected sub-population was then plated on YPDS plates supplemented with either: i) No antibiotics, allowing growth of both strains (total cells); ii) 350 mg/L G418, allowing growth of the Ab-F strain, and iii) 200 mg/L Zeocin, allowing growth of the Ab-E strain. The numbers of cells expressing each antibody and total cells were determined based upon counting the plated cells. Upon flow cytometry, the proportion of Ab-E-secreting cells was found to increase from <1% to ˜20% as a proportion of the total cells (Table 4), representing a 20-fold enrichment.

TABLE 4
Enrichment of high-producing cells (Ab-E strain) by
antibody capture, detection, and cell sorting (FACS).

| | Proportion of Total colonies | |
|---|---|---|
| | | After FACS sorting |
| | Before FACS sorting | (Top 0.25% cells) |
| Low-Producing Strain | >99% | ~80% |
| (Ab-F producing) Colonies | | |
| (G418-resistant) | | |
| High-Producing Strain | <1% | ~20% |
| (Ab-E producing) Colonies | | |
| (Zeocin-resistant) | | |

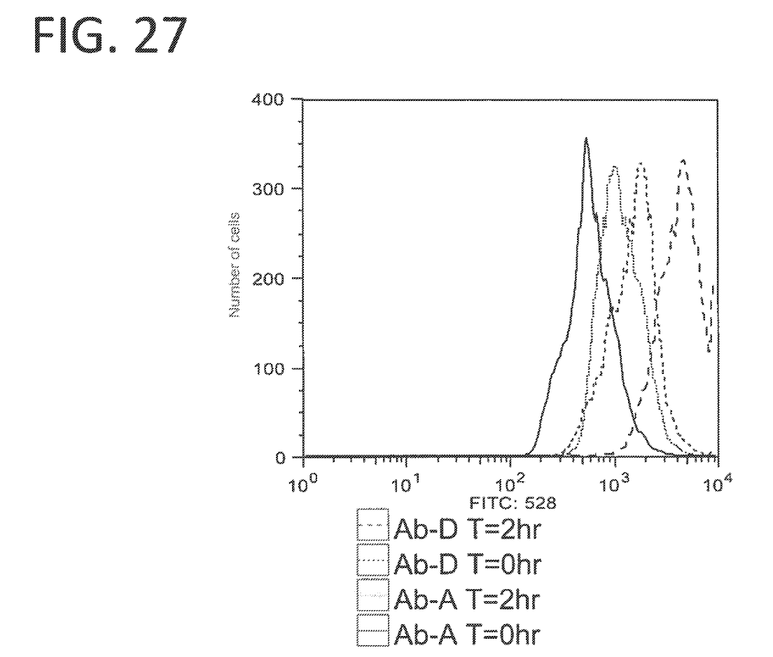

In second experiment, a 99.9:0.1 ratio mixed culture was prepared by adding high-producing Ab-D secreted cells to a culture containing about 999 times the number of low-productivity Ab-A secreting cells. Ab-D secreting cells had previously been observed to secrete a much higher-level of antibody than the Ab-A secreting cells. To confirm that this difference in production was detectable in the capture and sorting assay used in this example, antibody production by the individual strains was characterized by processing the cells after 0 or 2 hours in culture (FIG. 27). The results demonstrated that Ab-D producing cells showed higher fluorescence intensity at each time-point, confirming that the higher production by Ab-D was detectable in this assay. The mixed-culture was similarly labeled with the surface-capture matrix, allowed to secrete the antibodies in 10% PEG8000-supplemented media, followed by washing and staining. However, the flow cytometric selection criterion was made more stringent by selecting only the top 0.025% of the cells with the highest fluorescent signal. The hypothesis was that the stringent selection criterion would provide for greater enrichment. The selected sub-population was then plated on YPDS plates supplemented with either: i) No antibiotics, allowing growth of both strains (Total cells); ii) 350 mg/L G418, allowing growth of the Ab-A strain; or iii) 200 mg/L Zeocin, allowing growth of the Ab-D strain. The numbers of cells expressing each antibody and total cells were determined based upon counting the plated cells. Upon flow cytometric sorting, the proportion of Ab-D-secreting cells was found to increase from <0.1% to ˜15% as a proportion of the total cells, representing about a 150-fold enrichment (Table 5). This result confirmed that even more stringent gating criteria could result in an even greater fold-enrichment, and could be effective even when the higher-producing strain was present as less than 0.1% of the starting cell population.

TABLE 5
Enrichment of high-producing cells (Ab-D strain) by
antibody capture, detection, and cell sorting (FACS).

| | Proportion of Total colonies | |
|---|---|---|
| | | After FACS sorting |
| | Before FACS sorting | (Top 0.025% cells) |
| Low-Producing Strain | >99.9% | ~85% |
| (Ab-A Producing) Colonies | | |
| (G418-resistant) | | |
| High-Producing Strain | <0.1% | ~15% |
| (Ab-D producing) Colonies | | |
| (Zeocin-resistant) | | |

### CONCLUSION

The results indicate that successful enrichment of higher-producing strains can be effectuated using an anti-glycoprotein antibody such as Ab-1 in an antibody capture strategy followed by antibody detection and cell sorting. In these proof-of-concept experiments, known high-producing and low-producing strains were mixed, so that enrichment could be readily quantified by detecting the enrichment of the starting strains (which were differentiated by antibiotic resistance markers). In one experiment, the high-producing strain was enriched from less than 1% of the starting population to about 20% of the final population after sorting, indicating about a 20-fold enrichment. In another experiment, the high-producing strain was enriched from less than 0.1% of the starting population to about 15% of the final population after sorting, indicating about a 150-fold enrichment. From these results it is predicted that these methods can be effectuated in the context of a screening assay to isolate higher-producing cells. Genetic variation may be introduced into the population, such as by chemical mutagenesis, transformation with an expression library, systematic or random-gene knock-out. Cells producing an elevated expression level may be recovered and further processed. High-producing cells may be grown from single colonies in order to produce a genetically homogenous population, or mixed populations of enriched cells may be used. Increased expression levels can be confirmed by directly measuring the level of expression from the resulting cells as compared to starting cells or other known standards. Genetic differences from the starting cells may be determined, and may be introduced into a production strain in order to produce cells having defined genetic differences that result in the increased expression.

The subject methods may also be used to measure the effects of different treatments on production levels. For example, cells may be subjected to differences in chemical treatment, growth conditions, or other conditions to be tested for potential influence on production levels, differentially labeled, mixed, and then subjected to the capture and sorting methods above. Relative proportions of the differentially labeled cells indicate the effects of the treatment or treatments on antibody production.

### Example 11

### Use of an Anti-Glycoprotein Antibody to Reduce Glycovariant Levels

To assess the possibility of using an AGV antibody such as Ab-1 to reduce or eliminate glycovariant levels using the antibody in a chromatographic step, Ab-1 was immobilized onto chromatographic resins using two different methods. These affinity resins were then used to assess reduction of glycovariant levels in a lot of Ab-A (lot 9).

Methods

GE NHS-activated Sepharose® 4 Fast Flow resin

The pre-activated resin was prepared following manufacturers guidelines using cold 1 mM HCl to activate resin and covalently functionalized with Ab-1 by incubation with gentle agitation at room temp for up to 5 hours. Coupling reactions were terminated by addition of Tris pH8 at a final concentration of 0.1M. The functionalized resin was rinsed with alternating washes of 0.1 M Tris at pH 8 and 0.1M Arginine at pH 4 to remove any uncoupled protein. The amount of Ab-1 used for coupling ranged from 0.7 to 25 milligram of antibody per milliliter of settled resin.

Pierce Streptavidin Plus Ultralink resin

The resin (made up of beaded polyacrylamide) was prepared with a procedure similar to the manufacturers guidelines. The resin was rinsed with DPBS (without calcium or magnesium). Ab-1 was biotinylated using Pierce sulfo-NHS biotin at 20:1 or 40:1 molar ratios of biotin:antibody and the buffer was exchanged to remove free biotin per the manufacturer's recommendations. Biotinylated Ab-1 was incubated with streptavidin resin using agitation at room temp for approximately 1 hr or at 4 degrees C. overnight or a combination of room temperature and 4 degree C. incubation. The amount of Ab-1 used for coupling was approximately 1 milligram per milliliter of settled resin.

Glycovariant levels in the samples were measured using the AGV HTRF assay, as described in Example 6, above.

Results

In the first experiment, 20 milligrams of Ab-A lot 9 (eluate from a ceramic hydroxyapatite column) was diluted to 0.5 mg/ml with DPBS and applied to a DPBS equilibrated column packed with 2 ml of the Ab-1 functionalized NHS resin at a flow rate of 0.3 ml/minute. In the second experiment, 20 milligrams of Ab-A lot 9 eluate from a ceramic hydroxyapatite column was diluted to 0.5 mg/ml with DPBS and applied to a DPBS equilibrated column packed with 2.2 ml of the Ab-1 functionalized Ultralink resin at a flow rate of 0.3 ml/minute. Both of these resins were used in flow-through mode. The flow-through fractions were pooled and glycovariant signal relative to that of the load material (Ab-A lot 9) determined using the AGV HTRF assay.

As shown in Table 6, in both experiments there was a significant reduction of glycovariant signal after passing Ab-A lot 9 material through a column containing immobilized Ab-1. These results demonstrate the feasibility of using Ab-1 in a chromatographic step to reduce the levels of glycovariant in a preparation of antibody produced in Pichia. Although in these experiments an intact form of the Ab-1 antibody was used, this approach could also be taken using a Fab or scFv form of Ab-1 instead of intact antibody. This approach could also be taken using a different AGV antibody.

TABLE 6
Depletion of glycovariant using Ab1 binding.

| | Glycovariant | Glycovariant |
|---|---|---|
| | signal in | signal in Flow- |
| Resin | Load (POC) | Through (POC) |
| Ab-1 functionalized GE | 9.3 | 6.3 |
| NHS-activated Sepharose ® | | |
| 4 Fast Flow Resin | | |
| Ab-1 functionalized Pierce | 9.3 | 1.2 |
| Streptavidin Plus Ultralink | | |
| Resin | | |

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

The invention may be practiced in ways other than those particularly described in the foregoing description and examples. Numerous modifications and variations of the invention are possible in light of the above teachings and, therefore, are within the scope of the appended claims.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

Certain teachings related to methods for obtaining a clonal population of antigen-specific B cells were disclosed in U.S. Provisional patent application No. 60/801,412, filed May 19, 2006, and U.S. Patent Application Pub. No. 2012/0141982, the disclosure of each of which is herein incorporated by reference in its entirety.

Certain teachings related to humanization of rabbit-derived monoclonal antibodies and preferred sequence modifications to maintain antigen binding affinity were disclosed in International Application No. PCT/US2008/064421, corresponding to International Publication No. WO/2008/144757, entitled “Novel Rabbit Antibody Humanization Methods and Humanized Rabbit Antibodies”, filed May 21, 2008, the disclosure of which is herein incorporated by reference in its entirety.

Certain teachings related to producing antibodies or fragments thereof using mating competent yeast and corresponding methods were disclosed in U.S. patent application Ser. No. 11/429,053, filed May 8, 2006, (U.S. Patent Application Publication No. US2006/0270045), the disclosure of which is herein incorporated by reference in its entirety.

The entire disclosure of each document cited herein (including patents, patent applications, journal articles, abstracts, manuals, books, or other disclosures), including each document cited in the Background, Summary, Detailed Description, and Examples, is hereby incorporated by reference herein in its entirety.

1. An anti-glycoprotein antibody or antibody fragment which specifically binds to the same or overlapping linear or conformational epitope(s) on a glycoprotein and/or competes for binding to the same or overlapping linear or conformational epitope(s) on a glycoprotein as an anti-glycoprotein antibody selected from Ab1, Ab2, Ab3, Ab4, or Ab5.

2. The anti-glycoprotein antibody or antibody fragment of claim 1, wherein:
(a) said antibody or antibody fragment specifically binds to the same or overlapping linear or conformational epitope(s) and/or competes for binding to the same or overlapping linear or conformational epitope(s) on a glycoprotein as the anti-glycoprotein antibody Ab1;
(b) said antibody fragment is selected from an Fab fragment, an Fab′ fragment, an F(ab′)2 fragment, a monovalent antibody, or a metMab;
(c) said antibody fragment is a Fab fragment;
(d) said antibody or antibody fragment comprises the same complementarity determining regions (CDRs) as an anti-glycoprotein antibody selected from Ab1, Ab2, Ab3, Ab4, or Ab5;
(e) said antibody or antibody fragment comprises a Fab fragment of comprising a variable heavy (VH) chain comprising the CDR 1 sequence of SEQ ID NO:4, the CDR 2 sequence of SEQ ID NO:6, and the CDR 3 sequence of SEQ ID NO:8, and/or a variable light (VL) chain comprising the CDR 1 sequence of SEQ ID NO:24, the CDR 2 sequence of SEQ ID NO:26, and the CDR 3 sequence of SEQ ID NO:28;
(f) said antibody or antibody fragment comprises at least 2 CDRs in each of the VL and the VH regions which are identical to those contained in an anti-glycoprotein antibody selected from Ab1, Ab2, Ab3, Ab4, or Ab5;
(g) said antibody or antibody fragment comprises a humanized, single chain, or chimeric antibody;
(h) said antibody or antibody fragment is a rabbit antibody or antibody fragment;
(i) said antibody or antibody fragment is bound to a support;
(j) said antibody or antibody fragment comprises one or more amino acid sequence modifications relative to an antibody or antibody fragment isolated from a host animal; and/or
(k) said antibody or antibody fragment is directly or indirectly attached to a detectable label or therapeutic agent.

3. An isolated anti-glycoprotein antibody or antibody fragment according to claim 1 comprising:
(a) a VH polypeptide sequence selected from: SEQ ID NO: 2, 42, 82, 122, or 162, or a variant thereof that exhibits at least 90% sequence identity therewith; and/or a VL polypeptide sequence selected from: SEQ ID NO: 22, 62, 102, 142, or 182, or a variant thereof that exhibits at least 90% sequence identity therewith, wherein said anti-glycoprotein antibody specifically binds one or more glycoproteins; or
(b) a VH polypeptide sequence selected from: SEQ ID NO: 2, 42, 82, 122, or 162, or a variant thereof that exhibits at least 90% sequence identity therewith; and/or a VL polypeptide sequence selected from: SEQ ID NO: 22, 62, 102, 142, or 182, or a variant thereof that exhibits at least 90% sequence identity therewith, wherein one or more of the framework (FR) or CDR residues in said VH or VL polypeptide has been substituted with another amino acid residue resulting in an anti-glycoprotein antibody that specifically binds one or more glycoproteins.

4. The isolated anti-glycoprotein antibody or antibody fragment of claim 1, wherein one or more framework (FR) residues of said antibody or antibody fragment are substituted with an amino acid present at the corresponding site in a parent rabbit anti-glycoprotein antibody from which the CDRs contained in said VH or VL polypeptides have been derived or by a conservative amino acid substitution; wherein optionally
(a) at most 1 or 2 of the residues in the CDRs of said VL polypeptide sequence are modified;
(b) at most 1 or 2 of the residues in the CDRs of said VH polypeptide sequence are modified;
(c) said antibody is humanized;
(d) said antibody is chimeric;
(e) said antibody comprises a single chain antibody;
(f) said antibody comprises a human Fc; and/or
(g) said antibody comprises one or more framework and/or constant domain sequences derived from a human IgG1, IgG2, IgG3, or IgG4.

5. The isolated anti-glycoprotein antibody or antibody fragment of claim 1, wherein said antibody specifically binds to one or more glycoproteins, wherein optionally said antibody specifically binds to one or more mannosylated proteins or specifically binds to a mannosylated antibody heavy-chain or light chain.

6. The isolated anti-glycoprotein antibody or antibody fragment of claim 1, wherein said antibody specifically binds to a mannosylated human IgG1 antibody or antibody fragment comprising a heavy chain constant polypeptide having the sequence of SEQ ID NO: 201, 205, or 209 or a mannosylated fragment thereof and/or a mannosylated human IgG1 antibody light chain constant polypeptide comprising the sequence of SEQ ID NO: 203, 207, or 211 or a mannosylated fragment thereof.

7. The isolated anti-glycoprotein antibody or antibody fragment of claim 1, wherein said antibody specifically binds to one or more mannosylated antibodies or antibody fragments produced in:
(a) a yeast species;
(b) a yeast species selected from the selected from the group consisting of Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia hpolytica, and Pichia pastoris (Komagataella pastoris);
(c) a filamentous fungus species;
(d) a filamentous fungus species selected from the group consisting of: Trichoderma reesei, Aspergillus spp., Aspergillus niger, Aspergillus nidulans, Aspergillus awamori, Aspergillus oryzae, Neurospora crassa, Penicillium spp., Penicillium chrysogenum, Penicillium purpurogenum, Penicillium funiculosum, Penicillium emersonii, Rhizopus spp., Rhizopus miehei, Rhizopus oryzae, Rhizopus pusillus, Rhizopus arrhizus, Phanerochaete chrysosporium, and Fusarium graminearum; or
(e) Pichia pastoris.

8. A nucleic acid sequence or nucleic acid sequences which encode an anti-glycoprotein antibody or antibody fragment according to claim 1, or a vector comprising said nucleic acid sequence or sequences, which optionally is a plasmid or recombinant viral vector.

9. A cultured or recombinant cell which expresses an antibody or antibody fragment according to claim 1, wherein optionally said cell:
(a) is selected from a mammalian, yeast, bacterial, fungal, or insect cell;
(b) is a yeast cell;
(c) is a diploid yeast cell;
(d) is a yeast cell of the genus Pichia; and/or
(e) is Pichia pastoris.

10. A method of detecting a glycoprotein in a sample, comprising: contacting said sample with an anti-glycoprotein antibody according to claim 1, and detecting the binding of said glycoprotein with said anti-glycoprotein antibody, wherein optionally said glycoprotein is a mannosylated.

11. (canceled)

12. The method of claim 10, wherein said glycoprotein:
(a) is produced in a yeast species;
(b) is produced in a yeast species selected from the selected from the group consisting of: Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia hpolytica, and Pichia pastoris (Komagataella pastoris);
(c) is produced in a filamentous fungus species;
(d) is produced in a filamentous fungus species selected from the group consisting of: Trichoderma reesei, Aspergillus spp., Aspergillus niger, Aspergillus nidulans, Aspergillus awamori, Aspergillus oryzae, Neurospora crassa, Penicillium spp., Penicillium chrysogenum, Penicillium purpurogenum, Penicillium funiculosum, Penicillium emersonii, Rhizopus spp., Rhizopus miehei, Rhizopus oryzae, Rhizopus pusillus, Rhizopus arrhizus, Phanerochaete chrysosporium, and Fusarium graminearum; or
(e) is produced in Pichia pastoris.

13. The method of claim 10, wherein said step of detecting the binding of said glycoprotein with said anti-glycoprotein antibody comprises:
(a) an ELISA assay, wherein optionally said ELISA assay utilizes horseradish peroxidase or europium detection;
(b) said anti-glycoprotein antibody is bound to a support;
(c) the detection step uses a protein-protein interaction monitoring process; and/or
(d) the detection step uses a protein-protein interaction monitoring process that uses light interferometry, dual polarization interferometry, static light scattering, dynamic light scattering, multi-angle light scattering, surface plasmon resonance, ELISA, chemiluminescent ELISA, europium ELISA, far western, or electroluminescence.

14. The method of claim 10, which is effected on multiple fractions from a purification column, wherein based on the detected level of glycoproteins, multiple fractions are pooled to:
(a) produce a purified product depleted for glycoproteins that bind to said anti-glycoprotein antibody, wherein optionally said purified product is suitable for pharmaceutical administration; or
(b) produce a purified product enriched for glycoproteins that bind to said anti-glycoprotein antibody, wherein optionally said purified product is suitable for pharmaceutical administration
wherein, optionally the purity is determined by measuring the mass of glycosylated polypeptide as a percentage of total mass the polypeptide.

15. The method of claim 14, wherein:
(a) the detected glycoprotein is the result of O-linked glycosylation;
(b) the detected glycoprotein is a glycovariant of a polypeptide;
(c) the detected glycoprotein is a hormone, growth factor, receptor, antibody, cytokine, receptor ligand, transcription factor or enzyme;
(d) the detected glycoprotein comprises an antibody or an antibody fragment, wherein, optionally the purity is determined by measuring the mass of glycosylated heavy chain polypeptide and/or glycosylated light chain polypeptide as a percentage of total mass of heavy chain polypeptide and/or light chain polypeptide;
(e) the detected glycoprotein comprises a human antibody or a humanized antibody or fragment thereof;
(f) the detected glycoprotein comprises an antibody of mouse, rat, rabbit, goat, sheep, or cow origin;
(g) the detected glycoprotein comprises an antibody of rabbit origin;
(h) the detected glycoprotein comprises a monovalent, bivalent, or multivalent antibody; and/or
(i) the detected glycoprotein comprises an antibody of that specifically binds to IL-2, IL-4, IL-6, IL-10, IL-12, IL-13, IL-17, IL-18, IFN-alpha, IFN-gamma, BAFF, CXCL13, IP-10, CBP, angiotensin, angiotensin I, angiotensin II, Nav1.7, Nav1.8, VEGF, PDGF, EPO, EGF, FSH, TSH, hCG, CGRP, NGF, TNF, HGF, BMP2, BMP7, PCSK9 or HRG.

16. The method of claim 14, wherein:
(a) samples or eluate or fractions thereof comprising less than 10% glycoprotein are pooled;
(b) samples or eluate or fractions thereof comprising less than 5% glycoprotein are pooled;
(c) samples or eluate or fractions thereof comprising less than 1% glycoprotein are pooled; and/or
(d) samples or eluate or fractions thereof comprising less than 0.5% glycoprotein are pooled.

17. The method of claim 14, wherein:
(a) samples or eluate or fractions thereof comprising greater than 90% glycoprotein are pooled;
(b) samples or eluate or fractions thereof comprising greater than 95% glycoprotein are pooled;
(c) samples or eluate or fractions thereof comprising greater than 99% glycoprotein are pooled; or
(d) samples or eluate or fractions thereof comprising greater than 99.5% glycoprotein are pooled.

18. The method of claim 14, further comprising pooling different samples or eluate or fractions thereof based on the purity of the desired polypeptide, wherein optionally:
(a) samples or eluate or fractions thereof comprising greater than 91% purity are pooled;
(b) samples or eluate or fractions thereof comprising greater than 97% purity are pooled; or
(c) samples or eluate or fractions thereof comprising greater than 99% purity are pooled.

19. The method of claim 10, wherein the desired polypeptide is purified using a chromatographic support; optionally comprising:
(a) an affinity ligand;
(b) Protein A and/or Protein G;
(c) a lectin;
(d) a mixed mode chromatographic support;
(e) a mixed mode chromatographic support selected from ceramic hydroxyapatite, ceramic fluoroapatite, crystalline hydroxyapatite, crystalline fluoroapatite, CaptoAdhere, Capto MMC, HEA Hypercel, PPA Hypercel and Toyopearl MX-Trp-650M;
(f) a mixed mode chromatographic support comprising a ceramic hydroxyapatite;
(g) a hydrophobic interaction chromatographic support;
(h) a hydrophobic interaction chromatographic support selected from Butyl Sepharose 4 FF, Butyl-S Sepharose FF, Octyl Sepharose 4 FF, Phenyl Sepharose BB, Phenyl Sepharose HP, Phenyl Sepharose 6 FF High Sub, Phenyl Sepharose 6 FF Low Sub, Source 15ETH, Source 15ISO, Source 15PHE, Capto Phenyl, Capto Butyl, Streamline Phenyl, TSK Ether 5PW (20 um and 30 um), TSK Phenyl 5PW (20 um and 30 um), Phenyl 650S, M, and C, Butyl 650S, M and C, Hexyl-650M and C, Ether-650S and M, Butyl-600M, Super Butyl-550C, Phenyl-600M, PPG-600M; YMC-Pack Octyl Columns-3, 5, 10P, 15 and 25 um with pore sizes 120, 200, 300 A, YMC-Pack Phenyl Columns-3, 5, 10P, 15 and 25 um with pore sizes 120, 200 and 300 A, YMC-Pack Butyl Columns-3, 5, 10P, 15 and 25 um with pore sizes 120, 200 and 300 A, Cellufine Butyl, Cellufine Octyl, Cellufine Phenyl; WP HI-Propyl (C3); Macroprep t-Butyl or Macroprep methyl; and High Density Phenyl—HP2 20 um; and/or
(i) a hydrophobic interaction chromatographic support comprising polypropylene glycol (PPG) 600M or Phenyl Sepharose HP.

20. The method of claim 10, further comprising analysis of one or more samples by size exclusion chromatography to monitor impurities, wherein optionally said size exclusion chromatographic support is GS3000SW.

21. A method of decreasing the concentration of a glycoprotein in a sample, comprising: (i) contacting said sample with an anti-glycoprotein antibody or antigen-binding fragment thereof, thereby allowing said antibody or fragment to bind to said glycoprotein, and (ii) separating said antibody or fragment and said glycoprotein bound thereto from the remainder of said sample, thereby decreasing the concentration of a glycoprotein in the sample, wherein optionally said sample comprises a pharmaceutical reagent suitable for in vivo administration, and/or optionally said method is effected on pooled fractions from a purification column.

22. The method of claim 21, wherein said anti-glycoprotein antibody or fragments is an anti-glycoprotein antibody or antibody fragment which specifically binds to the same or overlapping linear or conformation epitope(s) on a glycoprotein and/or competes for binding to the same or overlapping linear or conformational epitope(s) on a glycoprotein as an anti-glycoprotein antibody selected from Ab1, Ab2, Ab3, Ab4, or Ab5.

23. The method of claim 21, wherein:
(a) is produced in a yeast species;
(b) is produced in a yeast species selected from the selected from the group consisting of: Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia lipolytica, and Pichia pastoris (Komagataella pastoris);
(c) is produced in a filamentous fungus species;
(d) is produced in a filamentous fungus species selected from the group consisting of: Trichoderma reesei, Aspergillus spp., Aspergillus niger, Aspergillus nidulans, Aspergillus awamori, Aspergillus oryzae, Neurospora crassa, Penicillium spp., Penicillium chrysogenum, Penicillium purpurogenum, Penicillium funiculosum, Penicillium emersonii, Rhizopus spp., Rhizopus miehei, Rhizopus oryzae, Rhizopus pusillus, Rhizopus arrhizus, Phanerochaete chrysosporium, and Fusarium graminearum; or
(e) is produced in Pichia pastoris.

24. The method of claim 21, wherein:
(a) said anti-glycoprotein antibody is bound to a support;
(b) said anti-glycoprotein antibody is bound to a comprising a resin; or
(c) said anti-glycoprotein antibody is bound to a comprising a resin comprising agarose, cross-linked agarose, polyacrylamide, a derivative thereof, or another resin or polymer to which functional groups, peptides, or proteins can be immobilized.

25. The method of claim 21, wherein:
(a) the detected glycoprotein is the result of O-linked glycosylation;
(b) the detected glycoprotein is a glycovariant of a polypeptide;
(c) the detected glycoprotein is a hormone, growth factor, receptor, antibody, cytokine, receptor ligand, transcription factor or enzyme;
(d) the detected glycoprotein comprises an antibody or an antibody fragment, wherein, optionally the purity is determined by measuring the mass of glycosylated heavy chain polypeptide and/or glycosylated light chain polypeptide as a percentage of total mass of heavy chain polypeptide and/or light chain polypeptide;
(e) the detected glycoprotein comprises a human antibody or a humanized antibody or fragment thereof;
the detected glycoprotein comprises an antibody of mouse, rat, rabbit, goat, sheep, or cow origin;
(g) the detected glycoprotein comprises an antibody of rabbit origin;
(h) the detected glycoprotein comprises a monovalent, bivalent, or multivalent antibody; and/or
(i) the detected glycoprotein comprises an antibody of that specifically binds to IL-2, IL-4, IL-6, IL-10, IL-12, IL-13, IL-17, IL-18, IFN-alpha, IFN-gamma, BAFF, CXCL13, IP-10, CBP, angiotensin, angiotensin I, angiotensin II, Nav1.7, Nav1.8, VEGF, PDGF, EPO, EGF, FSH, TSH, hCG, CGRP, NGF, TNF, HGF, BMP2, BMP7, PCSK9 or HRG.

26. The method of claim 21, wherein:
(a) the concentration of glycoprotein in the sample is decreased to less than 10% of the total protein in the sample;
(b) the concentration of glycoprotein in the sample is decreased to less than 5% of the total protein in the sample;
(c) the concentration of glycoprotein in the sample is decreased to less than 1% of the total protein in the sample;
(d) the concentration of glycoprotein in the sample is decreased to less than 0.5% of the total protein in the sample;
(e) the concentration of glycoprotein in the sample is decreased to less than 0.10% of the total protein in the sample; or
(f) the concentration of glycoprotein in the sample is decreased to less than 0.01% of the total protein in the sample;
wherein optionally the concentration of glycoprotein in the sample is determined by measuring the mass of glycosylated polypeptide and/or as a percentage of total mass of polypeptide in the sample.

27. The method of claim 21, wherein the desired polypeptide is purified using a chromatographic support; optionally comprising:
(a) an affinity ligand;
(b) Protein A and/or Protein G;
(c) a lectin;
(d) a mixed mode chromatographic support;
(e) a mixed mode chromatographic support selected from ceramic hydroxyapatite, ceramic fluoroapatite, crystalline hydroxyapatite, crystalline fluoroapatite, CaptoAdhere, Capto MMC, HEA Hypercel, PPA Hypercel and Toyopearl MX-Trp-650M;
(f) a mixed mode chromatographic support comprising a ceramic hydroxyapatite;
(g) a hydrophobic interaction chromatographic support;
(h) a hydrophobic interaction chromatographic support selected from Butyl Sepharose 4 FF , Butyl-S Sepharose FF, Octyl Sepharose 4 FF, Phenyl Sepharose BB, Phenyl Sepharose HP, Phenyl Sepharose 6 FF High Sub, Phenyl Sepharose 6 FF Low Sub, Source 15ETH, Source 151SO, Source 15PHE, Capto Phenyl, Capto Butyl, Streamline Phenyl, TSK Ether 5PW (20 um and 30 um), TSK Phenyl 5PW (20 um and 30 um), Phenyl 650S, M, and C, Butyl 650S, M and C, Hexyl-650M and C, Ether-650S and M, Butyl-600M, Super Butyl-550C, Phenyl-600M, PPG-600M; YMC-Pack Octyl Columns-3, 5, 10P, 15 and 25 um with pore sizes 120, 200, 300 A, YMC-Pack Phenyl Columns-3, 5, 10P, 15 and 25 um with pore sizes 120, 200 and 300 A, YMC-Pack Butyl Columns-3, 5, 10P, 15 and 25 um with pore sizes 120, 200 and 300 A, Cellufine Butyl, Cellufine Octyl, Cellufine Phenyl; WP HI-Propyl (C3); Macroprep t-Butyl or Macroprep methyl; and High Density Phenyl—HP2 20 um; and/or
(i) a hydrophobic interaction chromatographic support comprising polypropylene glycol (PPG) 600M or Phenyl Sepharose HP.

28. The method of claim 21, further comprising analysis of one or more samples by size exclusion chromatography to monitor impurities, wherein optionally said size exclusion chromatographic support is GS3000SW.

29. A method of detecting the level of expression of a secreted polypeptide by a cell, comprising: (i) binding a capture reagent to said cell; (ii) culturing said cell, whereby said secreted polypeptide is expressed and secreted from said cell; (iii) contacting said cell with a detection reagent that binds to said secreted polypeptide; and (iv) detecting said detection reagent, thereby detecting the level of expression of the secreted polypeptide by said cell.

30. The method of claim 29, wherein:
(1) said capture reagent binds irreversibly to said cell;
(2) said capture reagent comprises an anti-glycoprotein antibody;
(3) said capture reagent further comprises a binding moiety that binds to said secreted polypeptide, which optionally comprises:
(a) an antibody specific for said secreted polypeptide;
(b) an anti-Fc antibody, wherein said secreted polypeptide comprises an Fc region or fragment thereof that is specifically bound by said binding moiety;
(c) biotin;
(4) said capture reagent comprises biotin and said binding moiety comprises an avidin or streptavidin, or wherein said capture reagent comprises an avidin or streptavidin and said binding moiety comprises biotin, wherein said capture reagent and said binding moiety are linked together interaction of the avidin and biotin;
(5) said cell is a yeast cell; said cell is a yeast cell of a species is selected from the selected from the group consisting of: Candida spp., Debaryomyces hansenii, Hansenula spp. (Ogataea spp.), Kluyveromyces lactis, Kluyveromyces marxianus, Lipomyces spp., Pichia stipitis (Scheffersomyces stipitis), Pichia sp. (Komagataella spp.), Saccharomyces cerevisiae, Schizosaccharomyces pombe, Saccharomycopsis spp., Schwanniomyces occidentalis, Yarrowia lipolytica, and Pichia pastoris (Komagataella pastoris); or said cell is Pichia pastoris;
(6) the secreted polypeptide is the result of O-linked glycosylation; or the secreted polypeptide is a glycovariant of a polypeptide; or the secreted polypeptide is a hormone, growth factor, receptor, antibody, cytokine, receptor ligand, transcription factor or enzyme; or the secreted polypeptide comprises an antibody or an antibody fragment, wherein, optionally the purity is determined by measuring the mass of glycosylated heavy chain polypeptide and/or glycosylated light chain polypeptide as a percentage of total mass of heavy chain polypeptide and/or light chain polypeptide; or the secreted polypeptide comprises a human antibody or a humanized antibody or fragment thereof; or the secreted polypeptide comprises an antibody of mouse, rat, rabbit, goat, sheep, or cow origin; or the secreted polypeptide comprises an antibody of rabbit origin; or the secreted polypeptide comprises a monovalent, bivalent, or multivalent antibody; and/or the secreted polypeptide comprises an antibody of that specifically binds to IL-2, IL-4, IL-6, IL-10, IL-12, IL-13, IL-17, IL-18, IFN-alpha, IFN-gamma, BAFF, CXCL13, IP-10, CBP, angiotensin, angiotensin I, angiotensin II, Nav1.7, Nav1.8, VEGF, PDGF, EPO, EGF, FSH, TSH, hCG, CGRP, NGF, TNF, HGF, BMP2, BMP7, PCSK9 or HRG;
(7) step (ii) is conducted in a medium comprising polyethylene glycol or another molecular crowding agent, wherein optionally:
(a) said polyethylene glycol is of an average molecular weight between about 1000 Da and about 100 kDa;
(b) said polyethylene glycol is of an average molecular weight between about 5000 Da and about 15 kDa;
(c) said polyethylene glycol is of an average molecular weight between about 7000 Da and about 9000 Da; or
(d) said polyethylene glycol is of an average molecular weight of about 8000;
(e) wherein in (a) to (d) optionally said polyethylene glycol is present at a concentration of between about 1% and about 20% (w/v); between about 5% and about 15% (w/v); between about 8% and about 12% (w/v); or at a concentration of about 10% (w/v);
(8) step (ii) is conducted in a medium comprising one or more of: Dextrans, Ficoll, and/or BSA;
(9) the detection reagent comprises a fluorescent moiety;
(10) in step (iv) detecting is effected by fluorescence activated cell sorting;
(11) the method is effected on a heterogeneous population of cells, and optionally the method further comprises enriching said heterogeneous population of cells for cells that express an increased level of said secreted polypeptide; wherein said heterogeneous population of cells optionally comprises cells genetically modified cells, wherein further optionally said genetically modified cells comprise cells mutagenized by chemical, radiological, or insertional mutagenesis;
or said genetically modified cells comprise a library of genetic knockout cells; or said genetically modified cells comprise cells transformed with a plasmid library; or said genetically modified cells comprise cells transformed with a cDNA library; or said genetically modified cells comprise cells transformed with a cDNA library comprising plasmids containing cDNA sequences operably linked to a high expression promoter; or said genetically modified cells comprise cells transformed with a cDNA library comprising high-copy plasmids; and/or said genetically modified cells comprise cells transformed with a plasmid library comprising genomic DNA or cDNA obtained from a yeast species, optionally Pichia pastoris.

31-43. (canceled)

44. A cell that expresses an increased level of a secreted polypeptide, or a cell comprising a genetic modification that increases the expression level of a secreted polypeptide, wherein said cell detected by the method of claim 29.

45. (canceled)


SEQUENCE LISTING (212 sequences)

SEQ ID NO: 1 - protein, 443 aa, Artificial
  Modified antibody sequence
QEQLVESGGGLVQPGASLTLTCTASGFSFSNTNYMCWVRQAPGRGLEWVGCMPVGFIASTFYATWAKGRSAISKSSSTAVTLQMTSLTVADTATYFCARESGSGWALNLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 2 - protein, 120 aa, Artificial
  Modified antibody sequence
QEQLVESGGGLVQPGASLTLTCTASGFSFSNTNYMCWVRQAPGRGLEWVGCMPVGFIASTFYATWAKGRSAISKSSSTAVTLQMTSLTVADTATYFCARESGSGWALNLWGQGTLVTVSS

SEQ ID NO: 3 - protein, 30 aa, Oryctolagus cuniculus
QEQLVESGGGLVQPGASLTLTCTASGFSFS

SEQ ID NO: 4 - protein, 6 aa, Oryctolagus cuniculus
NTNYMC

SEQ ID NO: 5 - protein, 14 aa, Oryctolagus cuniculus
WVRQAPGRGLEWVG

SEQ ID NO: 6 - protein, 18 aa, Oryctolagus cuniculus
CMPVGFIASTFYATWAKG

SEQ ID NO: 7 - protein, 31 aa, Oryctolagus cuniculus
RSAISKSSSTAVTLQMTSLTVADTATYFCAR

SEQ ID NO: 8 - protein, 10 aa, Oryctolagus cuniculus
ESGSGWALNL

SEQ ID NO: 9 - protein, 11 aa, Artificial
  Modified antibody sequence
WGQGTLVTVSS

SEQ ID NO: 10 - protein, 323 aa, Artificial
  Modified antibody sequence
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 11 - DNA, 1329 bp, Artificial
  Modified antibody sequence
caggagcagttggtggagtccgggggaggcctggtccagcctggggcatccctgacactcacctgcacagcttctggattctccttcagtaacaccaattacatgtgctgggtccgccaggctccagggaggggcctggagtgggtcggatgcatgcccgttggttttattgccagcactttctacgcgacctgggcgaaaggccgatccgccatctccaagtcctcgtcgaccgcggtgactctgcaaatgaccagtctgacagtcgcggacacggccacctatttctgtgcgagagaaagcggtagtggctgggcgcttaacttgtggggccaagggaccctggtcaccgtctcgagcgggcaacctaaggctccatcagtcttcccactggccccctgctgcggggacacaccctctagcacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 12 - DNA, 360 bp, Artificial
  Modified antibody sequence
caggagcagttggtggagtccgggggaggcctggtccagcctggggcatccctgacactcacctgcacagcttctggattctccttcagtaacaccaattacatgtgctgggtccgccaggctccagggaggggcctggagtgggtcggatgcatgcccgttggttttattgccagcactttctacgcgacctgggcgaaaggccgatccgccatctccaagtcctcgtcgaccgcggtgactctgcaaatgaccagtctgacagtcgcggacacggccacctatttctgtgcgagagaaagcggtagtggctgggcgcttaacttgtggggccaagggaccctggtcaccgtctcgagc

SEQ ID NO: 13 - DNA, 90 bp, Oryctolagus cuniculus
caggagcagttggtggagtccgggggaggcctggtccagcctggggcatccctgacactcacctgcacagcttctggattctccttcagt

SEQ ID NO: 14 - DNA, 18 bp, Oryctolagus cuniculus
aacaccaattacatgtgc

SEQ ID NO: 15 - DNA, 42 bp, Oryctolagus cuniculus
tgggtccgccaggctccagggaggggcctggagtgggtcgga

SEQ ID NO: 16 - DNA, 54 bp, Oryctolagus cuniculus
tgcatgcccgttggttttattgccagcactttctacgcgacctgggcgaaaggc

SEQ ID NO: 17 - DNA, 93 bp, Oryctolagus cuniculus
cgatccgccatctccaagtcctcgtcgaccgcggtgactctgcaaatgaccagtctgacagtcgcggacacggccacctatttctgtgcgaga

SEQ ID NO: 18 - DNA, 30 bp, Oryctolagus cuniculus
gaaagcggtagtggctgggcgcttaacttg

SEQ ID NO: 19 - DNA, 33 bp, Artificial
  Modified antibody sequence
tggggccaagggaccctggtcaccgtctcgagc

SEQ ID NO: 20 - DNA, 969 bp, Artificial
  Modified antibody sequence
gggcaacctaaggctccatcagtcttcccactggccccctgctgcggggacacaccctctagcacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 21 - protein, 213 aa, Artificial
  Modified antibody sequence
DPVLTQTPSPVSAAVGGTVTISCQASESVESGNWLAWYQQKPGQPPKLLIYYTSTLASGVPSRFKGSGSGAHFTLTISGVQCDDAATYYCQGAFYGVNTFGGGTEVVVKRTPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 22 - protein, 109 aa, Artificial
  Modified antibody sequence
DPVLTQTPSPVSAAVGGTVTISCQASESVESGNWLAWYQQKPGQPPKLLIYYTSTLASGVPSRFKGSGSGAHFTLTISGVQCDDAATYYCQGAFYGVNTFGGGTEVVVK

SEQ ID NO: 23 - protein, 23 aa, Oryctolagus cuniculus
DPVLTQTPSPVSAAVGGTVTISC

SEQ ID NO: 24 - protein, 13 aa, Oryctolagus cuniculus
QASESVESGNWLA

SEQ ID NO: 25 - protein, 15 aa, Oryctolagus cuniculus
WYQQKPGQPPKLLIY

SEQ ID NO: 26 - protein, 7 aa, Oryctolagus cuniculus
YTSTLAS

SEQ ID NO: 27 - protein, 32 aa, Oryctolagus cuniculus
GVPSRFKGSGSGAHFTLTISGVQCDDAATYYC

SEQ ID NO: 28 - protein, 9 aa, Oryctolagus cuniculus
QGAFYGVNT

SEQ ID NO: 29 - protein, 10 aa, Artificial
  Modified antibody sequence
FGGGTEVVVK

SEQ ID NO: 30 - protein, 104 aa, Artificial
  Modified antibody sequence
RTPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - DNA, 639 bp, Artificial
  Modified antibody sequence
gaccctgtgctgacccagactccatcccccgtgtctgcagctgtgggaggcacagtcaccatcagttgccaggccagtgagagtgttgagagtggcaactggttagcctggtatcagcagaaaccagggcagcctcccaagctcctgatctattatacatccactctggcatctggggtcccatcgcggttcaaaggcagtggatctggggcacacttcactctcaccatcagcggcgtgcagtgtgacgatgctgccacttactactgtcaaggcgctttttatggtgtgaatactttcggcggagggaccgaggtggtggtcaaacgtacgccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 32 - DNA, 327 bp, Artificial
  Modified antibody sequence
gaccctgtgctgacccagactccatcccccgtgtctgcagctgtgggaggcacagtcaccatcagttgccaggccagtgagagtgttgagagtggcaactggttagcctggtatcagcagaaaccagggcagcctcccaagctcctgatctattatacatccactctggcatctggggtcccatcgcggttcaaaggcagtggatctggggcacacttcactctcaccatcagcggcgtgcagtgtgacgatgctgccacttactactgtcaaggcgctttttatggtgtgaatactttcggcggagggaccgaggtggtggtcaaa

SEQ ID NO: 33 - DNA, 69 bp, Oryctolagus cuniculus
gaccctgtgctgacccagactccatcccccgtgtctgcagctgtgggaggcacagtcaccatcagttgc

SEQ ID NO: 34 - DNA, 39 bp, Oryctolagus cuniculus
caggccagtgagagtgttgagagtggcaactggttagcc

SEQ ID NO: 35 - DNA, 45 bp, Oryctolagus cuniculus
tggtatcagcagaaaccagggcagcctcccaagctcctgatctat

SEQ ID NO: 36 - DNA, 21 bp, Oryctolagus cuniculus
tatacatccactctggcatct

SEQ ID NO: 37 - DNA, 96 bp, Oryctolagus cuniculus
ggggtcccatcgcggttcaaaggcagtggatctggggcacacttcactctcaccatcagcggcgtgcagtgtgacgatgctgccacttactactgt

SEQ ID NO: 38 - DNA, 27 bp, Oryctolagus cuniculus
caaggcgctttttatggtgtgaatact

SEQ ID NO: 39 - DNA, 30 bp, Artificial
  Modified antibody sequence
ttcggcggagggaccgaggtggtggtcaaa

SEQ ID NO: 40 - DNA, 312 bp, Artificial
  Modified antibody sequence
cgtacgccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 41 - protein, 442 aa, Artificial
  Modified antibody sequence
QSLEESGGGLVKPEGSLTLTCKASGFSFTGAHYMCWVRQAPGKGLEWIACIYGGSVDITFYASWAKGRFAISKSSSTAVTLQMTSLTAADTATYVCARESGSGWALNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 42 - protein, 119 aa, Artificial
  Modified antibody sequence
QSLEESGGGLVKPEGSLTLTCKASGFSFTGAHYMCWVRQAPGKGLEWIACIYGGSVDITFYASWAKGRFAISKSSSTAVTLQMTSLTAADTATYVCARESGSGWALNLWGPGTLVTVSS

SEQ ID NO: 43 - protein, 29 aa, Oryctolagus cuniculus
QSLEESGGGLVKPEGSLTLTCKASGFSFT

SEQ ID NO: 44 - protein, 6 aa, Oryctolagus cuniculus
GAHYMC

SEQ ID NO: 45 - protein, 14 aa, Oryctolagus cuniculus
WVRQAPGKGLEWIA

SEQ ID NO: 46 - protein, 18 aa, Oryctolagus cuniculus
CIYGGSVDITFYASWAKG

SEQ ID NO: 47 - protein, 31 aa, Oryctolagus cuniculus
RFAISKSSSTAVTLQMTSLTAADTATYVCAR

SEQ ID NO: 48 - protein, 10 aa, Oryctolagus cuniculus
ESGSGWALNL

SEQ ID NO: 49 - protein, 11 aa, Artificial
  Modified antibody sequence
WGPGTLVTVSS

SEQ ID NO: 50 - protein, 323 aa, Artificial; identical to SEQ ID NO: 10
  Modified antibody sequence

SEQ ID NO: 51 - DNA, 1326 bp, Artificial
  Modified antibody sequence
cagtcgttggaggagtccgggggaggcctggtcaagcctgagggatccctgacactcacctgcaaagcctctggattctccttcactggcgcccactacatgtgctgggtccgccaggctccagggaaggggctggagtggatcgcatgtatttatggtggtagtgttgatataactttctacgcgagctgggcgaaaggccgattcgccatctccaagtcctcgtcgaccgcggtgactctgcaaatgaccagtctgacagccgcggacacggccacctatgtctgtgcgagagaaagcggtagtggctgggcgcttaacttgtggggcccggggaccctagtcaccgtctcgagcgggcaacctaaggctccatcagtcttcccactggccccctgctgcggggacacaccctctagcacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 52 - DNA, 357 bp, Artificial
  Modified antibody sequence
cagtcgttggaggagtccgggggaggcctggtcaagcctgagggatccctgacactcacctgcaaagcctctggattctccttcactggcgcccactacatgtgctgggtccgccaggctccagggaaggggctggagtggatcgcatgtatttatggtggtagtgttgatataactttctacgcgagctgggcgaaaggccgattcgccatctccaagtcctcgtcgaccgcggtgactctgcaaatgaccagtctgacagccgcggacacggccacctatgtctgtgcgagagaaagcggtagtggctgggcgcttaacttgtggggcccggggaccctagtcaccgtctcgagc

SEQ ID NO: 53 - DNA, 87 bp, Oryctolagus cuniculus
cagtcgttggaggagtccgggggaggcctggtcaagcctgagggatccctgacactcacctgcaaagcctctggattctccttcact

SEQ ID NO: 54 - DNA, 18 bp, Oryctolagus cuniculus
ggcgcccactacatgtgc

SEQ ID NO: 55 - DNA, 42 bp, Oryctolagus cuniculus
tgggtccgccaggctccagggaaggggctggagtggatcgca

SEQ ID NO: 56 - DNA, 54 bp, Oryctolagus cuniculus
tgtatttatggtggtagtgttgatataactttctacgcgagctgggcgaaaggc

SEQ ID NO: 57 - DNA, 93 bp, Oryctolagus cuniculus
cgattcgccatctccaagtcctcgtcgaccgcggtgactctgcaaatgaccagtctgacagccgcggacacggccacctatgtctgtgcgaga

SEQ ID NO: 58 - DNA, 30 bp, Oryctolagus cuniculus; identical to SEQ ID NO: 18

SEQ ID NO: 59 - DNA, 33 bp, Artificial
  Modified antibody sequence
tggggcccggggaccctagtcaccgtctcgagc

SEQ ID NO: 60 - DNA, 969 bp, Artificial; identical to SEQ ID NO: 20
  Modified antibody sequence

SEQ ID NO: 61 - protein, 212 aa, Artificial
  Modified antibody sequence
QVLTQTASPVSAAVGGTVTISCQSSQSVENGNWLAWYQQKPGQPPKLLIYLASTLESGVPSRFKGSGSGTQFTLTISGVQCDDAATYYCQGAYSGINVFGGGTEVVVKRTPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 62 - protein, 108 aa, Artificial
  Modified antibody sequence
QVLTQTASPVSAAVGGTVTISCQSSQSVENGNWLAWYQQKPGQPPKLLIYLASTLESGVPSRFKGSGSGTQFTLTISGVQCDDAATYYCQGAYSGINVFGGGTEVVVK

SEQ ID NO: 63 - protein, 22 aa, Oryctolagus cuniculus
QVLTQTASPVSAAVGGTVTISC

SEQ ID NO: 64 - protein, 13 aa, Oryctolagus cuniculus
QSSQSVENGNWLA

SEQ ID NO: 65 - protein, 15 aa, Oryctolagus cuniculus
WYQQKPGQPPKLLIY

SEQ ID NO: 66 - protein, 7 aa, Oryctolagus cuniculus
LASTLES

SEQ ID NO: 67 - protein, 32 aa, Oryctolagus cuniculus
GVPSRFKGSGSGTQFTLTISGVQCDDAATYYC

SEQ ID NO: 68 - protein, 9 aa, Oryctolagus cuniculus
QGAYSGINV

SEQ ID NO: 69 - protein, 10 aa, Artificial
  Modified antibody sequence
FGGGTEVVVK

SEQ ID NO: 70 - protein, 104 aa, Artificial; identical to SEQ ID NO: 30
  Modified antibody sequence

SEQ ID NO: 71 - DNA, 636 bp, Artificial
  Modified antibody sequence
caagtgctgacccagactgcatcgcccgtgtctgccgctgtgggaggcacagtcaccatcagttgccagtccagtcagagtgttgagaatggcaactggttagcctggtatcagcagaaaccagggcagcctcccaagctcctgatctatctggcatccactctggaatctggggtcccatcgcggttcaaaggcagtggatctgggacacagttcactctcaccatcagcggcgtacagtgtgacgatgctgccacttactactgtcagggcgcttatagtggtattaatgttttcggcggagggaccgaggtggtggtcaaacgtacgccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 72 - DNA, 324 bp, Artificial
  Modified antibody sequence
caagtgctgacccagactgcatcgcccgtgtctgccgctgtgggaggcacagtcaccatcagttgccagtccagtcagagtgttgagaatggcaactggttagcctggtatcagcagaaaccagggcagcctcccaagctcctgatctatctggcatccactctggaatctggggtcccatcgcggttcaaaggcagtggatctgggacacagttcactctcaccatcagcggcgtacagtgtgacgatgctgccacttactactgtcagggcgcttatagtggtattaatgttttcggcggagggaccgaggtggtggtcaaa

SEQ ID NO: 73 - DNA, 66 bp, Oryctolagus cuniculus
caagtgctgacccagactgcatcgcccgtgtctgccgctgtgggaggcacagtcaccatcagttgc

SEQ ID NO: 74 - DNA, 39 bp, Oryctolagus cuniculus
cagtccagtcagagtgttgagaatggcaactggttagcc

SEQ ID NO: 75 - DNA, 45 bp, Oryctolagus cuniculus; identical to SEQ ID NO: 35

SEQ ID NO: 76 - DNA, 21 bp, Oryctolagus cuniculus
ctggcatccactctggaatct

SEQ ID NO: 77 - DNA, 96 bp, Oryctolagus cuniculus
ggggtcccatcgcggttcaaaggcagtggatctgggacacagttcactctcaccatcagcggcgtacagtgtgacgatgctgccacttactactgt

SEQ ID NO: 78 - DNA, 27 bp, Oryctolagus cuniculus
cagggcgcttatagtggtattaatgtt

SEQ ID NO: 79 - DNA, 30 bp, Artificial; identical to SEQ ID NO: 39
  Modified antibody sequence

SEQ ID NO: 80 - DNA, 312 bp, Artificial; identical to SEQ ID NO: 40
  Modified antibody sequence

SEQ ID NO: 81 - protein, 442 aa, Artificial
  Modified antibody sequence
QSLEESGGGLVQPEGSLTLTCTASGFFFSGAHYMCWVRQAPGQGLEWIGCTYGGSVDITFYASWAKGRFAISKTSSTTVTLQLTSLTAADTATYVCARESGSGWALNLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 82 - protein, 119 aa, Artificial
  Modified antibody sequence
QSLEESGGGLVQPEGSLTLTCTASGFFFSGAHYMCWVRQAPGQGLEWIGCTYGGSVDITFYASWAKGRFAISKTSSTTVTLQLTSLTAADTATYVCARESGSGWALNLWGQGTLVTVSS

SEQ ID NO: 83 - protein, 29 aa, Oryctolagus cuniculus
QSLEESGGGLVQPEGSLTLTCTASGFFFS

SEQ ID NO: 84 - protein, 6 aa, Oryctolagus cuniculus
GAHYMC

SEQ ID NO: 85 - protein, 14 aa, Oryctolagus cuniculus
WVRQAPGQGLEWIG

SEQ ID NO: 86 - protein, 18 aa, Oryctolagus cuniculus
CTYGGSVDITFYASWAKG

SEQ ID NO: 87 - protein, 31 aa, Oryctolagus cuniculus
RFAISKTSSTTVTLQLTSLTAADTATYVCAR

SEQ ID NO: 88 - protein, 10 aa, Oryctolagus cuniculus
ESGSGWALNL

SEQ ID NO: 89 - protein, 11 aa, Artificial
  Modified antibody sequence
WGQGTLVTVSS

SEQ ID NO: 90 - protein, 323 aa, Artificial; identical to SEQ ID NO: 10
  Modified antibody sequence

SEQ ID NO: 91 - DNA, 1326 bp, Artificial
  Modified antibody sequence
cagtcgttggaggagtccgggggaggcctggtccagcctgagggatccctgacactcacctgtacagcctctggattcttcttcagtggcgcccactacatgtgctgggtccgccaggctccagggcaggggctggagtggatcggatgcacttatggtggtagtgttgatatcactttctacgcgagctgggcgaaaggccgattcgccatctccaaaacctcgtcgaccacggtgactctgcaactgaccagtctgacagccgcggacacggccacctatgtctgtgcgagagaaagcggtagtggctgggcacttaacttgtggggccaggggaccctcgtcaccgtctcgagcgggcaacctaaggctccatcagtcttcccactggccccctgctgcggggacacaccctctagcacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 92 - DNA, 357 bp, Artificial
  Modified antibody sequence
cagtcgttggaggagtccgggggaggcctggtccagcctgagggatccctgacactcacctgtacagcctctggattcttcttcagtggcgcccactacatgtgctgggtccgccaggctccagggcaggggctggagtggatcggatgcacttatggtggtagtgttgatatcactttctacgcgagctgggcgaaaggccgattcgccatctccaaaacctcgtcgaccacggtgactctgcaactgaccagtctgacagccgcggacacggccacctatgtctgtgcgagagaaagcggtagtggctgggcacttaacttgtggggccaggggaccctcgtcaccgtctcgagc

SEQ ID NO: 93 - DNA, 87 bp, Oryctolagus cuniculus
cagtcgttggaggagtccgggggaggcctggtccagcctgagggatccctgacactcacctgtacagcctctggattcttcttcagt

SEQ ID NO: 94 - DNA, 18 bp, Oryctolagus cuniculus
ggcgcccactacatgtgc

SEQ ID NO: 95 - DNA, 42 bp, Oryctolagus cuniculus
tgggtccgccaggctccagggcaggggctggagtggatcgga

SEQ ID NO: 96 - DNA, 54 bp, Oryctolagus cuniculus
tgcacttatggtggtagtgttgatatcactttctacgcgagctgggcgaaaggc

SEQ ID NO: 97 - DNA, 93 bp, Oryctolagus cuniculus
cgattcgccatctccaaaacctcgtcgaccacggtgactctgcaactgaccagtctgacagccgcggacacggccacctatgtctgtgcgaga

SEQ ID NO: 98 - DNA, 30 bp, Oryctolagus cuniculus
gaaagcggtagtggctgggcacttaacttg

SEQ ID NO: 99 - DNA, 33 bp, Artificial
  Modified antibody sequence
tggggccaggggaccctcgtcaccgtctcgagc

SEQ ID NO: 100 - DNA, 969 bp, Artificial; identical to SEQ ID NO: 20
  Modified antibody sequence

SEQ ID NO: 101 - protein, 212 aa, Artificial
  Modified antibody sequence
QVLTQTPSPVSAAVGGAVTINCQSSQSVENGNWLGWYQQKPGQPPKLLIYLASTLASGVPSRFTGSGSGTQFTLTISGVQCDDAATYYCQGAYSGINAFGGGTEVVVKRTPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 102 - protein, 108 aa, Artificial
  Modified antibody sequence
QVLTQTPSPVSAAVGGAVTINCQSSQSVENGNWLGWYQQKPGQPPKLLIYLASTLASGVPSRFTGSGSGTQFTLTISGVQCDDAATYYCQGAYSGINAFGGGTEVVVK

SEQ ID NO: 103 - protein, 22 aa, Oryctolagus cuniculus
QVLTQTPSPVSAAVGGAVTINC

SEQ ID NO: 104 - protein, 13 aa, Oryctolagus cuniculus
QSSQSVENGNWLG

SEQ ID NO: 105 - protein, 15 aa, Oryctolagus cuniculus
WYQQKPGQPPKLLIY

SEQ ID NO: 106 - protein, 7 aa, Oryctolagus cuniculus
LASTLAS

SEQ ID NO: 107 - protein, 32 aa, Oryctolagus cuniculus
GVPSRFTGSGSGTQFTLTISGVQCDDAATYYC

SEQ ID NO: 108 - protein, 9 aa, Oryctolagus cuniculus
QGAYSGINA

SEQ ID NO: 109 - protein, 10 aa, Artificial
  Modified antibody sequence
FGGGTEVVVK

SEQ ID NO: 110 - protein, 104 aa, Artificial; identical to SEQ ID NO: 30
  Modified antibody sequence

SEQ ID NO: 111 - DNA, 636 bp, Artificial
  Modified antibody sequence
caggtgctgacccagactccatcccccgtgtctgcagctgtgggaggcgcagtcaccatcaattgccagtccagtcagagtgttgagaatggcaactggttaggctggtatcagcagaaaccagggcagcctcccaagctcctgatctatctggcatccactctggcatctggggtcccttcgcggttcaccggcagcggatctgggacacagttcactctcaccatcagcggcgtgcagtgtgacgatgctgccacttactattgtcaaggcgcttatagtggtattaatgctttcggcggagggaccgaggtggtggtcaaacgtacgccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 112 - DNA, 324 bp, Artificial
  Modified antibody sequence
caggtgctgacccagactccatcccccgtgtctgcagctgtgggaggcgcagtcaccatcaattgccagtccagtcagagtgttgagaatggcaactggttaggctggtatcagcagaaaccagggcagcctcccaagctcctgatctatctggcatccactctggcatctggggtcccttcgcggttcaccggcagcggatctgggacacagttcactctcaccatcagcggcgtgcagtgtgacgatgctgccacttactattgtcaaggcgcttatagtggtattaatgctttcggcggagggaccgaggtggtggtcaaa

SEQ ID NO: 113 - DNA, 66 bp, Oryctolagus cuniculus
caggtgctgacccagactccatcccccgtgtctgcagctgtgggaggcgcagtcaccatcaattgc

SEQ ID NO: 114 - DNA, 39 bp, Oryctolagus cuniculus
cagtccagtcagagtgttgagaatggcaactggttaggc

SEQ ID NO: 115 - DNA, 45 bp, Oryctolagus cuniculus; identical to SEQ ID NO: 35

SEQ ID NO: 116 - DNA, 21 bp, Oryctolagus cuniculus
ctggcatccactctggcatct

SEQ ID NO: 117 - DNA, 96 bp, Oryctolagus cuniculus
ggggtcccttcgcggttcaccggcagcggatctgggacacagttcactctcaccatcagcggcgtgcagtgtgacgatgctgccacttactattgt

SEQ ID NO: 118 - DNA, 27 bp, Oryctolagus cuniculus
caaggcgcttatagtggtattaatgct

SEQ ID NO: 119 - DNA, 30 bp, Artificial; identical to SEQ ID NO: 39
  Modified antibody sequence

SEQ ID NO: 120 - DNA, 312 bp, Artificial; identical to SEQ ID NO: 40
  Modified antibody sequence

SEQ ID NO: 121 - protein, 441 aa, Artificial
  Modified antibody sequence
QSLEESGGDLVKPGASLTLTCTASGFSFSSGYDMCWVRQAPGKGLEWIACIYPNNPVTYYASWAKGRFTISKTSSTTVTLQMTSLTAADTATYFCGRSDSNGHTFNLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 122 - protein, 118 aa, Artificial
  Modified antibody sequence
QSLEESGGDLVKPGASLTLTCTASGFSFSSGYDMCWVRQAPGKGLEWIACIYPNNPVTYYASWAKGRFTISKTSSTTVTLQMTSLTAADTATYFCGRSDSNGHTFNLWGQGTLVTVSS

SEQ ID NO: 123 - protein, 29 aa, Oryctolagus cuniculus
QSLEESGGDLVKPGASLTLTCTASGFSFS

SEQ ID NO: 124 - protein, 6 aa, Oryctolagus cuniculus
SGYDMC

SEQ ID NO: 125 - protein, 14 aa, Oryctolagus cuniculus
WVRQAPGKGLEWIA

SEQ ID NO: 126 - protein, 17 aa, Oryctolagus cuniculus
CIYPNNPVTYYASWAKG

SEQ ID NO: 127 - protein, 31 aa, Oryctolagus cuniculus
RFTISKTSSTTVTLQMTSLTAADTATYFCGR

SEQ ID NO: 128 - protein, 10 aa, Oryctolagus cuniculus
SDSNGHTFNL

SEQ ID NO: 129 - protein, 11 aa, Artificial
  Modified antibody sequence
WGQGTLVTVSS

SEQ ID NO: 130 - protein, 323 aa, Artificial; identical to SEQ ID NO: 10
  Modified antibody sequence

SEQ ID NO: 131 - DNA, 1323 bp, Artificial
  Modified antibody sequence
cagtcgttggaggagtccgggggagacctggtcaagcctggggcatccctgacactcacctgcacagcctctggattctccttcagtagcggctacgacatgtgttgggtccgccaggctccagggaaggggctggagtggatcgcctgtatttaccctaataatcctgtcacttactacgcgagctgggcgaaaggccgattcaccatctccaaaacctcgtcgaccacggtgactctgcaaatgaccagtctgacagccgcggacacggccacctatttctgtgggagatctgatagtaatggtcatacctttaacttgtggggccaaggcaccctcgtcaccgtctcgagcgggcaacctaaggctccatcagtcttcccactggccccctgctgcggggacacaccctctagcacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 132 - DNA, 354 bp, Artificial
  Modified antibody sequence
cagtcgttggaggagtccgggggagacctggtcaagcctggggcatccctgacactcacctgcacagcctctggattctccttcagtagcggctacgacatgtgttgggtccgccaggctccagggaaggggctggagtggatcgcctgtatttaccctaataatcctgtcacttactacgcgagctgggcgaaaggccgattcaccatctccaaaacctcgtcgaccacggtgactctgcaaatgaccagtctgacagccgcggacacggccacctatttctgtgggagatctgatagtaatggtcatacctttaacttgtggggccaaggcaccctcgtcaccgtctcgagc

SEQ ID NO: 133 - DNA, 87 bp, Oryctolagus cuniculus
cagtcgttggaggagtccgggggagacctggtcaagcctggggcatccctgacactcacctgcacagcctctggattctccttcagt

SEQ ID NO: 134 - DNA, 18 bp, Oryctolagus cuniculus
agcggctacgacatgtgt

SEQ ID NO: 135 - DNA, 42 bp, Oryctolagus cuniculus
tgggtccgccaggctccagggaaggggctggagtggatcgcc

SEQ ID NO: 136 - DNA, 51 bp, Oryctolagus cuniculus
tgtatttaccctaataatcctgtcacttactacgcgagctgggcgaaaggc

SEQ ID NO: 137 - DNA, 93 bp, Oryctolagus cuniculus
cgattcaccatctccaaaacctcgtcgaccacggtgactctgcaaatgaccagtctgacagccgcggacacggccacctatttctgtgggaga

SEQ ID NO: 138 - DNA, 30 bp, Oryctolagus cuniculus
tctgatagtaatggtcatacctttaacttg

SEQ ID NO: 139 - DNA, 33 bp, Artificial
  Modified antibody sequence
tggggccaaggcaccctcgtcaccgtctcgagc

SEQ ID NO: 140 - DNA, 969 bp, Artificial; identical to SEQ ID NO: 20
  Modified antibody sequence

SEQ ID NO: 141 - protein, 215 aa, Artificial
  Modified antibody sequence
DPVMTQTPSSVSAAVGGTVTINCQSSQSVNQNDLSWYQQKPGQPPKRLIYYASTLASGVSSRFKGSGSGTQFTLTISDMQCDDAATYYCQGSFRVSGWYWAFGGGTEVVVKRTPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 142 - protein, 111 aa, Artificial
  Modified antibody sequence
DPVMTQTPSSVSAAVGGTVTINCQSSQSVNQNDLSWYQQKPGQPPKRLIYYASTLASGVSSRFKGSGSGTQFTLTISDMQCDDAATYYCQGSFRVSGWYWAFGGGTEVVVK

SEQ ID NO: 143 - protein, 23 aa, Oryctolagus cuniculus
DPVMTQTPSSVSAAVGGTVTINC

SEQ ID NO: 144 - protein, 12 aa, Oryctolagus cuniculus
QSSQSVNQNDLS

SEQ ID NO: 145 - protein, 15 aa, Oryctolagus cuniculus
WYQQKPGQPPKRLIY

SEQ ID NO: 146 - protein, 7 aa, Oryctolagus cuniculus
YASTLAS

SEQ ID NO: 147 - protein, 32 aa, Oryctolagus cuniculus
GVSSRFKGSGSGTQFTLTISDMQCDDAATYYC

SEQ ID NO: 148 - protein, 12 aa, Oryctolagus cuniculus
QGSFRVSGWYWA

SEQ ID NO: 149 - protein, 10 aa, Artificial
  Modified antibody sequence
FGGGTEVVVK

SEQ ID NO: 150 - protein, 104 aa, Artificial; identical to SEQ ID NO: 30
  Modified antibody sequence

SEQ ID NO: 151 - DNA, 645 bp, Artificial
  Modified antibody sequence
gaccctgtgatgacccagactccatcctccgtgtctgcagctgtgggaggcacagtcaccatcaattgccagtccagtcagagtgttaatcagaacgacttatcctggtatcagcagaaaccagggcagcctcccaagcgcctgatctattatgcatccactctggcatctggggtctcatcgcggttcaaaggcagtggatctgggacacagttcactctcaccatcagcgacatgcagtgtgacgatgctgccacttactactgtcaaggcagttttcgtgttagtggttggtactgggctttcggcggagggaccgaggtggtggtcaaacgtacgccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 152 - DNA, 333 bp, Artificial
  Modified antibody sequence
gaccctgtgatgacccagactccatcctccgtgtctgcagctgtgggaggcacagtcaccatcaattgccagtccagtcagagtgttaatcagaacgacttatcctggtatcagcagaaaccagggcagcctcccaagcgcctgatctattatgcatccactctggcatctggggtctcatcgcggttcaaaggcagtggatctgggacacagttcactctcaccatcagcgacatgcagtgtgacgatgctgccacttactactgtcaaggcagttttcgtgttagtggttggtactgggctttcggcggagggaccgaggtggtggtcaaa

SEQ ID NO: 153 - DNA, 69 bp, Oryctolagus cuniculus
gaccctgtgatgacccagactccatcctccgtgtctgcagctgtgggaggcacagtcaccatcaattgc

SEQ ID NO: 154 - DNA, 36 bp, Oryctolagus cuniculus
cagtccagtcagagtgttaatcagaacgacttatcc

SEQ ID NO: 155 - DNA, 45 bp, Oryctolagus cuniculus
tggtatcagcagaaaccagggcagcctcccaagcgcctgatctat

SEQ ID NO: 156 - DNA, 21 bp, Oryctolagus cuniculus
tatgcatccactctggcatct

SEQ ID NO: 157 - DNA, 96 bp, Oryctolagus cuniculus
ggggtctcatcgcggttcaaaggcagtggatctgggacacagttcactctcaccatcagcgacatgcagtgtgacgatgctgccacttactactgt

SEQ ID NO: 158 - DNA, 36 bp, Oryctolagus cuniculus
caaggcagttttcgtgttagtggttggtactgggct

SEQ ID NO: 159 - DNA, 30 bp, Artificial; identical to SEQ ID NO: 39
  Modified antibody sequence

SEQ ID NO: 160 - DNA, 312 bp, Artificial; identical to SEQ ID NO: 40
  Modified antibody sequence

SEQ ID NO: 161 - protein, 447 aa, Artificial
  Modified antibody sequence
QQQLLESGGGLVQPEGSLALTCTASGFSFSSGYDMCWVRQPPGKGLEWVGCIYSGDDNDITYYASWARGRFTISNPSSTTVTLQMTSLTVADTATYFCARGHAIYDNYDSVHLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 162 - protein, 124 aa, Artificial
  Modified antibody sequence
QQQLLESGGGLVQPEGSLALTCTASGFSFSSGYDMCWVRQPPGKGLEWVGCIYSGDDNDITYYASWARGRFTISNPSSTTVTLQMTSLTVADTATYFCARGHAIYDNYDSVHLWGQGTLVTVSS

SEQ ID NO: 163 - protein, 30 aa, Oryctolagus cuniculus
QQQLLESGGGLVQPEGSLALTCTASGFSFS

SEQ ID NO: 164 - protein, 6 aa, Oryctolagus cuniculus
SGYDMC

SEQ ID NO: 165 - protein, 14 aa, Oryctolagus cuniculus
WVRQPPGKGLEWVG

SEQ ID NO: 166 - protein, 19 aa, Oryctolagus cuniculus
CIYSGDDNDITYYASWARG

SEQ ID NO: 167 - protein, 31 aa, Oryctolagus cuniculus
RFTISNPSSTTVTLQMTSLTVADTATYFCAR

SEQ ID NO: 168 - protein, 13 aa, Oryctolagus cuniculus
GHAIYDNYDSVHL

SEQ ID NO: 169 - protein, 11 aa, Artificial
  Modified antibody sequence
WGQGTLVTVSS

SEQ ID NO: 170 - protein, 323 aa, Artificial; identical to SEQ ID NO: 10
  Modified antibody sequence

SEQ ID NO: 171 - DNA, 1341 bp, Artificial
  Modified antibody sequence
cagcagcagttgctggagtccgggggaggcctggtccagcctgagggatccctggcactcacctgcacagcttctggattctccttcagtagcggctacgacatgtgctgggtccgccagcctccagggaaggggctggagtgggtcggctgcatttatagtggtgatgataatgatattacttattacgcgagctgggcgagaggccgattcaccatctccaacccctcgtcgaccactgtgactctgcaaatgaccagtctgacagtcgcggacacggccacctatttctgtgcgcgaggtcatgctatttatgataattatgatagtgtccacttgtggggccaggggaccctcgtcaccgtctcgagcgggcaacctaaggctccatcagtcttcccactggccccctgctgcggggacacaccctctagcacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 172 - DNA, 372 bp, Artificial
  Modified antibody sequence
cagcagcagttgctggagtccgggggaggcctggtccagcctgagggatccctggcactcacctgcacagcttctggattctccttcagtagcggctacgacatgtgctgggtccgccagcctccagggaaggggctggagtgggtcggctgcatttatagtggtgatgataatgatattacttattacgcgagctgggcgagaggccgattcaccatctccaacccctcgtcgaccactgtgactctgcaaatgaccagtctgacagtcgcggacacggccacctatttctgtgcgcgaggtcatgctatttatgataattatgatagtgtccacttgtggggccaggggaccctcgtcaccgtctcgagc

SEQ ID NO: 173 - DNA, 90 bp, Oryctolagus cuniculus
cagcagcagttgctggagtccgggggaggcctggtccagcctgagggatccctggcactcacctgcacagcttctggattctccttcagt

SEQ ID NO: 174 - DNA, 18 bp, Oryctolagus cuniculus
agcggctacgacatgtgc

SEQ ID NO: 175 - DNA, 42 bp, Oryctolagus cuniculus
tgggtccgccagcctccagggaaggggctggagtgggtcggc

SEQ ID NO: 176 - DNA, 57 bp, Oryctolagus cuniculus
tgcatttatagtggtgatgataatgatattacttattacgcgagctgggcgagaggc

SEQ ID NO: 177 - DNA, 93 bp, Oryctolagus cuniculus
cgattcaccatctccaacccctcgtcgaccactgtgactctgcaaatgaccagtctgacagtcgcggacacggccacctatttctgtgcgcga

SEQ ID NO: 178 - DNA, 39 bp, Oryctolagus cuniculus
ggtcatgctatttatgataattatgatagtgtccacttg

SEQ ID NO: 179 - DNA, 33 bp, Artificial; identical to SEQ ID NO: 99
  Modified antibody sequence

SEQ ID NO: 180 - DNA, 969 bp, Artificial; identical to SEQ ID NO: 20
  Modified antibody sequence

SEQ ID NO: 181 - protein, 215 aa, Artificial
  Modified antibody sequence
IVMTQTPSSRSVPVGGTVTINCQASEIVNRNNRLAWFQQKPGQPPKLLMYLASTPASGVPSRFRGSGSGTQFTLTISDVVCDDAATYYCTAYKSSNTDGIAFGGGTEVVVKRTPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 182 - protein, 111 aa, Artificial
  Modified antibody sequence
IVMTQTPSSRSVPVGGTVTINCQASEIVNRNNRLAWFQQKPGQPPKLLMYLASTPASGVPSRFRGSGSGTQFTLTISDVVCDDAATYYCTAYKSSNTDGIAFGGGTEVVVK

SEQ ID NO: 183 - protein, 22 aa, Oryctolagus cuniculus
IVMTQTPSSRSVPVGGTVTINC

SEQ ID NO: 184 - protein, 13 aa, Oryctolagus cuniculus
QASEIVNRNNRLA

SEQ ID NO: 185 - protein, 15 aa, Oryctolagus cuniculus
WFQQKPGQPPKLLMY

SEQ ID NO: 186 - protein, 7 aa, Oryctolagus cuniculus
LASTPAS

SEQ ID NO: 187 - protein, 32 aa, Oryctolagus cuniculus
GVPSRFRGSGSGTQFTLTISDVVCDDAATYYC

SEQ ID NO: 188 - protein, 12 aa, Oryctolagus cuniculus
TAYKSSNTDGIA

SEQ ID NO: 189 - protein, 10 aa, Artificial
  Modified antibody sequence
FGGGTEVVVK

SEQ ID NO: 190 - protein, 104 aa, Artificial; identical to SEQ ID NO: 30
  Modified antibody sequence

SEQ ID NO: 191 - DNA, 645 bp, Artificial
  Modified antibody sequence
atcgtgatgacccagactccatcttccaggtctgtccctgtgggaggcacagtcaccatcaattgccaggccagtgaaattgttaatagaaacaaccgcttagcctggtttcaacagaaaccagggcagcctcccaagctcctgatgtatctggcttccactccggcatctggggtcccatcgcggtttagaggcagtggatctgggacacagttcactctcaccatcagcgatgtggtgtgtgacgatgctgccacttattattgtacagcatataagagtagtaatactgatggtattgctttcggcggagggaccgaggtggtggtcaaacgtacgccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 192 - DNA, 333 bp, Artificial
  Modified antibody sequence
atcgtgatgacccagactccatcttccaggtctgtccctgtgggaggcacagtcaccatcaattgccaggccagtgaaattgttaatagaaacaaccgcttagcctggtttcaacagaaaccagggcagcctcccaagctcctgatgtatctggcttccactccggcatctggggtcccatcgcggtttagaggcagtggatctgggacacagttcactctcaccatcagcgatgtggtgtgtgacgatgctgccacttattattgtacagcatataagagtagtaatactgatggtattgctttcggcggagggaccgaggtggtggtcaaa

SEQ ID NO: 193 - DNA, 66 bp, Oryctolagus cuniculus
atcgtgatgacccagactccatcttccaggtctgtccctgtgggaggcacagtcaccatcaattgc

SEQ ID NO: 194 - DNA, 39 bp, Oryctolagus cuniculus
caggccagtgaaattgttaatagaaacaaccgcttagcc

SEQ ID NO: 195 - DNA, 45 bp, Oryctolagus cuniculus
tggtttcaacagaaaccagggcagcctcccaagctcctgatgtat

SEQ ID NO: 196 - DNA, 21 bp, Oryctolagus cuniculus
ctggcttccactccggcatct

SEQ ID NO: 197 - DNA, 96 bp, Oryctolagus cuniculus
ggggtcccatcgcggtttagaggcagtggatctgggacacagttcactctcaccatcagcgatgtggtgtgtgacgatgctgccacttattattgt

SEQ ID NO: 198 - DNA, 36 bp, Oryctolagus cuniculus
acagcatataagagtagtaatactgatggtattgct

SEQ ID NO: 199 - DNA, 30 bp, Artificial; identical to SEQ ID NO: 39
  Modified antibody sequence

SEQ ID NO: 200 - DNA, 312 bp, Artificial; identical to SEQ ID NO: 40
  Modified antibody sequence

SEQ ID NO: 201 - protein, 330 aa, Artificial
  Modified antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - DNA, 990 bp, Artificial
  Modified antibody sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 203 - protein, 107 aa, Artificial [sequence omitted]

SEQ ID NO: 204 - DNA, 321 bp, Artificial
  Modified antibody sequence
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 205 - protein, 330 aa, Artificial; identical to SEQ ID NO: 201
  Modified antibody sequence

SEQ ID NO: 206 - DNA, 990 bp, Artificial; identical to SEQ ID NO: 202
  Modified antibody sequence

SEQ ID NO: 207 - protein, 107 aa, Artificial [sequence omitted]

SEQ ID NO: 208 - DNA, 321 bp, Artificial; identical to SEQ ID NO: 204
  Modified antibody sequence

SEQ ID NO: 209 - protein, 330 aa, Artificial; identical to SEQ ID NO: 201
  Modified antibody sequence

SEQ ID NO: 210 - DNA, 990 bp, Artificial; identical to SEQ ID NO: 202
  Modified antibody sequence

SEQ ID NO: 211 - protein, 107 aa, Artificial [sequence omitted]

SEQ ID NO: 212 - DNA, 321 bp, Artificial; identical to SEQ ID NO: 204
  Modified antibody sequence